SEQUENCE LISTING (2 sequences)

SEQ ID NO: 1 - protein, 8 aa, Homo sapiens [sequence omitted]

SEQ ID NO: 2 - protein, 17 aa, Homo sapiens
  mat_peptide (1)..(17)
  MOD_RES (1)..(1): PYRROLIDONE CARBOXYLIC ACID
EGPWLEEEEEAYGWMDF

(19) United States
(12) Patent Application Publication
(10) Pub. No.: US 2017/0008928 A1
(43) Pub. Date: Jan. 12, 2017

(54) NOVEL PEPTIDES AND PEPTIDE DERIVATIVES AND USES THEREOF

(71) Applicant: Novo Nordisk A/S, Bagsvaerd (DK)

(72) Inventors: Thomas Kruse, Herlev (DK); Ulrich Sensfuss, Vanloese (DK); Trine Ryberg Clausen, Stenloese (DK)

(21) Appl. No.: 15/202,696

(22) Filed: Jul. 6, 2016

(30) Foreign Application Priority Data
Jul. 6, 2015 (EP) 15175449.6

(51) Int. Cl. C07K 7/06; A61K 47/10; A61K 38/10; A61K 47/18; C07K 7/08; A61K 38/08; A61K 47/24

(52) U.S. Cl. CPC C07K 7/06; A61K 38/08; A61K 47/10; A61K 47/24; A61K 47/18; C07K 7/08; A61K 38/10

(57) ABSTRACT

The invention relates to cholecystokinin (CCK) derivatives of the general formula I: P-L-X1-X2-X3-Gly-Trp-X6-DMeAsp-X8-NH₂, wherein P is Chem. 1: HOOC—(CH₂)ₓ—CO—*, wherein x is an integer in the range of 12-18; L is absent, or L comprises at least one linker element selected from Chem. 2: *—NH—CH(COOH)—(CH₂)₂—CO—*, Chem. 3: *—NH—(CH₂)₂—[O—(CH₂)₂]ₖ—O—[CH₂]ₙ—CO—* wherein k is an integer in the range of 1-11 and n is an integer in the range of 1-5, Chem. 4: *—NH—CH(CH₂OH)—CO—*, Chem. 5: *—NH—CH₂—CO—*, and/or Chem. 6: *—NH—CH[(CH₂)₄—NH₂]—CO—*; X1 is absent, Asp, DAsp, bAsp, DbAsp, Glu, or DGlu; X2 is Phe(4-sulfomethyl) or sTyr; X3 is Nle, Leu, Ala, Ile, Lys, Arg, Pro, Met, Phe, Ser, His, or Val; X6 is Nle, Ile, Gln, Met, Met(O2), Leu, Val, Pro, Hpg, or Ala; X8 is Phe, MePhe, 1Nal, Me1Nal, 2Nal, Me2Nal, Trp, or MeTrp. The invention also relates to the corresponding CCK peptides, as well as to methods for preparing the peptides and the derivatives. The invention also relates to pharmaceutical compositions and uses of the derivatives, in particular for treatment of obesity and related health problems, where the compounds may be used either alone or in combination with other weight reducing agents and optionally in addition to diet and exercise. The derivatives have a high potency on the CCK-1 receptor (CCK-1R), a high selectivity for the CCK-1R as compared to the CCK-2R, effect on food intake in vivo, very long half-lives, and are chemically very stable.

### TECHNICAL FIELD

The present invention relates to novel peptide derivatives, the corresponding novel peptides, and the use thereof, in particular for the treatment of obesity and related health care problems. The invention also relates to pharmaceutical compositions and uses in relation to these novel compounds.

The peptides and the peptide derivatives of the invention are cholecystokinin (CCK) compounds.

### CROSS-REFERENCE TO RELATED APPLICATIONS

This application claims priority under 35 U.S.C. §119 to European Patent Application 15175449.6, filed Jul. 6, 2015, the contents of which are incorporated herein by reference.

### SEQUENCE LISTING

The instant application contains a Sequence Listing which has been submitted in ASCII format via EFS-Web and is hereby incorporated by reference in its entirety. Said ASCII copy, created on Jun. 31, 2016, is named SeqList_150033US01.txt and is 969 bytes in size.

### BACKGROUND

Obesity is a significant healthcare problem and along with it co-morbidities such as type 2 diabetes, cardiovascular disease, and cancer. Although the health benefits of weight reduction are well-recognised, weight loss by diet and exercise fail in most patients. Presently, the treatment that eliminates obesity with highest efficacy is bariatric surgery, but this treatment is costly and risky. Pharmacological intervention is generally less efficacious and may be associated with side effects.

The present invention provides new cholecystokinin (CCK) peptide derivatives with a potential for use in the treatment of obesity and related health care problems.

CCK is a gut peptide which is known to inhibit food intake. CCK is rapidly released from I cells in the duodenum and the ileum in response to the intraluminal presence of the digestive products of fats and proteins.

One of several bioactive forms of CCK is “CCK-8s” which is an octapeptide (hence the number 8), and where the “s” refers to a tyrosine residue that is sulphated. The sequence of CCK-8s is Asp-sTyr-Met-Gly-Trp-Met-Asp-Phe-amide (SEQ ID NO: 1).

There are two known CCK receptors: The CCK-1 receptor (CCK-1R) and the CCK-2 receptor (CCK-2R). The CCK-1R is the predominant receptor involved with food intake and satiety, whereas the CCK-2R mediates other effects including Central Nervous System (CNS) effects such as anxiety and panic attacks.

WO 2002/070546 A2 and WO 2010/009872 A1 disclose the preparation of a few CCK-8 analogues and the effect thereof in various animal models.

U.S. Pat. No. 3,892,726 discloses the preparation of a number of CCK-8 analogues.

Int. J. Peptide Protein Res. 1992, vol. 39, p. 48-57 discloses the preparation of four CCK-8 analogues, as well as the biological activities of these compounds compared to native CCK-8s and other CCK-8 analogues.

Bioconjugate Chem. 2010, vol. 21, p. 663-670 discloses the preparation of a few CCK-8 analogues N-terminally conjugated with the macrocyclic chelator DOTA.

US 2004/0185510 A1 discloses a number of unsulfated CCK-7, CCK-8, CCK-9, and CCK-10 analogues which after labelling with a radioactive isotope can be used for detection of tissues having CCK-2 (CCK-B) receptors.

U.S. Pat. No. 4,490,364 discloses the preparation and testing of a number of CCK-8 analogues.

U.S. Pat. No. 5,631,230 discloses the preparation of twelve analogues of CCK-8 and their inhibition of the binding of CCK-8 in the brain and in pancreas in three animal species.

U.S. Pat. No. 3,705,140 discloses the preparation of seventeen analogues of CCK-8 and reports the results of studies of seven of these on some animal species and tissues, as compared to cholecystokinin-pancreozymin.

WO 2013/098408 A1 relates to peptide conjugates comprising a glucagon receptor agonist peptide moiety and a CCK-derived peptide moiety and their pharmaceutical use. Fifteen specific conjugates are disclosed together with their in vitro activity on the glucagon, the CCK-1, and the CCK-2 receptor. Also some results of mice PK and PD studies are reported.

WO 2012/136792 A2 relates to fusions of various insulinotropic, incretin, or gut peptide molecules with certain immunoglobulin single variable domains including one specific CCK-8 modified AlbudAb conjugate.

J. Med. Chem. 1997, vol. 40, p. 4302-4307 reports studies of eight CCK-1 (CCK-A) receptor agonists.

WO 2011/131646 A1 relates to long-acting gastrin derivatives.

### SUMMARY

The present invention relates to a peptide derivative of the general formula I: P-L-X1-X2-X3-Gly-Trp-X6-DMeAsp-X8-NH₂, wherein P is Chem. 1: HOOC—(CH₂)ₓ—CO—*, wherein x is an integer in the range of 12-18; L is absent, or L comprises at least one linker element selected from Chem. 2: *—NH—CH(COOH)—(CH₂)₂—CO—*, Chem. 3: *—NH—(CH₂)₂—[O—(CH₂)₂]ₖ—O—[CH₂]ₙ—CO—* wherein k is an integer in the range of 1-11 and n is an integer in the range of 1-5, Chem. 4: *—NH—CH(CH₂OH)—CO—*, Chem. 5: *—NH—CH₂—CO—*, and/or Chem. 6: *—NH—CH[(CH₂)₄—NH₂]—CO—*; X1 is absent, Asp, DAsp, bAsp, DbAsp, Glu, or DGlu; X2 is Phe(4-sulfomethyl) or sTyr; X3 is Nle, Leu, Ala, Ile, Lys, Arg, Pro, Met, Phe, Ser, His, or Val; X6 is Nle, Ile, Gln, Met, Met(O2), Leu, Val, Pro, Hpg, or Ala; X8 is Phe, MePhe, 1Nal, Me1Nal, 2Nal, Me2Nal, Trp, or MeTrp; and the NH₂ group at the C-terminus of formula I means that X8 is C-terminally amidated; or a pharmaceutically acceptable salt, amide, or ester thereof.

The invention also relates to the particular CCK-derivatives of formulas Chem. 21, Chem. 22, Chem. 23, Chem. 24, Chem. 25, Chem. 26, Chem. 27, Chem. 28, Chem. 29, Chem. 30, Chem. 31, Chem. 32, Chem. 33, Chem. 34, Chem. 35, Chem. 36, Chem. 37, Chem. 38, Chem. 39, Chem. 40, Chem. 41, Chem. 42, Chem. 43, Chem. 44, Chem. 45, Chem. 46, Chem. 47, Chem. 48, Chem. 49, Chem. 50, Chem. 51, Chem. 52, Chem. 53, Chem. 54, Chem. 55, Chem. 56, Chem. 57, Chem. 58, Chem. 59, Chem. 60, Chem. 61, Chem. 62, Chem. 63, Chem. 64, Chem. 65, Chem. 66, Chem. 67, Chem. 68, Chem. 69, Chem. 70, Chem. 71, and Chem. 72; as well as their pharmaceutically acceptable salts, amides, and esters.

The invention furthermore relates to a CCK-peptide of the general formula I′: X1-X2-X3-Gly-Trp-X6-DMeAsp-X8-NH₂, wherein X1 is absent, Asp, DAsp, bAsp, DbAsp, Glu, or DGlu; X2 is Phe(4-sulfomethyl) or sTyr; X3 is Nle, Leu, Ala, Ile, Lys, Arg, Pro, Met, Phe, Ser, His, or Val; X6 is Nle, Ile, Gln, Met, Met(O2), Leu, Val, Pro, Hpg, or Ala; X8 is Phe, MePhe, 1Nal, Me1Nal, 2Nal, Me2Nal, Trp, or MeTrp; and the NH₂ group at the C-terminus of formula I′ means that X8 is C-terminally amidated; as well as the pharmaceutically acceptable, salts, amides, and esters thereof. This peptide is an intermediate of the derivative of the general formula I.

Particular CCK-peptides of the invention have the amino acid sequences denoted herein as Seq 21, Seq 22, Seq 23, Seq 24, Seq 25, Seq 27, Seq 28, Seq 29, Seq 30, Seq 31, Seq 32, Seq 33, Seq 34, Seq 35, Seq 36, Seq 37, Seq 38, Seq 39, Seq 40, Seq 41, Seq 42, Seq 43, Seq 44, Seq 45, Seq 46, Seq 47, Seq 48, Seq 49, Seq 50, Seq 51, Seq 52, Seq 53, Seq 54, Seq 55, Seq 56, Seq 57, and Seq 58; and include their pharmaceutically acceptable salt, amides, or esters.

The invention furthermore relates to methods for preparing the peptides and the derivatives of the invention.

The invention also relates to pharmaceutical compositions comprising a derivative or a peptide of the invention, together with a pharmaceutically acceptable excipient; as well as an injection device with content thereof.

Lastly, the invention relates to the derivative or the peptide of the invention for use as a medicament, in particular for the treatment or prevention of overweight, for reduction of appetite and/or for reduction of food intake; and also to corresponding methods of using these compounds.

In some embodiments the peptides and derivatives of the invention have a high potency on the CCK-1R.

In some embodiments the peptides and derivatives of the invention have a high selectivity for the CCK-1R as compared to the CCK-2R.

In some embodiments the derivatives of the invention potently reduce food intake and body weight in relevant animal models.

In some embodiments further reductions in food intake and body weight are obtained by combination with another weight reducing agent.

In some embodiments the derivatives of the invention have a favourable pharmacokinetics (PK) profile with a high terminal half-life and consequently a long duration of action in vivo.

In some embodiments, the derivatives of the invention have an excellent chemical stability and also solubility in a relevant pharmaceutical composition over a range of relevant temperatures, for several weeks or even longer.

These features are generally very important in the development of efficient and beneficial active pharmaceutical ingredients, and the CCK-1R selectivity is of particular importance in order to avoid centrally mediated side effects such as anxiety.

### DESCRIPTION

In what follows, Greek letters may be represented by their symbol or the corresponding written name, for example: α=alpha; β=beta; ε=epsilon; γ=gamma; ω=omega; etc. Also, the Greek letter of may be represented by “u”, e.g. in μl=ul, or in μM=uM.

An asterisk (*) in a chemical formula designates i) a point of attachment, ii) a radical, and/or iii) an unshared electron.

Unless otherwise indicated the term “about” means+/−10%.

The present invention relates to a peptide derivative of general formula I (first aspect), a peptide of general formula I′ (second aspect), methods for preparing these (third aspect), a pharmaceutical composition comprising a derivative or a peptide of the invention together with a pharmaceutically acceptable excipient (fourth aspect), an injection device containing a pharmaceutical composition of the invention (fifth aspect), and the pharmaceutical use of the derivative or the peptide of the invention, in particular for the treatment or prevention of overweight, for reduction of appetite, and/or for reduction of food intake (sixth aspect).

These aspects are discussed one-by-one in the following.

### Compounds

In a first aspect the present invention relates to a peptide derivative of the general formula I: P-L-X1-X2-X3-Gly-Trp-X6-DMeAsp-X8-NH₂, wherein P is Chem. 1: HOOC—(CH₂)ₓ—CO—*, wherein x is an integer in the range of 12-18; L is absent, or L comprises at least one linker element selected from Chem. 2: *—NH—CH(COOH)—(CH₂)₂—CO—*, Chem. 3: *—NH—(CH₂)₂—[O—(CH₂)₂]ₖ—O—[CH₂]ₙ—CO—* wherein k is an integer in the range of 1-11 and n is an integer in the range of 1-5, Chem. 4: *—NH—CH(CH₂OH)—CO—*, Chem. 5: *—NH—CH₂—CO—*, and/or Chem. 6: *—NH—CH[(CH₂)₄—NH₂]—CO—*; X1 is absent, Asp, DAsp, bAsp, DbAsp, Glu, or DGlu; X2 is Phe(4-sulfomethyl) or sTyr; X3 is Nle, Leu, Ala, Ile, Lys, Arg, Pro, Met, Phe, Ser, His, or Val; X6 is Nle, Ile, Gln, Met, Met(O2), Leu, Val, Pro, Hpg, or Ala; X8 is Phe, MePhe, 1Nal, Me1Nal, 2Nal, Me2Nal, Trp, or MeTrp; and the NH₂ group at the C-terminus of formula I means that X8 is C-terminally amidated; or a pharmaceutically acceptable salt, amide, or ester thereof.

In a second aspect, the invention relates to a peptide of the general formula I′: X1-X2-X3-Gly-Trp-X6-DMeAsp-X8-NH₂, wherein X1 is absent, Asp, DAsp, bAsp, DbAsp, Glu, or DGlu; X2 is Phe(4-sulfomethyl) or sTyr; X3 is Nle, Leu, Ala, Ile, Lys, Arg, Pro, Met, Phe, Ser, His, or Val; X6 is Nle, Ile, Gln, Met, Met(O2), Leu, Val, Pro, Hpg, or Ala; X8 is Phe, MePhe, 1Nal, Me1Nal, 2Nal, Me2Nal, Trp, or MeTrp; and the NH₂ group at the C-terminus of formula I′ means that X8 is C-terminally amidated; or a pharmaceutically acceptable salt, amide, or ester thereof.

This peptide is an intermediate of the derivative of the general formula I. The derivatives of the invention may be prepared by a stepwise synthesis method comprising (i) preparation of the intermediate peptide of the invention, followed by (ii) attachment of the P-L side chain to the N-terminus thereof. Step (i) of this method can be achieved using standard solid phase synthesis as described in the experimental section using protected amino acids; after cleavage from the resin, the peptide can be subjected to purification using preparative HPLC as described in the experimental section herein to give the intermediate peptide. The P-L side chain can be prepared separately as its C-terminal carboxylic acid and then activated with a coupling agent to give an acylating agent, e.g. an N-hydroxysuccinimide ester (NHS ester). This acylating agent can be used to attach the side chain to the N-terminus of the CCK peptide, by formation of an amide bond. The attachment of the side chain to the intermediate peptide, as well as preparation of the side chain itself can be achieved using methods described in WO 2009/083549 A1.

In some embodiments the peptide derivative of the invention is selected from the particular CCK-derivatives of formulas Chem. 21-Chem. 72, as well as their pharmaceutically acceptable salts, amide, and esters.

In some embodiments the peptide of the invention is selected from the particular CCK-peptides of the invention having the amino acid sequences denoted herein as Seq 21-Seq 58.

### Amino Acids, Peptides and Peptide Derivatives

A peptide is a short chain of amino acid monomers linked by amide bonds.

An amino acid residue is a radical of an amino acid as or when incorporated into a peptide or protein.

In a peptide derivative a well-defined number of short side chains of a well-defined structure are covalently attached to one or more specific amino acid residues of the peptide.

The compound of formula I is a derivative of a peptide of formula I′:

X1-X2-X3-Gly-Trp-X6-DMeAsp-X8-NH₂ (formula I′),

where X1, X2, X3, X6, and X8 are amino acid monomers as defined above, and where a side chain of the structure P-L, as defined above, is covalently attached to the N-terminus of the peptide, via a carboxylic amide bond.

Accordingly, the peptide of formula I′ may be designated the “peptide part” of the derivative of formula I.

In all peptide and peptide derivative formulas herein the N-terminal amino acid is shown to the left and the C-terminal amino acid is shown to the right, as is standard in the art.

The amino acids of the peptide of the invention include coded amino acids as well as non-coded amino acids.

The coded amino acids are defined by IUPAC (Table 1 in section 3AA-1): www.chem.qmul.ac.uk/iupac/AminoAcid/AA1n2.html#AA1), which gives structure, trivial name, systematic name, one- and three-letter symbols for the 20 coded amino acids.

The non-coded amino acids being incorporated in the peptide of formula I′ and in the peptide part of the derivative of formula I are defined in Table 1, below.

TABLE 1
Non-coded amino acid residues

| | | Chemical structure of the | |
|---|---|---|---|
| | | residue with attachment | |
| Abbreviation | Name(s) of the amino acid | points | Chem. no. |
| bAsp | beta-aspartic acid β-aspartic acid iso-aspartic acid | 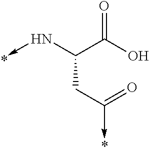 | 80 |
| DAsp | (D)-aspartic acid | 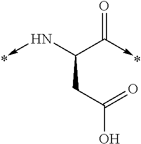 | 6 |
| DbAsp | D-beta-aspartic acid D-β-aspartic acid D-iso-aspartic acid | 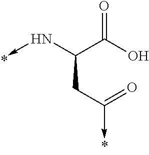 | 81 |
| DGlu | (D)-glutamic acid | 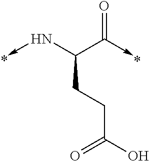 | 7 |
| Hpg | L-homopropargylglycine (2S)-2-aminohex-5-ynoic acid | 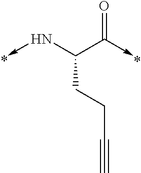 | 83 |
| DMeAsp | (D)-N-methylaspartic acid | 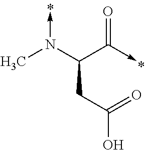 | 8 |
| Me1Nal | N-methyl-1-naphthylalanine | 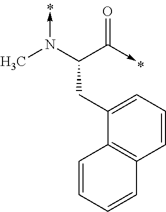 | 14 |
| Me2Nal | N-methyl-2-naphthylalanine | 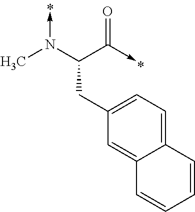 | 16 |
| MePhe | N-methylphenylalanine | 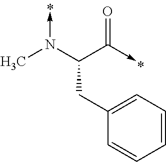 | 12 |
| Met(O2) | methionine sulfone (2S)-2-amino-4- methylsulfonyl-butanoic acid | 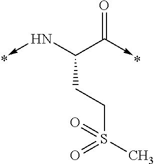 | 82 |
| MeTrp | N^{a}-methyltryptophan | 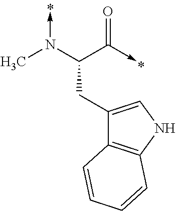 | 17 |
| 1Nal | 1-naphthylalanine | 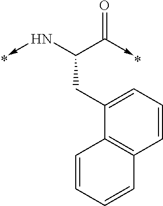 | 13 |
| 2Nal | 2-naphthylalanine | 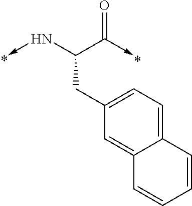 | 15 |
| Nle | norleucine; (S)-2-aminohexanoic acid | 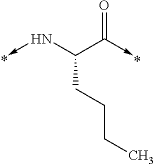 | 11 |
| Phe(4-sulfomethyl) | 4-sulfomethylphenylalanine | 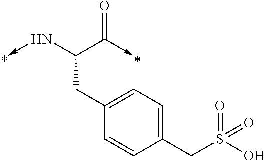 | 9 |
| sTyr | sulfotyrosine; sulfated tyrosine | 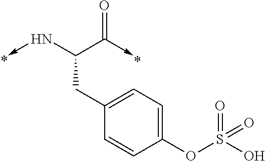 | 10 |

Amino acids X1, X2, X3, Trp, X6, and X8 incorporated in the peptides and the peptide part of the peptide derivatives of the invention may exist in either of two enantiomeric forms, the L-form or the D-form. For the present purposes each of these amino acids is in the L-form, unless otherwise noted. As a non-limiting example, the amino acid X1 may be “Asp” where accordingly the L-form is implied, or it may be “DAsp” where the D-form is explicitly noted.

In all other contexts, i.e. in case of an optically active compound or radical which is not an amino acid residue of the peptide of formula I′, and for which the specific stereoisomer is not explicitly indicated in the formula and/or name of the compound or radical in question, this formula and/or name is intended to cover all isomers including any mixture thereof. As a non-limiting example, the structural formula Chem. 2 is a di-radical of the optically active amino acid Glu, and since no stereo chemistry is shown in Chem. 2, this formula covers the L-form, the D-form, and any mixture thereof.

### CCK Peptides, Analogues, and Derivatives

The term “CCK peptide” as used herein refers to any of the forms in which cholecystokinin exists in human beings, or an analogue thereof. Non-limiting examples of CCK peptides include CCK-8s, CCK-22s, CCK-33s, CCK-58s, CCK-83s, and analogues thereof.

The term “CCK derivative” as used herein refers to a derivative of a CCK peptide, where a well-defined number of short side chains of a well-defined structure are covalently attached to one or more specific amino acid residues of the CCK peptide.

The CCK peptide which is herein designated “CCK-8s” refers to the following octa-peptide: Asp-sTyr-Met-Gly-Trp-Met-Asp-Phe-amide (SEQ ID NO: 1). The name and structural formula for the sTyr amino acid residue are shown in Table 1. Alternative names to “CCK-8s” are “native CCK-8s”, “sulfated CCK-8”, and “native human CCK-8s”.

The term “analogue of CCK-8s” (alternatively “CCK-8s analogue”) as used herein refers to a peptide which is a variant of native CCK-8s (SEQ ID NO: 1). The CCK-8s analogue of the invention differs from native CCK-8s by virtue of one or more amino acid changes, where the term “amino acid changes” refers to substitutions, extensions, deletions, insertions, or combinations thereof. In one embodiment the amino acid changes are selected from substitutions and deletions. In another embodiment the amino acid changes are substitutions. In one embodiment the CCK-8s analogue of the invention has between three and six amino acid changes as compared to native CCK-8s. The amino acid changes relevant for the CCK-8s analogue of the invention are defined in formulas I and I′.

For counting the number of amino acid changes, for identifying the precise kind of amino acid change(s), and/or for identifying equivalent or corresponding positions a peptide of interest is aligned with native CCK-8s (SEQ ID NO: 1). The alignment can be done by simple handwriting and visual inspection (“eyeballing”).

### Derivatives

In a peptide derivative a well-defined number of short side chains of a well-defined structure are covalently attached to one or more specific amino acid residues of a peptide.

The peptide derivative of the invention (formula I) has one short side chain consisting of a Protractor (P) and a Linker (L), where P and L are interconnected via an amide bond, and L is covalently attached to the N-terminal amino acid of the peptide of the invention, also via an amide bond.

The Protractor (P) is a C14-C20 di-acid of formula Chem. 1.

The Linker (L) comprises at least one linker element of formula Chem. 2, Chem. 3, Chem. 4, Chem. 5, and/or Chem. 6.

In some embodiments the Chem. 3 linker element is a linker element of Chem. 3a: *—NH—(CH₂)₂—O—(CH₂)₂—O—CH₂—CO—*, which is herein designated Ado (a di-radical of 8-amino-3,6-dioxaoctanoic acid).

In some embodiments the Chem. 2 linker element: *—NH—CH(COOH)—(CH₂)₂—CO—*, is a linker element of Chem. 2a:

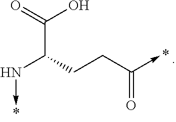

The Chem. 2 as well as the Chem. 2a linker element is a di-radical of Glu, herein referred to as gamma-Glu, or just gGlu, due to the fact that it is the gamma carboxy group of the amino acid glutamic acid which is used for connection to the amino group of the N-terminal amino acid of the peptide of the invention, or to the one or more additional linker elements selected from Chem. 2, Chem. 3, Chem. 4, and/or Chem. 5. While the structure of Chem. 2 covers all enantiomer forms of gGlu and any mixture thereof, the structure of Chem. 2a is the L-form of gGlu.

### Pharmaceutically Acceptable Salt, Amide, or Ester

The derivatives, analogues, and peptides of the invention may be in the form of a pharmaceutically acceptable salt, amide, or ester.

Salts are e.g. formed by a chemical reaction between a base and an acid, e.g.: 2NH₃+H₂SO₄→(NH₄)₂SO₄.

The salt may be a basic salt, an acid salt, or it may be neither nor (i.e. a neutral salt). Basic salts produce hydroxide ions and acid salts hydronium ions in water.

The salts of the derivatives of the invention may be formed with added cations or anions between anionic or cationic groups, respectively. These groups may be situated in the peptide part, and/or in the side chain of the derivatives of the invention.

Non-limiting examples of anionic groups of the derivatives of the invention include free carboxylic groups in the side chain, if any, as well as in the peptide moiety. The peptide moiety often includes a free carboxylic acid group at the C-terminus, and it may also include free carboxylic groups at internal acid amino acid residues such as Asp and Glu.

Non-limiting examples of cationic groups in the peptide moiety include the free amino group at the N-terminus, if present, as well as any free amino group of internal basic amino acid residues such as His, Arg, and Lys.

The ester of the derivatives of the invention may, e.g., be formed by the reaction of a free carboxylic acid group with an alcohol or a phenol, which leads to replacement of at least one hydroxyl group by an alkoxy or aryloxy group

The ester formation may involve the free carboxylic group at the C-terminus of the peptide, and/or any free carboxylic group in the side chain.

The amide of the derivatives of the invention may, e.g., be formed by the reaction of a free carboxylic acid group with an amine or a substituted amine, or by reaction of a free or substituted amino group with a carboxylic acid.

The amide formation may involve the free carboxylic group at the C-terminus of the peptide, any free carboxylic group in the side chain, the free amino group at the N-terminus of the peptide, and/or any free or substituted amino group of the peptide in the peptide and/or the side chain.

In a particular embodiment, the peptide or derivative is in the form of a pharmaceutically acceptable salt. In another particular embodiment, the derivative is in the form of a pharmaceutically acceptable amide, preferably with an amide group at the C-terminus of the peptide. In a still further particular embodiment, the peptide or derivative is in the form a pharmaceutically acceptable ester. In a particular embodiment, the peptide or derivative is in the form of a pharmaceutically acceptable salt or ester.

### Functional Properties

The CCK peptides and CCK derivatives of the invention have important and interesting functions and effects that make them particularly suitable and promising for being developed into efficient and beneficial medicaments for the treatment of obesity and related health problems.

Non-limiting examples of such important and interesting functions and effects include the following, as well as any combination of two or more of these functions and effects:

In some embodiments the peptides and derivatives of the invention have a high potency on the CCK-1R.

In some embodiments the peptides and derivatives of the invention have a high selectivity for the CCK-1R as compared to the CCK-2R.

In some embodiments the peptides and derivatives of the invention have a high affinity for the CCK-1R.

In some embodiments the peptides and derivatives of the invention selectively bind to the CCK-1R as compared to the CCK-2R.

In some embodiments the derivatives of the invention potently reduce food intake and body weight in a relevant animal model, as monotherapy and in combination with another weight reducing agent.

In some embodiments the derivatives of the invention have a favourable pharmacokinetics (PK) profile with long terminal half-lives and consequently a long duration of action in vivo.

In some embodiments, the derivatives of the invention have an excellent chemical stability and also solubility in a relevant pharmaceutical composition over a range of relevant temperatures, for several weeks or even longer.

### Biological Activity—In Vitro Potency

In some embodiments the peptides and derivatives of the invention have a high potency on the CCK-1R.

### CCK-1R Agonist

In some embodiments the peptide of the invention it is a CCK-1R agonist. In some embodiments it is a full CCK-1R agonist.

In some embodiments the derivative of the invention it is a CCK-1R agonist. In some embodiments it is a full CCK-1R agonist.

A receptor agonist may be defined as a compound that binds to a receptor and elicits a response typical of the natural ligand. A full agonist may be defined as one that elicits a response of the same magnitude as the natural ligand (see e.g. “Principles of Biochemistry”, A L Lehninger, D L Nelson, M M Cox, Second Edition, Worth Publishers, 1993, page 763).

Thus, for example, a “CCK-1R agonist” may be defined as a compound which is capable of binding to the CCK-1R and capable of activating it. A “full” CCK-1R agonist may be defined as a CCK-1R agonist which is capable of eliciting a magnitude of CCK-1R response that is similar to that of CCK-8s (SEQ ID NO: 1).

### In Vitro Potency, EC₅₀ Value

The in vitro potency on a receptor may be determined as the EC₅₀ value, which refers to the concentration of the compound in question which in a receptor activation assay induces a response half way between the basal response and the maximal response. The lower the EC₅₀ value, the higher the in vitro potency.

### CCK-1R

Thus, the in vitro potency on the CCK-1R of a peptide or a derivative of the invention may be determined in a CCK-1R activation assay, as the EC₅₀ value.

Any suitable CCK-1R activation assay may be used, for example the one described in Example 91.

In some embodiments native CCK-8s (SEQ ID NO: 1) is used as a positive reference.

In some embodiments Gastrin-17 is used as a negative reference. Gastrin-17 has virtually no activity on the CCK-1R but is active on the CCK-2R.

The sequence of Gastrin-17 is: pEGPWLEEEEEAYGWMDF (SEQ ID NO: 2), where the C-terminal amino acid residue (F) is amidated, and the N-terminus, pE, represents pyroglutamic acid, for which the 3-letter abbreviation can be Pyr:

### Chem. 18:

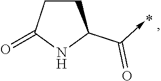

As it is clear from SEQ ID NO: 2 above, the Gastrin-17 compound is non-sulfated i.e. it incorporates a non-sulfated tyrosine residue. Even if the derivative of the invention may have a somewhat lower in vitro potency (higher EC₅₀ value) on the CCK-1R than native CCK-8s, the in vitro potency on the CCK-1R of the derivatives of the invention is fully satisfactory.

In some embodiments, the derivatives and peptides of the invention have a very good in vitro potency on the CCK-1R, corresponding to an EC₅₀ value which is equal to or lower than 1000 pM.

### CCK-2R

The in vitro potency on the CCK-2R of a peptide or a derivative of the invention may be determined in a CCK-2R activation assay, as the EC₅₀ value.

Any suitable CCK-2R activation assay may be used, for example the one described in Example 91.

In some embodiments native CCK-8s (SEQ ID NO: 1) is used as a positive reference.

In some embodiments Gastrin-17 is used as a positive reference. Gastrin-17 has a very high activity on the CCK-2R, and CCK-8s also has a good activity on this receptor.

The derivatives and peptides of the invention have a very low in vitro potency on the CCK-2R, definitely lower than native CCK-8s, in some embodiments much lower (a low in vitro potency corresponds to a high EC₅₀ value).

### CCK-1R Selectivity

In some embodiments the peptides and derivatives of the invention have a high selectivity for the CCK-1R as compared to the CCK-2R.

In some embodiments the CCK-1R selectivity is defined by the ratio [EC₅₀ (CCK-2R)/EC₅₀ (CCK-1R)]. In some embodiments this ratio is above 1. In some embodiments the ratio is much higher than 1, e.g., without limitation, at least 25, or at least 500.

As explained above the high CCK-1R selectivity is desirable in order to avoid adverse CCK-2R mediated side effects, e.g. on the CNS.

### Biological Activity—In Vitro Receptor Binding

In some embodiments the peptides and derivatives of the invention have a high binding affinity to the CCK-1R.

### In Vitro Receptor Binding, IC₅₀ Value

The in vitro receptor binding or affinity may be determined as the IC₅₀ value, which refers to the concentration of a compound which is required for 50% inhibition of maximal radio ligand binding to the receptor. Thus, a lower IC₅₀ value corresponds to a higher affinity of the compound.

### CCK-1R and CCK-2R

Any suitable CCK-1R and CCK-2R binding assays may be used, for example those described in Example 92.

In some embodiments native CCK-8s (SEQ ID NO: 1) and/or Gastrin-17 is/are used as references. CCK-8s binds well to the CCK-1R and also to the CCK-2R, whereas Gastrin-17 binds well to the CCK-2R but not to the CCK-1R.

In some embodiments, the derivatives and peptides of the invention have a very good affinity to the CCK-1R, corresponding to an IC₅₀ value which is equal to or lower than 1000 pM.

The derivatives and peptides of the invention have a very low affinity to the CCK-2R, definitely lower than native CCK-8s, in some embodiments much lower (corresponding to a high IC₅₀ value).

### CCK-1R Binding Selectivity

In some embodiments the peptides and derivatives of the invention have a high binding selectivity for the CCK-1R as compared to the CCK-2R.

In some embodiments the CCK-1R binding selectivity is defined by the ratio [IC₅₀ (CCK-2R)/IC₅₀ (CCK-1R)]. In some embodiments this ratio is above 1. In some embodiments the ratio is much higher than 1, e.g., without limitation, at least 200, or at least 1000.

As explained above high CCK-1R binding selectivity is desirable in order to avoid adverse CCK-2R mediated side effects, e.g. on the CNS.

### Biological Activity—In Vivo Pharmacology—Pharmacodynamic Profile

The derivatives of the invention are potent in vivo. The in vivo potency refers to the fact that the compounds have a relevant biological effect in vivo. Two examples, without limitation, of relevant biological effects are reduction of food intake, and reduction of body weight.

The biological effect in vivo may be determined as is known in the art in any suitable animal model, as well as in clinical trials.

In some embodiments the derivatives of the invention potently reduce food intake in a relevant animal model.

In some embodiments the derivatives of the invention potently reduce body weight in a relevant animal model.

In some embodiments a relevant animal model is a pig.

In some embodiments the pigs are LYD pigs and/or Large White hybrid pigs. In some embodiments reduction in food intake weight may be determined in a PD study in such pigs using any suitable method known in the art. Examples, without limitation, of such methods are described in Examples 94 and 97.

In some embodiments the pigs are diet-induced obese (DIO) mini-pigs. In some embodiments reduction in food intake weight and/or body weight may be determined in a PD study in such pigs using any suitable method known in the art. In some embodiments the PD study is a mono therapy study. In some embodiments the PD study includes an add-on study with combined treatment with another weight reducing agent, for example a GLP-1 receptor agonist. An example, without limitation, of such method is described in Example 96.

The derivatives of the invention are very potent in vivo, which, for example, without limitation, is evidenced by a fine reduction in food intake up to several days after administration of a single s.c. dose of the derivative. For more details, see Examples 94 and 97. Also very fine reductions in food intake and body weight were obtained in the sub-chronic PD study with DIO pigs described in Example 96.

### Pharmacokinetics Profile

In some embodiments the derivatives of the invention have improved pharmacokinetic properties such as increased terminal half-life and/or decreased clearance.

Increasing terminal half-life and/or decreasing of the clearance means that the compound in question is eliminated slower from the body. For the derivatives of the invention this entails an extended duration of pharmacological effect.

The pharmacokinetic properties of the derivatives of the invention may suitably be determined in-vivo in pharmacokinetic (PK) studies. Such studies are conducted to evaluate how pharmaceutical compounds are absorbed, distributed, and eliminated in the body, and how these processes affect the concentration of the compound in the body, over the course of time.

In the discovery and preclinical phase of pharmaceutical drug development, animal models such as the mouse, rat, monkey, dog, or pig, may be used to perform this characterisation. Any of these models can be used to test the pharmacokinetic properties of the derivatives of the invention.

In such studies, animals are typically administered a single dose of the drug, in a suitable formulation and via a relevant administration route. Blood samples are drawn at predefined time points after dosing, and samples are analysed for concentration of drug with a relevant quantitative assay. Based on these measurements, time-plasma concentration profiles for the compound of study are plotted and a so-called non-compartmental pharmacokinetic analysis of the data is performed.

For most compounds, the terminal part of the plasma-concentration profiles will be linear when drawn in a semi-logarithmic plot, reflecting that after the initial absorption and distribution, drug is removed from the body at a constant fractional rate. The rate (lambda Z or λ_{z}) is equal to minus the slope of the terminal part of the plot. From this rate, also a terminal half-life may be calculated, as t^{1/2}=ln(2)/λ_{z} (see, e.g., Johan Gabrielsson and Daniel Weiner: Pharmacokinetics and Pharmacodynamic Data Analysis. Concepts & Applications, 3rd Ed., Swedish Pharmaceutical Press, Stockholm (2000)).

Clearance can be determined after i.v. administration and is defined as the dose (D) divided by area under the curve (AUC) on the plasma concentration versus time profile (Rowland, M and Tozer T N: Clinical Pharmacokinetics: Concepts and Applications, 3^{rd} edition, 1995 Williams Wilkins).

The estimate of terminal half-life and/or clearance is relevant for evaluation of dosing regimens and an important parameter in drug development, in the evaluation of new drug compounds.

### Pharmacokinetics Profile—Half Life In Vivo in Minipigs

In some embodiments the derivatives of the invention have improved pharmacokinetic (PK) properties. For example, without limitation, the PK properties are improved relative to native CCK peptides with reported half-lives of less than 5 minutes (see, e.g., Gastroenterology, 1993, Vol. 105(6), pp. 1732-1736).

In some embodiments, the PK properties are determined as terminal half-life (T_{1/2}) in vivo in minipigs after i.v. administration. Such studies may be conducted as is known in the art. One example, without limitation, of a suitable study description is found in Example 93.

The derivatives of the invention have a more protracted profile of action than, for example, without limitation, native CCK-8s (SEQ ID NO: 1).

In some embodiments, the terminal half-life (T_{1/2}) after i.v. administration to mini-pigs is at least 2 hours. In some embodiments, the terminal half-life (T_{1/2}) after i.v. administration to mini-pigs is at least 30 hours, or at least 90 hours.

For more details, see Example 93.

### Chemical Stability

In some embodiments, the derivatives of the invention have an excellent chemical stability in a relevant pharmaceutical composition over a range of relevant temperatures, for several weeks or even longer.

In some embodiments the derivative of the invention is chemically stable. For example, without limitation, it is very stable in aqueous solutions at physiological pH. In some embodiments it is stable in a pH 7.4 phosphate buffer at 5° C., 25° C., and/or 37° C. for 2 weeks; and/or stable under the same conditions for 6 weeks. The chemical stability may be determined using any method known in the art. A useful method is described in Example 95 herein, which also compares with the chemical stability of CCK-8s, demonstrating an improved stability after two weeks at 37° C. of a derivative of the invention.

### Production Processes

In a third aspect, the invention relates to methods for preparing the peptides and the derivatives of the invention.

The peptides and derivatives of the invention may be produced using any method known in the art. Examples of such methods, without limitation, are included in the experimental part.

The present invention relates to an SPPS-based method of preparing the peptide and/or the derivative of the invention. In some embodiments, Fmoc based chemistry is used. In some embodiments, the peptide and/or the derivative is cleaved from the resin and de-protected. In some embodiments, the method comprises a step of removal of a neopentyl group from Tyr(SO₃-neopentyl). In some embodiments, the method comprises a step of removal of a trichloroethyl (TCE) group from Phe(4-sulfomethyl-TCE). In some embodiments the peptide and/or derivative is dissolved in neutral aqueous ammonium acetate and acetonitrile and purified by reversed-phase preparative HPLC. Non-limiting examples of each of these steps are disclosed in the section “Methods for Synthesis, Purification and Analysis of Example Compounds” in the experimental part.

The invention also relates to a method of preparing the derivative of the invention, which method comprises the following steps: (i) Preparation of the intermediate peptide of formula I′ using Solid Phase Peptide Synthesis (SPPS); (ii) preparation of the P-L side chain of formula I in the form of its C-terminal carboxylic acid, and activation thereof with a coupling agent resulting in the formation of an acylating agent; and (iii) attachment of the acylating agent from step (ii) to the N-terminus of the intermediate peptide from step (i). In some embodiments, the peptide resulting from step (i) is purified. In some embodiments it is purified using preparative HPLC.

### Pharmaceutical Compositions

In a fourth aspect, the invention also relates to pharmaceutical compositions comprising a derivative or a peptide of the invention, together with a pharmaceutically acceptable excipient.

The present invention also relates to a pharmaceutical composition comprising a derivative and/or a peptide of the invention together with a pharmaceutically acceptable excipient.

Such pharmaceutical compositions may be produced using any method known in the art. Examples of such compositions, without limitation, are included in the experimental part.

The term “excipient” broadly refers to any component other than the active therapeutic ingredient(s). The excipient may be an inert substance, an inactive substance, and/or a not medicinally active substance.

The excipient may serve various purposes, e.g. as a carrier, vehicle, diluent, tablet aid, and/or to improve administration, and/or absorption of the active substance.

The formulation of pharmaceutically active ingredients with various excipients is known in the art, see e.g. Remington: The Science and Practice of Pharmacy (e.g. 19^{th} edition (1995), and any later editions).

Non-limiting examples of excipients are: Solvents, diluents, buffers, preservatives, tonicity regulating agents, chelating agents, and stabilisers.

Examples of formulations include liquid formulations, i.e. aqueous formulations comprising water. A liquid formulation may be a solution, or a suspension. An aqueous formulation typically comprises at least 50% w/w water, or at least 60%, 70%, 80%, or even at least 90% w/w of water.

Alternatively, a pharmaceutical composition may be a solid formulation, e.g. a freeze-dried or spray-dried composition, which may be used as is, or whereto the physician or the patient adds solvents, and/or diluents prior to use.

The pH in an aqueous formulation may be anything between pH 3 and pH 10, for example from about 7.0 to about 9.5; or from about 3.0 to about 7.0.

A pharmaceutical composition may comprise a buffer. The buffer may e.g. be selected from sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid, and mixtures thereof.

A pharmaceutical composition may comprise a preservative. The preservative may e.g. be selected from phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol), and mixtures thereof. The preservative may be present in a concentration from 0.1 mg/ml to 20 mg/ml.

A pharmaceutical composition may comprise an isotonic agent. The isotonic agent may e.g. be selected from a salt (e.g. sodium chloride), a sugar or sugar alcohol, an amino acid (e.g. glycine, histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), and mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alfa and beta HPCD, soluble starch, hydroxyethyl starch and carboxymethylcellulose-Na may be used. Sugar alcohol is defined as a C4-C8 hydrocarbon having at least one —OH group and includes, for example, mannitol, sorbitol, inositol, galactitol, dulcitol, xylitol, and arabitol. In one embodiment, the sugar alcohol additive is mannitol.

In some embodiments the pharmaceutical composition of the invention is a solution comprising a preservative. In some embodiments the preservative is m-cresol. In some embodiments the preservative is phenol.

A pharmaceutical composition may comprise a chelating agent. The chelating agent may e.g. be selected from salts of ethylenediaminetetraacetic acid (EDTA), citric acid, and aspartic acid, and mixtures thereof.

A pharmaceutical composition may comprise a stabiliser. The stabiliser may e.g. be one or more oxidation inhibitors, aggregation inhibitors, surfactants, and/or one or more protease inhibitors. Non-limiting examples of these various kinds of stabilisers are disclosed in the following.

The term “aggregate formation” refers to a physical interaction between the polypeptide molecules resulting in formation of oligomers, which may remain soluble, or large visible aggregates that precipitate from the solution. Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

A pharmaceutical composition may comprise an amount of an amino acid base sufficient to decrease aggregate formation of the polypeptide during storage of the composition. The term “amino acid base” refers to one or more amino acids (such as methionine, histidine, imidazole, arginine, lysine, isoleucine, aspartic acid, tryptophan, threonine), or analogues thereof. Any amino acid may be present either in its free base form or in its salt form. Any stereoisomer (i.e., L, D, or a mixture thereof) of the amino acid base may be present.

Methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. Any stereoisomer of methionine (L or D) or combinations thereof can be used.

A pharmaceutical composition may comprise a stabiliser selected from high molecular weight polymers or low molecular compounds. The stabiliser may e.g. be selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). A pharmaceutical composition may comprise additional stabilising agents such as, but not limited to, methionine and EDTA, which protect the polypeptide against methionine oxidation, and a nonionic surfactant, which protects the polypeptide against aggregation associated with freeze-thawing or mechanical shearing.

A pharmaceutical composition may comprise one or more surfactants. The term “surfactant” refers to any molecules or ions that are comprised of a water-soluble (hydrophilic) part, and a fat-soluble (lipophilic) part. The surfactant may e.g. be selected from anionic surfactants, cationic surfactants, nonionic surfactants, and/or zwitterionic surfactants. In some embodiments the surfactant is a polysorbate such as polysorbate-20 or polysorbate-80. In some embodiments the surfactant is a poloxamer, such as poloxamer-188.

A pharmaceutical composition may comprise one or more protease inhibitors, such as, e.g., EDTA (ethylenediamine tetraacetic acid), and/or benzamidineHCl.

Additional, optional, ingredients of a pharmaceutical composition include, e.g., wetting agents, emulsifiers, antioxidants, bulking agents, metal ions, oily vehicles, proteins (e.g., human serum albumin, gelatine), and/or a zwitterion (e.g., an amino acid such as betaine, taurine, arginine, glycine, lysine and histidine).

In some embodiments the derivative of the invention is administered in a dose of from 0.01-10000 nmol/kg/day, preferably 0.1-1000 nmol/kg/day.

The derivative may be administered in the form of a pharmaceutical composition. It may be administered to a patient in need thereof at several sites, for example, at topical sites such as skin or mucosal sites; at sites which bypass absorption such as in an artery, in a vein, or in the heart; and at sites which involve absorption, such as in the skin, under the skin, in a muscle, or in the abdomen.

The route of administration may be, for example, lingual; sublingual; buccal; in the mouth; oral; in the stomach; in the intestine; nasal; pulmonary, such as through the bronchioles, the alveoli, or a combination thereof; parenteral, epidermal; dermal; transdermal; conjunctival; uretal; vaginal; rectal; and/or ocular.

A composition may be administered in several dosage forms, for example as a solution; a suspension; an emulsion; a microemulsion; multiple emulsions; a foam; a salve; a paste; a plaster; an ointment; a tablet; a coated tablet; a chewing gum; a rinse; a capsule such as hard or soft gelatine capsules; a suppositorium; a rectal capsule; drops; a gel; a spray; a powder; an aerosol; an inhalant; eye drops; an ophthalmic ointment; an ophthalmic rinse; a vaginal pessary; a vaginal ring; a vaginal ointment; an injection solution; an in situ transforming solution such as in situ gelling, setting, precipitating, and in situ crystallisation; an infusion solution; or as an implant.

A composition may further be compounded in a drug carrier or drug delivery system, e.g. in order to improve stability, bioavailability, and/or solubility. In a particular embodiment a composition may be attached to such system through covalent, hydrophobic, and/or electrostatic interactions. The purpose of such compounding may be, e.g., to decrease adverse effects, achieve chronotherapy, and/or increase patient compliance.

A composition may also be used in the formulation of controlled, sustained, protracting, retarded, and/or slow release drug delivery systems.

Parenteral administration may be performed by subcutaneous, intramuscular, intraperitoneal, or intravenous injection by means of a syringe, optionally a pen-like syringe, or by means of an infusion pump. In one embodiment the syringe is of the multiple dose administration type. In another embodiment the syringe is of the fixed dose administration type. In some embodiments the syringe is a multiple dose pen-device, or a fixed dose ready to use pen-device.

In some embodiments the pharmaceutical composition of the invention is a solution. In some embodiments the pharmaceutical composition of the invention is a solution. In some embodiments it is a solution for once daily s.c. administration. In some embodiments it is a solution for once weekly subcutaneous administration.

In some embodiments the concentration of the derivative of the invention in the solution is 0.2-50000 nmol/ml, in other embodiments it is 5.0-20000 nmol/ml.

In a fifth aspect, the invention relates to an injection device with content thereof.

In some embodiments the pharmaceutical composition of the invention is intended for use and/or contained in an injection device. In some embodiments, the injection device is a disposable, pre-filled, multi-dose pen of the FlexTouch® type (supplier Novo Nordisk A/S, Denmark). In some embodiments the injection device is a single shot device.

In some embodiments the injection device is a fixed dose device, such as one configured to deliver multiple predetermined doses of drug, sometimes referred to as a multiple fixed dose device or a fixed dose, multi-shot device.

A composition may be administered nasally in the form of a solution, a suspension, or a powder; or it may be administered pulmonally in the form of a liquid or powder spray.

Transdermal administration is a still further option, e.g. by needle-free injection, from a patch such as an iontophoretic patch, or via a transmucosal route, e.g. buccally.

A composition may be a stabilised formulation. The term “stabilised formulation” refers to a formulation with increased physical and/or chemical stability, preferably both.

In general, a formulation must be stable during use and storage (in compliance with recommended use and storage conditions) until the expiration date is reached.

The term “physical stability” refers to the tendency of the polypeptide to form biologically inactive and/or insoluble aggregates as a result of exposure to thermo-mechanical stress, and/or interaction with destabilising interfaces and surfaces (such as hydrophobic surfaces). The physical stability of an aqueous polypeptide formulation may be evaluated by means of visual inspection, and/or by turbidity measurements after exposure to mechanical/physical stress (e.g. agitation) at different temperatures for various time periods. Alternatively, the physical stability may be evaluated using a spectroscopic agent or probe of the conformational status of the polypeptide such as e.g. Thioflavin T or “hydrophobic patch” probes.

The term “chemical stability” refers to chemical (in particular covalent) changes in the polypeptide structure leading to formation of chemical degradation products potentially having a reduced biological potency, and/or increased immunogenic effect as compared to the intact polypeptide. The chemical stability can be evaluated by measuring the amount of chemical degradation products at various time-points after exposure to different environmental conditions, e.g. by SEC-HPLC, RP-UPLC, and/or RP-HPLC. See also Example 95 herein.

### Pharmaceutical Indications

In a sixth aspect the invention relates to the derivative or the peptide of the invention for use as a medicament, in particular for (a) the treatment or prevention of pancreatic disorders, obesity and obesity related disorders, diabetes, other metabolic diseases, gall stone, and/or overweight; and/or for (b) the diagnosis of pancreatic and/or gall bladder mal-function; as well as the corresponding (a) and (b) methods of using these compounds.

Some of these therapeutic indications may be at least partly explained by one or more of the following observed, known or anticipated effects of CCK-1R agonists: Gall bladder contraction, increased secretion of other appetite-regulating gut hormones such as GLP-1 and PYY, reduction of appetite, increased energy expenditure, reduced uptake of certain macro nutrients such as lipids, regulation of plasma lipids (cholesterol, triglycerides), reduction of food intake, reduction of appetite, increased satiety, increased gut motility, and/or reduced gastric emptying.

In some embodiments the invention relates to a method for weight management. In some embodiments the invention relates to a method for reduction of appetite. In some embodiments the invention relates to a method for reduction of food intake.

Generally, all subjects suffering from obesity are also considered to be suffering from overweight. In some embodiments the invention relates to a method for treatment or prevention of obesity. In some embodiments the invention relates to use of the derivative of the invention for treatment or prevention of obesity. In some embodiments the subject suffering from obesity is human, such as an adult human or a paediatric human (including infants, children, and adolescents). Body mass index (BMI) is a measure of body fat based on height and weight. The formula for calculation is BMI=weight in kilograms/(height in meters)². A human subject suffering from obesity may have a BMI of ≧30; this subject may also be referred to as obese. In some embodiments the human subject suffering from obesity may have a BMI of ≧35 or a BMI in the range of ≧30 to <40. In some embodiments the obesity is severe obesity or morbid obesity, wherein the human subject may have a BMI of ≧40.

In some embodiments the invention relates to a method for treatment or prevention of overweight, optionally in the presence of at least one weight-related comorbidity. In some embodiments the invention relates to use of the derivative or the peptide of the invention for treatment or prevention of overweight, optionally in the presence of at least one weight-related comorbidity. In some embodiments the subject suffering from overweight is human, such as an adult human or a paediatric human (including infants, children, and adolescents). In some embodiments a human subject suffering from overweight may have a BMI of ≧25, such as a BMI of ≧27. In some embodiments a human subject suffering from overweight has a BMI in the range of 25 to <30 or in the range of 27 to <30. In some embodiments the weight-related comorbidity is selected from the group consisting of hypertension, diabetes (such as type 2 diabetes), dyslipidaemia, high cholesterol, and obstructive sleep apnoea.

In some embodiments the invention relates to a method for reduction of body weight. In some embodiments the invention relates to use of the derivative or the peptide of the invention for reduction of body weight. A human to be subjected to reduction of body weight according to the present invention may have a BMI of ≧25, such as a BMI of ≧27 or a BMI of ≧30. In some embodiments the human to be subjected to reduction of body weight according to the present invention may have a BMI of ≧35 or a BMI of ≧40. The term “reduction of body weight” may include treatment or prevention of obesity and/or overweight.

In some embodiments the derivative or peptide of the invention is for use in adults with a body mass index (BMI) of 30 or greater (obesity), or adults with a BMI of 27 or greater (overweight) who have at least one weight-related condition such as hypertension, type 2 diabetes, or dyslipidaemia. In some embodiments the derivative of the invention (alone or in combination with other weight reducing agents) is a treatment option for chronic weight management in addition to diet and exercise.

In some embodiments the derivative or the peptide of the invention may be used as monotherapy, for chronic weight management.

In some embodiments the derivative or the peptide of the invention may be used in combination with other weight reducing agents, for chronic weight management.

In some embodiments, the use in monotherapy is in addition to diet and exercise.

In some embodiments, the use in combination with other weight reducing agents is in addition to diet and exercise.

In some embodiments the administration is once daily.

In some embodiments the administration is once weekly.

In some embodiments the administration is once monthly.

### Combinations—Other Weight Reducing Agents

In the combined use of the derivative or the peptide of the invention with other weight reducing agents the combination may be effectuated in any suitable way.

In some non-limiting embodiments the derivative or the peptide and the desired agent(s) are (a) co-administered as a mixture; (b) co-administered as a co-formulated composition; and/or (c) administered as separate formulations either (i) substantially simultaneously, and/or (ii) with suitable time interval(s).

In the combined use of the derivative or the peptide of the invention with other weight reducing agents any other weight reducing agent may be used.

In some non-limiting embodiments the derivative or the peptide of the invention is combined with (a) at least one other weight reducing agent, (b) one or more other weight reducing agents, (c) one or two other weight reducing agents, (d) one other weight reducing agent, and/or (e) two other weight reducing agents.

Weight reducing agents are defined as compounds which when administered results in a weight loss (negative delta body weight) in at least one relevant in vivo study.

In some non-limiting embodiments the relevant in vivo study is an animal study, and/or a clinical trial with human beings.

In some embodiments the animals/human beings subjected to the study/trial are healthy. In some embodiments the animals/human beings subjected to the study/trial are overweight or obese.

In some embodiments the animal study is performed generally as described in any of Examples 94 and 97 herein. In some embodiments the animal study is performed generally as described in Example 96 herein.

Non-limiting examples of other weight reducing agents for the combined use with the derivatives or derivatives of the invention are GLP-1 receptor agonists, amylin receptor agonists, leptin receptor agonists, PYY receptor agonists, MIC-1 receptor agonists, and/or glucagon receptor agonists.

Additional non-limiting examples of other weight reducing agents for combined use with the derivatives or the peptides of the invention are estrogene, bupropion+naltrexone (Contrave®/Mysimba®), Cetilistat®, locaserin (Belviq®), phentermine+topira mate (Qsymia®), phentermine, orlistat (Xenical®/Alli®), and/or canagliflozin+phentermine.

A receptor agonist may be defined as an analogue that binds to a receptor and elicits a response typical of the natural ligand. A full agonist may be defined as one that elicits a response of the same magnitude as the natural ligand (see e.g. “Principles of Biochemistry”, A L Lehninger, D L Nelson, M M Cox, Second Edition, Worth Publishers, 1993, page 763). Thus, for example, a “GLP-1 receptor agonist” may be defined as a compound which is capable of binding to the GLP-1 receptor and capable of activating it. And a “full” GLP-1 receptor agonist may be defined as a GLP-1 receptor agonist which is capable of eliciting a magnitude of GLP-1 receptor response that is similar to native GLP-1.

In some embodiments the other weight reducing agent for use in combination with the derivative or the peptide of the invention is an agonist of the relevant receptor. In some embodiments it is a full agonist of the relevant receptor.

In the combined use of the derivative or the peptide of the invention with other weight reducing agents, one non-limiting example of another weight reducing agent is a GLP-1 receptor agonist. Any GLP-1 receptor agonist may be used.

As explained above, a GLP-1 receptor agonist is capable of binding to and activating the GLP-1 receptor which may be determined in any suitable way. In some non-limiting embodiments the binding to the GLP-1 receptor is determined using the assay of Example 36 in WO 2014/202727 A1. In some embodiments the Example 36 assay is performed (a) in a low concentration of serum albumin, and/or (b) in a higher concentration of serum albumin, both as defined in Example 36. In some non-limiting embodiments the activation of the GLP-1 receptor is determined using the assay of Example 35 in WO 2014/202727 A1.

In some embodiments the GLP-1 receptor agonist is a derivative of a GLP-1 analogue. In some embodiments the derivative is mono-, or di-acylated. In some embodiments the GLP-1 analogue has a maximum of 10 amino acid changes as compared to native GLP-1(7-37). In some embodiments the derivative has a side chain attached to the epsilon amino group of one or two Lys residues of the GLP-1 analogue. In some embodiments, a side chain is attached to the epsilon amino group of a Lys residue at a position corresponding to position a) 26, b) 27, c) 36, d) 37, e) 38, and/or f) 42 of GLP-1(7-37). In some embodiments, one side chain only is attached to the epsilon-amino group of a Lys residue at a position corresponding to a) position 26, or d) position 37 of GLP-1(7-37). In some embodiments, two identical side chains are attached to the epsilon-amino group of two Lys residues at positions corresponding to positions 26,37; 27,36; or 38,42 of GLP-1(7-37). In some embodiments, the side chain comprises a protracting moiety selected from a C16 fatty acid group (CH₃—(CH₂)₁₄—CO—), a C18 or C20 alpha,omega fatty di-acid group (HOOC—(CH₂)ₓ—CO— where x is 16 or 18), or a 4-COOH-PhO-C10 acid group (HOOC—C₆H₅—O—(CH₂)_{y}—CO— where y is 9 and HOOC—C₆H₅—O is carboxy phenoxy). In some embodiments, the protracting moiety is connected to epsilon-Lys via a linker. In some embodiments the linker comprises gGlu (Chem. 2 herein), Ado (Chem. 3a herein), Trx (Chem. 4 of WO 2015/000942 A1), or eps-Lys (Chem. 3 of WO 2012/062804 A1 where q is 4 and w is 0).

Non-limiting examples of such GLP-1 receptor agonists are disclosed in, i.a., the patent applications referred to above as well as in WO 98/08871 A1, WO 2006/097537 A1, WO 2009/030771 A1, WO 2011/080103 A1, and WO 2012/140117 A1, all of which are incorporated by reference herein.

In some embodiments the GLP-1 agonist is liraglutide. In some embodiments the GLP-1 agonist is Victoza® liraglutide. In some embodiments the GLP-1 agonist is liraglutide 3 mg (Saxenda®). In some embodiments the GLP-1 agonist is semaglutide (the compound of Example 4 of WO 2006/097537 A1).

Additional non-limiting embodiments of a GLP-1 receptor agonist are exenatide (Byetta®), exenatide once weekly (Bydureon®), lixisenatide (Lyxumia®), albiglutide (Eperzan®/Tanzeum®), and dulaglutide (Trulicity®).

Non-limiting examples of suitable other receptor agonists are disclosed in WO2016/034604 A1, WO2012/168430 A1, WO2012/168431 A1, and WO2012/168432 A1 (amylin receptor agonists); WO 2012/101124 A1 (leptin receptor agonists); WO2015/071355 A1 and WO2015/071356 A1 (PYY receptor agonists); U.S. Pat. No. 9,272,019 B2 (MIC-1 receptor agonists); and WO 2014/170496 A1, WO 2016/055610 A1, WO 2015/124612 A1 (glucagon receptor agonists); which are all incorporated by reference herein.

### Particular Embodiments

The following are particular embodiments of the invention:

1. A peptide derivative of the general formula I:

P-L-X1-X2-X3-Gly-Trp-X6-DMeAsp-X8-NH₂ (formula I),

wherein P is Chem. 1:

HOOC—(CH₂)ₓ—CO—*, Chem. 1:

wherein x is an integer in the range of 12-18; L is absent, or L comprises at least one linker element of formula Chem. 2, Chem. 3, Chem. 4, Chem. 5, and/or Chem. 6:

*—NH—CH(COOH)—(CH₂)₂—CO—*, Chem. 2:

*—NH—(CH₂)₂—[O—(CH₂)₂]ₖ—O—[CH₂]ₙ—CO—*, Chem. 3:

wherein k is an integer in the range of 1-11, and n is an integer in the range of 1-5,

*—NH—CH(CH₂OH)—CO—*, Chem. 4:

*—NH—CH₂—CO—*, Chem. 5:

and/or

*—NH—CH[(CH₂)₄—NH₂]—CO—*; Chem. 6:

X1 is absent, Asp, DAsp, bAsp, DbAsp, Glu, or DGlu;
X2 is Phe(4-sulfomethyl) or sTyr;

### X3 is Nle, Leu, Ala, Ile, Lys, Arg, Pro, Met, Phe, Ser, His, or Val;

### X6 is Nle, Ile, Gln, Met, Met(O2), Leu, Val, Pro, Hpg, or Ala;

X8 is Phe, MePhe, 1Nal, Me1Nal, 2Nal, Me2Nal, Trp, or MeTrp; and the NH₂ group at the C-terminus of formula I means that X8 is C-terminally amidated; or a pharmaceutically acceptable salt, amide, or ester thereof.
2. The derivative of embodiment 1, wherein P and L are connected via an amide bond; or if L is absent P and X1 are connected via an amide bond; or if L is absent and X1 is absent, P and X2 are connected via an amide bond.
3. The derivative of any of embodiments 1-2, wherein L is connected to X1 via an amide bond, or if X1 is absent L is connected to X2 via an amide bond.
4. The derivative of any of embodiments 1-3, wherein X1, X2, X3, Gly, Trp, X6, DMeAsp, and X8 are mutually connected via amide bonds; or if X1 is absent X2, X3, Gly, Trp, X6, DMeAsp, and X8 are mutually connected via amide bonds.
5. The derivative of any of embodiments 1-4, wherein L comprises at least one linker element of formula Chem. 2, Chem. 3, Chem. 4, Chem. 5, and/or Chem. 6:

*—NH—CH(COOH)—(CH₂)₂—CO—*, Chem. 2:

*—NH—(CH₂)₂—[O—(CH₂)₂]ₖ—O—[CH₂]ₙ—CO—*, Chem. 3:

wherein k is an integer in the range of 1-11, and n is an integer in the range of 1-5,

*—NH—CH(CH₂OH)—CO—*, Chem. 4:

*—NH—CH₂—CO—*, Chem. 5:

and/or

*—NH—CH[(CH₂)₄—NH₂]—CO—*. Chem. 6:

6. The derivative of any of embodiments 1-5, wherein L comprises at least two linker elements.
7. The derivative of any of embodiments 1-6, wherein L comprises at least three linker elements.
8. The derivative of any of embodiments 1-7, wherein L comprises at least four linker elements.
9. The derivative of any of embodiments 1-8, wherein L comprises at least five linker elements.
10. The derivative of any of embodiments 1-9, wherein L comprises at least six linker elements.
11. The derivative of any of embodiments 1-10, wherein L comprises two linker elements.
12. The derivative of any of embodiments 1-10, wherein L comprises three linker elements.
13. The derivative of any of embodiments 1-10, wherein L comprises four linker elements.
14. The derivative of any of embodiments 1-10, wherein L comprises five linker elements.
15. The derivative of any of embodiments 1-10, wherein L comprises six linker elements.
16. The derivative of any of embodiments 1-15, wherein L comprises Chem. 2.
17. The derivative of any of embodiments 1-16, wherein Chem. 2 is represented by Chem. 2a:

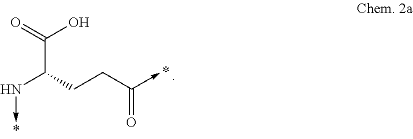

18. The derivative of any of embodiments 1-17, wherein L comprises at least one linker element of Chem. 2.
19. The derivative of any of embodiments 1-18, wherein L comprises at least two linker elements of Chem. 2.
20. The derivative of any of embodiments 1-19, wherein L comprises at least three linker elements of Chem. 2.
21. The derivative of any of embodiments 1-18, wherein L comprises one linker element of Chem. 2.
22. The derivative of any of embodiments 1-19, wherein L comprises two linker elements of Chem. 2.
23. The derivative of any of embodiments 1-20, wherein L comprises three linker elements of Chem. 2.
24. The derivative of any of embodiments 1-23, wherein L comprises Chem. 3.
25. The derivative of any of embodiments 1-24, wherein n is 1.
26. The derivative of any of embodiments 1-25, wherein k is 1.
27. The derivative of any of embodiments 1-26, wherein Chem. 3 is represented by Chem. 3a:

*—NH—(CH₂)₂—O—(CH₂)₂—O—CH₂—CO—*. Chem. 3a:

28. The derivative of any of embodiments 1-24, wherein n is 2.
29. The derivative of any of embodiments 1-24 and 28, wherein k is 9.
30. The derivative of any of embodiments 1-24 and 28-29, wherein Chem. 3 is represented by Chem. 3b:

*—NH—(CH₂)₂—[O—(CH₂)₂]₉—O—[CH₂]₂—CO—*. Chem. 3b:

31. The derivative of any of embodiments 1-30, wherein L comprises at least one linker element of Chem. 3.
32. The derivative of any of embodiments 1-31, wherein L comprises at least two linker elements of Chem. 3.
33. The derivative of any of embodiments 1-32, wherein L comprises at least three linker elements of Chem. 3.
34. The derivative of any of embodiments 1-33, wherein L comprises at least four linker elements of Chem. 3.
35. The derivative of any of embodiments 1-31, wherein L comprises one time Chem. 3.
36. The derivative of any of embodiments 1-32, wherein L comprises two times Chem. 3.
37. The derivative of any of embodiments 1-33, wherein L comprises three times Chem. 3.
38. The derivative of any of embodiments 1-34, wherein L comprises four times Chem. 3.
39. The derivative of any of embodiments 1-38, wherein L comprises Chem. 4.
40. The derivative of any of embodiments 1-39, wherein Chem. 4 is represented by Chem. 4a:

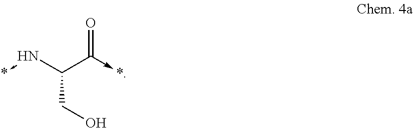

41. The derivative of any of embodiments 1-40, wherein L comprises at least one linker element of Chem. 4.
42. The derivative of any of embodiments 1-41, wherein L comprises at least two linker elements of Chem. 4.
43. The derivative of any of embodiments 1-42, wherein L comprises at least three linker elements of Chem. 4.
44. The derivative of any of embodiments 1-43, wherein L comprises at least four linker elements of Chem. 4.
45. The derivative of any of embodiments 1-44, wherein L comprises four linker elements of Chem. 4.
46. The derivative of any of embodiments 1-45, wherein L comprises Chem. 5.
47. The derivative of any of embodiments 1-46, wherein L comprises at least one linker element of Chem. 5.
48. The derivative of any of embodiments 1-47, wherein L comprises at least two linker elements of Chem. 5.
49. The derivative of any of embodiments 1-48, wherein L comprises two linker elements of Chem. 4.
50. The derivative of any of embodiments 1-49, wherein L comprises Chem. 6.
51. The derivative of any of embodiments 1-50, wherein Chem. 6 is represented by Chem. 6a:

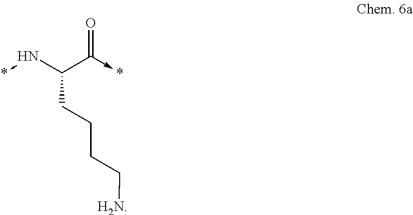

52. The derivative of any of embodiments 1-51, wherein L comprises at least one linker element of Chem. 6.
53. The derivative of any of embodiments 1-52, wherein L comprises one linker element of Chem. 6.
53a. The derivative of any of embodiments 1-53 which has the general formula I:

P-L-X1-X2-X3-Gly-Trp-X6-DMeAsp-X8-NH₂ (formula I),

wherein P is Chem. 1:

HOOC—(CH₂)ₓ—CO—*, Chem. 1:

wherein x is an integer in the range of 14-16; L comprises at least one linker element of formula Chem. 2, Chem. 3, and/or Chem. 6:

*—NH—CH(COOH)—(CH₂)₂—CO—*, Chem. 2:

*—NH—(CH₂)₂—[O—(CH₂)₂]ₖ—O—[CH₂]ₙ—CO—*, Chem. 3:

wherein k is an integer in the range of 1-11, and n is an integer in the range of 1-5, and/or

*—NH—CH[(CH₂)₄—NH₂]—CO—*; Chem. 6:

X1 is absent, Asp, DAsp, bAsp, DbAsp, Glu, or DGlu;
X2 is Phe(4-sulfomethyl) or sTyr;

### X3 is Nle, Leu, Ala, Ile, Lys, Arg, Pro, Met, Phe, Ser, His, or Val;

### X6 is Nle, Ile, Gln, Met, Met(O2), Leu, Val, Pro, Hpg, or Ala;

X8 is Phe, MePhe, 1Nal, Me1Nal, 2Nal, Me2Nal, Trp, or MeTrp; and the NH₂ group at the C-terminus of formula I means that X8 is C-terminally amidated; or a pharmaceutically acceptable salt, amide, or ester thereof.
53b. The derivative of embodiment 53a, which comprises at least one linker element of Chem. 2.
53c. The derivative of any of embodiments 53a-53b, wherein
X1 is absent, Asp, DAsp, bAsp, DbAsp, Glu, or DGlu;
X2 is Phe(4-sulfomethyl) or sTyr;

### X3 is Nle, Leu, Ala, Ile, Lys, Arg, Pro, Met, Phe, Ser, or His;

### X6 is Nle, Ile, Gln, Met, Met(O2), Leu, Val, Pro, or Ala;

### X8 is Phe, MePhe, 1Nal, Me1Nal, 2Nal, or Trp.

54. The derivative of any of embodiments 1-53c, wherein L comprises Chem. 2a and Chem. 3.
55. The derivative of any of embodiments 1-54, wherein L comprises Chem. 2a and Chem. 3a.
56. The derivative of any of embodiments 1-54, wherein L comprises Chem. 2a and Chem. 3b.
57. The derivative of any of embodiments 1-55, wherein L comprises one Chem. 2a linker element and two Chem. 3a linker elements.
58. The derivative of any of embodiments 1-55 and 57, wherein L consists of one Chem. 2a element and two Chem. 3a elements (Chem. 2a-2×Chem. 3a), interconnected via amide bonds and in the sequence indicated, L being connected at its *—NH end to the CO—* end of P, and at its CO—* end to the amino group of X1, or if X1 is absent to the amino group of X2.
59. The derivative of any of embodiments 1-55, wherein L comprises one Chem. 2a linker element and two Chem. 3b linker elements.
60. The derivative of any of embodiments 1-55, 56, and 59, wherein L consists of one Chem. 2a element and two Chem. 3b elements (Chem. 2a-2×Chem. 3b), interconnected via amide bonds and in the sequence indicated, L being connected at its *—NH end to the CO—* end of P, and at its CO—* end to the amino group of X1, or if X1 is absent to the amino group of X2.
61. The derivative of any of embodiments 1-55, wherein L comprises two Chem. 2a linker elements and two Chem. 3a linker elements.
62. The derivative of any of embodiments 1-55, and 61, wherein L consists of two Chem. 2a elements and two Chem. 3a elements (2×Chem. 2a-2×Chem. 3a), interconnected via amide bonds and in the sequence indicated, L being connected at its *—NH end to the CO—* end of P, and at its CO—* end to the amino group of X1, or if X1 is absent to the amino group of X2.
63. The derivative of any of embodiments 1-55, wherein L comprises one Chem. 2a linker element and four Chem. 3a linker elements.
64. The derivative of any of embodiments 1-55, and 63, wherein L consists of one Chem. 2a element and four Chem. 3a elements (Chem. 2a-4×Chem. 3a), interconnected via amide bonds and in the sequence indicated, L being connected at its *—NH end to the CO—* end of P, and at its CO—* end to the amino group of X1, or if X1 is absent to the amino group of X2.
65. The derivative of any of embodiments 1-55, 61, and 63, wherein L consists of one Chem. 2a element, two Chem. 3a elements, and one Chem. 2a element (Chem. 2a-2×Chem. 3a-Chem. 2a), interconnected via amide bonds and in the sequence indicated, L being connected at its *—NH end to the CO—* end of P, and at its CO—* end to the amino group of X1, or if X1 is absent to the amino group of X2.
66. The derivative of any of embodiments 1-53c, wherein L comprises two Chem. 3a linker elements.
67. The derivative of any of embodiments 1-53c, and 66, wherein L consists of two Chem. 3a elements (2×Chem. 3a), interconnected via amide bonds and in the sequence indicated, L being connected at its *—NH end to the CO—* end of P, and at its CO—* end to the amino group of X1, or if X1 is absent to the amino group of X2.
68. The derivative of any of embodiments 1-53c, wherein L comprises three Chem. 2a linker elements.
69. The derivative of any of embodiments 1-53, and 68, wherein L consists of three Chem. 2a elements (3×Chem. 2a), interconnected via amide bonds and in the sequence indicated, L being connected at its *—NH end to the CO—* end of P, and at its CO—* end to the amino group of X1, or if X1 is absent to the amino group of X2.
70. The derivative of any of embodiments 1-53, wherein L comprises Chem. 5 and Chem. 4a.
71. The derivative of any of embodiments 1-53, and 70, wherein L consists of Chem. and Chem. 4a elements.
72. The derivative of any of embodiments 1-53, and 70-71, wherein L consists of linker elements (Chem. 5-Chem. 4a-Chem. 4a-Chem. 5-Chem. 4a-Chem. 4a), interconnected via amide bonds and in the sequence indicated, L being connected at its *—NH end to the CO—* end of P, and at its CO—* end to the amino group of X1, or if X1 is absent to the amino group of X2.
73. The derivative of any of embodiments 1-53c, wherein L comprises Chem. 2a, Chem. 3a, Chem. 6a.
74. The derivative of any of embodiments 1-53c, and 73, wherein L consists of Chem. 2a, Chem. 3a, and Chem. 6a elements.
75. The derivative of any of embodiments 1-53c, and 73-74, wherein L consists of linker elements (Chem. 2a-2×Chem. 3a-Chem. 6a-Chem. 3a), interconnected via amide bonds and in the sequence indicated, L being connected at its *—NH end to the CO—* end of P, and at its CO—* end to the amino group of X1, or if X1 is absent to the amino group of X2.
76. The derivative of any of embodiments 1-75, wherein x is 12, 14, 16, or 18.
77. The derivative of any of embodiments 1-76, wherein x is an integer in the range of 14-18.
78. The derivative of any of embodiments 1-77, wherein x is 14, 16, or 18.
79. The derivative of any of embodiments 1-78, wherein x is 14.
80. The derivative of any of embodiments 1-78, wherein x is 16.
81. The derivative of any of embodiments 1-78, wherein x is 18.
82. The derivative of any of embodiments 1-81, wherein
X1 is absent, Asp, DAsp, bAsp, DbAsp, Glu, or DGlu;
X2 is Phe(4-sulfomethyl) or sTyr;

### X3 is Nle, Leu, Ala, Ile, Lys, Arg, Pro, Met, Phe, Ser, or His;

### X6 is Nle, Ile, Gln, Met, Met(O2), Leu, Val, Pro, or Ala; and

### X8 is Phe, MePhe, 1Nal, Me1Nal, 2Nal, or Trp.

83. The derivative of any of embodiments 1-82, wherein X1 is absent.
84. The derivative of any of embodiments 1-82, wherein X1 is Asp.
85. The derivative of any of embodiments 1-82, wherein X1 is DAsp.
86. The derivative of any of embodiments 1-82, wherein X1 is bAsp.
87. The derivative of any of embodiments 1-82, wherein X1 is Glu.
88. The derivative of any of embodiments 1-82, wherein X1 is DGlu.
89. The derivative of any of embodiments 1-88, wherein X2 is Phe(4-sulfomethyl).
90. The derivative of any of embodiments 1-88, wherein X2 is sTyr.
91. The derivative of any of embodiments 1-90, wherein X3 is Nle or Leu.
92. The derivative of any of embodiments 1-91, wherein X3 is Nle.
93. The derivative of any of embodiments 1-91, wherein X3 is Leu.
94. The derivative of any of embodiments 1-90, wherein X3 is Ala.
95. The derivative of any of embodiments 1-90, wherein X3 is Ile.
96. The derivative of any of embodiments 1-90, wherein X3 is Lys.
97. The derivative of any of embodiments 1-90, wherein X3 is Arg.
98. The derivative of any of embodiments 1-90, wherein X3 is Pro.
99. The derivative of any of embodiments 1-90, wherein X3 is Met.
100. The derivative of any of embodiments 1-90, wherein X3 is Phe.
101. The derivative of any of embodiments 1-90, wherein X3 is Ser.
102. The derivative of any of embodiments 1-90, wherein X3 is His.
103. The derivative of any of embodiments 1-102, wherein X6 is Nle.
104. The derivative of any of embodiments 1-102, wherein X6 is Ile.
105. The derivative of any of embodiments 1-102, wherein X6 is Gin.
106. The derivative of any of embodiments 1-102, wherein X6 is Met.
107. The derivative of any of embodiments 1-102, wherein X6 is Met(O2).
108. The derivative of any of embodiments 1-102, wherein X6 is Leu.
109. The derivative of any of embodiments 1-102, wherein X6 is Val.
110. The derivative of any of embodiments 1-102, wherein X6 is Pro.
111. The derivative of any of embodiments 1-102, wherein X6 is Ala.
112. The derivative of any of embodiments 1-111, wherein X8 is Phe, MePhe, 1Nal, Me1Nal, Trp, or 2Nal.
113. The derivative of any of embodiments 1-112, wherein X8 is Phe.
114. The derivative of any of embodiments 1-112, wherein X8 is MePhe.
115. The derivative of any of embodiments 1-112, wherein X8 is 1Nal.
116. The derivative of any of embodiments 112, wherein X8 is Me1Nal.
117. The derivative of any of embodiments 112, wherein X8 is Trp.
118. The derivative of any of embodiments 1-112, wherein X8 is 2Nal.
119. The derivative of any of embodiments 1-118, which has a maximum of six amino acid changes as compared to CCK-8s (SEQ ID NO: 1).
120. The derivative of any of embodiments 1-119, which has a maximum of five amino acid changes as compared to CCK-8s (SEQ ID NO: 1).
121. The derivative of any of embodiments 1-120, which has a maximum of four amino acid changes as compared to CCK-8s (SEQ ID NO: 1).
122. The derivative of any of embodiments 1-121, which has a maximum of three amino acid changes as compared to CCK-8s (SEQ ID NO: 1).
123. The derivative of any of embodiments 1-122, which has a maximum of two amino acid changes as compared to CCK-8s (SEQ ID NO: 1).
124. The derivative of any of embodiments 1-123, which has a minimum of two amino acid changes as compared to CCK-8s (SEQ ID NO: 1).
125. The derivative of any of embodiments 1-123, which has a minimum of three amino acid changes as compared to CCK-8s (SEQ ID NO: 1).
126. The derivative of any of embodiments 1-123, which has a minimum of four amino acid changes as compared to CCK-8s (SEQ ID NO: 1).
127. The derivative of any of embodiments 1-123, which has a minimum of five amino acid changes as compared to CCK-8s (SEQ ID NO: 1).
128. The derivative of any of embodiments 1-123, which has a minimum of six amino acid changes as compared to CCK-8s (SEQ ID NO: 1).
129. The derivative of any of embodiments 1-128, which has six amino acid changes as compared to CCK-8s (SEQ ID NO: 1).
130. The derivative of any of embodiments 1-128, which has five amino acid changes as compared to CCK-8s (SEQ ID NO: 1).
131. The derivative of any of embodiments 1-128, which has four amino acid changes as compared to CCK-8s (SEQ ID NO: 1).
132. The derivative of any of embodiments 1-128, which has three amino acid changes as compared to CCK-8s (SEQ ID NO: 1).
133. The derivative of any of embodiments 1-128, which has two amino acid changes as compared to CCK-8s (SEQ ID NO: 1).
134. A peptide derivative selected from the following:
2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Nle-Gly-Trp-Nle-DMeAsp-Trp-amide,

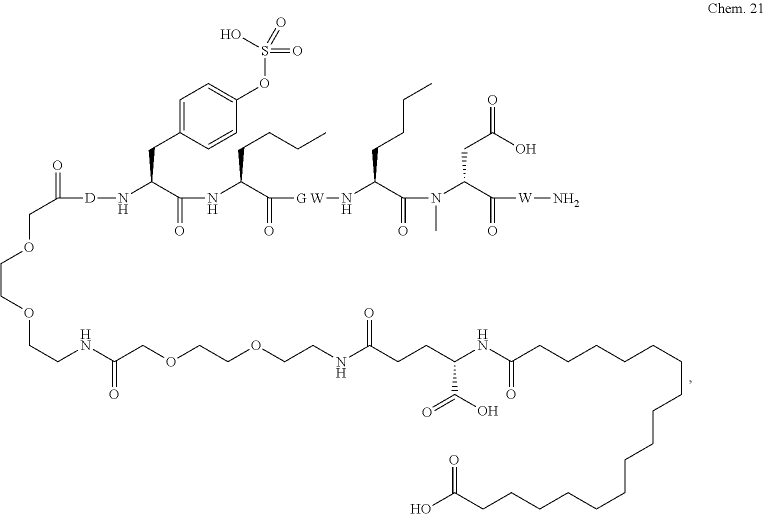

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Nle-Gly-Trp-Nle-DMeAsp-1Nal-amide,

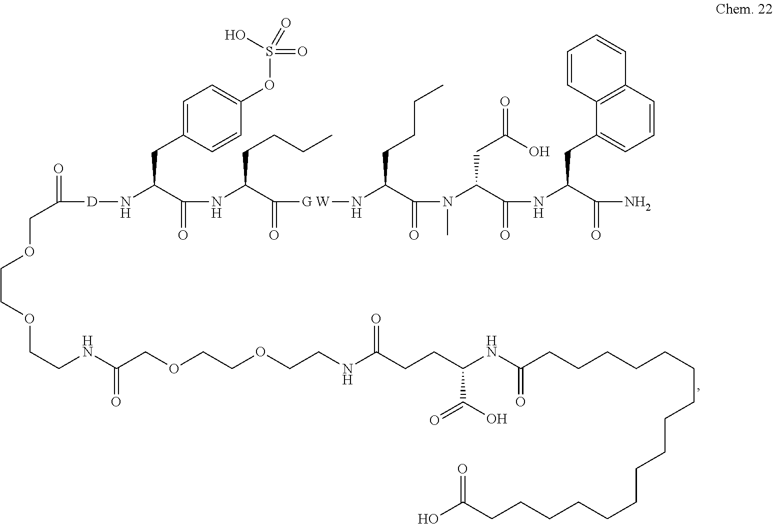

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-Phe(4-sulfomethyl)-Nle-Gly-Trp-Nle-DMeAsp-MePhe-amide,

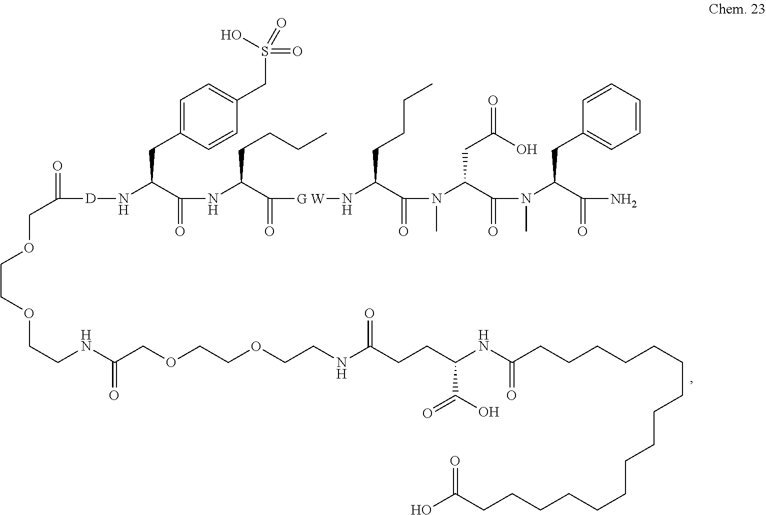

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Nle-DMeAsp-Me1Nal-amide,

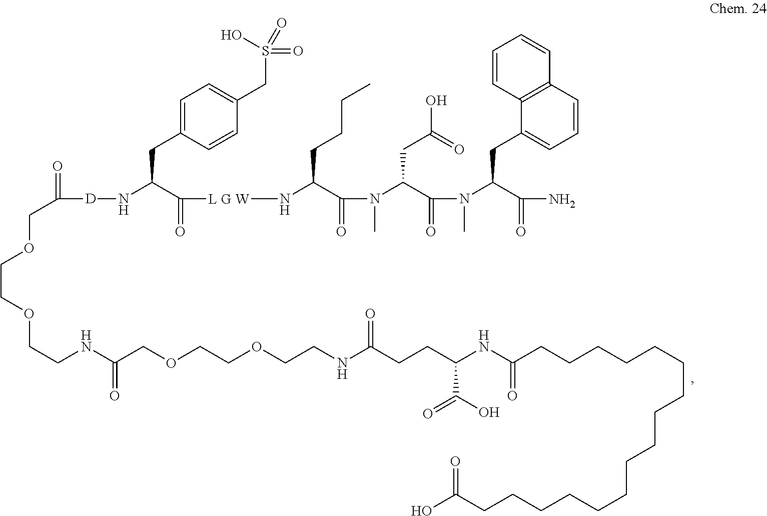

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-Phe(4-sulfomethyl)-Leu-Gly-Trp-Nle-DMeAsp-MePhe-amide,

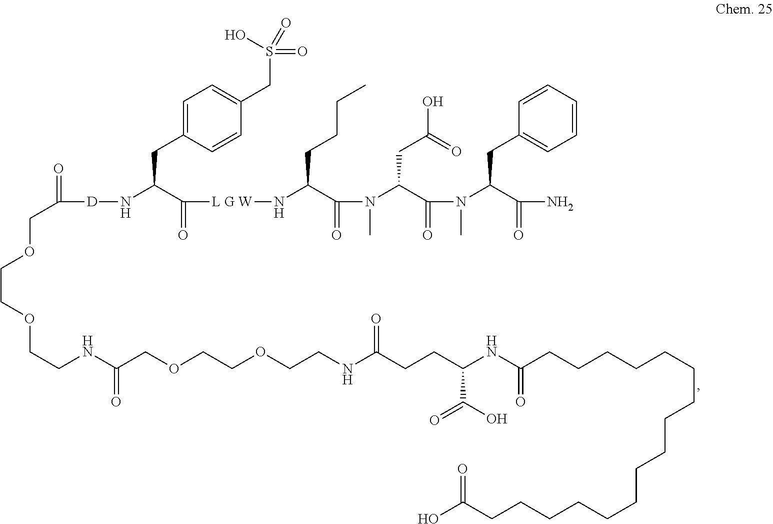

2-[2-[2-[[2-[2-[2-[[(4S)-4-Carboxy-4-(15-carboxypentadecanoylamino)butanoyl]amino]ethoxy]ethoxy]acetyl]amino]ethoxy]ethoxy]acetyl-Asp-Phe(4-sulfomethyl)-Leu-Gly-Trp-Nle-DMeAsp-MePhe-amide,

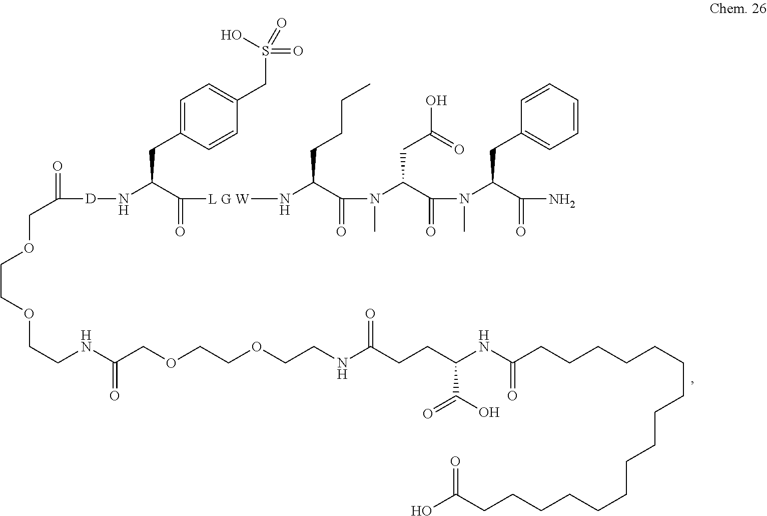

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Nle-DMeAsp-Phe-amide,

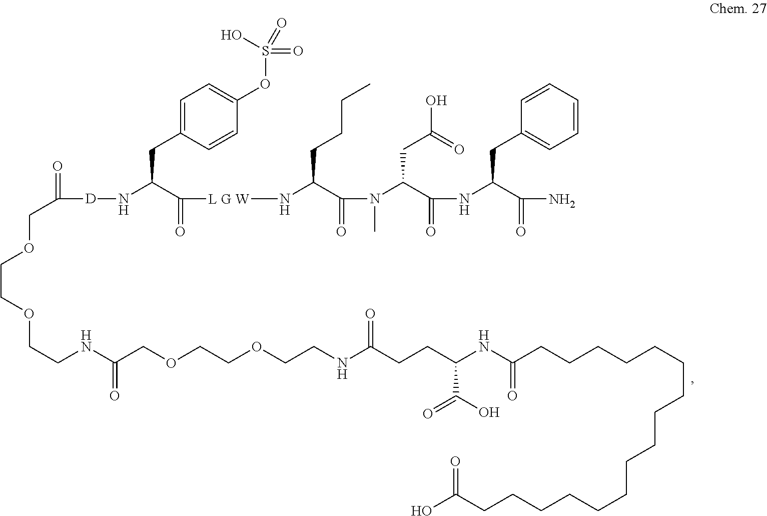

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Nle-Gly-Trp-Nle-DMeAsp-MePhe-amide,

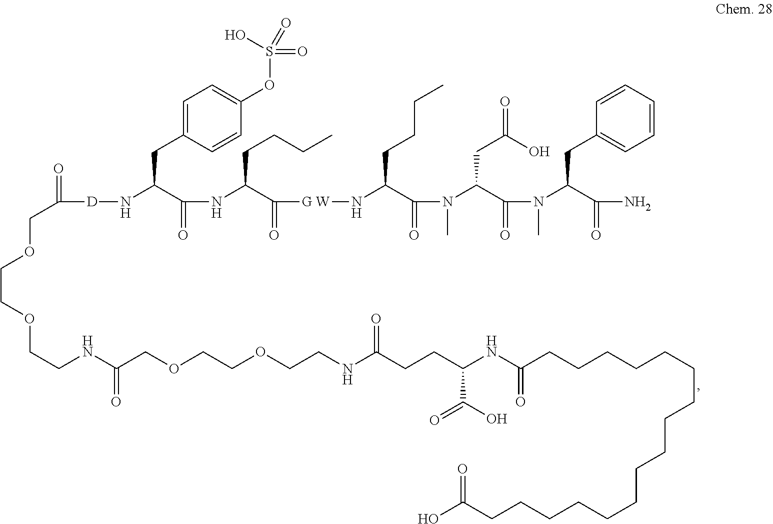

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Nle-DMeAsp-MePhe-amide,

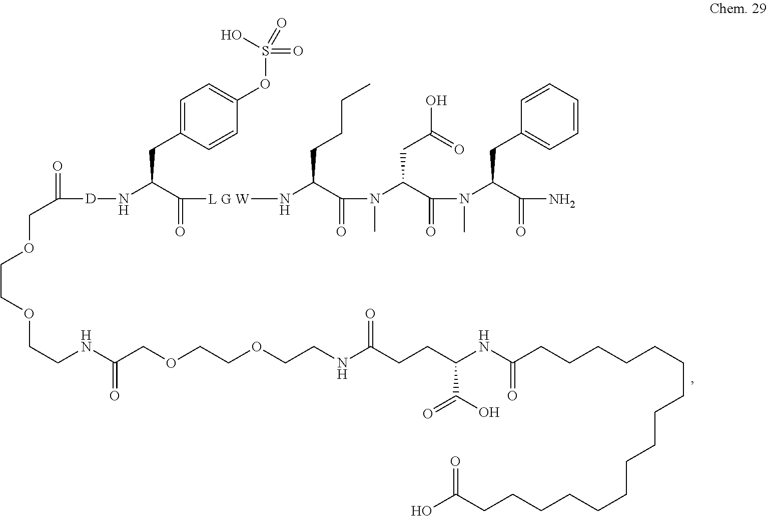

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Ile-DMeAsp-MePhe-amide,

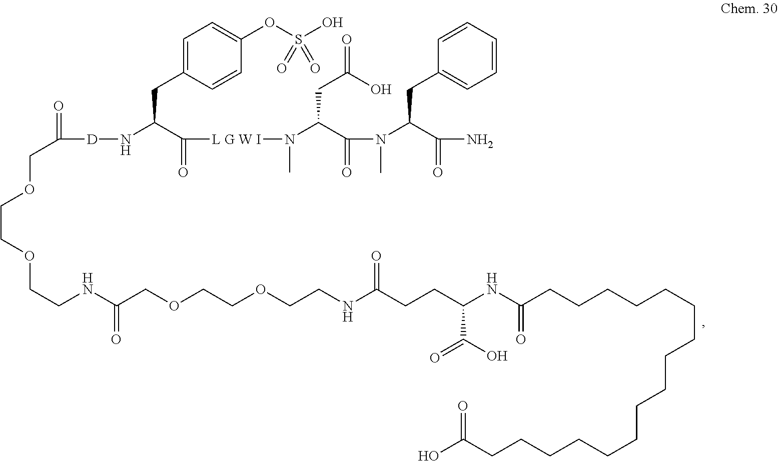

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Gln-DMeAsp-MePhe-amide,

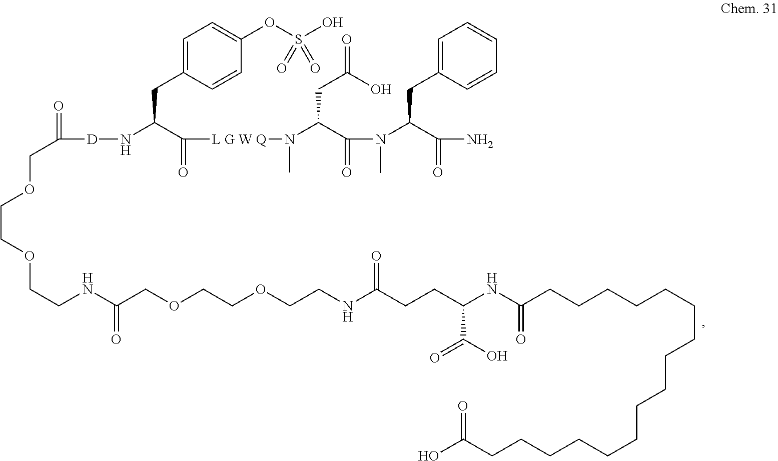

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Met(O2)-DMeAsp-MePhe-amide,

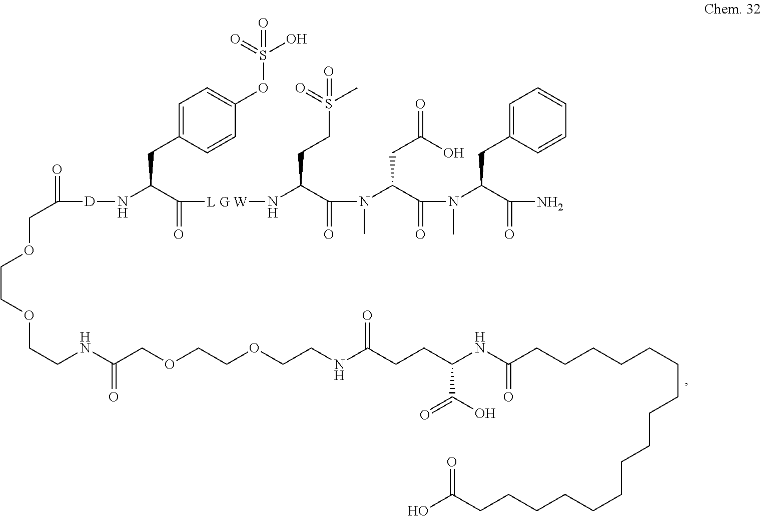

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Leu-DMeAsp-MePhe-amide,

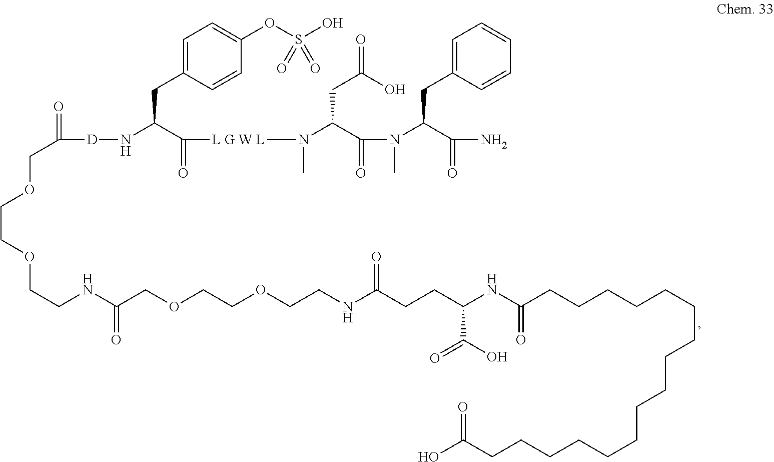

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Val-DMeAsp-MePhe-amide,

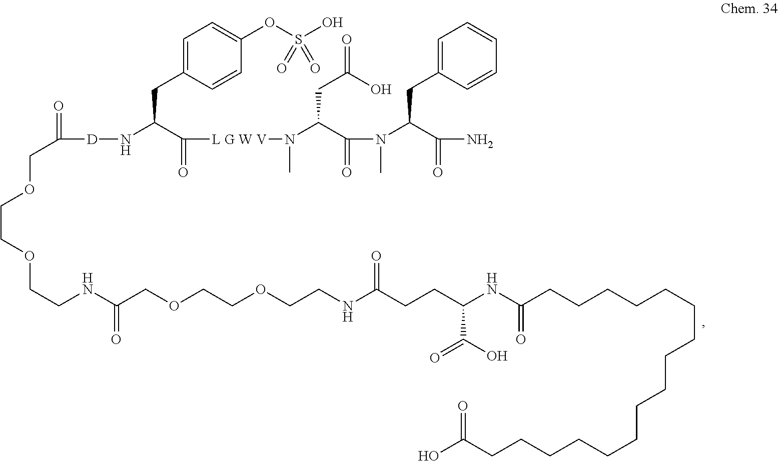

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Pro-DMeAsp-MePhe-amide,

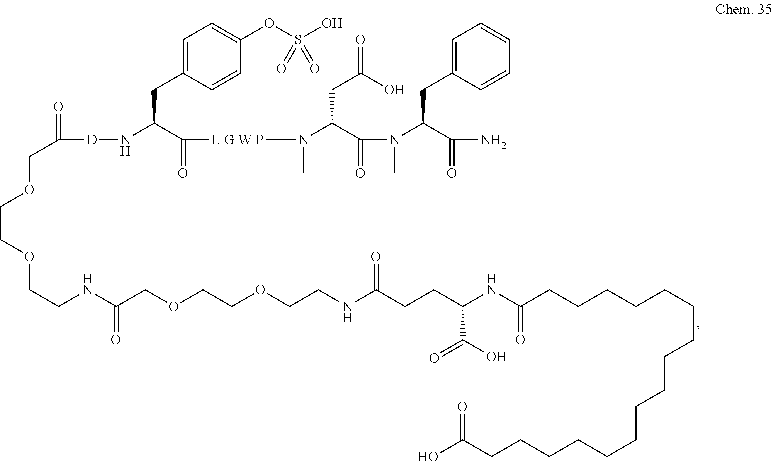

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Lys-Gly-Trp-Nle-DMeAsp-MePhe-amide,

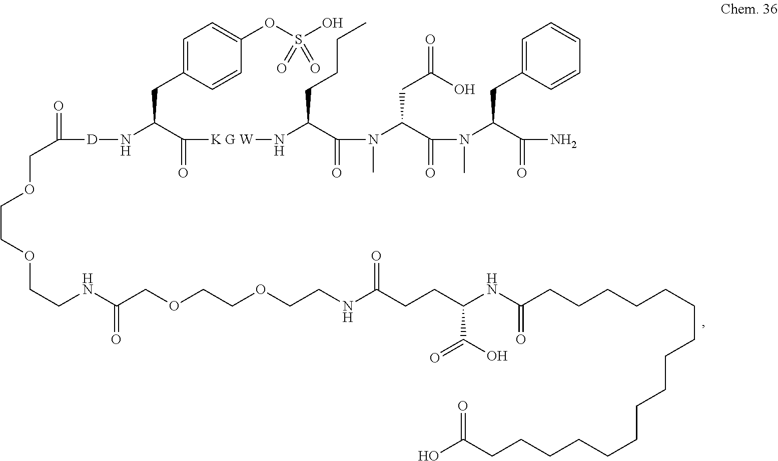

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Arg-Gly-Trp-Nle-DMeAsp-MePhe-amide,

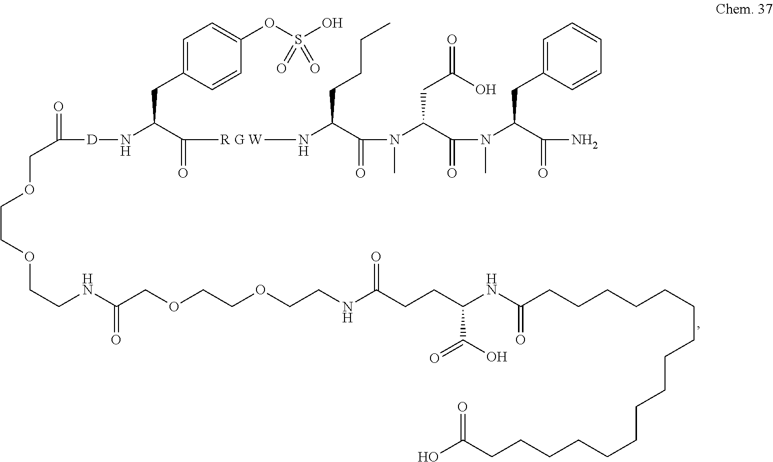

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Pro-Gly-Trp-Nle-DMeAsp-MePhe-amide,

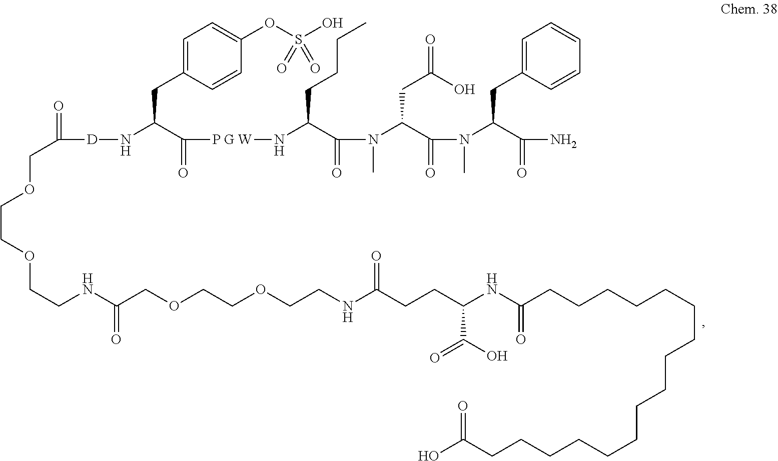

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Lys-2-[2-(2-aminoethoxy)ethoxy]acetyl-Asp-Phe(4-sulfomethyl)-Nle-Gly-Trp-Nle-DMeAsp-MePhe-amide,

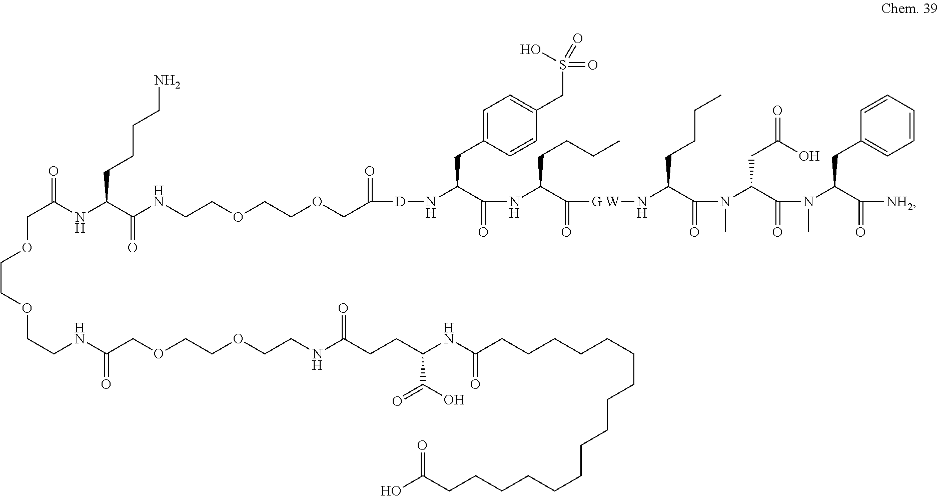

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Met-Gly-Trp-Nle-DMeAsp-MePhe-amide,

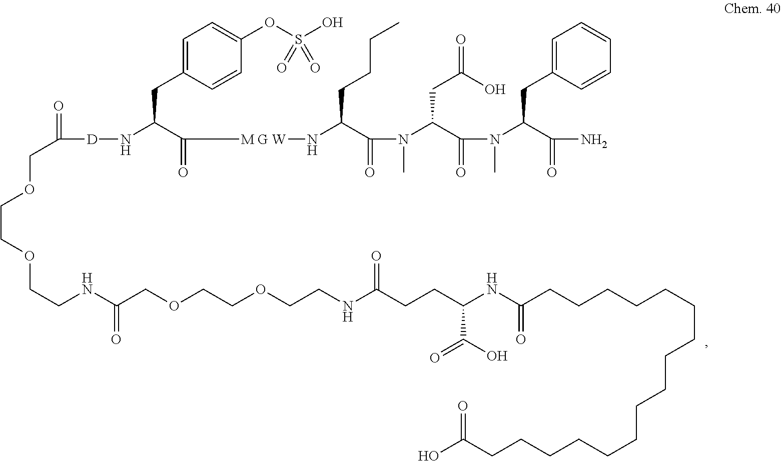

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Nle-Gly-Trp-Met-DMeAsp-MePhe-amide,

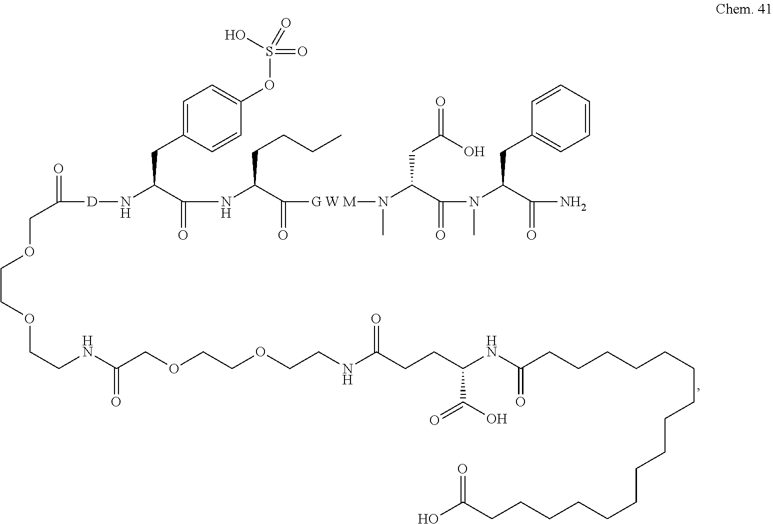

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-sTyr-Nle-Gly-Trp-Nle-DMeAsp-MePhe-amide,

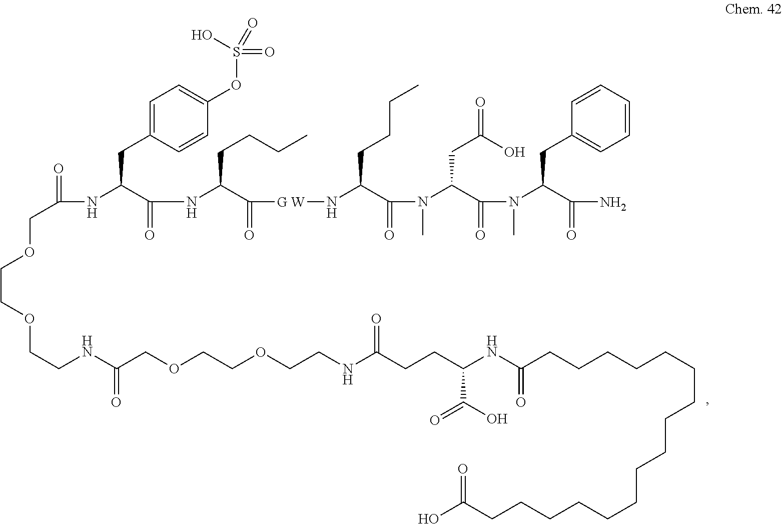

3-[2-[2-[2-[2-[2-[2-[2-[2-[2-[2-[3-[2-[2-[2-[2-[2-[2-[2-[2- [2-[2-[[(4S)-4-Carboxy-4-(17-carboxyheptadecanoylamino)butanoyl]amino]ethoxy]ethoxy]ethoxy]ethoxy]ethoxy]ethoxy]ethoxy]ethoxy]ethoxy]ethoxy]propanoylamino]ethoxy]ethoxy]ethoxy]ethoxy]ethoxy]ethoxy]ethoxy]ethoxy]ethoxy]ethoxy]propanoyl-Asp-sTyr-Nle-Gly-Trp-Nle-DMeAsp-MePhe-amide,

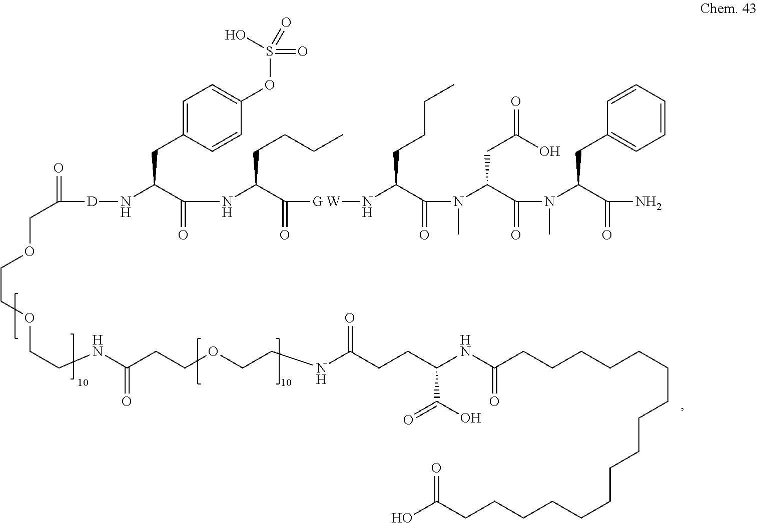

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Met-Gly-Trp-Met-DMeAsp-MePhe-amide,

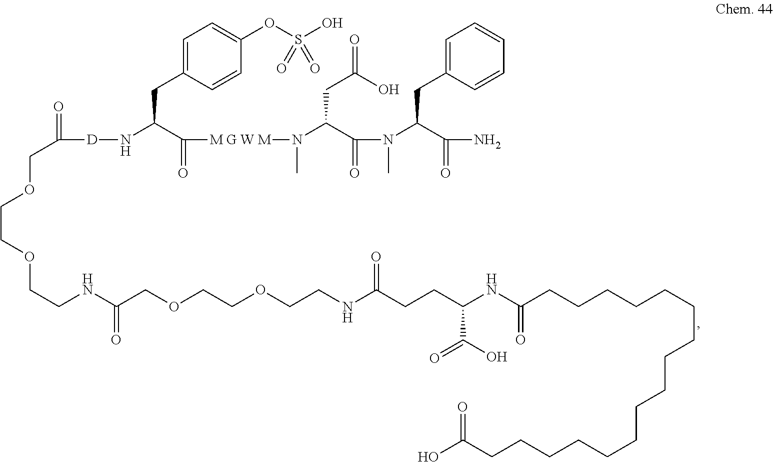

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-DAsp-Phe(4-sulfomethyl)-Nle-Gly-Trp-Nle-DMeAsp-MePhe-amide,

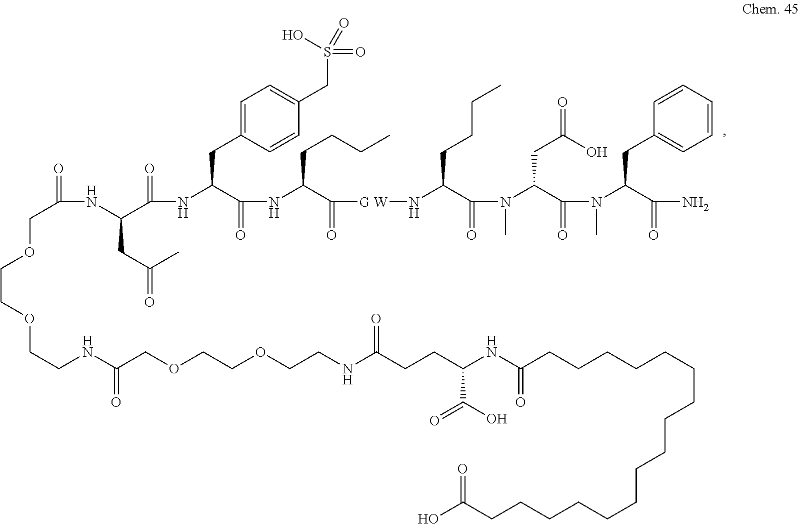

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-bAsp-Phe(4-sulfomethyl)-Nle-Gly-Trp-Nle-DMeAsp-MePhe-amide,

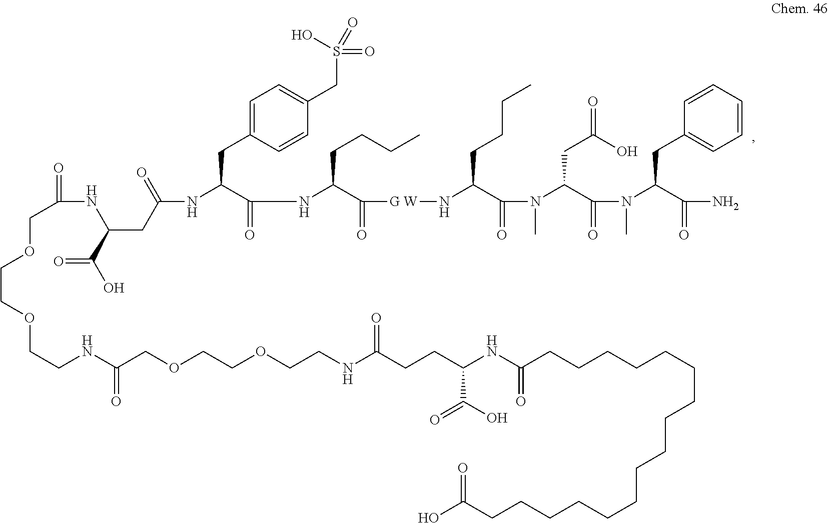

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-DbAsp-Phe(4-sulfomethyl)-Nle-Gly-Trp-Nle-DMeAsp-MePhe-amide,

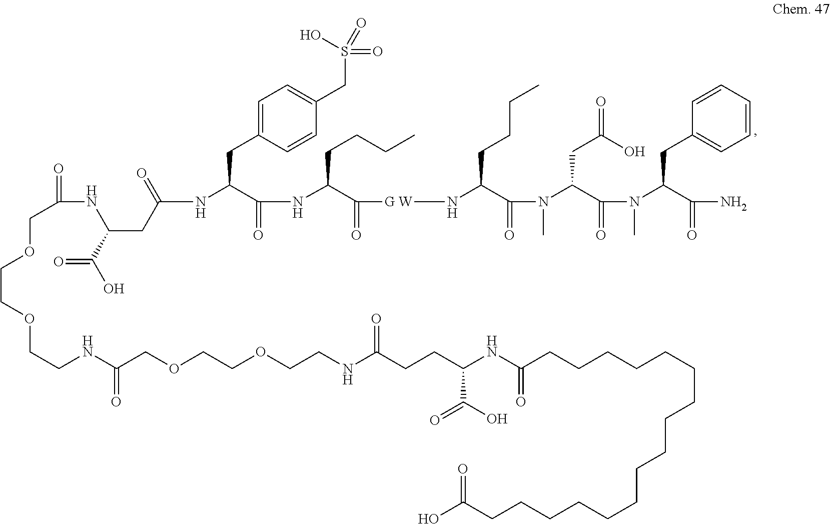

2-[2-[2-[[2-[2-[2-[[(4S)-4-Carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl-Asp-sTyr-Leu-Gly-Trp-Nle-DMeAsp-MePhe-amide,

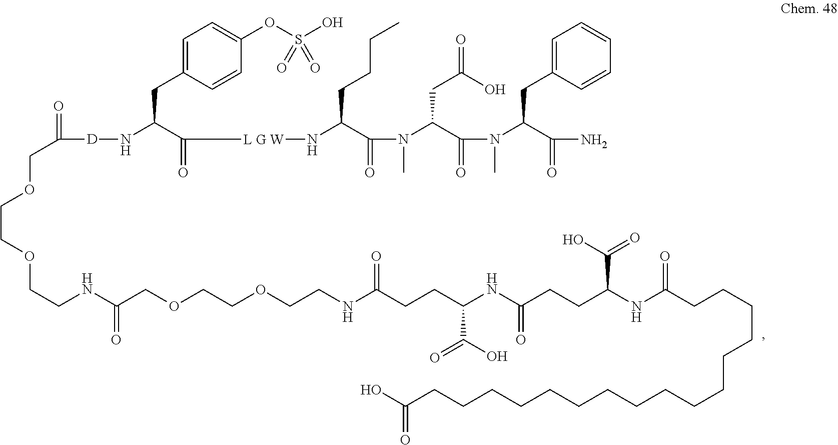

17-Carboxyheptadecanoyl-Gly-Ser-Ser-Gly-Ser-Ser-Asp-sTyr-Leu-Gly-Trp-Nle-DMeAsp-MePhe-amide,

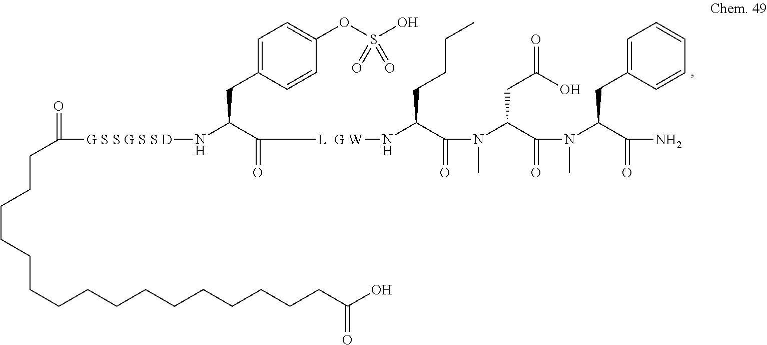

2-[2-[2-[[2-[2-[2-(17-Carboxyheptadecanoylamino)ethoxy]ethoxy]acetyl]amino]ethoxy]ethoxy]-Asp-sTyr-Leu-Gly-Trp-Nle-DMeAsp-MePhe-amide,

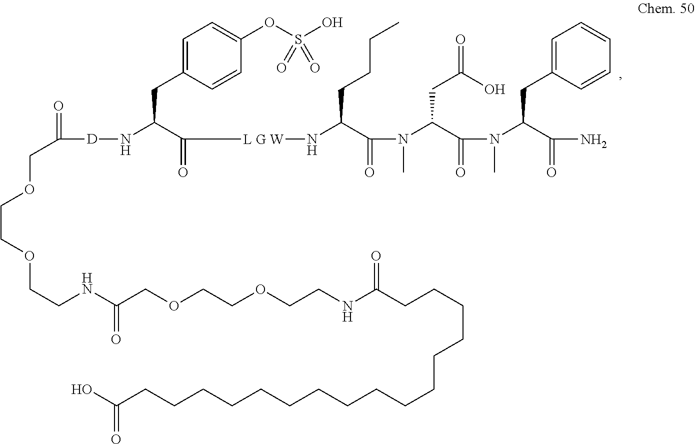

2-[2-[2-[[2-[2-[2-[[2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl]amino]ethoxy]ethoxy]acetyl]amino]ethoxy]ethoxy]acetyl-Asp-sTyr-Leu-Gly-Trp-Nle-DMeAsp-MePhe-amide,

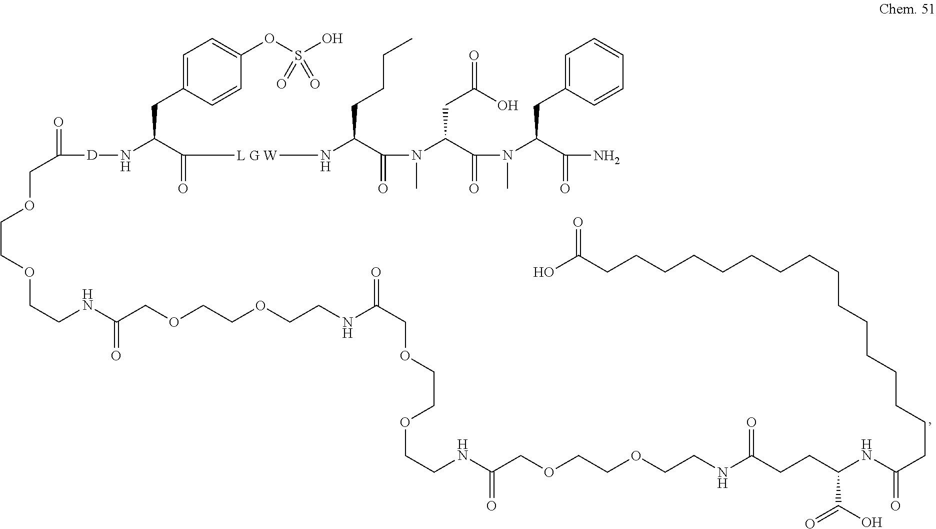

(4S)-4-Carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl-Asp-sTyr-Leu-Gly-Trp-Nle-DMeAsp-MePhe-amide,

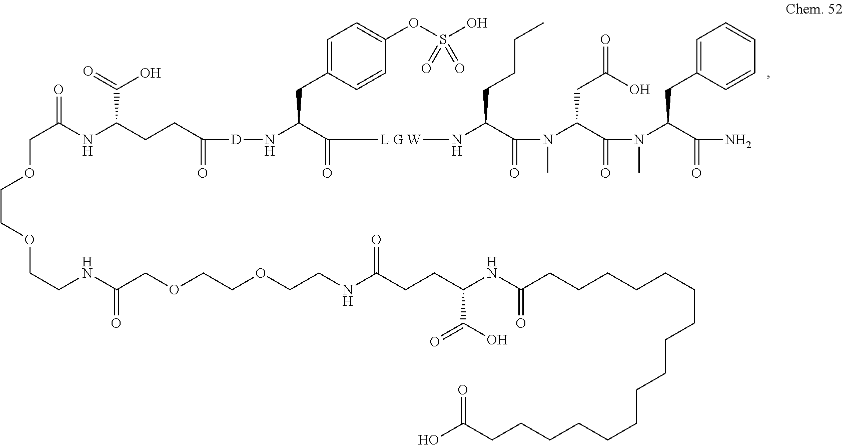

17-Carboxyheptadecanoyl-gGlu-gGlu-gGlu-Asp-sTyr-Leu-Gly-Trp-Nle-DMeAsp-MePhe-amide,

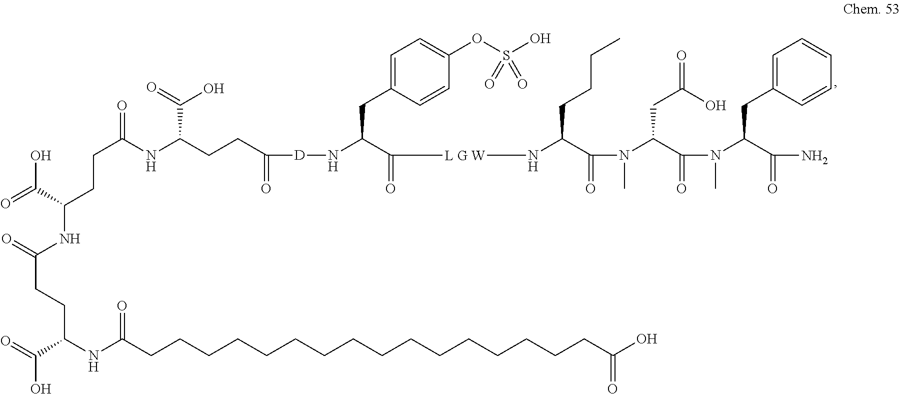

2-[2-[2-[[2-[2-[2-[[(4S)-4-Carboxy-4-(13-carboxytridecanoylamino)butanoyl]amino]ethoxy]ethoxy]acetyl]amino]ethoxy]ethoxy]acetyl-Asp-Phe(4-sulfomethyl)-Leu-Gly-Trp-Nle-DMeAsp-MePhe-amide,

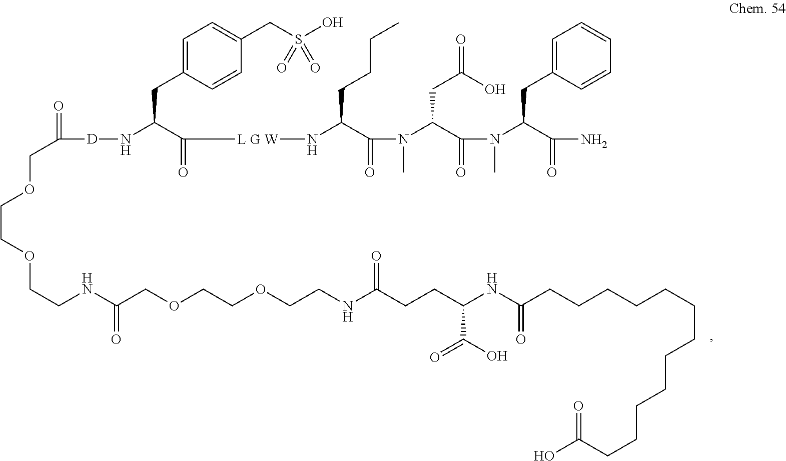

2-[2-[2-[[2-[2-[2-[[(4S)-4-Carboxy-4-(19-carboxynonadecanoylamino)butanoyl]amino]ethoxy]ethoxy]acetyl]amino]ethoxy]ethoxy]acetyl-Asp-Phe(4-sulfomethyl)-Leu-Gly-Trp-Nle-DMeAsp-MePhe-amide,

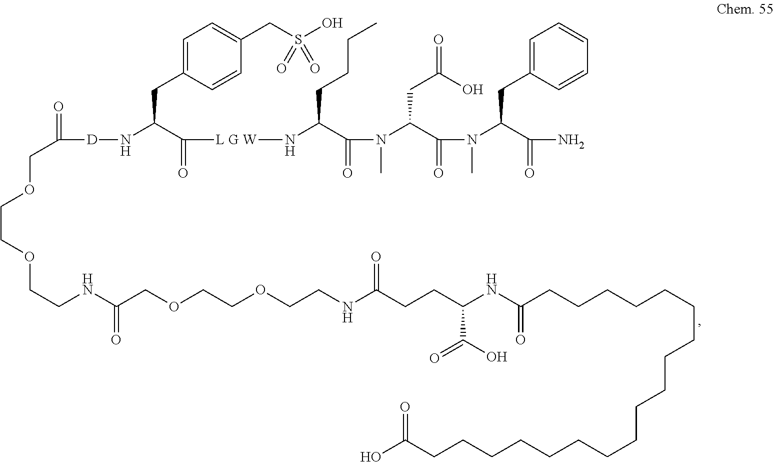

2-[2-[2-[[2-[2-[2-[[(4S)-4-Carboxy-4-(13-carboxytridecanoylamino)butanoyl]amino]ethoxy]ethoxy]acetyl]amino]ethoxy]ethoxy]acetyl-GIu-sTyr-Leu-Gly-Trp-Nle-DMeAsp-MePhe-amide,

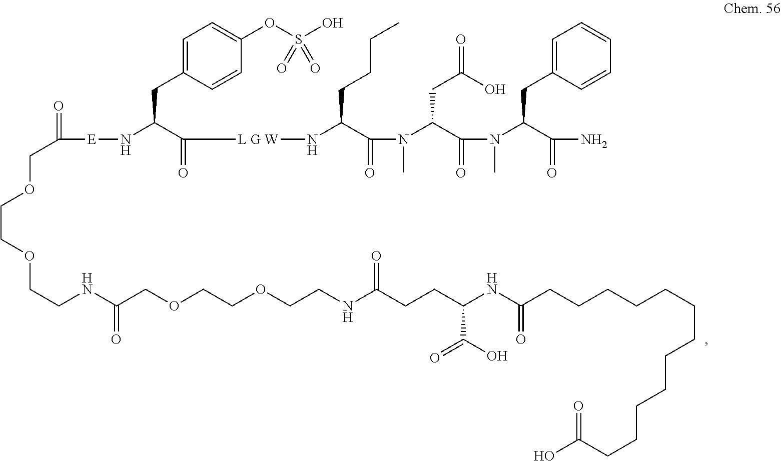

2-[2-[2-[[2-[2-[2-[[(4S)-4-Carboxy-4-(19-carboxynonadecanoylamino)butanoyl]amino]ethoxy]ethoxy]acetyl]amino]ethoxy]ethoxy]acetyl-Glu-sTyr-Leu-Gly-Trp-Nle-DMeAsp-MePhe-amide,

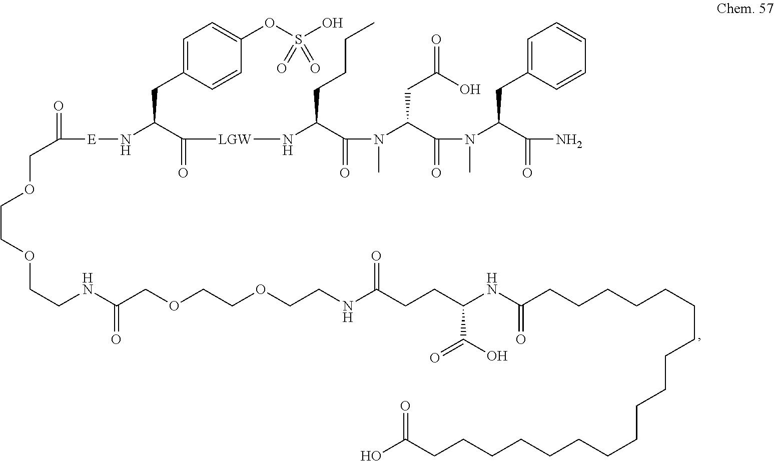

17-Carboxyheptadecanoyl-Asp-sTyr-Leu-Gly-Trp-Nle-DMeAsp-MePhe-amide,

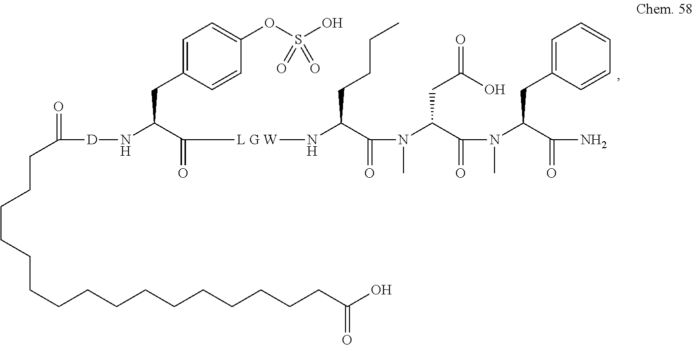

2-[2-[2-[[2-[2-[2-[[(4S)-4-Carboxy-4-(15-carboxypentadecanoylamino)butanoyl]amino]ethoxy]ethoxy]acetyl]amino]ethoxy]ethoxy]acetyl-Glu-sTyr-Leu-Gly-Trp-Nle-DMeAsp-MePhe-amide,

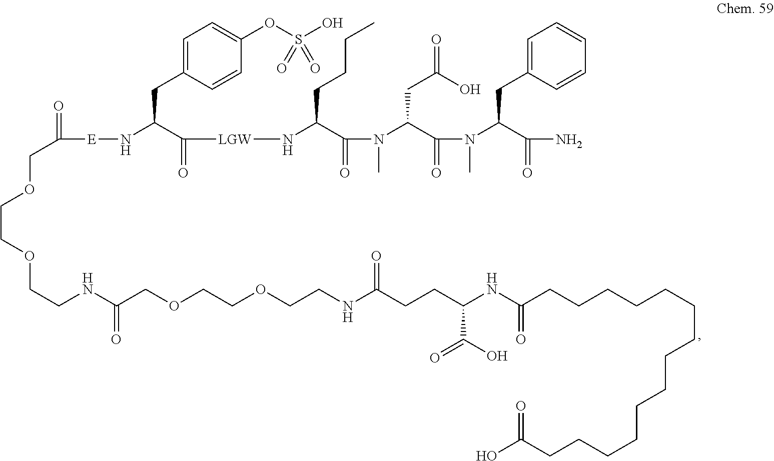

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Ala-DMeAsp-MePhe-amide,

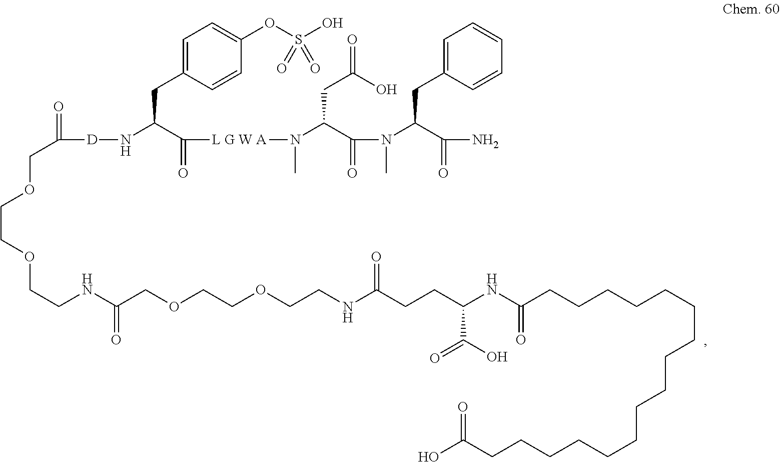

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Glu-sTyr-Leu-Gly-Trp-Gln-DMeAsp-Phe-amide,

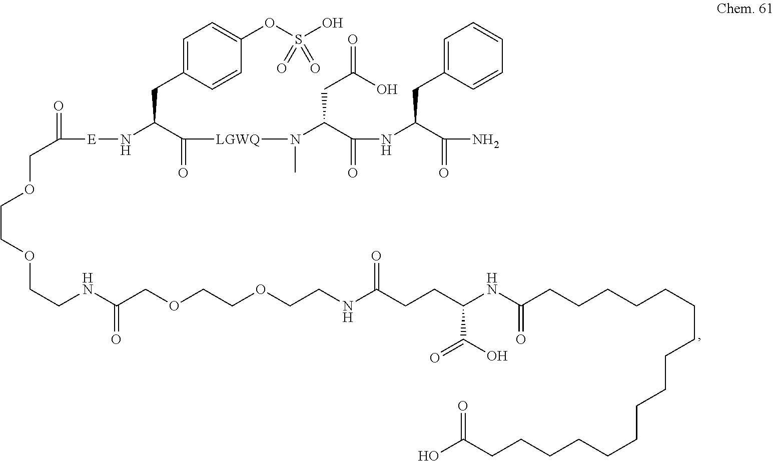

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Glu-sTyr-Leu-Gly-Trp-Gln-DMeAsp-MePhe-amide,

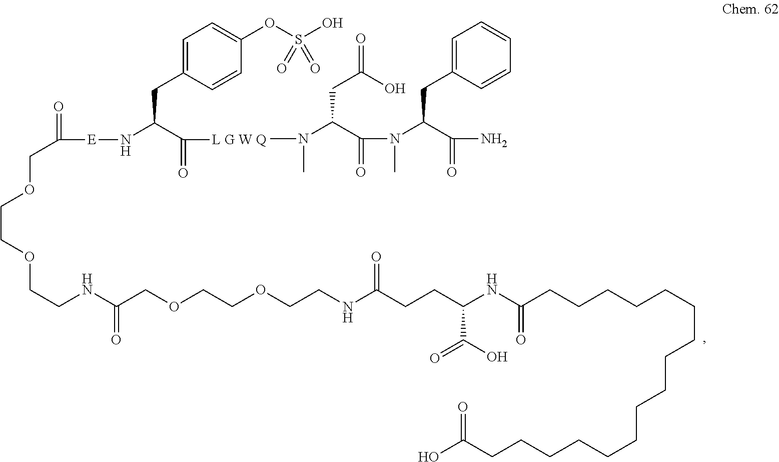

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Glu-sTyr-Leu-Gly-Trp-Nle-DMeAsp-MePhe-amide,

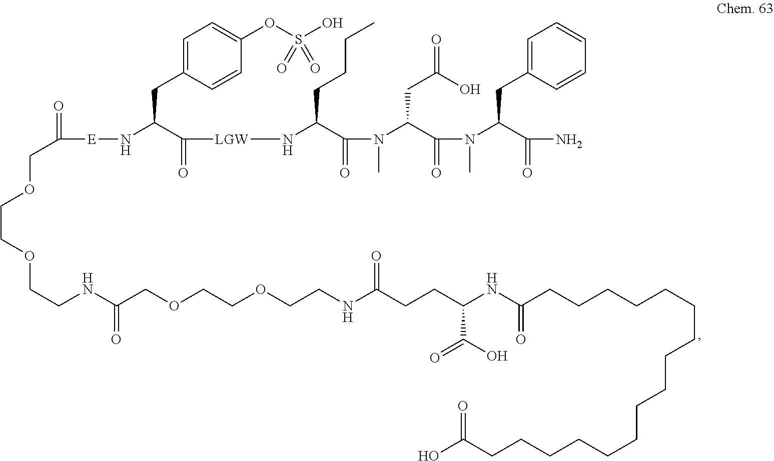

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Phe-Gly-Trp-Nle-DMeAsp-MePhe-amide,

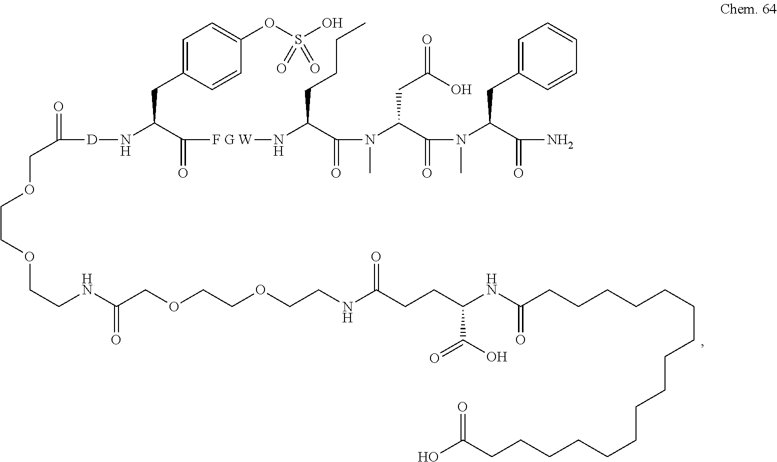

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Ser-Gly-Trp-Nle-DMeAsp-MePhe-amide,

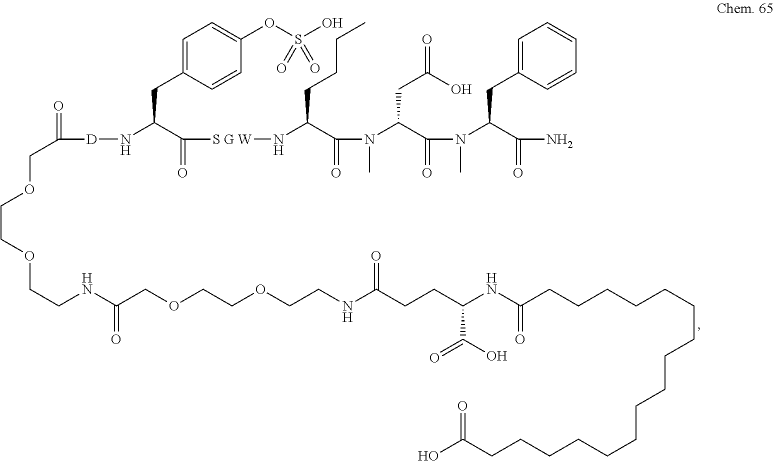

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Ala-Gly-Trp-Nle-DMeAsp-MePhe-amide,

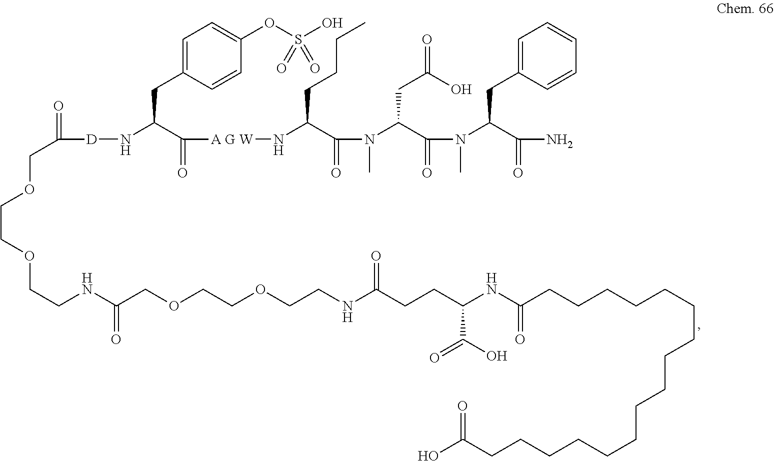

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Ile-Gly-Trp-Nle-DMeAsp-MePhe-amide,

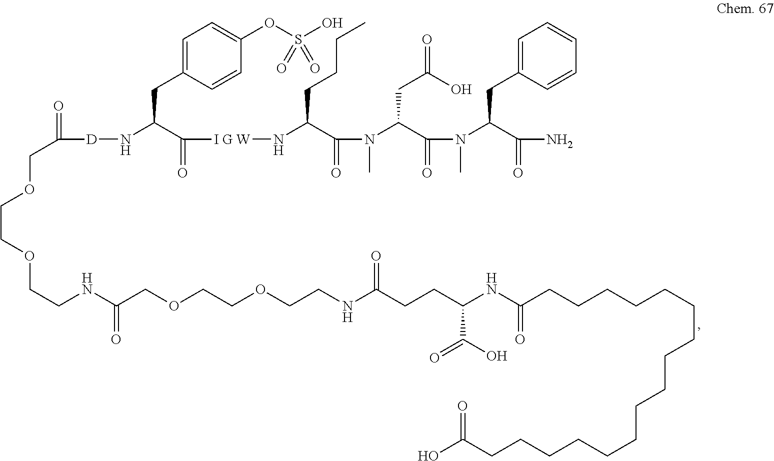

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-His-Gly-Trp-Nle-DMeAsp-MePhe-amide,

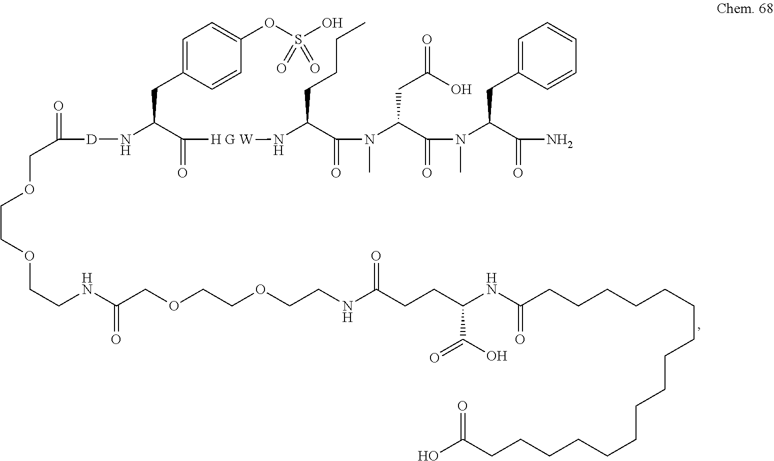

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-DGlu-sTyr-Leu-Gly-Trp-Nle-DMeAsp-MePhe-amide,

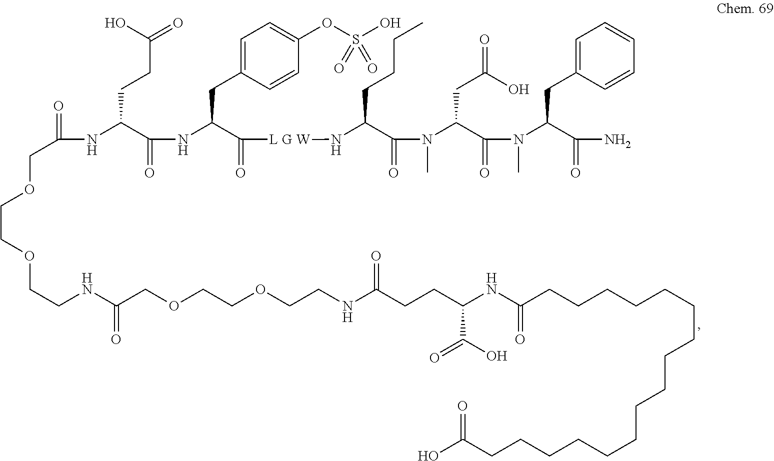

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Nle-DMeAsp-2Nal-amide,

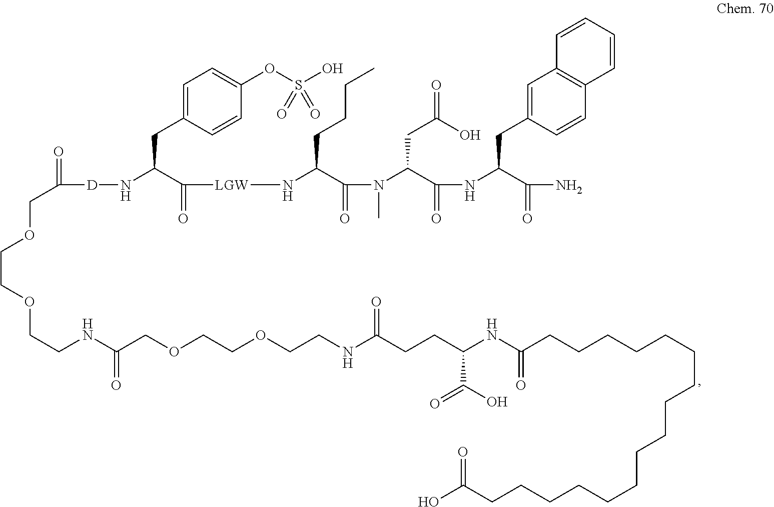

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Met-Gly-Trp-Met-DMeAsp-Phe-amide,

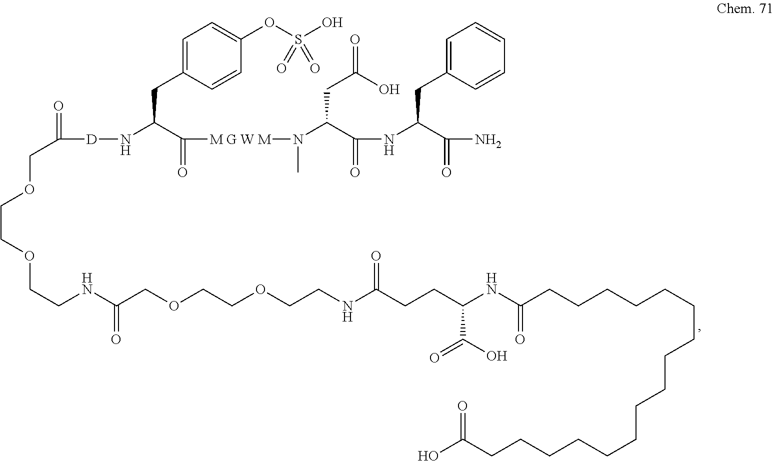

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Glu-sTyr-Leu-Gly-Trp-Nle-DMeAsp-Phe-amide, and

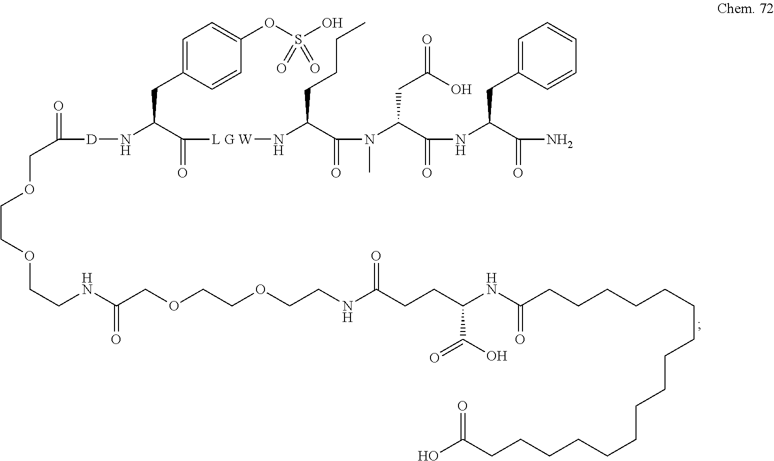

or a pharmaceutically acceptable salt, amide, or ester thereof.
135. The derivative of embodiment 134, which is a derivative of any of embodiments 1-133.
136. The derivative of any of embodiments 1-135, wherein X1-X2-X3-Gly-Trp-X6-DMeAsp-X8-NH₂ constitutes a peptide part of formula I.
137. The derivative of any of embodiments 1-136, wherein the peptide part is a CCK peptide.
138. The derivative of any of embodiments 1-137 which is a CCK derivative.
139. The derivative of any of embodiments 1-138, wherein the peptide part is an analogue of CCK-8s.
140. The derivative of any of embodiments 1-139, wherein the peptide part consists of 7 or 8 amino acid residues.
141. The derivative of any of embodiments 1-140, wherein the peptide part consists of 7 amino acid residues.
142. The derivative of any of embodiments 1-140, wherein the peptide part consists of 8 amino acid residues.
143. The derivative of any of embodiments 1-142 which is a CCK-1R agonist.
144. The derivative of any of embodiments 1-143 which is a full CCK-1R agonist. 145.

The derivative of any of embodiments 1-144, which has an in vitro potency on the CCK-1R which is similar to or only moderately decreased as compared to that of native human CCK-8s (SEQ ID NO: 1).

146. The derivative of any of embodiments 1-145, wherein in vitro potency on the CCK-1R is determined as the EC₅₀ value.
147. The derivative of any of embodiments 1-146, wherein the EC₅₀ value refers to the concentration of the derivative which in a CCK-1R activation assay induces a response half way between the basal (or bottom) response and the maximal (or top) response.
148. The derivative of any of embodiments 1-147, wherein the CCK-1R activation assay is a whole cell functional assay.
149. The derivative of any of embodiments 1-148, wherein the CCK-1R activation assay determines accumulation of inositol (1) phosphate (IP1).
150. The derivative of any of embodiments 1-149, wherein IP1 is determined using an assay kit which is commercially available from CisBio Bioassays, France (catalogue no. 62IPAPEB).
151. The derivative of any of embodiments 1-150, wherein the cells expressing the CCK-1R are 1321-N1 cells, for example from Perkin Elmer (product number ES-530-A).
152. The derivative of any of embodiments 1-151, wherein the cells are cultivated essentially as described in Example 91.
153. The derivative of any of embodiments 1-152, wherein IP1 is determined essentially as described in Example 91.
154. The derivative of any of embodiments 1-153, wherein the EC₅₀ value is determined essentially as described in Example 91.
155. The derivative of any of embodiments 1-154, wherein CCK-8s (SEQ ID NO: 1) is used as a positive reference.
156. The derivative of any of embodiments 1-155, wherein Gastrin-17 (SEQ ID NO: 2) is used as a negative reference.
157. The derivative of any of embodiments 1-156, wherein suitable serial dilutions of the derivative and the reference compounds are used.
158. The derivative of any of embodiments 1-157, wherein the serial dilutions are from 10⁻⁶ M (one μM) to 10⁻¹² M (one pM).
159. The derivative of any of embodiments 1-158, wherein the in vitro potency (EC₅₀ value) on the CCK-1R is determined essentially as described in Example 91.
160. The derivative of any of embodiments 1-159, which has an EC₅₀ value on the CCK-1R which is no more than 15 times the CCK-1R EC₅₀ value for CCK-8s (SEQ ID NO: 1).
161. The derivative of any of embodiments 1-160, which has an EC₅₀ value on the CCK-1R which is no more than 14 times the CCK-1R EC₅₀ value for CCK-8s (SEQ ID NO: 1).
162. The derivative of any of embodiments 1-161, which has an EC₅₀ value on the CCK-1R which is no more than 10 times the CCK-1R EC₅₀ value for CCK-8s (SEQ ID NO: 1).
163. The derivative of any of embodiments 1-162, which has an in EC₅₀ value on the CCK-1R which is no more than 8 times the CCK-1R EC₅₀ value for CCK-8s (SEQ ID NO: 1).
164. The derivative of any of embodiments 1-163, which has an in EC₅₀ value on the CCK-1R which is no more than 6 times the CCK-1R EC₅₀ value for CCK-8s (SEQ ID NO: 1).
165. The derivative of any of embodiments 1-164, which has an EC₅₀ value on the CCK-1R which is no more than 4 times the CCK-1R EC₅₀ value for CCK-8s (SEQ ID NO: 1).
166. The derivative of any of embodiments 1-165, which has an EC₅₀ value on the CCK-1R which is no more than 3 times the CCK-1R EC₅₀ value for CCK-8s (SEQ ID NO: 1).
167. The derivative of any of embodiments 1-166, which has an EC₅₀ value on the CCK-1R which is no more than 2 times the CCK-1R EC₅₀ value for CCK-8s (SEQ ID NO: 1).
168. The derivative of any of embodiments 1-167, which has an EC₅₀ value on the CCK-1R which is no more than 1.5 times the CCK-1R EC₅₀ value for CCK-8s (SEQ ID NO: 1).
169. The derivative of any of embodiments 1-168, which has an EC₅₀ value on the CCK-1R which is equal to or lower than the CCK-1R EC₅₀ value for CCK-8s (SEQ ID NO: 1).
170. The derivative of any of embodiments 1-169, which has an EC₅₀ value on the CCK-1R which is equal to or lower than 2000 pM.
171. The derivative of any of embodiments 1-170, which has an EC₅₀ value on the CCK-1R which is equal to or lower than 1600 pM.
172. The derivative of any of embodiments 1-78, which has an EC₅₀ value on the CCK-1R which is equal to or lower than 1300 pM.
173. The derivative of any of embodiments 1-171, which has an EC₅₀ value on the CCK-1R which is equal to or lower than 1000 pM.
174. The derivative of any of embodiments 1-172, which has an EC₅₀ value on the CCK-1R which is equal to or lower than 900 pM.
175. The derivative of any of embodiments 1-173, which has an EC₅₀ value on the CCK-1R which is equal to or lower than 800 pM.
176. The derivative of any of embodiments 1-174, which has an EC₅₀ value on the CCK-1R which is equal to or lower than 700 pM.
177. The derivative of any of embodiments 1-175, which has an EC₅₀ value on the CCK-1R which is equal to or lower than 600 pM.
178. The derivative of any of embodiments 1-176, which has an EC₅₀ value on the CCK-1R which is equal to or lower than 500 pM.
179. The derivative of any of embodiments 1-177, which has an EC₅₀ value on the CCK-1R which is equal to or lower than 400 pM.
180. The derivative of any of embodiments 1-178, which has an EC₅₀ value on the CCK-1R which is equal to or lower than 300 pM.
181. The derivative of any of embodiments 1-179, which has an EC₅₀ value on the CCK-1R which is equal to or lower than 150 pM.
182. The derivative of any of embodiments 1-181, which has an EC₅₀ value on the CCK-2R which is much higher than that of native human CCK-8s (SEQ ID NO: 1).
183. The derivative of any of embodiments 1-182, wherein in vitro potency on the CCK-2R is determined as the EC₅₀ value.
184. The derivative of any of embodiments 1-183, wherein the EC₅₀ value refers to the concentration of the derivative which in a CCK-2R activation assay induces a response half way between the basal (or bottom) response and the maximal (or top) response.
185. The derivative of any of embodiments 1-184, wherein the CCK-2R activation assay is a whole cell functional assay.
186. The derivative of any of embodiments 1-185, wherein the CCK-2R activation assay determines accumulation of inositol (1) phosphate (IP1).
187. The derivative of any of embodiments 1-186, wherein following CCK-2R activation IP1 is determined using an assay kit which is commercially available from CisBio Bioassays, France (catalogue no. 62IPAPEB).
188. The derivative of any of embodiments 1-187, wherein the cells expressing the CCK-2R are 1321-N1 cells, for example from Perkin Elmer (product number ES-531-A).
189. The derivative of any of embodiments 1-188, wherein the CCK-2R expressing cells are cultivated essentially as described in Example 91.
190. The derivative of any of embodiments 1-189, wherein IP1 generated after CCK-2R activation is determined essentially as described in Example 91.
191. The derivative of any of embodiments 1-190, wherein the EC₅₀ value on the CCK-2R is determined essentially as described in Example 91.
192. The derivative of any of embodiments 1-191, wherein CCK-8s (SEQ ID NO: 1) is used as a positive reference for CCK-2R activity.
193. The derivative of any of embodiments 1-192, wherein Gastrin-17 (SEQ ID NO: 2) is used as a positive reference for CCK-2R activity.
194. The derivative of any of embodiments 1-193, wherein for CCK-2R activity determination suitable serial dilutions of the derivative and the reference compounds are used.
195. The derivative of any of embodiments 1-194, wherein for CCK-2R activity the serial dilutions are from 10⁻⁶ M (one μM) to 10⁻¹² M (one pM).
196. The derivative of any of embodiments 1-195, wherein the in vitro potency (EC₅₀ value) on the CCK-2R is determined essentially as described in Example 91.
197. The derivative of any of embodiments 1-196, which has an in vitro potency on the CCK-2R (EC₅₀ value) which is at least 20 times the CCK-2R EC₅₀ value for CCK-8s (SEQ ID NO: 1).
198. The derivative of any of embodiments 1-197, which has an in vitro potency on the CCK-2R (EC₅₀ value) which is at least 40 times the CCK-2R EC₅₀ value for CCK-8s (SEQ ID NO: 1).
199. The derivative of any of embodiments 1-198, which has an in vitro potency on the CCK-2R (EC₅₀ value) which is at least 100 times the CCK-2R EC₅₀ value for CCK-8s (SEQ ID NO: 1).
200. The derivative of any of embodiments 1-199, which has an in vitro potency on the CCK-2R (EC₅₀ value) which is at least 500 times the CCK-2R EC₅₀ value for CCK-8s (SEQ ID NO: 1).
201. The derivative of any of embodiments 1-200, which has an in vitro potency on the CCK-2R (EC₅₀ value) which is at least 1000 times the CCK-2R EC₅₀ value for CCK-8s (SEQ ID NO: 1).
202. The derivative of any of embodiments 1-201, which has an in vitro potency on the CCK-2R (EC₅₀ value) which is at least 1500 times the CCK-2R EC₅₀ value for CCK-8s (SEQ ID NO: 1).
203. The derivative of any of embodiments 1-202, which has an in vitro potency on the CCK-2R (EC₅₀ value) which is at least 2000 times the CCK-2R EC₅₀ value for CCK-8s (SEQ ID NO: 1).
204. The derivative of any of embodiments 1-203, which has an in vitro potency on the CCK-2R (EC₅₀ value) which is at least 2500 times the CCK-2R EC₅₀ value for CCK-8s (SEQ ID NO: 1).
205. The derivative of any of embodiments 1-204, which has an in vitro potency on the CCK-2R (EC₅₀ value) which is at least 3000 times the CCK-2R EC₅₀ value for CCK-8s (SEQ ID NO: 1).
206. The derivative of any of embodiments 1-205, which has an in vitro potency on the CCK-2R (EC₅₀ value) which is at least 10000 pM.
207. The derivative of any of embodiments 1-206, which has an in vitro potency on the CCK-2R (EC₅₀ value) which is at least 30000 pM.
208. The derivative of any of embodiments 1-207, which has an in vitro potency on the CCK-2R (EC₅₀ value) which is at least 50000 pM.
209. The derivative of any of embodiments 1-208, which has an in vitro potency on the CCK-2R (EC₅₀ value) which is at least 100000 pM.
210. The derivative of any of embodiments 1-200, which has an in vitro potency on the CCK-2R (EC₅₀ value) which is at least 200000 pM.
211. The derivative of any of embodiments 1-210, which has an in vitro potency on the CCK-2R (EC₅₀ value) which is at least 400000 pM.
212. The derivative of any of embodiments 1-211, which has an in vitro potency on the CCK-2R (EC₅₀ value) which is at least 600000 pM.
213. The derivative of any of embodiments 1-212, which has an in vitro potency on the CCK-2R (EC₅₀ value) which is at least 800000 pM.
214. The derivative of any of embodiments 1-213, which has an in vitro potency on the CCK-2R (EC₅₀ value) which is at least 1000000 pM.
215. The derivative of any of embodiments 1-214, which is CCK-1R selective.
216. The derivative of any of embodiments 1-215, which has a ratio [EC₅₀ (CCK-2R) /EC₅₀ (CCK-1R)] of above 1.
217. The derivative of embodiment 216, where EC₅₀ (CCK-2R) and EC₅₀ (CCK-1R) refer to the potency of the derivative in question on the CCK-2R and the CCK-1R, respectively, determined as described in any of embodiments 185-196 and 148-159, respectively.
218. The derivative of any of embodiments 216-217, wherein the EC₅₀ (CCK-2R) and the EC₅₀ (CCK-1R) values are determined essentially as described in Example 91.
219. The derivative of any of embodiments 215-218, which has a ratio [EC₅₀ (CCK-2R)/EC₅₀ (CCK-1R)] of at least 5.
220. The derivative of any of embodiments 215-219, which has a ratio [EC₅₀ (CCK-2R)/EC₅₀ (CCK-1R)] of at least 20.
221. The derivative of any of embodiments 215-220, which has a ratio [EC₅₀ (CCK-2R)/EC₅₀ (CCK-1R)] of at least 50.
222. The derivative of any of embodiments 215-221, which has a ratio [EC₅₀ (CCK-2R)/EC₅₀ (CCK-1R)] of at least 100.
223. The derivative of any of embodiments 215-222, which has a ratio [EC₅₀ (CCK-2R)/EC₅₀ (CCK-1R)] of at least 200.
224. The derivative of any of embodiments 215-223, which has a ratio [EC₅₀ (CCK-2R)/EC₅₀ (CCK-1R)] of at least 300.
225. The derivative of any of embodiments 215-224, which has a ratio [EC₅₀ (CCK-2R)/EC₅₀ (CCK-1R)] of at least 500.
226. The derivative of any of embodiments 215-225, which has a ratio [EC₅₀ (CCK-2R)/EC₅₀ (CCK-1R)] of at least 1000.
227. The derivative of any of embodiments 216-226, which has a ratio [EC₅₀ (CCK-2R)/EC₅₀ (CCK-1R)] of at least 1500.
228. The derivative of any of embodiments 215-227, which has a ratio [EC₅₀ (CCK-2R)/EC₅₀ (CCK-1R)] of at least 2000.
229. The derivative of any of embodiments 215-228, which has a ratio [EC₅₀ (CCK-2R)/EC₅₀ (CCK-1R)] of at least 3000.
230. The derivative of any of embodiments 215-229, which has a ratio [EC₅₀ (CCK-2R)/EC₅₀ (CCK-1R)] of at least 4000.
231. The derivative of any of embodiments 215-230, which has a ratio [EC₅₀ (CCK-2R)/EC₅₀ (CCK-1R)] of at least 5000.
232. The derivative of any of embodiments 215-231, for which the ratio [EC₅₀ (CCK-2R)/EC₅₀ (CCK-1R)] is at least 2 times the corresponding ratio for CCK-8s (SEQ ID NO: 1).
233. The derivative of any of embodiments 215-232, for which the ratio [EC₅₀ (CCK-2R)/EC₅₀ (CCK-1R)] is at least 10 times the corresponding ratio for CCK-8s (SEQ ID NO: 1).
234. The derivative of any of embodiments 215-233, for which the ratio [EC₅₀ (CCK-2R)/EC₅₀ (CCK-1R)] is at least 50 times the corresponding ratio for CCK-8s (SEQ ID NO: 1).
235. The derivative of any of embodiments 215-234, for which the ratio [EC₅₀ (CCK-2R)/EC₅₀ (CCK-1R)] is at least 100 times the corresponding ratio for CCK-8s (SEQ ID NO: 1).
236. The derivative of any of embodiments 215-235, for which the ratio [EC₅₀ (CCK-2R)/EC₅₀ (CCK-1R)] is at least 500 times the corresponding ratio for CCK-8s (SEQ ID NO: 1).
237. The derivative of any of embodiments 215-236, for which the ratio [EC₅₀ (CCK-2R)/EC₅₀ (CCK-1R)] is at least 1000 times the corresponding ratio for CCK-8s (SEQ ID NO: 1).
238. The derivative of any of embodiments 215-237, for which the ratio [EC₅₀ (CCK-2R)/EC₅₀ (CCK-1R)] is at least 2000 times the corresponding ratio for CCK-8s (SEQ ID NO: 1).
239. The derivative of any of embodiments 232-238 wherein for calculation of the ratio [EC₅₀ (CCK-2R)/EC₅₀ (CCK-1R)] the EC₅₀ (CCK-2R) and the EC₅₀ (CCK-1R) values are determined for the derivative and for CCK-8s (SEQ ID NO: 1), using the same assays, respectively.
240. The derivative of embodiment 239, wherein the assays are as described in any of embodiments 185-196 (EC₅₀ (CCK-2R)) and 148-159 (EC₅₀ (CCK-1R)). 241. The derivative of embodiment 240, wherein the assay is the assay of Example 91.
242. The derivative of any of embodiments 1-241, which has an in vitro receptor binding (affinity) to the CCK-1R which is similar to that of native human CCK-8s (SEQ ID NO: 1).
243. The derivative of any of embodiments 1-242, which has an in vitro affinity to the CCK-1R which is better than that of native human CCK-8s (SEQ ID NO: 1).
244. The derivative of any of embodiments 1-242, which has an in vitro affinity to the CCK-1R which is only moderately decreased as compared to that of native human CCK-8s (SEQ ID NO: 1).
245. The derivative of any of embodiments 1-244, wherein in vitro affinity to the CCK-1R is determined as the IC₅₀ value.
246. The derivative of any of embodiments 1-245, wherein the IC₅₀ value refers to the concentration of the derivative which in a competitive CCK-1R binding assay induces a response half way between the basal (or bottom) response and the maximal (or top) response.
247. The derivative of any of embodiments 1-246, wherein the binding affinity to the CCK-1R is measured by way of the ability to displace [¹²⁵I]-CCK-8s from the receptors in a plasma membrane based receptor binding assay.
248. The derivative of any of embodiments 1-247, wherein the membranes are derived from cells expressing the CCK-1R, such as 1321-N1 cells, for example from Perkin Elmer (product number ES-530-A).
249. The derivative of any of embodiments 1-248, wherein the cells are cultivated essentially as described in Example 92.
250. The derivative of any of embodiments 1-249, wherein the membranes are purified essentially as described in Example 92.
251. The derivative of any of embodiments 1-250, wherein the CCK-1R binding assay is performed essentially as described in Example 92.
252. The derivative of any of embodiments 1-251, wherein CCK-8s (SEQ ID NO: 1) is used as a positive reference.
253. The derivative of any of embodiments 1-252, wherein Gastrin-17 (SEQ ID NO: 2) is used as a negative reference.
254. The derivative of any of embodiments 1-253, wherein suitable serial dilutions of the derivative and the reference compounds are used.
255. The derivative of any of embodiments 1-254, wherein the serial dilutions are from 10⁻⁵ M (ten μM) to 10⁻¹² M (one pM).
256. The derivative of any of embodiments 1-255, wherein the in vitro affinity (IC₅₀ value) on the CCK-1R is determined essentially as described in Example 92.
257. The derivative of any of embodiments 1-256, which has a CCK-1R IC₅₀ value which is no more than 10 times the CCK-1R IC₅₀ value for CCK-8s (SEQ ID NO: 1).
258. The derivative of any of embodiments 1-257, which has a CCK-1R IC₅₀ value on the CCK-1R which is no more than 8 times the CCK-1R IC₅₀ value for CCK-8s (SEQ ID NO: 1).
259. The derivative of any of embodiments 1-258, which has an CCK-1R IC₅₀ value on the CCK-1R which is no more than 6 times the CCK-1R EC₅₀ value for CCK-8s (SEQ ID NO: 1).
260. The derivative of any of embodiments 1-259, which has a CCK-1R IC₅₀ value on the CCK-1R which is no more than 5 times the CCK-1R IC₅₀ value for CCK-8s (SEQ ID NO: 1).
261. The derivative of any of embodiments 1-260, which has a CCK-1R IC₅₀ value on the CCK-1R which is no more than 4 times the CCK-1R IC₅₀ value for CCK-8s (SEQ ID NO: 1).
262. The derivative of any of embodiments 1-261, which has a CCK-1R IC₅₀ value on the CCK-1R which is no more than 3 times the CCK-1R IC₅₀ value for CCK-8s (SEQ ID NO: 1).
263. The derivative of any of embodiments 1-262, which has a CCK-1R IC₅₀ value on the CCK-1R which is no more than 2 times the CCK-1R IC₅₀ value for CCK-8s (SEQ ID NO: 1).
264. The derivative of any of embodiments 1-263, which has a CCK-1R IC₅₀ value on the CCK-1R which is no more than 1 times the CCK-1R IC₅₀ value for CCK-8s (SEQ ID NO: 1).
265. The derivative of any of embodiments 1-264, which has a CCK-1R IC₅₀ value on the CCK-1R which is no more than 0.5 times the CCK-1R IC₅₀ value for CCK-8s (SEQ ID NO: 1).
266. The derivative of any of embodiments 1-265, which has a CCK-1R IC₅₀ value on the CCK-1R which is equal to or lower than 0.25 times the CCK-1R IC₅₀ value for CCK-8s (SEQ ID NO: 1).
267. The derivative of any of embodiments 1-266, which has an IC₅₀ value on the CCK-1R which is equal to or lower than 2000 pM.
268. The derivative of any of embodiments 1-267, which has an IC₅₀ value on the CCK-1R which is equal to or lower than 1500 pM.
269. The derivative of any of embodiments 1-268, which has an IC₅₀ value on the CCK-1R which is equal to or lower than 1000 pM.
270. The derivative of any of embodiments 1-269, which has an IC₅₀ value on the CCK-1R which is equal to or lower than 750 pM.
271. The derivative of any of embodiments 1-270, which has an IC₅₀ value on the CCK-1R which is equal to or lower than 500 pM.
272. The derivative of any of embodiments 1-271, which has an IC₅₀ value on the CCK-1R which is equal to or lower than 400 pM.
273. The derivative of any of embodiments 1-272, which has an IC₅₀ value on the CCK-1R which is equal to or lower than 300 pM.
274. The derivative of any of embodiments 1-273, which has an IC₅₀ value on the CCK-1R which is equal to or lower than 200 pM.
275. The derivative of any of embodiments 1-274, which has an IC₅₀ value on the CCK-1R which is equal to or lower than 150 pM.
276. The derivative of any of embodiments 1-275, which has an IC₅₀ value on the CCK-1R which is equal to or lower than 100 pM.
277. The derivative of any of embodiments 1-276, which has an IC₅₀ value on the CCK-1R which is equal to or lower than 75 pM.
278. The derivative of any of embodiments 1-277, which has an IC₅₀ value on the CCK-1R which is equal to or lower than 50 pM.
279. The derivative of any of embodiments 1-278, which has an in vitro receptor binding (affinity) to the CCK-2R which is significant lower than that of native human CCK-8s (SEQ ID NO: 1).
280. The derivative of any of embodiments 1-279, wherein in vitro affinity to the CCK-2R is determined as the IC₅₀ value.
281. The derivative of any of embodiments 1-280, wherein the IC₅₀ value refers to the concentration of the derivative which in a competitive CCK-2R binding assay induces a response half way between the basal (or bottom) response and the maximal (or top) response.
282. The derivative of any of embodiments 1-281, wherein the binding affinity to the CCK-2R is measured by way of the ability to displace [¹²⁵I]-CCK-8s from the receptors in a plasma membrane based receptor binding assay.
283. The derivative of any of embodiments 1-282, wherein the membranes are derived from cells expressing the CCK-2R, such as 1321-N1 cells, for example from Perkin Elmer (product number ES-531-A).
284. The derivative of any of embodiments 1-283, wherein the cells are cultivated essentially as described in Example 92.
285. The derivative of any of embodiments 1-284, wherein the membranes are purified essentially as described in Example 92.
286. The derivative of any of embodiments 1-285, wherein the CCK-2R binding assay is performed essentially as described in Example 92.
287. The derivative of any of embodiments 1-286, wherein CCK-8s (SEQ ID NO: 1) is used as a reference.
288. The derivative of any of embodiments 1-287, wherein Gastrin-17 (SEQ ID NO: 2) is used as a positive reference.
289. The derivative of any of embodiments 1-288, wherein suitable serial dilutions of the derivative and the reference compounds are used.
290. The derivative of any of embodiments 1-289, wherein the serial dilutions are from 10⁻⁵ M (ten μM) to 10⁻¹² M (one pM).
291. The derivative of any of embodiments 1-290, wherein the in vitro affinity (IC₅₀ value) on the CCK-2R is determined essentially as described in Example 92.
292. The derivative of any of embodiments 1-291, which has a CCK-2R IC₅₀ value which is at least 100 times the CCK-2R IC₅₀ value for CCK-8s (SEQ ID NO: 1).
293. The derivative of any of embodiments 1-292, which has a CCK-2R IC₅₀ value on the CCK-2R which is at least 200 times the CCK-2R IC₅₀ value for CCK-8s (SEQ ID NO: 1).
294. The derivative of any of embodiments 1-293, which has an CCK-2R IC₅₀ value on the CCK-2R which is at least 300 times the CCK-2R EC₅₀ value for CCK-8s (SEQ ID NO: 1).
295. The derivative of any of embodiments 1-294, which has a CCK-2R IC₅₀ value on the CCK-2R which is at least 500 times the CCK-2R IC₅₀ value for CCK-8s (SEQ ID NO: 1).
296. The derivative of any of embodiments 1-295, which has a CCK-2R IC₅₀ value on the CCK-2R which is at least 1000 times the CCK-2R IC₅₀ value for CCK-8s (SEQ ID NO: 1).
297. The derivative of any of embodiments 1-296, which has a CCK-2R IC₅₀ value on the CCK-2R which is at least 2000 times the CCK-2R IC₅₀ value for CCK-8s (SEQ ID NO: 1).
298. The derivative of any of embodiments 1-297, which has a CCK-2R IC₅₀ value on the CCK-2R which is at least 5000 times the CCK-2R IC₅₀ value for CCK-8s (SEQ ID NO: 1).
299. The derivative of any of embodiments 1-298, which has a CCK-2R IC₅₀ value on the CCK-2R which is at least 10000 times the CCK-2R IC₅₀ value for CCK-8s (SEQ ID NO: 1).
300. The derivative of any of embodiments 1-299, which has a CCK-2R IC₅₀ value on the CCK-2R which is at least 20000 times the CCK-2R IC₅₀ value for CCK-8s (SEQ ID NO: 1).
301. The derivative of any of embodiments 1-300, which has a CCK-2R IC₅₀ value on the CCK-2R which is similar to or higher than the CCK-2R IC₅₀ value for Gastrin-17 (SEQ ID NO: 2).
302. The derivative of any of embodiments 1-301, which has an IC₅₀ value on the CCK-2R which is at least 500 pM.
303. The derivative of any of embodiments 1-302, which has an IC₅₀ value on the CCK-2R which is at least 5000 pM.
304. The derivative of any of embodiments 1-303, which has an IC₅₀ value on the CCK-2R which is at least 30000 pM.
305. The derivative of any of embodiments 1-304, which has an IC₅₀ value on the CCK-2R which is at least 50000 pM.
306. The derivative of any of embodiments 1-305, which has an IC₅₀ value on the CCK-2R which is at least 80000 pM.
307. The derivative of any of embodiments 1-306, which has an IC₅₀ value on the CCK-2R which is at least 1×10⁶ pM.
308. The derivative of any of embodiments 1-307, which has an IC₅₀ value on the CCK-2R which is at least 2×10⁶ pM.
309. The derivative of any of embodiments 1-308, which has an IC₅₀ value on the CCK-2R which is at least 3×10⁶ pM.
310. The derivative of any of embodiments 1-309, which has an IC₅₀ value on the CCK-2R which is at least 4×10⁶ pM.
311. The derivative of any of embodiments 1-310, which has an IC₅₀ value on the CCK-2R which is at least 6×10⁶ pM.
312. The derivative of any of embodiments 1-311, which has an IC₅₀ value on the CCK-2R which is at least 8×10⁶ pM.
313. The derivative of any of embodiments 1-312, which has an IC₅₀ value on the CCK-2R which is at least 1×10⁷ pM.
314. The derivative of any of embodiments 1-313, which selectively binds to the CCK-1R.
315. The derivative of any of embodiments 1-314, which has a ratio [IC₅₀ (CCK-2R) /IC₅₀ (CCK-1R)] of above 1.
316. The derivative of embodiment 315, where IC₅₀ (CCK-2R) and IC₅₀ (CCK-1R) refer to the affinity of the derivative in question to the CCK-2R and the CCK-1R, respectively, determined as described in any of embodiments 281-291 and 246-256, respectively.
317. The derivative of any of embodiments 315-316, wherein the IC₅₀ (CCK-2R) and the IC₅₀ (CCK-1R) values are determined essentially as described in Example 92.
318. The derivative of any of embodiments 315-317, which has a ratio [IC₅₀ (CCK-2R)/IC₅₀ (CCK-1R)] of at least 50.
319. The derivative of any of embodiments 315-318, which has a ratio [IC₅₀ (CCK-2R)/IC₅₀ (CCK-1R)] of at least 500.
320. The derivative of any of embodiments 315-319, which has a ratio [IC₅₀ (CCK-2R)/IC₅₀ (CCK-1R)] of at least 4000.
321. The derivative of any of embodiments 315-320, which has a ratio [IC₅₀ (CCK-2R)/IC₅₀ (CCK-1R)] of at least 6000.
322. The derivative of any of embodiments 315-321, which has a ratio [IC₅₀ (CCK-2R)/IC₅₀ (CCK-1R)] of at least 8000.
323. The derivative of any of embodiments 315-322, which has a ratio [IC₅₀ (CCK-2R)/IC₅₀ (CCK-1R)] of at least 10000.
324. The derivative of any of embodiments 315-323, which has a ratio [IC₅₀ (CCK-2R)/IC₅₀ (CCK-1R)] of at least 20000.
325. The derivative of any of embodiments 315-324, which has a ratio [IC₅₀ (CCK-2R)/IC₅₀ (CCK-1R)] of at least 40000.
326. The derivative of any of embodiments 315-325, which has a ratio [IC₅₀ (CCK-2R)/IC₅₀ (CCK-1R)] of at least 60000.
327. The derivative of any of embodiments 315-326, which has a ratio [IC₅₀ (CCK-2R)/IC₅₀ (CCK-1R)] of at least 80000.
328. The derivative of any of embodiments 315-327, which has a ratio [IC₅₀ (CCK-2R)/IC₅₀ (CCK-1R)] of at least 100000.
329. The derivative of any of embodiments 315-328, which has a ratio [IC₅₀ (CCK-2R)/IC₅₀ (CCK-1R)] of at least 150000.
330. The derivative of any of embodiments 315-329, which has a ratio [IC₅₀ (CCK-2R)/IC₅₀ (CCK-1R)] of at least 200000.
331. The derivative of any of embodiments 315-330, for which the ratio [IC₅₀ (CCK-2R)/IC₅₀ (CCK-1R)] is at least 100 times the corresponding ratio for CCK-8s (SEQ ID NO: 1).
332. The derivative of any of embodiments 315-331, for which the ratio [IC₅₀ (CCK-2R)/IC₅₀ (CCK-1R)] is at least 1000 times the corresponding ratio for CCK-8s (SEQ ID NO: 1).
333. The derivative of any of embodiments 315-332, for which the ratio [IC₅₀ (CCK-2R)/IC₅₀ (CCK-1R)] is at least 2000 times the corresponding ratio for CCK-8s (SEQ ID NO: 1).
334. The derivative of any of embodiments 315-333, for which the ratio [IC₅₀ (CCK-2R)/IC₅₀ (CCK-1R)] is at least 10000 times the corresponding ratio for CCK-8s (SEQ ID NO: 1).
335. The derivative of any of embodiments 315-334, for which the ratio [IC₅₀ (CCK-2R)/IC₅₀ (CCK-1R)] is at least 25000 times the corresponding ratio for CCK-8s (SEQ ID NO: 1).
336. The derivative of any of embodiments 315-335, for which the ratio [IC₅₀ (CCK-2R)/IC₅₀ (CCK-1R)] is at least 50000 times the corresponding ratio for CCK-8s (SEQ ID NO: 1).
337. The derivative of any of embodiments 315-336, for which the ratio [IC₅₀ (CCK-2R)/IC₅₀ (CCK-1R)] is at least 70000 times the corresponding ratio for CCK-8s (SEQ ID NO: 1).
338. The derivative of any of embodiments 315-337 wherein for calculation of the ratio [IC₅₀ (CCK-2R)/IC₅₀ (CCK-1R)] the IC₅₀ (CCK-2R) and the IC₅₀ (CCK-1R) values are determined for the derivative and for CCK-8s (SEQ ID NO: 1), using the same assays, respectively.
339. The derivative of embodiment 338, wherein the assays are as described in any of embodiments 281-291 (IC₅₀ (CCK-2R)) and 246-256 (IC₅₀ (CCK-1R)).
340. The derivative of embodiment 339, wherein the assays are those of Example 92.
341. The derivative of any of embodiments 1-340, which has a more protracted profile of action than native CCK-8s (SEQ ID NO: 1).
342. The derivative of embodiment 341, wherein protracted profile of action refers to terminal half-life in vivo in a relevant animal species, such as mouse, rat, pig, and dog, preferably mini-pig; wherein the derivative is administered i) s.c., and/or ii) i.v.; preferably ii) s.c.
343. The derivative of any of embodiments 1-340, wherein the terminal half-life (T_{1/2}) after i.v. administration to mini-pigs is at least 2 hours.
344. The derivative of any of embodiments 1-343, wherein the terminal half-life (T_{1/2}) after i.v. administration to mini-pigs is at least 3 hours.
345. The derivative of any of embodiments 1-344, wherein the terminal half-life (T_{1/2}) after i.v. administration to mini-pigs is at least 12 hours.
346. The derivative of any of embodiments 1-345, wherein the terminal half-life (T_{1/2}) after i.v. administration to mini-pigs is at least 24 hours.
347. The derivative of any of embodiments 1-346, wherein the terminal half-life (T_{1/2}) after i.v. administration to mini-pigs is at least 36 hours.
348. The derivative of any of embodiments 1-347, wherein the terminal half-life (T_{1/2}) after i.v. administration to mini-pigs is at least 60 hours.
349. The derivative of any of embodiments 1-348, wherein the terminal half-life (T_{1/2}) after i.v. administration to mini-pigs is at least 80 hours.
350. The derivative of any of embodiments 1-349, wherein the terminal half-life (T_{1/2}) after i.v. administration to mini-pigs is at least 100 hours.
351. The derivative of any of embodiments 1-350, wherein the terminal half-life (T_{1/2}) after i.v. administration to mini-pigs is at least 110 hours.
352. The derivative of any of embodiments 1-351, wherein the terminal half-life (T_{1/2}) after i.v. administration to mini-pigs is at least 120 hours.
353. The derivative of any of embodiments 1-352, wherein the terminal half-life (T_{1/2}) after i.v. administration to mini-pigs is at least 140 hours.
354. The derivative of any of embodiments 1-353, wherein the terminal half-life (T_{1/2}) after i.v. administration to mini-pigs is at least 160 hours.
355. The derivative of any of embodiments 342-354, wherein the mini-pigs are female, preferably obtained from Ellegaard Gottingen Minipigs.
356. The derivative of any of embodiments 342-355, wherein the mini-pigs are approximately 6 months of age, and preferably weighing approximately 17 kg.
357. The derivative of any of embodiments 342-356, wherein the derivative is dissolved in 50 mM sodium phosphate buffer containing 70 mM sodium chloride, and 0.05% polysorbate 80, at pH 7.4.
358. The derivative of any of embodiments 342-357, wherein an intravenous injection of the derivative is given in a dosage corresponding to, e.g., 5 nmol/kg body weight.
359. The derivative of any of embodiments 342-358, wherein the terminal half-life (T_{1/2}) is determined in vivo pharmacokinetic studies in mini-pig, for example as described in Example 93.
360. The derivative of any of embodiments 1-359, which in a pharmacodynamic (PD) study in pigs and when administered as a single dose s.c. has the effect of reducing food intake for (i) at least 24 hours, (ii) at least 48 hours, (iii) at least 72 hours, and/or (iv) at least 96 hours, as compared to baseline, where baseline refers to mean food intake on three successive days before dosing.
361. The derivative of any of embodiments 1-360, which in a pharmacodynamic (PD) study in pigs and when administered as a single dose s.c. has the effect of reducing food intake for (i) at least 24 hours, (ii) at least 48 hours, (iii) at least 72 hours, and/or (iv) at least 96 hours, as compared to a vehicle-treated control group.
362. The derivative of any of embodiments 360-361, wherein the pigs are female Landrace Yorkshire Duroc (LYD) pigs or Large White hybrid, approximately 3 months of age, and weighing approximately 30-35 kg (preferably n=4 per group).
363. The derivative of any of embodiments 360-362, wherein the pigs are housed in a group for approximately 1 week during acclimatisation to the animal facilities, and subsequently, during the experimental period, the animals are placed in individual pens approximately 1 week before dosing and during the entire experiment for measurement of individual food intake.
364. The derivative of any of embodiments 360-363, wherein the pigs are fed ad libitum with pig fodder (Svinefoder Danish Top; supplier Danish Agro) at all times both during the acclimatisation and the experimental period.
365. The derivative of any of embodiments 360-364, wherein food intake is monitored, e.g. on line by logging the weight of fodder every 15 minutes, preferably using the Mpigwin system (available e.g. from Ellegaard Systems, Faaborg, Denmark); any uneaten fodder is removed and weighed (preferably manually) on the following morning and replaced with fresh fodder; a baseline food intake is measured for each pig on three successive days before dosing of the derivative in question; and a mean baseline food intake value is calculated for each pig.
366. The derivative of any of embodiments 360-365, which is dissolved in a phosphate buffer (50 mM sodium phosphate, 70 mM sodium chloride, 0.05 v/v % polysorbate 80, pH 7.4) at concentrations of (i) approximately 5300 nmol/ml corresponding to doses of 100 nmol/kg body weight, and/or (ii) approximately 1200 nmol/ml corresponding to doses of 30 nmol/kg.
367. The derivative of embodiment 366, wherein the phosphate buffer serves as vehicle.
368. The derivative of any of embodiments 360-367, wherein the pigs are dosed with a single subcutaneous dose of the derivative or vehicle (dose volume 0.02 ml/kg) on the morning of day 1, and food intake is measured for 48, 72, and/or 96 hours after dosing.
369. The derivative of any of embodiments 360-368, wherein food intake (FI) is calculated in 24 h intervals (such as 0-24 h, and 24-48 h), and the resulting mean difference in food intake (ΔFI, in kg) is calculated on the basis of the mean baseline food intake for each pig (ΔFI₀₋₂₄ₕ=FI₀₋₂₄ₕ-FI_{baseline}, and vice-versa for the 24-48 hours' time interval and any later time interval).
370. The derivative of any of embodiments 360-369, wherein the food intake is determined essentially as described in any of Examples 94 or 97.
371. The derivative of any of embodiments 360-370, wherein the food intake for hours 0-24 is reduced by at least 0.10 kg (ΔFI₀₋₂₄ₕ is −0.10 kg or lower).
372. The derivative of any of embodiments 360-371, wherein the food intake for hours 0-24 is reduced by at least 0.20 kg (ΔFI₀₋₂₄ₕ is −0.20 kg or lower).
373. The derivative of any of embodiments 360-372, wherein the food intake for hours 0-24 is reduced by at least 0.30 kg (ΔFI₀₋₂₄ₕ is −0.30 kg or lower).
374. The derivative of any of embodiments 360-373, wherein the food intake for hours 0-24 is reduced by at least 0.40 kg (ΔFI₀₋₂₄ₕ is −0.40 kg or lower).
375. The derivative of any of embodiments 360-374, wherein the food intake for hours 0-24 is reduced by at least 0.50 kg (ΔFI₀₋₂₄ₕ is −0.50 kg or lower).
376. The derivative of any of embodiments 360-375, wherein the food intake for hours 0-24 is reduced by at least 0.60 kg (ΔFI₀₋₂₄ₕ is −0.60 kg or lower).
377. The derivative of any of embodiments 360-376, wherein the food intake for hours 0-24 is reduced by at least 0.70 kg (ΔFI₀₋₂₄ₕ is −0.70 kg or lower).
378. The derivative of any of embodiments 360-377, wherein the food intake for hours 0-24 is reduced by at least 0.80 kg (ΔFI₀₋₂₄ₕ is −0.80 kg or lower).
379. The derivative of any of embodiments 360-378, wherein the food intake for hours 0-24 is reduced by at least 0.90 kg (ΔFI₀₋₂₄ₕ is −0.90 kg or lower).
380. The derivative of any of embodiments 360-379, wherein the food intake for hours 0-24 is reduced by at least 1.0 kg (ΔFI₀₋₂₄ₕ is −1.0 kg or lower).
381. The derivative of any of embodiments 360-380, wherein the food intake for hours 24-48 is reduced by at least 0.10 kg (ΔFI₂₄₋₄₈ₕ is −0.10 kg or lower).
382. The derivative of any of embodiments 360-381, wherein the food intake for hours 24-48 is reduced by at least 0.20 kg (ΔFI₂₄₋₄₈ₕ is −0.20 kg or lower).
383. The derivative of any of embodiments 360-382, wherein the food intake for hours 24-48 is reduced by at least 0.30 kg (ΔFI₂₄₋₄₈ₕ is −0.30 kg or lower).
384. The derivative of any of embodiments 360-383, wherein the food intake for hours 24-48 is reduced by at least 0.40 kg (ΔFI₂₄₋₄₈ₕ is −0.40 kg or lower).
385. The derivative of any of embodiments 360-384, wherein the food intake for hours 24-48 is reduced by at least 0.50 kg (ΔFI₂₄₋₄₈ₕ is −0.50 kg or lower).
386. The derivative of any of embodiments 360-385, wherein the food intake for hours 24-48 is reduced by at least 0.60 kg (ΔFI₂₄₋₄₈ₕ is −0.60 kg or lower).
387. The derivative of any of embodiments 360-386, wherein the food intake for hours 48-72 is reduced by at least 0.10 kg (ΔFI₄₈₋₇₂ₕ is −0.10 kg or lower).
388. The derivative of any of embodiments 360-387, wherein the food intake for hours 48-72 is reduced by at least 0.20 kg (ΔFI₄₈₋₇₂ₕ is −0.20 kg or lower).
389. The derivative of any of embodiments 360-388, wherein the food intake for hours 48-72 is reduced by at least 0.25 kg (ΔFI₄₈₋₇₂ₕ is −0.25 kg or lower).
390. The derivative of any of embodiments 360-389, wherein the food intake for hours 72-96 is reduced by at least 0.05 kg (ΔFI₇₂₋₉₆ₕ is −0.05 kg or lower).
391. The derivative of any of embodiments 360-390, wherein the food intake for hours 72-96 is reduced by at least 0.10 kg (ΔFI₇₂₋₉₆ₕ is −0.10 kg or lower).
392. The derivative of any of embodiments 360-391, wherein the food intake for hours 72-96 is reduced by at least 0.15 kg (ΔFI₇₂₋₉₆ₕ is −0.15 kg or lower).
393. The derivative of any of embodiments 1-392, which is stable in a pH 7.4 phosphate buffer at 5° C. for at least 6 weeks.
394. The derivative of any of embodiments 1-393, which is stable in a pH 7.4 phosphate buffer at 25° C. for at least 6 weeks.
395. The derivative of any of embodiments 1-394, which is stable in a pH 7.4 phosphate buffer at 37° C. for at least 6 weeks.
396. The derivative of any of embodiments 393-395, wherein the phosphate buffer is 50 mM sodium phosphate containing 70 mM NaCl and 0.05 v/v % polysorbate 80.
397. The derivative of any of embodiments 393-396, wherein the concentration of the derivative is 1 mg/ml.
398. The derivative of any of embodiments 393-397, wherein the concentration of the derivative is approximately 0.5 mM.
399. The derivative of any of embodiments 393-398 which is stable in a pH 7.4 phosphate buffer (50 mM sodium phosphate containing 70 mM NaCl and 0.05 v/v % polysorbate 80) at 25° C. for at least three days in a concentration of (i) 25 μM, (ii) 100 μM, and/or (iii) 500 μM.
400. The derivative of any of embodiments 393-399, wherein the term stable refers to a relative purity loss of no more than 2.0%.
401. The derivative of any of embodiments 393-400, wherein the term stable refers to a relative purity loss of no more than 1.8%.
402. The derivative of any of embodiments 393-401, wherein the term stable refers to a relative purity loss of no more than 1.6%.
403. The derivative of any of embodiments 393-402, wherein the term stable refers to a relative purity loss of no more than 1.4%.
404. The derivative of any of embodiments 393-403, wherein the term stable refers to a relative purity loss of no more than 1.2%.
405. The derivative of any of embodiments 393-404, wherein the term stable refers to a relative purity loss of no more than 1.0%.
406. The derivative of any of embodiments 393-405, wherein the term stable refers to a relative purity loss of no more than 0.80%.
407. The derivative of any of embodiments 393-406, wherein the term stable refers to a relative purity loss of no more than 0.60%.
408. The derivative of any of embodiments 393-407, wherein the term stable refers to a relative purity loss of no more than 0.40%.
409. The derivative of any of embodiments 393-408, wherein the term stable refers to a relative purity loss of no more than 0.25%.
410. The derivative of any of embodiments 393-409, wherein the relative purity loss (in %) is the ratio of absolute purity loss to the purity determined in the start sample, where absolute purity loss (in percentage points, pp) is determined as the difference in purity between the start sample and the sample incubated for the indicated time (such as 6 weeks) at the indicated temperature.
411. The derivative of any of embodiments 393-410, wherein the purity of the start sample and the purity of the sample incubated for the indicated time at the indicated temperature is determined using RP-UPLC analysis, and wherein purity is defined as the area percentage of the compound peak in relation to the total area of all integrated peaks in the chromatogram.
412. The derivative of any of embodiments 393-411, wherein the purity loss is determined essentially as described in Example 95, part 1.
413. The derivative of any of embodiments 393-412, wherein the stability and/or purity is determined essentially as described in Example 95, part 1.
414. The derivative of any of embodiments 393-413, which is stable in a pH 7.4 phosphate buffer at 5° C. for at least 2 weeks.
415. The derivative of any of embodiments 393-414, which is stable in a pH 7.4 phosphate buffer at 25° C. for at least 2 weeks.
416. The derivative of any of embodiments 393-415, which is stable in a pH 7.4 phosphate buffer at 37° C. for at least 2 weeks.
417. The derivative of any of embodiments 393-416, where stability is determined as indicated in any of embodiments 393-416.
418. The derivative of any of embodiments 414-417, wherein the term stable refers to a relative purity loss of no more than 0.20%.
419. The derivative of any of embodiments 414-417, wherein the term stable refers to a relative purity loss of no more than 0.16%.
420. A peptide of the general formula I′:

X1-X2-X3-Gly-Trp-X6-DMeAsp-X8-NH₂ (formula I′),

wherein

X1 is absent, Asp, DAsp, bAsp, DbAsp, Glu, or DGlu;

X2 is Phe(4-sulfomethyl) or sTyr;

X3 is Nle, Leu, Ala, Ile, Lys, Arg, Pro, Met, Phe, Ser, His, or Val;

X6 is Nle, Ile, Gln, Met, Met(O2), Leu, Val, Pro, Hpg, or Ala;

X8 is Phe, MePhe, 1Nal, Me1Nal, 2Nal, Me2Nal, Trp, or MeTrp; and

the NH₂ group at the C-terminus of formula I′ means that X8 is C-terminally amidated;

or a pharmaceutically acceptable salt, amide, or ester thereof.

421. The peptide of embodiment 420 which is an intermediate to the derivative of any of embodiments 1-419.
422. The peptide of any of embodiments 420-421, wherein
X1 is absent, Asp, DAsp, bAsp, DbAsp, Glu, or DGlu;
X2 is Phe(4-sulfomethyl) or sTyr;

### X3 is Nle, Leu, Ala, Ile, Lys, Arg, Pro, Met, Phe, Ser, or His;

### X6 is Nle, Ile, Gln, Met, Met(O2), Leu, Val, Pro, or Ala; and

### X8 is Phe, MePhe, 1Nal, Me1Nal, 2Nal, or Trp.

423. The peptide of any of embodiments 420-422, wherein X1 is absent.
424. The peptide of any of embodiments 420-422, wherein X1 is Asp.
425. The peptide of any of embodiments 420-422, wherein X1 is DAsp.
426. The peptide of any of embodiments 420-422, wherein X1 is bAsp.
427. The peptide of any of embodiments 420-422, wherein X1 is Glu.
428. The peptide of any of embodiments 420-422, wherein X1 is DGlu.
429. The peptide of any of embodiments 420-428, wherein X2 is Phe(4-sulfomethyl).
430. The peptide of any of embodiments 420-428, wherein X2 is sTyr.
431. The peptide of any of embodiments 420-430, wherein X3 is Nle or Leu.
432. The peptide of any of embodiments 420-431, wherein X3 is Nle.
433. The peptide of any of embodiments 420-431, wherein X3 is Leu.
434. The peptide of any of embodiments 420-430, wherein X3 is Ala.
435. The peptide of any of embodiments 420-430, wherein X3 is Ile.
436. The peptide of any of embodiments 420-430, wherein X3 is Lys.
437. The peptide of any of embodiments 420-430, wherein X3 is Arg.
438. The peptide of any of embodiments 420-430, wherein X3 is Pro.
439. The peptide of any of embodiments 420-430, wherein X3 is Met.
440. The peptide of any of embodiments 420-430, wherein X3 is Phe.
441. The peptide of any of embodiments 420-430, wherein X3 is Ser.
442. The peptide of any of embodiments 420-430, wherein X3 is His.
443. The peptide of any of embodiments 420-442, wherein X6 is Nle.
444. The peptide of any of embodiments 420-442, wherein X6 is Ile.
445. The peptide of any of embodiments 420-442, wherein X6 is Gin.
446. The peptide of any of embodiments 420-442, wherein X6 is Met.
447. The peptide of any of embodiments 420-442, wherein X6 is Met(O2).
448. The peptide of any of embodiments 420-442, wherein X6 is Leu.
449. The peptide of any of embodiments 420-442, wherein X6 is Val.
450. The peptide of any of embodiments 420-442, wherein X6 is Pro.
451. The peptide of any of embodiments 420-442, wherein X6 is Ala.
452. The peptide of any of embodiments 420-451, wherein X8 is Phe, MePhe, 1Nal, Me1Nal, Trp, or 2Nal.
453. The peptide of any of embodiments 420-452, wherein X8 is Phe.
454. The peptide of any of embodiments 420-452, wherein X8 is MePhe.
455. The peptide of any of embodiments 420-452, wherein X8 is 1Nal.
456. The peptide of any of embodiments 420-452, wherein X8 is Me1Nal.
457. The peptide of any of embodiments 420-452, wherein X8 is Trp.
458. The peptide of any of embodiments 420-452, wherein X8 is 2Nal.
459. The peptide of any of embodiments 420-458, which has a maximum of six amino acid changes as compared to CCK-8s (SEQ ID NO: 1).
460. The peptide of any of embodiments 420-459, which has a maximum of five amino acid changes as compared to CCK-8s (SEQ ID NO: 1).
461. The peptide of any of embodiments 420-460, which has a maximum of four amino acid changes as compared to CCK-8s (SEQ ID NO: 1).
462. The peptide of any of embodiments 420-461, which has a maximum of three amino acid changes as compared to CCK-8s (SEQ ID NO: 1).
463. The peptide of any of embodiments 420-462, which has a maximum of two amino acid changes as compared to CCK-8s (SEQ ID NO: 1).
464. The peptide of any of embodiments 420-463, which has a minimum of two amino acid changes as compared to CCK-8s (SEQ ID NO: 1).
465. The peptide of any of embodiments 420-463, which has a minimum of three amino acid changes as compared to CCK-8s (SEQ ID NO: 1).
466. The peptide of any of embodiments 420-463, which has a minimum of four amino acid changes as compared to CCK-8s (SEQ ID NO: 1).
467. The peptide of any of embodiments 420-463, which has a minimum of five amino acid changes as compared to CCK-8s (SEQ ID NO: 1).
468. The peptide of any of embodiments 420-463, which has a minimum of six amino acid changes as compared to CCK-8s (SEQ ID NO: 1).
469. The peptide of any of embodiments 420-468, which has six amino acid changes as compared to CCK-8s (SEQ ID NO: 1).
470. The peptide of any of embodiments 420-468, which has five amino acid changes as compared to CCK-8s (SEQ ID NO: 1).
471. The peptide of any of embodiments 420-468, which has four amino acid changes as compared to CCK-8s (SEQ ID NO: 1).
472. The peptide of any of embodiments 420-468, which has three amino acid changes as compared to CCK-8s (SEQ ID NO: 1).
473. The peptide of any of embodiments 420-468, which has two amino acid changes as compared to CCK-8s (SEQ ID NO: 1).
474. A peptide selected from the following:

(Seq 21)
Asp-sTyr-Nle-Gly-Trp-Nle-DMeAsp-Trp-amide,
(Seq 22)
Asp-sTyr-Nle-Gly-Trp-Nle-DMeAsp-1Nal-amide,
(Seq 23)
Asp-Phe(4-sulfomethyl)-Nle-Gly-Trp-Nle-DMeAsp-
MePhe-amide,
(Seq 24)
Asp-sTyr-Leu-Gly-Trp-Nle-DMeAsp-Me1Nal-amide,
(Seq 25)
Asp-Phe(4-sulfomethyl)-Leu-Gly-Trp-Nle-DMeAsp-
MePhe-amide,
(Seq 27)
Asp-sTyr-Leu-Gly-Trp-Nle-DMeAsp-Phe-amide,
(Seq 28)
Asp-sTyr-Nle-Gly-Trp-Nle-DMeAsp-MePhe-amide,
(Seq 29)
Asp-sTyr-Leu-Gly-Trp-Nle-DMeAsp-MePhe-amide,
(Seq 26)
Asp-sTyr-Leu-Gly-Trp-Ile-DMeAsp-MePhe-amide
(Seq 30)
Asp-sTyr-Leu-Gly-Trp-Gln-DMeAsp-MePhe-amide
(Seq 31)
Asp-sTyr-Leu-Gly-Trp-Met(O2)-DMeAsp-MePhe-amide
(Seq 32)
Asp-sTyr-Leu-Gly-Trp-Leu-DMeAsp-MePhe-amide
(Seq 33)
Asp-sTyr-Leu-Gly-Trp-Val-DMeAsp-MePhe-amide
(Seq 34)
Asp-sTyr-Leu-Gly-Trp-Pro-DMeAsp-MePhe-amide
(Seq 35)
Asp-sTyr-Lys-Gly-Trp-Nle-DMeAsp-MePhe-amide
(Seq 36)
Asp-sTyr-Arg-Gly-Trp-Nle-DMeAsp-MePhe-amide
(Seq 37)
Asp-sTyr-Pro-Gly-Trp-Nle-DMeAsp-MePhe-amide
(Seq 38)
Asp-sTyr-Met-Gly-Trp-Nle-DMeAsp-MePhe-amide
(Seq 39)
Asp-sTyr-Nle-Gly-Trp-Met-DMeAsp-MePhe-amide
(Seq 40)
sTyr-Nle-Gly-Trp-Nle-DMeAsp-MePhe-amide
(Seq 41)
Asp-sTyr-Met-Gly-Trp-Met-DMeAsp-MePhe-amide
(Seq 42)
DAsp-Phe(4-sulfomethyl)-Nle-Gly-Trp-Nle-DMeAsp-
MePhe-amide
(Seq 43)
bAsp-Phe(4-sulfomethyl)-Nle-Gly-Trp-Nle-DMeAsp-
MePhe-amide
(Seq 44)
DbAsp-Phe(4-sulfomethyl)-Nle-Gly-Trp-Nle-DMeAsp-
MePhe-amide
(Seq 45)
Glu-sTyr-Leu-Gly-Trp-Nle-DMeAsp-MePhe-amide
(Seq 46)
Asp-sTyr-Phe-Gly-Trp-Nle-DMeAsp-MePhe-amide
(Seq 47)
Asp-sTyr-Ser-Gly-Trp-Nle-DMeAsp-MePhe-amide
(Seq 48)
Asp-sTyr-Ala-Gly-Trp-Nle-DMeAsp-MePhe-amide
(Seq 49)
Asp-sTyr-Ile-Gly-Trp-Nle-DMeAsp-MePhe-amide
(Seq 50)
Asp-sTyr-His-Gly-Trp-Nle-DMeAsp-MePhe-amide
(Seq 51)
Gly-Ser-Ser-Gly-Ser-Ser-Asp-sTyr-Leu-Gly-Trp-
Nle-DMeAsp-MePhe-amide
(Seq 52)
Asp-sTyr-Leu-Gly-Trp-Ala-DMeAsp-MePhe-amide
(Seq 53)
Glu-sTyr-Leu-Gly-Trp-Gln-DMeAsp-Phe-amide
(Seq 54)
Glu-sTyr-Leu-Gly-Trp-Gln-DMeAsp-MePhe-amide
(Seq 55)
DGlu-sTyr-Leu-Gly-Trp-Nle-DMeAsp-MePhe-amide
(Seq 56)
Asp-sTyr-Leu-Gly-Trp-Nle-DMeAsp-2Nal-amide
(Seq 57)
Asp-sTyr-Met-Gly-Trp-Met-DMeAsp-Phe-amide
(Seq 58)
Glu-sTyr-Leu-Gly-Trp-Nle-DMeAsp-Phe-amide;

or a pharmaceutically acceptable salt, amide, or ester thereof.
475. The peptide of any of embodiments 420-474 which is not the peptide of Seq 22.
476. The peptide of any of embodiments 420-475 which is not the peptide of Seq 29.
477. The peptide of any of embodiments 420-476 which is not the peptide of Seq 36.
478. The peptide of any of embodiments 420-477 which is not the peptide of Seq 45.
479. The peptide of any of embodiments 420-478 which is not the peptide of Seq 51.
480. The peptide of any of embodiments 474-479, which is a peptide of any of embodiments 420-473.
481. The peptide of any of embodiments 420-480 which is a CCK peptide.
482. The peptide of any of embodiments 420-481 which is an analogue of CCK-8s (SEQ ID NO: 1).
483. The peptide of any of embodiments 420-482, which consists of 7 or 8 amino acid residues.
484. The peptide of any of embodiments 420-483, which consists of 8 amino acid residues.
485. The peptide of any of embodiments 420-484 which has an in vitro potency on the CCK-1R which is similar to that of CCK-8s (SEQ ID NO: 1).
486. The peptide of any of embodiments 420-485, which has an EC₅₀ value on the CCK-1R which is no more than 10 times the CCK-1R EC₅₀ value for CCK-8s (SEQ ID NO: 1).
487. The peptide of any of embodiments 420-486, which has an EC₅₀ value on the CCK-1R which is no more than 6 times the CCK-1R EC₅₀ value for CCK-8s (SEQ ID NO: 1).
488. The peptide of any of embodiments 420-487, which has an EC₅₀ value on the CCK-1R which is no more than 4 times the CCK-1R EC₅₀ value for CCK-8s (SEQ ID NO: 1).
489. The peptide of any of embodiments 420-488, which has an EC₅₀ value on the CCK-1R which is no more than 2 times the CCK-1R EC₅₀ value for CCK-8s (SEQ ID NO: 1).
490. The peptide of any of embodiments 420-489, which has an EC₅₀ value on the CCK-1R which is no more than 1.5 times the CCK-1R EC₅₀ value for CCK-8s (SEQ ID NO: 1).
491. The peptide of any of embodiments 486-490, wherein the EC₅₀ value on the CCK-1R is determined essentially as described in embodiments 148-159 for the derivative of the invention, preferably essentially as described in Example 91.
492. The peptide of any of embodiments 420-491 which has an in vitro potency on the CCK-2R which is significantly lower than that of CCK-8s (SEQ ID NO: 1).
493. The peptide of any of embodiments 420-492, which has an EC₅₀ value on the CCK-2R which is at least 500 times the CCK-2R EC₅₀ value for CCK-8s (SEQ ID NO: 1).
494. The peptide of any of embodiments 420-493, which has an EC₅₀ value on the CCK-2R which is at least 1000 times the CCK-2R EC₅₀ value for CCK-8s (SEQ ID NO: 1).
495. The peptide of any of embodiments 420-494, which has an EC₅₀ value on the CCK-2R which is at least 1500 times the CCK-2R EC₅₀ value for CCK-8s (SEQ ID NO: 1).
496. The peptide of any of embodiments 420-495, which has an EC₅₀ value on the CCK-2R which is at least 2000 times the CCK-2R EC₅₀ value for CCK-8s (SEQ ID NO: 1).
497. The peptide of any of embodiments 420-496, which has an EC₅₀ value on the CCK-2R which is at least 2500 times the CCK-2R EC₅₀ value for CCK-8s (SEQ ID NO: 1).
498. The peptide of any of embodiments 486-497, wherein the EC₅₀ value on the CCK-2R is determined essentially as described in embodiments 185-196 for the derivative of the invention, preferably essentially as described in Example 91.
499. The peptide of any of embodiments 420-498 which is selective for the CCK-1R.
500. The peptide of embodiment 499, wherein the CCK-1R selectivity is determined by the ratio [EC₅₀ (CCK-2R)/EC₅₀ (CCK-1R)] being greater than 1.
501. The peptide of any of embodiments 420-500, for which the ratio [EC₅₀ (CCK-2R)/EC₅₀ (CCK-1R)] is at least 2 times the corresponding ratio for CCK-8s (SEQ ID NO: 1).
502. The peptide of any of embodiments 420-501, for which the ratio [EC₅₀ (CCK-2R)/EC₅₀ (CCK-1R)] is at least 10 times the corresponding ratio for CCK-8s (SEQ ID NO: 1).
503. The peptide of any of embodiments 420-502, for which the ratio [EC₅₀ (CCK-2R)/EC₅₀ (CCK-1R)] is at least 50 times the corresponding ratio for CCK-8s (SEQ ID NO: 1).
504. The peptide of any of embodiments 420-503, for which the ratio [EC₅₀ (CCK-2R)/EC₅₀ (CCK-1R)] is at least 100 times the corresponding ratio for CCK-8s (SEQ ID NO: 1).
505. The peptide of any of embodiments 420-504, for which the ratio [EC₅₀ (CCK-2R)/EC₅₀ (CCK-1R)] is at least 500 times the corresponding ratio for CCK-8s (SEQ ID NO: 1).
506. The peptide of any of embodiments 420-505, for which the ratio [EC₅₀ (CCK-2R)/EC₅₀ (CCK-1R)] is at least 1000 times the corresponding ratio for CCK-8s (SEQ ID NO: 1).
507. The peptide of any of embodiments 420-506, for which the ratio [EC₅₀ (CCK-2R)/EC₅₀ (CCK-1R)] is at least 2300 times the corresponding ratio for CCK-8s (SEQ ID NO: 1).
508. The peptide of any of embodiments 420-507 wherein for calculation of the ratio [EC₅₀ (CCK-2R)/EC₅₀ (CCK-1R)] the EC₅₀ (CCK-2R) and the EC₅₀ (CCK-1R) values are determined for the peptide and for CCK-8s (SEQ ID NO: 1), using the same assays, respectively.
509. The peptide of embodiment 508, wherein the assays are as described in any of embodiments 185-196 (EC₅₀ (CCK-2R)) and 148-159 (EC₅₀ (CCK-1R) for the derivative of the invention, preferably essentially as described in Example 91.
510. The peptide of any of embodiments 1-509, which has an in vitro receptor binding (affinity) to the CCK-1R which is similar to that of native human CCK-8s (SEQ ID NO: 1).
511. The peptide of any of embodiments 1-510, wherein the in vitro affinity to the CCK-1R is determined as the IC₅₀ value.
512. The peptide of any of embodiments 1-511, which has a CCK-1R IC₅₀ value which is no more than 10 times the CCK-1R IC₅₀ value for CCK-8s (SEQ ID NO: 1).
513. The peptide of any of embodiments 1-512, which has a CCK-1R IC₅₀ value on the CCK-1R which is no more than 8 times the CCK-1R IC₅₀ value for CCK-8s (SEQ ID NO: 1).
514. The peptide of any of embodiments 1-513, which has an CCK-1R IC₅₀ value on the CCK-1R which is no more than 6 times the CCK-1R EC₅₀ value for CCK-8s (SEQ ID NO: 1).
515. The peptide of any of embodiments 1-514, which has a CCK-1R IC₅₀ value on the CCK-1R which is no more than 5 times the CCK-1R IC₅₀ value for CCK-8s (SEQ ID NO: 1).
516. The peptide of any of embodiments 1-515, which has a CCK-1R IC₅₀ value on the CCK-1R which is no more than 4 times the CCK-1R IC₅₀ value for CCK-8s (SEQ ID NO: 1).
517. The peptide of any of embodiments 1-516, which has a CCK-1R IC₅₀ value on the CCK-1R which is no more than 3 times the CCK-1R IC₅₀ value for CCK-8s (SEQ ID NO: 1).
518. The peptide of any of embodiments 1-517, which has a CCK-1R IC₅₀ value on the CCK-1R which is no more than 2 times the CCK-1R IC₅₀ value for CCK-8s (SEQ ID NO: 1).
519. The peptide of any of embodiments 1-518, which has a CCK-1R IC₅₀ value on the CCK-1R which is no more than 1 times the CCK-1R IC₅₀ value for CCK-8s (SEQ ID NO: 1).
520. The peptide of any of embodiments 1-519, which has a CCK-1R IC₅₀ value on the CCK-1R which is no more than 0.5 times the CCK-1R IC₅₀ value for CCK-8s (SEQ ID NO: 1).
521. The peptide of any of embodiments 1-520, which has a CCK-1R IC₅₀ value on the CCK-1R which is equal to or lower than 0.25 times the CCK-1R IC₅₀ value for CCK-8s (SEQ ID NO: 1).
522. The peptide of any of embodiments 510-521, wherein the IC₅₀ value on the CCK-1R is determined essentially as described in embodiments 247-256 for the derivative of the invention, preferably essentially as described in Example 92.
523. The peptide of any of embodiments 420-522, which has an in vitro receptor binding (affinity) to the CCK-2R which is significant lower than that of native human CCK-8s (SEQ ID NO: 1).
524. The peptide of any of embodiments 420-523, wherein the in vitro affinity to the CCK-2R is determined as the IC₅₀ value.
525. The peptide of any of embodiments 420-524, which has a CCK-2R IC₅₀ value which is at least 100 times the CCK-2R IC₅₀ value for CCK-8s (SEQ ID NO: 1).
526. The peptide of any of embodiments 420-525, which has a CCK-2R IC₅₀ value on the CCK-2R which is at least 200 times the CCK-2R IC₅₀ value for CCK-8s (SEQ ID NO: 1).
527. The peptide of any of embodiments 420-526, which has an CCK-2R IC₅₀ value on the CCK-2R which is at least 300 times the CCK-2R EC₅₀ value for CCK-8s (SEQ ID NO: 1).
528. The peptide of any of embodiments 420-527, which has a CCK-2R IC₅₀ value on the CCK-2R which is at least 500 times the CCK-2R IC₅₀ value for CCK-8s (SEQ ID NO: 1).
529. The peptide of any of embodiments 420-528, which has a CCK-2R ICso value on the CCK-2R which is at least 1000 times the CCK-2R IC₅₀ value for CCK-8s (SEQ ID NO: 1).
530. The peptide of any of embodiments 420-529, which has a CCK-2R IC₅₀ value on the CCK-2R which is at least 2000 times the CCK-2R IC₅₀ value for CCK-8s (SEQ ID NO: 1).
531. The peptide of any of embodiments 420-530, which has a CCK-2R IC₅₀ value on the CCK-2R which is at least 5000 times the CCK-2R IC₅₀ value for CCK-8s (SEQ ID NO: 1).
532. The peptide of any of embodiments 420-531, which has a CCK-2R IC₅₀ value on the CCK-2R which is at least 10000 times the CCK-2R IC₅₀ value for CCK-8s (SEQ ID NO: 1).
533. The peptide of any of embodiments 420-532, which has a CCK-2R IC₅₀ value on the CCK-2R which is at least 20000 times the CCK-2R IC₅₀ value for CCK-8s (SEQ ID NO: 1).
534. The peptide of any of embodiments 420-533, which has a CCK-2R IC₅₀ value on the CCK-2R which is similar to or higher than the CCK-2R IC₅₀ value for Gastrin-17 (SEQ ID NO: 2).
535. The peptide of any of embodiments 523-533, wherein the IC₅₀ value on the CCK-2R is determined essentially as described in embodiments 282-291 for the derivative of the invention, preferably essentially as described in Example 92.
536. The peptide of any of embodiments 420-535, which selectively binds to the CCK-1R.
537. The peptide of any of embodiments 420-536, which has a ratio [IC₅₀ (CCK-2R) /IC₅₀ (CCK-1R)] of above 1.
538. The peptide of any of embodiments 420-537, for which the ratio [IC₅₀ (CCK-2R)/IC₅₀ (CCK-1R)] is at least 100 times the corresponding ratio for CCK-8s (SEQ ID NO: 1).
539. The peptide of any of embodiments 420-538, for which the ratio [IC₅₀ (CCK-2R)/IC₅₀ (CCK-1R)] is at least 1000 times the corresponding ratio for CCK-8s (SEQ ID NO: 1).
540. The peptide of any of embodiments 420-539, for which the ratio [IC₅₀ (CCK-2R)/IC₅₀ (CCK-1R)] is at least 2000 times the corresponding ratio for CCK-8s (SEQ ID NO: 1).
541. The peptide of any of embodiments 420-540, for which the ratio [IC₅₀ (CCK-2R)/IC₅₀ (CCK-1R)] is at least 10000 times the corresponding ratio for CCK-8s (SEQ ID NO: 1).
542. The peptide of any of embodiments 420-541, for which the ratio [IC₅₀ (CCK-2R)/IC₅₀ (CCK-1R)] is at least 23000 times the corresponding ratio for CCK-8s (SEQ ID NO: 1).
543. The peptide of any of embodiments 420-542, for which the ratio [IC₅₀ (CCK-2R)/IC₅₀ (CCK-1R)] is at least 50000 times the corresponding ratio for CCK-8s (SEQ ID NO: 1).
544. The peptide of any of embodiments 420-543, for which the ratio [IC₅₀ (CCK-2R)/IC₅₀ (CCK-1R)] is at least 70000 times the corresponding ratio for CCK-8s (SEQ ID NO: 1).
545. The peptide of any of embodiments 420-544 wherein for calculation of the ratio [IC₅₀ (CCK-2R)/IC₅₀ (CCK-1R)] the IC₅₀ (CCK-2R) and the IC₅₀ (CCK-1R) values are determined for the peptide and for CCK-8s (SEQ ID NO: 1), using the same assays, respectively.
546. The peptide of embodiment 545, wherein the assays are as described in any of embodiments 281-291 (IC₅₀ (CCK-2R)) and 246-256 (IC₅₀ (CCK-1R)) for the derivative of the invention, preferably essentially as described in Example 92.
547. A method of preparing the peptide of any of embodiments 420-446, which is an SPPS method.
548. The method of embodiment 547, wherein Fmoc based chemistry is used.
549. The method of any of embodiments 547-548, which is performed essentially as described in the experimental section herein, in particular in the section headed “Methods for Synthesis, Purification and Analysis of Example Compounds”.
550. A method of preparing the derivative of any of embodiments 1-419, wherein the peptide backbone is prepared as described in any of embodiments 547-549, and the P-L part of formula I is added by SPPS.
551. The method of embodiment 550, wherein Fmoc based chemistry is used.
552. The method of any of embodiments 550-551, which is performed essentially as described in the experimental section herein, in particular in the section headed “Methods for Synthesis, Purification and Analysis of Example Compounds”, more in particular in the subsection headed “Attachment of linker and fatty acid derivative”.
553. The method of any of embodiments 550-552, wherein the derivative is cleaved from the resin and the side chains deprotected.
554. The method of embodiment 553, which is performed essentially as described in the experimental section herein, in particular in the section headed “Methods for Synthesis, Purification and Analysis of Example Compounds”, more in particular in the subsection headed “Cleavage from the resin and side-chain deprotection”.
555. The method of any of embodiments 550-554, which comprises a step of removal of a neopentyl group from Tyr(SO₃-neopentyl).
556. The method of embodiment 555, wherein the neopentyl group is removed from Tyr(SO₃-neopentyl) essentially as described in the section headed “Methods for Synthesis, Purification and Analysis of Example Compounds”, more in particular in the subsection headed “Removal of the neopentyl group from Tyr(SO₃-neopentyl)”.
557. The method of any of embodiments 550-554, which comprises a step of removal of a trichloroethyl (TCE) group from Phe(4-sulfomethyl-TCE).
558. The method of embodiment 557, wherein the trichloroethyl (TCE) group is removed from Phe(4-sulfomethyl-TCE) essentially as described in the section headed “Methods for Synthesis, Purification and Analysis of Example Compounds”, more in particular in the subsection headed “Removal of a trichloroethyl (TCE) group from Phe(4-sulfomethyl-TCE)”.
559. The method of any of embodiments 547-558, wherein the peptide or derivative is dissolved in neutral aqueous ammonium acetate and acetonitrile and purified by reversed-phase preparative HPLC.
560. The method of embodiment 559, wherein the purification is performed essentially as described in the section headed “Methods for Synthesis, Purification and Analysis of Example Compounds”, more in particular in the subsection headed “Purification and Quantification”.
561. A method of preparing the derivative of any of embodiments 1-419, which method comprises the following steps:

(i) preparation of the intermediate peptide of any of embodiments 420-546 using Solid Phase Peptide Synthesis (SPPS);

(ii) preparation of the P-L side chain of any of embodiments 1-81 in the form of its C-terminal carboxylic acid, and activation thereof with a coupling agent resulting in the formation of an acylating agent; and

(iii) attachment of the acylating agent from step (ii) to the N-terminus of the intermediate peptide from step (i).

562. The method of embodiment 561, wherein Fmoc based chemistry is used.
563. The method of any of embodiments 561-562, wherein the intermediate peptide is cleaved from the resin and optionally purified.
564. The method of any of embodiments 561-563, wherein the derivative is cleaved from the resin and the side chain deprotected.
565. The method of any of embodiments 561-564, which comprises a step of removal of a neopentyl group from Tyr(SO₃-neopentyl).
566. The method of embodiment 565, wherein the neopentyl group is removed from Tyr(SO₃-neopentyl) essentially as described in the section headed “Methods for Synthesis, Purification and Analysis of Example Compounds”, more in particular in the subsection headed “Removal of the neopentyl group from Tyr(SO₃-neopentyl)”.
567. The method of any of embodiments 561-566, which comprises a step of removal of a trichloroethyl (TCE) group from Phe(4-sulfomethyl-TCE).
568. The method of embodiment 567, wherein the trichloroethyl (TCE) group is removed from Phe(4-sulfomethyl-TCE) essentially as described in the section headed “Methods for Synthesis, Purification and Analysis of Example Compounds”, more in particular in the subsection headed “Removal of a trichloroethyl (TCE) group from Phe(4-sulfomethyl-TCE)”.
569. The method of any of embodiments 547-558, wherein the peptide or derivative is dissolved in neutral aqueous ammonium acetate and acetonitrile and purified by reversed-phase preparative HPLC.
570. The method of embodiment 569, wherein the purification is performed essentially as described in the section headed “Methods for Synthesis, Purification and Analysis of Example Compounds”, more in particular in the subsection headed “Purification and Quantification”.
571. The method of any of embodiments 561-570, wherein the attachment of step (iii) is effected by formation of an amide bond.
572. The method of any of embodiments 561-571, wherein the coupling agent is an N-hydroxysuccinimide ester.
573. The method of any of embodiments 561-572, wherein the preparation of the side chain is performed as described in WO 2009/083549 A1.
574. The method of any of embodiments 561-573, wherein the attachment of the side chain to the intermediate peptide is performed as described in WO 2009/083549 A1.
575. A pharmaceutical composition which comprises a derivative of any of embodiments 1-419 or a peptide of any of embodiments 420-546, together with a pharmaceutically acceptable excipient.
576. The pharmaceutical composition of embodiment 575, which is a solution.
577. The pharmaceutical composition of any of embodiments 575-576, which is an aqueous solution for subcutaneous injection.
578. The pharmaceutical composition of any of embodiments 575-577, which is a phosphate buffered solution.
579. The pharmaceutical composition of any of embodiments 575-577, which is a Tris(hydroxymethyl)aminomethane buffered solution.
580. The pharmaceutical composition of any of embodiments 575-579, which has a pH about 7.4.
581. The pharmaceutical composition of any of embodiments 575-580, wherein the concentration of the buffer is about 8.0 mM.
582. The pharmaceutical composition of any of embodiments 575-581, which further contains an isotonic agent.
583. The pharmaceutical composition of embodiment 582, wherein the isotonic agent is propylene glycol.
584. The pharmaceutical composition of embodiment 583, wherein the concentration of propylene glycol is about 14 mg/mL.
585. The pharmaceutical composition of any of embodiments 575-584 which further comprises a preservative.
586. The pharmaceutical composition of any of embodiments 575-585 wherein the preservative is selected from m-cresol and phenol.
587. The pharmaceutical composition of embodiment 586 wherein the preservative is phenol.
588. The pharmaceutical composition of embodiment 587, wherein the phenol concentration is about 58 mM.
589. The pharmaceutical composition of any of embodiments 575-588, wherein the concentration of the derivative is in the range of 0.10 mM to 10.0 mM, end-points included.
590. The pharmaceutical composition of any of embodiments 575-589, wherein the concentration of the derivative is in the range of 0.2-50000 nmol/ml (0.0002-50 mM), end-points included.
591. The pharmaceutical composition of any of embodiments 575-590, wherein the concentration of the derivative is in the range of 5.0-20000 nmol/ml (0.005-20 mM), end-points included.
592. The pharmaceutical composition of any of embodiments 575-591, wherein the concentration of the derivative is at least 1.0 mM.
593. The pharmaceutical composition of any of embodiments 575-592, wherein the concentration of the derivative is 1.0-10 mM.
594. The pharmaceutical composition of any of embodiments 575-593, wherein the concentration of the derivative is 5 mM.
595. A pharmaceutical composition which has the following formulation:
5.0 mM (9.25 mg/mL) of the compound of Example 3
pH 7.4
8.0 mM phosphate
14 mg/mL propylene glycol
58 mM phenol.
596. A pharmaceutical composition which has the following formulation:
10.0 mM (18.5 mg/mL) of the compound of Example 3
pH 7.4
8.0 mM phosphate
14 mg/mL propylene glycol
58 mM phenol.
597. A pharmaceutical composition which has the following formulation:
5.0 mM (9.25 mg/mL) of the compound of Example 3
pH 7.4

### 8.0 mM Tris(hydroxymethyl)aminomethane

14 mg/mL propylene glycol
58 mM phenol.
598. An injection device containing the pharmaceutical composition of any of embodiments 575-597.
599. The injection device of embodiment 598 which is a disposable, pre-filled, multi-dose pen.
600. The injection device of embodiment 598 which is a fixed dose, multi-shot device.
601. A derivative of any of embodiments 1-419 or a peptide of any of embodiments 420-546 for use as a medicament.
602. A derivative of any of embodiments 1-419 or a peptide of any of embodiments 420-546 for (a) the treatment or prevention of pancreatic disorders, obesity and obesity related disorders, diabetes, other metabolic diseases, gall stone, and/or, overweight; and/or for (b) the diagnosis of pancreatic and/or gall bladder mal-function.
603. The derivative of any of embodiments 1-419 or a peptide of any of embodiments 420-546 for
(i) Weight management, preferably in addition to diet and exercise;
(ii) Treatment of obesity, preferably in adults with a body mass index (BMI) of 30 or greater;
(iii) Treatment of overweight, preferably in adults with a BMI of 27 or greater who, more preferably, have at least one weight-related co-morbidity or condition such as high cholesterol, obstructive sleep apnoea, hypertension, type 2 diabetes, or dyslipidaemia.
604. The derivative of any of embodiments 1-419 or the peptide of any of embodiments 420-546 for weight management monotherapy.
605. The derivative of any of embodiments 1-419 or the peptide of any of embodiments 420-546 for weight management in combination with other weight reducing agents.
605a. The derivative or peptide of any of embodiments 603-605 wherein the weight management is chronic.
606. The derivative or peptide of any of embodiments 604-605a where the use in monotherapy or in combination therapy is in addition to diet and exercise.
607. The derivative or peptide of any of embodiments 604-606, where the administration is once daily.
608. The derivative or peptide of any of embodiments 604-606, where the administration is once weekly.
608a. The derivative or peptide of any of embodiments 604-606, where the administration is once monthly.
609. The derivative or peptide of any of embodiments 604-608a, wherein the other weight reducing agents are selected from GLP-1 receptor agonists, amylin receptor agonists, leptin receptor agonists, PYY receptor agonists, MIC-1 receptor agonists, and/or glucagon receptor agonists.
610. The derivative or peptide of any of embodiments 604-609, wherein the other weight reducing agents are selected from estrogene, bupropion+naltrexone (Contrave®/Mysimba®), Cetilistat®, locaserin (Belviq®), phentermine+topiramate (Qsymia®), phentermine, orlistat (Xenical®/Alli®), and/or canagliflozin+phentermine.
611. The derivative or peptide of any of embodiments 604-610, wherein the GLP-1 receptor agonist is selected from liraglutide, Victoza®, Saxenda®, and/or semaglutide.
612. The derivative or peptide of any of embodiments 604-611, wherein the GLP-1 receptor agonist is selected from exenatide (Byetta®), exenatide once weekly (Bydureon®), lixisenatide (Lyxumia®), albiglutide (Eperzan®/Tanzeum®), and/or dulaglutide (Trulicity®).
613. A method for (a) the treatment or prevention of pancreatic disorders, obesity and obesity related disorders, diabetes, other metabolic diseases, gall stone, and/or overweight; and/or for (b) the diagnosis of pancreatic and/or gall bladder mal-function; the method comprising the administration of a derivative of any of embodiments 1-419 or a peptide of any of embodiments 420-546.
614. A method for
(i) weight management, preferably in addition to diet and exercise;
(ii) treatment of obesity, preferably in adults with a body mass index (BMI) of 30 or greater;
(iii) treatment of overweight, preferably in adults with a BMI of 27 or greater who, more preferably, have at least one weight-related condition such as hypertension, type 2 diabetes, or dyslipidaemia;

the method comprising the administration of a derivative of any of embodiments 1-419 or a peptide of any of embodiments 420-546.

615. The method of any of embodiments 613-614 for weight management monotherapy.
616. The method of any of embodiments 613-614 for weight management in combination with other weight reducing agents.
617. The method of any of embodiments 613-616 which is in addition to diet and exercise.
618. The method of any of embodiments 613-617, wherein the administration is once daily.
619. The method of any of embodiments 613-618, wherein the administration is once weekly.
619a. The method of any of embodiments 613-619, wherein the administration is once monthly.
619b. The method of any of embodiments 615-619 for chronic weight management.
620. The method of any of embodiments 613-619b, wherein the other weight reducing agents are selected from GLP-1 receptor agonists, amylin receptor agonists, leptin receptor agonists, PYY receptor agonists, MIC-1 receptor agonists, and/or glucagon receptor agonists.
621. The method of any of embodiments 613-620, wherein the other weight reducing agents are selected from estrogene, bupropion+naltrexone (Contrave®/Mysimba®), Cetilistat®, locaserin (Belviq®), phentermine+topiramate (Qsymia®), phentermine, orlistat (Xenical®/Alli®), and/or canagliflozin+phentermine.
622. The method of any of embodiments 613-621, wherein the GLP-1 receptor agonist is selected from liraglutide, Victoza®, Saxenda®, and/or semaglutide.
623. The method of any of embodiments 613-622, wherein the GLP-1 receptor agonist is selected from exenatide (Byetta®), exenatide once weekly (Bydureon®), lixisenatide (Lyxumia®), albiglutide (Eperzan®/Tanzeum®), and/or dulaglutide (Trulicity®).

In some embodiments the derivative of the invention is not selected from Chem. 21, Chem. 22, Chem. 23, Chem. 24, Chem. 25, Chem. 26, Chem. 27, Chem. 28, Chem. 29, Chem. 30, Chem. 31, Chem. 32, Chem. 33, Chem. 34, Chem. 35, Chem. 36, Chem. 37, Chem. 38, Chem. 39, Chem. 40, Chem. 41, Chem. 42, Chem. 43, Chem. 44, Chem. 45, Chem. 46, Chem. 47, Chem. 48, Chem. 49, Chem. 50, Chem. 51, Chem. 52, Chem. 53, Chem. 54, Chem. 55, Chem. 56, Chem. 57, Chem. 58, Chem. 59, Chem. 60, Chem. 61, Chem. 62, Chem. 63, Chem. 64, Chem. 65, Chem. 66, Chem. 67, Chem. 68, Chem. 69, Chem. 70, Chem. 71, and Chem. 72; and is also not a pharmaceutically acceptable salt, amide, and ester thereof.

In some embodiments the peptide of the invention is not selected from Seq 21, Seq 22, Seq 23, Seq 24, Seq 25, Seq 27, Seq 28, Seq 29, Seq 30, Seq 31, Seq 32, Seq 33, Seq 34, Seq 35, Seq 36, Seq 37, Seq 38, Seq 39, Seq 40, Seq 41, Seq 42, Seq 43, Seq 44, Seq 45, Seq 46, Seq 47, Seq 48, Seq 49, Seq 50, Seq 51, Seq 52, Seq 53, Seq 54, Seq 55, Seq 56, Seq 57, and Seq 58; and is also not a pharmaceutically acceptable salt, amide, or ester thereof.

### Particular Embodiments

The following are additional particular embodiments of the invention:

1. A peptide derivative of the general formula I:

P-L-X1-X2-X3-Gly-Trp-X6-DMeAsp-X8-NH₂ (formula I),

wherein P is Chem. 1:

HOOC—(CH₂)ₓ—CO—*, Chem. 1:

wherein x is an integer in the range of 12-18; L is absent, or L comprises at least one linker element of formula Chem. 2, Chem. 3, Chem. 4, and/or Chem. 5:

*—NH—CH(COOH)—(CH₂)₂—CO—*, Chem. 2:

*—NH—(CH₂)₂—[O—(CH₂)₂]ₖ—O—[CH₂]ₙ—CO—*, Chem. 3:

wherein k is an integer in the range of 1-5, and n is an integer in the range of 1-5,

*—NH—CH(CH₂OH)—CO—*, Chem. 4:

and/or

*—NH—CH₂—CO—*; Chem. 5:

X1 is absent, Asp, D-Asp, Glu, or D-Glu;
X2 is Phe(4-sulfomethyl) or sTyr;

### X3 is Nle, Leu, Ala, Ile, or Val;

### X6 is Nle;

### X8 is Phe, MePhe, 1Nal, Me1Nal, 2Nal, Me2Nal, Trp, or MeTrp; and

the NH₂ group at the C-terminus of formula I means that X8 is C-terminally amidated;
or a pharmaceutically acceptable salt, amide, or ester thereof.
2. The derivative of embodiment 1, wherein P and L are connected via an amide bond; or if L is absent P and X1 are connected via an amide bond; or if L is absent and X1 is absent, P and X2 are connected via an amide bond.
3. The derivative of any of embodiments 1-2, wherein L is connected to X1 via an amide bond, or if X1 is absent L is connected to X2 via an amide bond.
4. The derivative of any of embodiments 1-3, wherein X1, X2, X3, Gly, Trp, X6, DMeAsp, and X8 are mutually connected via amide bonds; or if X1 is absent X2, X3, Gly, Trp, X6, DMeAsp, and X8 are mutually connected via amide bonds.
5. The derivative of any of embodiments 1-4, wherein L comprises at least two linker elements.
6. The derivative of any of embodiments 1-5, wherein L comprises at least three linker elements.
7. The derivative of any of embodiments 1-6, wherein L comprises three linker elements.
8. The derivative of any of embodiments 1-7, wherein L comprises Chem. 2.
9. The derivative of any of embodiments 1-8, wherein Chem. 2 is represented by Chem. 2a:

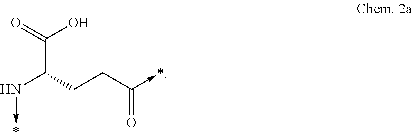

10. The derivative of any of embodiments 1-9, wherein L comprises Chem. 3.
11. The derivative of any of embodiments 1-10, wherein L comprises two times Chem. 3.
12. The derivative of any of embodiments 1-11, wherein n is 1.
13. The derivative of any of embodiments 1-12, wherein k is 1.
14. The derivative of any of embodiments 1-13, wherein Chem. 3 is represented by Chem. 3a:

*—NH—(CH₂)₂—O—(CH₂)₂—O—CH—CO—*. Chem. 3a:

15. The derivative of any of embodiments 1-14, wherein L comprises Chem. 2a and Chem. 3a.
16. The derivative of any of embodiments 1-15, wherein L comprises one Chem. 2a linker element and two Chem. 3a linker elements.
17. The derivative of any of embodiments 1-16, wherein L consists of one Chem. 2a element and two Chem. 3a elements (Chem. 2a-2×Chem. 3a), interconnected via amide bonds and in the sequence indicated, L being connected at its *—NH end to the CO—* end of P, and at its CO—* end to the amino group of X1, or if X1 is absent to the amino group of X2.
18. The derivative of any of embodiments 1-17, wherein x is 14 or 16.
19. The derivative of any of embodiments 1-18, wherein x is 14.
20. The derivative of any of embodiments 1-18, wherein x is 16.
21. The derivative of any of embodiments 1-20, wherein X1 is Asp.
22. The derivative of any of embodiments 1-21, wherein X2 is Phe(4-sulfomethyl).
23. The derivative of any of embodiments 1-21, wherein X2 is sTyr.
24. The derivative of any of embodiments 1-23, wherein X3 is Nle or Leu.
25. The derivative of any of embodiments 1-24, wherein X3 is Nle.
26. The derivative of any of embodiments 1-24, wherein X3 is Leu.
27. The derivative of any of embodiments 1-26, wherein X8 is Phe, MePhe, 1Nal, Me1Nal, or Trp.
28. The derivative of any of embodiments 1-27, wherein X8 is Phe.
29. The derivative of any of embodiments 1-27, wherein X8 is MePhe.
30. The derivative of any of embodiments 1-27, wherein X8 is 1Nal.
31. The derivative of any of embodiments 1-27, wherein X8 is Me1Nal.
32. The derivative of any of embodiments 1-27, wherein X8 is Trp.
33. The derivative of any of embodiments 1-32, which has a maximum of six amino acid changes as compared to CCK-8s (SEQ ID NO: 1).
34. The derivative of any of embodiments 1-32, which has a maximum of five amino acid changes as compared to CCK-8s (SEQ ID NO: 1).
35. The derivative of any of embodiments 1-32, which has a maximum of four amino acid changes as compared to CCK-8s (SEQ ID NO: 1).
36. The derivative of any of embodiments 1-32, which has a maximum of three amino acid changes as compared to CCK-8s (SEQ ID NO: 1).
37. The derivative of any of embodiments 1-36, which has a minimum of three amino acid changes as compared to CCK-8s (SEQ ID NO: 1).
38. The derivative of any of embodiments 1-36, which has a minimum of four amino acid changes as compared to CCK-8s (SEQ ID NO: 1).
39. The derivative of any of embodiments 1-36, which has a minimum of five amino acid changes as compared to CCK-8s (SEQ ID NO: 1).
40. The derivative of any of embodiments 1-36, which has a minimum of six amino acid changes as compared to CCK-8s (SEQ ID NO: 1).
41. The derivative of any of embodiments 1-40, which has five amino acid changes as compared to CCK-8s (SEQ ID NO: 1).
42. The derivative of any of embodiments 1-40, which has four amino acid changes as compared to CCK-8s (SEQ ID NO: 1).
43. The derivative of any of embodiments 1-40, which has three amino acid changes as compared to CCK-8s (SEQ ID NO: 1).
44. A peptide derivative selected from the following:

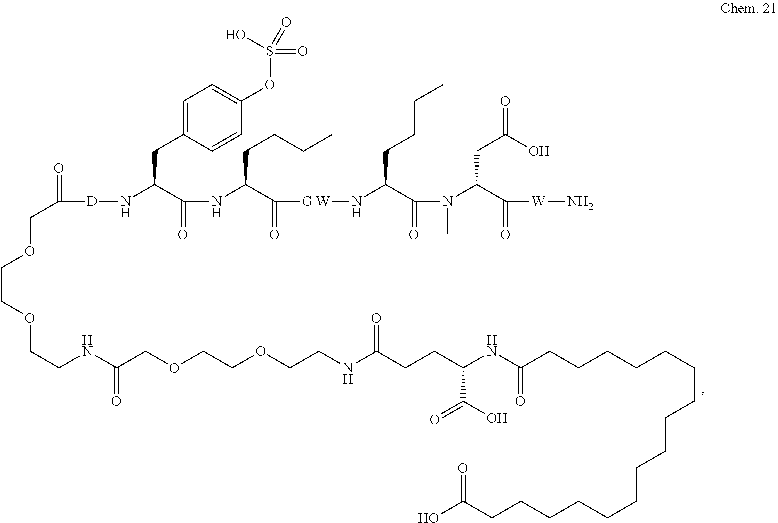

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Nle-Gly-Trp-Nle-DMeAsp-Trp-amide,

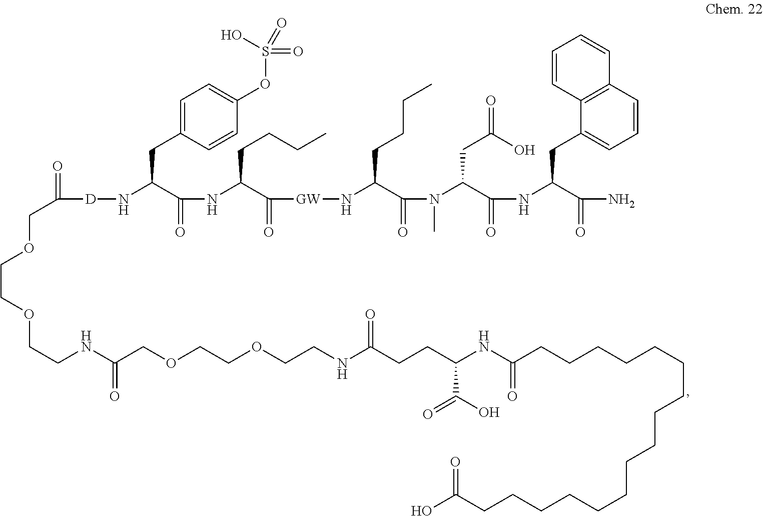

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Nle-Gly-Trp-Nle-DMeAsp-1Nal-amide,

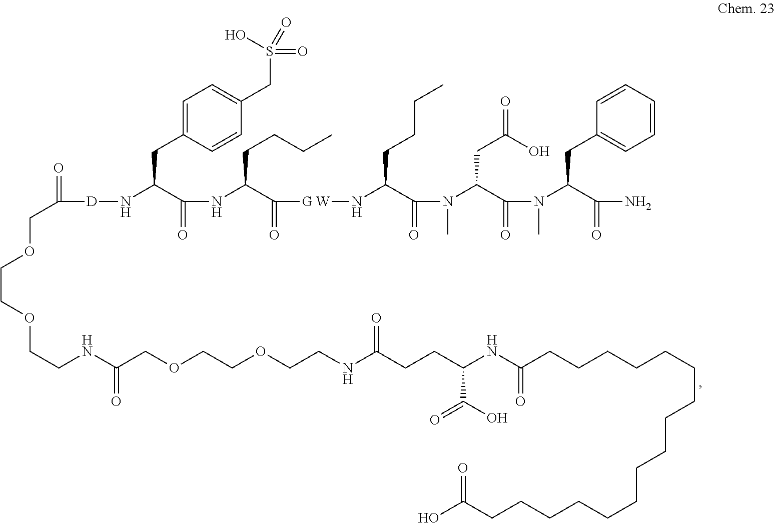

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-Phe(4-sulfomethyl)-Nle-Gly-Trp-Nle-DMeAsp-MePhe-amide,

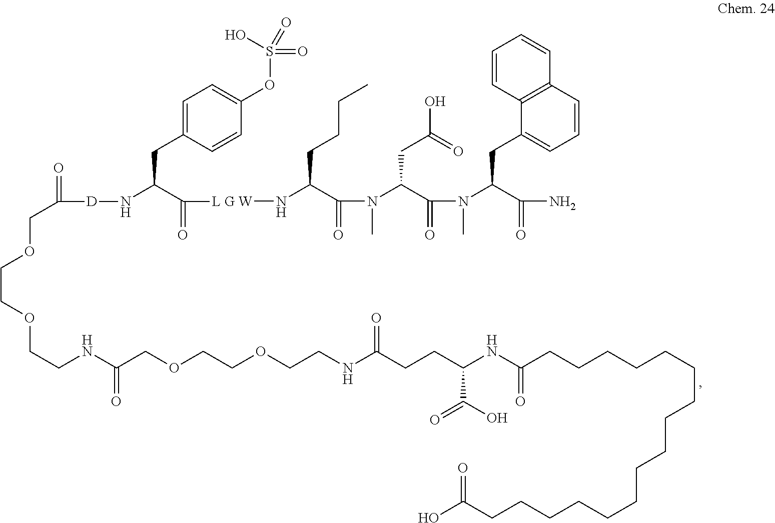

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Nle-DMeAsp-Me1Nal-amide,

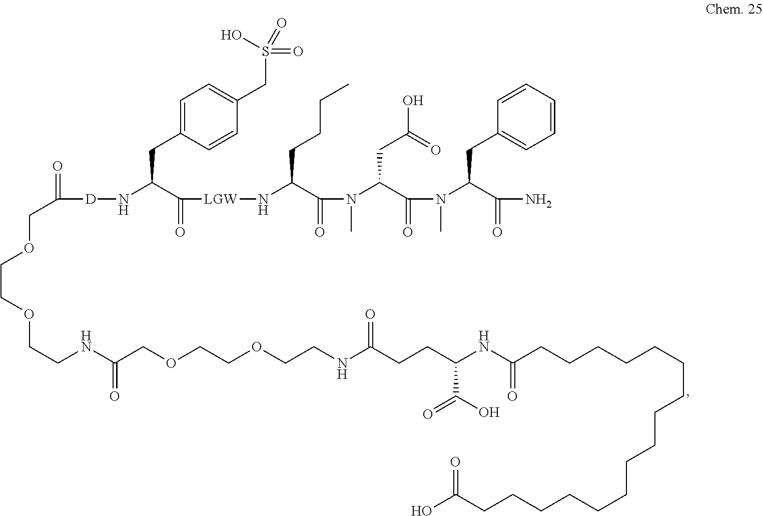

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-Phe(4-sulfomethyl)-Leu-Gly-Trp-Nle-DMeAsp-MePhe-amide,

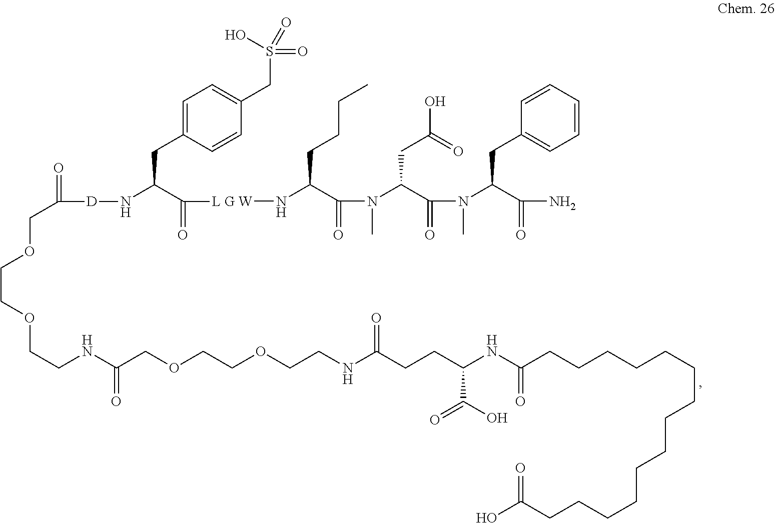

2-[2-[2-[[2-[2-[2-[[(4S)-4-Carboxy-4-(15-carboxypentadecanoylamino)butanoyl]amino]ethoxy]ethoxy]acetyl]amino]ethoxy]ethoxy]acetyl-Asp-Phe(4-sulfomethyl)-Leu-Gly-Trp-Nle-DMeAsp-MePhe-amide,

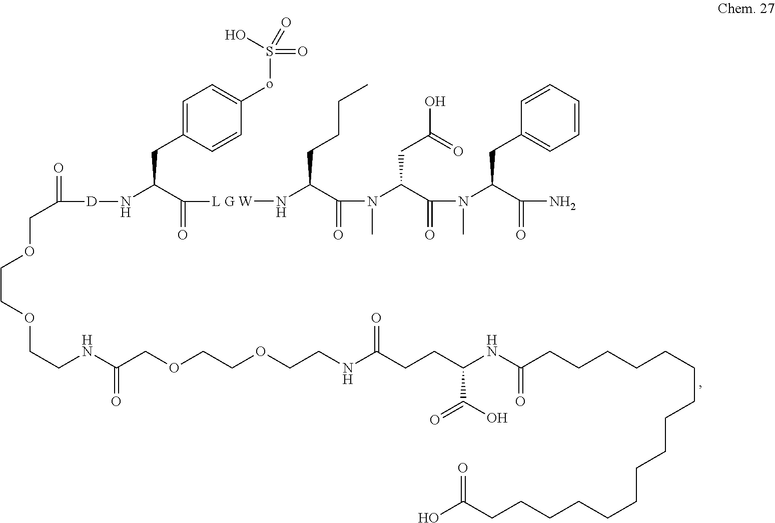

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Nle-DMeAsp-Phe-amide,

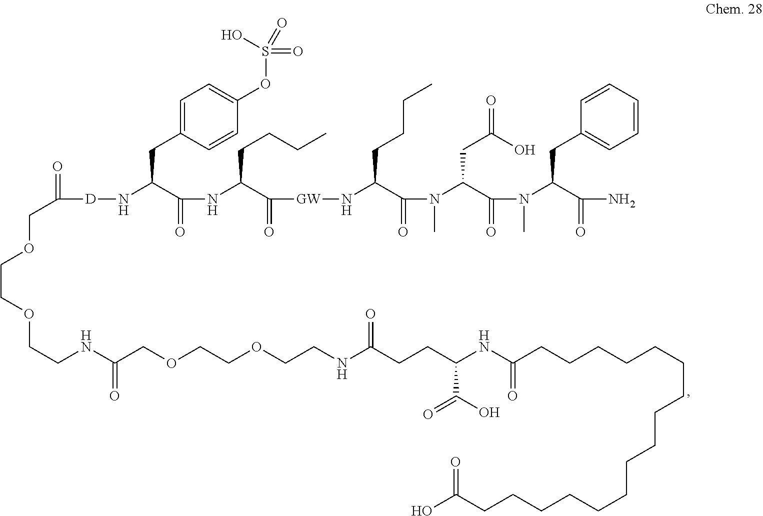

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Nle-Gly-Trp-Nle-DMeAsp-MePhe-amide,

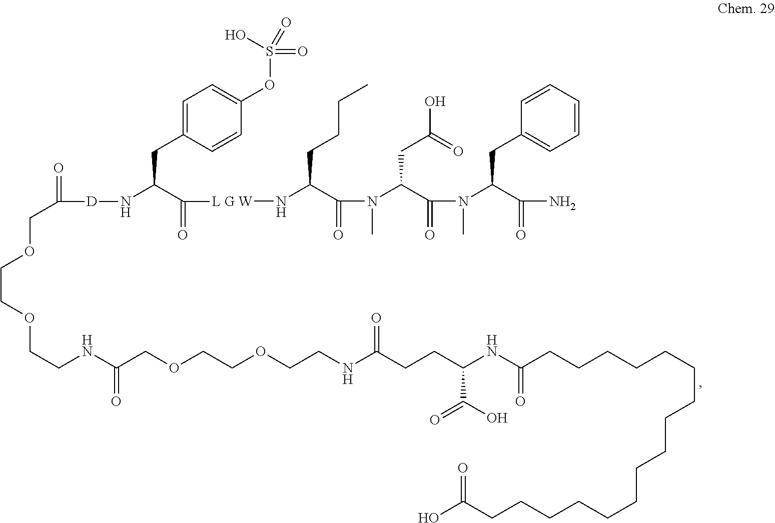

and
2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Nle-DMeAsp-MePhe-amide;
   or a pharmaceutically acceptable salt, amide, or ester thereof.
   45. The derivative of embodiment 44, which is a derivative of any of embodiments 1-43.
   46. The derivative of any of embodiments 1-45, wherein X1-X2-X3-Gly-Trp-X6-DMeAsp-X8-NH₂ constitutes a peptide part of formula I.
   47. The derivative of any of embodiments 1-46, wherein the peptide part is a CCK peptide.
   48. The derivative of any of embodiments 1-47 which is a CCK derivative.
   49. The derivative of any of embodiments 1-48, wherein the peptide part is an analogue of CCK-8s.
   50. The derivative of any of embodiments 1-49, wherein the peptide part consists of 7 or 8 amino acid residues.
   51. The derivative of any of embodiments 1-50, wherein the peptide part consists of 8 amino acid residues.
   52. The derivative of any of embodiments 1-51 which is a CCK-1R agonist.
   53. The derivative of any of embodiments 1-52 which is a full CCK-1R agonist.
   54. The derivative of any of embodiments 1-53, which has an in vitro potency on the CCK-1R which is similar to or only moderately decreased as compared to that of native human CCK-8s (SEQ ID NO: 1).
   55. The derivative of any of embodiments 1-54, wherein in vitro potency on the CCK-1R is determined as the EC₅₀ value.
   56. The derivative of any of embodiments 1-55, wherein the EC₅₀ value refers to the concentration of the derivative which in a CCK-1R activation assay induces a response half way between the basal (or bottom) response and the maximal (or top) response.
   57. The derivative of any of embodiments 1-56, wherein the CCK-1R activation assay is a whole cell functional assay.
   58. The derivative of any of embodiments 1-57, wherein the CCK-1R activation assay determines accumulation of inositol (1) phosphate (IP1).
   59. The derivative of any of embodiments 1-58, wherein IP1 is determined using an assay kit which is commercially available from CisBio Bioassays, France (catalogue 62IPAPEB).
   60. The derivative of any of embodiments 1-59, wherein the cells expressing the CCK-1R are 1321-N1 cells, for example from Perkin Elmer (product number ES-530-A).
   61. The derivative of any of embodiments 1-60, wherein the cells are cultivated essentially as described in Example 91.
   62. The derivative of any of embodiments 1-61, wherein IP1 is determined essentially as described in Example 91.
   63. The derivative of any of embodiments 1-62, wherein the EC₅₀ value is determined essentially as described in Example 91.
   64. The derivative of any of embodiments 1-63, wherein CCK-8s (SEQ ID NO: 1) is used as a positive reference.
   65. The derivative of any of embodiments 1-64, wherein Gastrin-17 (SEQ ID NO: 2) is used as a negative reference.
   66. The derivative of any of embodiments 1-65, wherein suitable serial dilutions of the derivative and the reference compounds are used.
   67. The derivative of any of embodiments 1-66, wherein the serial dilutions are from 10⁻⁶ M (one μM) to 10-12 M (one pM).
   68. The derivative of any of embodiments 1-67, wherein the in vitro potency (EC₅₀ value) on the CCK-1R is determined essentially as described in Example 91.
   69. The derivative of any of embodiments 1-68, which has an EC₅₀ value on the CCK-1R which is no more than 15 times the CCK-1R EC₅₀ value for CCK-8s (SEQ ID NO: 1).
   70. The derivative of any of embodiments 1-69, which has an EC₅₀ value on the CCK-1R which is no more than 14 times the CCK-1R EC₅₀ value for CCK-8s (SEQ ID NO: 1).
   71. The derivative of any of embodiments 1-70, which has an EC₅₀ value on the CCK-1R which is no more than 10 times the CCK-1R EC₅₀ value for CCK-8s (SEQ ID NO: 1).
   72. The derivative of any of embodiments 1-71, which has an in EC₅₀ value on the CCK-1R which is no more than 8 times the CCK-1R EC₅₀ value for CCK-8s (SEQ ID NO: 1).
   73. The derivative of any of embodiments 1-72, which has an in EC₅₀ value on the CCK-1R which is no more than 6 times the CCK-1R EC₅₀ value for CCK-8s (SEQ ID NO: 1).
   74. The derivative of any of embodiments 1-73, which has an EC₅₀ value on the CCK-1R which is no more than 4 times the CCK-1R EC₅₀ value for CCK-8s (SEQ ID NO: 1).
   75. The derivative of any of embodiments 1-74, which has an EC₅₀ value on the CCK-1R which is no more than 3 times the CCK-1R EC₅₀ value for CCK-8s (SEQ ID NO: 1).
   76. The derivative of any of embodiments 1-75, which has an EC₅₀ value on the CCK-1R which is no more than 2 times the CCK-1R EC₅₀ value for CCK-8s (SEQ ID NO: 1).
   77. The derivative of any of embodiments 1-76, which has an EC₅₀ value on the CCK-1R which is no more than 1 time the CCK-1R EC₅₀ value for CCK-8s (SEQ ID NO: 1).
   78. The derivative of any of embodiments 1-77, which has an EC₅₀ value on the CCK-1R which is equal to or lower than the CCK-1R EC₅₀ value for CCK-8s (SEQ ID NO: 1).
   79. The derivative of any of embodiments 1-78, which has an EC₅₀ value on the CCK-1R which is equal to or lower than 7000 pM.
   80. The derivative of any of embodiments 1-78, which has an EC₅₀ value on the CCK-1R which is equal to or lower than 5000 pM.
   81. The derivative of any of embodiments 1-78, which has an EC₅₀ value on the CCK-1R which is equal to or lower than 4000 pM.
   82. The derivative of any of embodiments 1-78, which has an EC₅₀ value on the CCK-1R which is equal to or lower than 3000 pM.
   83. The derivative of any of embodiments 1-78, which has an EC₅₀ value on the CCK-1R which is equal to or lower than 2000 pM.
   84. The derivative of any of embodiments 1-78, which has an EC₅₀ value on the CCK-1R which is equal to or lower than 1500 pM.
   85. The derivative of any of embodiments 1-78, which has an EC₅₀ value on the CCK-1R which is equal to or lower than 1200 pM.
   86. The derivative of any of embodiments 1-78, which has an EC₅₀ value on the CCK-1R which is equal to or lower than 1000 pM.
   87. The derivative of any of embodiments 1-78, which has an EC₅₀ value on the CCK-1R which is equal to or lower than 800 pM.
   88. The derivative of any of embodiments 1-78, which has an EC₅₀ value on the CCK-1R which is equal to or lower than 600 pM.
   89. The derivative of any of embodiments 1-78, which has an EC₅₀ value on the CCK-1R which is equal to or lower than 400 pM.
   90. The derivative of any of embodiments 1-78, which has an EC₅₀ value on the CCK-1R which is equal to or lower than 350 pM.
   91. The derivative of any of embodiments 1-90, which has an EC₅₀ value on the CCK-2R which is much lower than that of native human CCK-8s (SEQ ID NO: 1).
   92. The derivative of any of embodiments 1-91, wherein in vitro potency on the CCK-2R is determined as the EC₅₀ value.
   93. The derivative of any of embodiments 1-92, wherein the EC₅₀ value refers to the concentration of the derivative which in a CCK-2R activation assay induces a response half way between the basal (or bottom) response and the maximal (or top) response.
   94. The derivative of any of embodiments 1-93, wherein the CCK-2R activation assay is a whole cell functional assay.
   95. The derivative of any of embodiments 1-94, wherein the CCK-2R activation assay determines accumulation of inositol (1) phosphate (IP1).
   96. The derivative of any of embodiments 1-95, wherein following CCK-2R activation IP1 is determined using an assay kit which is commercially available from CisBio Bioassays, France (catalogue 62IPAPEB).
   97. The derivative of any of embodiments 1-59, wherein the cells expressing the CCK-2R are 1321-N1 cells, for example from Perkin Elmer (product number ES-531-A).
   98. The derivative of any of embodiments 1-97, wherein the CCK-2R expressing cells are cultivated essentially as described in Example 91.
   99. The derivative of any of embodiments 1-98, wherein IP1 generated after CCK-2R activation is determined essentially as described in Example 91.
   100. The derivative of any of embodiments 1-99, wherein the EC₅₀ value on the CCK-2R is determined essentially as described in Example 91.
   101. The derivative of any of embodiments 1-100, wherein CCK-8s (SEQ ID NO: 1) is used as a positive reference for CCK-2R activity.
   102. The derivative of any of embodiments 1-101, wherein Gastrin-17 (SEQ ID NO: 2) is used as a positive reference for CCK-2R activity.
   103. The derivative of any of embodiments 1-102, wherein for CCK-2R activity determination suitable serial dilutions of the derivative and the reference compounds are used.
   104. The derivative of any of embodiments 1-103, wherein for CCK-2R activity the serial dilutions are from 10⁻⁶ M (one μM) to 10⁻¹² M (one pM).
   105. The derivative of any of embodiments 1-104, wherein the in vitro potency (EC₅₀ value) on the CCK-2R is determined essentially as described in Example 91.
   106. The derivative of any of embodiments 1-105, which has an in vitro potency on the CCK-2R (EC₅₀ value) which is at least 200 times the CCK-2R EC₅₀ value for CCK-8s (SEQ ID NO: 1).
   107. The derivative of any of embodiments 1-106, which has an in vitro potency on the CCK-2R (EC₅₀ value) which is at least 250 times the CCK-2R EC₅₀ value for CCK-8s (SEQ ID NO: 1).
   108. The derivative of any of embodiments 1-107, which has an in vitro potency on the CCK-2R (EC₅₀ value) which is at least 300 times the CCK-2R EC₅₀ value for CCK-8s (SEQ ID NO: 1).
   109. The derivative of any of embodiments 1-108, which has an in vitro potency on the CCK-2R (EC₅₀ value) which is at least 500 times the CCK-2R EC₅₀ value for CCK-8s (SEQ ID NO: 1).
   110. The derivative of any of embodiments 1-109, which has an in vitro potency on the CCK-2R (EC₅₀ value) which is at least 1000 times the CCK-2R EC₅₀ value for CCK-8s (SEQ ID NO: 1).
   111. The derivative of any of embodiments 1-110, which has an in vitro potency on the CCK-2R (EC₅₀ value) which is at least 1500 times the CCK-2R EC₅₀ value for CCK-8s (SEQ ID NO: 1).
   112. The derivative of any of embodiments 1-111, which has an in vitro potency on the CCK-2R (EC₅₀ value) which is at least 2000 times the CCK-2R EC₅₀ value for CCK-8s (SEQ ID NO: 1).
   113. The derivative of any of embodiments 1-112, which has an in vitro potency on the CCK-2R (EC₅₀ value) which is at least 2500 times the CCK-2R EC₅₀ value for CCK-8s (SEQ ID NO: 1).
   114. The derivative of any of embodiments 1-113, which has an in vitro potency on the CCK-2R (EC₅₀ value) which is at least 3000 times the CCK-2R EC₅₀ value for CCK-8s (SEQ ID NO: 1).
   115. The derivative of any of embodiments 1-114, which has an in vitro potency on the CCK-2R (EC₅₀ value) which is at least 200000 pM.
   116. The derivative of any of embodiments 1-114, which has an in vitro potency on the CCK-2R (EC₅₀ value) which is at least 300000 pM.
   117. The derivative of any of embodiments 1-114, which has an in vitro potency on the CCK-2R (EC₅₀ value) which is at least 600000 pM.
   118. The derivative of any of embodiments 1-114, which has an in vitro potency on the CCK-2R (EC₅₀ value) which is at least 800000 pM.
   119. The derivative of any of embodiments 1-114, which has an in vitro potency on the CCK-2R (EC₅₀ value) which is at least 1000000 pM.
   120. The derivative of any of embodiments 1-119, which is CCK-1R selective.
   121. The derivative of any of embodiments 1-120, which has a ratio [EC₅₀ (CCK-2R) /EC₅₀ (CCK-1R)] of above 1.
   122. The derivative of embodiment 121, where EC₅₀ (CCK-2R) and EC₅₀ (CCK-1R) refer to the potency of the derivative in question on the CCK-2R and the CCK-1R, respectively, determined as described in any of embodiments 94-105 and 57-68, respectively.
   123. The derivative of any of embodiments 121-122, wherein the EC₅₀ (CCK-2R) and the EC₅₀ (CCK-1R) values are determined essentially as described in Example 91.
   124. The derivative of any of embodiments 120-123, which has a ratio [EC₅₀ (CCK-2R)/EC₅₀ (CCK-1R)] of at least 200.
   125. The derivative of any of embodiments 120-124, which has a ratio [EC₅₀ (CCK-2R)/EC₅₀ (CCK-1R)] of at least 300.
   126. The derivative of any of embodiments 120-125, which has a ratio [EC₅₀ (CCK-2R)/EC₅₀ (CCK-1R)] of at least 500.
   127. The derivative of any of embodiments 120-126, which has a ratio [EC₅₀ (CCK-2R)/EC₅₀ (CCK-1R)] of at least 1000.
   128. The derivative of any of embodiments 120-127, which has a ratio [EC₅₀ (CCK-2R)/EC₅₀ (CCK-1R)] of at least 1500.
   129. The derivative of any of embodiments 120-128, which has a ratio [EC₅₀ (CCK-2R)/EC₅₀ (CCK-1R)] of at least 2000.
   130. The derivative of any of embodiments 120-129, which has a ratio [EC₅₀ (CCK-2R)/EC₅₀ (CCK-1R)] of at least 5000.
   131. The derivative of any of embodiments 120-130, which has a ratio [EC₅₀ (CCK-2R)/EC₅₀ (CCK-1R)] of at least 10000.
   132. The derivative of any of embodiments 120-131, which has a ratio [EC₅₀ (CCK-2R)/EC₅₀ (CCK-1R)] of at least 20000.
   133. The derivative of any of embodiments 120-132, which has a ratio [EC₅₀ (CCK-2R)/EC₅₀ (CCK-1R)] of at least 30000.
   134. The derivative of any of embodiments 120-133, which has a ratio [EC₅₀ (CCK-2R)/EC₅₀ (CCK-1R)] of at least 40000.
   135. The derivative of any of embodiments 120-134, which has a ratio [EC₅₀ (CCK-2R)/EC₅₀ (CCK-1R)] of at least 50000.
   136. The derivative of any of embodiments 120-135, which has a ratio [EC₅₀ (CCK-2R)/EC₅₀ (CCK-1R)] of at least 60000.
   137. The derivative of any of embodiments 1-136, which has a more protracted profile of action than native CCK-8s (SEQ ID NO: 1).
   138. The derivative of embodiment 137, wherein protracted profile of action refers to terminal half-life in vivo in a relevant animal species, such as mouse, rat, pig, and dog, preferably mini-pig; wherein the derivative is administered i) s.c., and/or ii) i.v.; preferably ii) s.c.
   139. The derivative of any of embodiments 1-138, wherein the terminal half-life (T_{1/2}) after i.v. administration to mini-pigs is at least 12 hours.
   140. The derivative of any of embodiments 1-139, wherein the terminal half-life (T_{1/2}) after i.v. administration to mini-pigs is at least 15 hours.
   141. The derivative of any of embodiments 1-140, wherein the terminal half-life (T_{1/2}) after i.v. administration to mini-pigs is at least 20 hours.
   142. The derivative of any of embodiments 1-141, wherein the terminal half-life (T_{1/2}) after i.v. administration to mini-pigs is at least 30 hours.
   143. The derivative of any of embodiments 1-142, wherein the terminal half-life (T_{1/2}) after i.v. administration to mini-pigs is at least 40 hours.
   144. The derivative of any of embodiments 1-143, wherein the terminal half-life (T_{1/2}) after i.v. administration to mini-pigs is at least 50 hours.
   145. The derivative of any of embodiments 1-144, wherein the terminal half-life (T_{1/2}) after i.v. administration to mini-pigs is at least 60 hours.
   146. The derivative of any of embodiments 1-145, wherein the terminal half-life (T_{1/2}) after i.v. administration to mini-pigs is at least 70 hours.
   147. The derivative of any of embodiments 1-146, wherein the terminal half-life (T_{1/2}) after i.v. administration to mini-pigs is at least 80 hours.
   148. The derivative of any of embodiments 1-147, wherein the terminal half-life (T_{1/2}) after i.v. administration to mini-pigs is at least 90 hours.
   149. The derivative of any of embodiments 1-148, wherein the terminal half-life (T_{1/2}) after i.v. administration to mini-pigs is at least 100 hours.
   150. The derivative of any of embodiments 1-149, wherein the terminal half-life (T_{1/2}) after i.v. administration to mini-pigs is at least 110 hours.
   151. The derivative of any of embodiments 138-150, wherein the mini-pigs are female, preferably obtained from Ellegaard Gottingen Minipigs.
   152. The derivative of any of embodiments 138-151, wherein the mini-pigs are approximately 6 months of age, and preferably weighing approximately 17 kg.
   153. The derivative of any of embodiments 138-152, wherein the derivative is dissolved in 50 mM sodium phosphate buffer containing 70 mM sodium chloride, and 0.05% polysorbate 80, at pH 7.4.
   154. The derivative of any of embodiments 138-153, wherein an intravenous injection of the derivative is given in a dosage corresponding to, e.g., 5 nmol/kg body weight.
   155. The derivative of any of embodiments 138-154, wherein the terminal half-life (T₂) is determined in vivo pharmacokinetic studies in mini-pig, for example as described in Example 93.
   156. The derivative of any of embodiments 1-155, which in a pharmacodynamic (PD) study in pigs and when administered as a single dose s.c. has the effect of reducing food intake for (i) at least 24 hours, and/or (ii) for at least 48 hours, as compared to a vehicle-treated control group.
   157. The derivative of embodiment 156, wherein the pigs are female Landrace Yorkshire Duroc (LYD) pigs or Large White hybrid, approximately 3 months of age, and weighing approximately 30-35 kg (preferably n=4 per group).
   158. The derivative of any of embodiments 156-157, wherein the pigs are housed in a group for approximately 1 week during acclimatisation to the animal facilities, and subsequently, during the experimental period, the animals are placed in individual pens approximately 1 week before dosing and during the entire experiment for measurement of individual food intake.
   159. The derivative of any of embodiments 156-158, wherein the pigs are fed ad libitum with pig fodder (Svinefoder Danish Top; supplier Danish Agro) at all times both during the acclimatisation and the experimental period.
   160. The derivative of any of embodiments 156-159, wherein food intake is monitored, e.g. on line by logging the weight of fodder every 15 minutes, preferably using the Mpigwin system (available e.g. from Ellegaard Systems, Faaborg, Denmark); any uneaten fodder is removed and weighed (preferably manually) on the following morning and replaced with fresh fodder; a baseline food intake is measured for each pig on three successive days before dosing of the derivative in question; and a mean baseline food intake value is calculated for each pig.
   161. The derivative of any of embodiments 156-160, which is dissolved in a phosphate buffer (50 mM sodium phosphate, 70 mM sodium chloride, 0.05 v/v % polysorbate 80, pH 7.4) at concentrations of approximately 5300 nmol/ml corresponding to doses of 100 nmol/kg body weight.
   162. The derivative of embodiment 161, wherein the phosphate buffer serves as vehicle.
   163. The derivative of any of embodiments 156-162, wherein the pigs are dosed with a single subcutaneous dose of the derivative or vehicle (dose volume 0.02 ml/kg) on the morning of day 1, and food intake is measured for 48 hours after dosing.
   164. The derivative of any of embodiments 156-163, wherein food intake (FI) is calculated in 24 h intervals (such as 0-24 h, and 24-48 h), and the resulting mean difference in food intake (ΔFI, in kg) is calculated on the basis of the mean baseline food intake for each pig (ΔFI₀₋₂₄ₕ=FI₀₋₂₄ₕ-F_{baseline}, and vice-versa for the 24-48 hours' time interval).
   165. The derivative of any of embodiments 156-164, wherein the food intake is determined essentially as described in Example 94.
   166. The derivative of any of embodiments 156-165, wherein the food intake for hours 0-24 is reduced by at least 0.10 kg (ΔFI₀₋₂₄ₕ is −0.10 kg or lower).
   167. The derivative of any of embodiments 156-166, wherein the food intake for hours 0-24 is reduced by at least 0.20 kg (ΔFI₀₋₂₄ₕ is −0.20 kg or lower).
   168. The derivative of any of embodiments 156-167, wherein the food intake for hours 0-24 is reduced by at least 0.30 kg (ΔFI₀₋₂₄ₕ is −0.30 kg or lower).
   169. The derivative of any of embodiments 156-168, wherein the food intake for hours 0-24 is reduced by at least 0.40 kg (ΔFI₀₋₂₄ₕ is −0.40 kg or lower).
   170. The derivative of any of embodiments 156-169, wherein the food intake for hours 0-24 is reduced by at least 0.50 kg (ΔFI₀₋₂₄ₕ is −0.50 kg or lower).
   171. The derivative of any of embodiments 156-170, wherein the food intake for hours 0-24 is reduced by at least 0.60 kg (ΔFI₀₋₂₄ₕ is −0.60 kg or lower).
   172. The derivative of any of embodiments 156-171, wherein the food intake for hours 0-24 is reduced by at least 0.70 kg (ΔFI₀₋₂₄ₕ is −0.70 kg or lower).
   173. The derivative of any of embodiments 156-172, wherein the food intake for hours 0-24 is reduced by at least 0.80 kg (ΔFI₀₋₂₄ₕ is −0.80 kg or lower).
   174. The derivative of any of embodiments 156-173, wherein the food intake for hours 0-24 is reduced by at least 0.90 kg (ΔFI₀₋₂₄ₕ is −0.90 kg or lower).
   175. The derivative of any of embodiments 156-174, wherein the food intake for hours 0-24 is reduced by at least 1.0 kg (ΔFI₀₋₂₄ₕ is −1.0 kg or lower).
   176. The derivative of any of embodiments 156-175, wherein the food intake for hours 24-48 is reduced by at least 0.10 kg (ΔFI₀₋₂₄ₕ is −0.10 kg or lower).
   177. The derivative of any of embodiments 156-176, wherein the food intake for hours 24-48 is reduced by at least 0.20 kg (ΔFI₀₋₂₄ₕ is −0.20 kg or lower).
   178. The derivative of any of embodiments 156-177, wherein the food intake for hours 24-48 is reduced by at least 0.30 kg (ΔFI₀₋₂₄ₕ is −0.30 kg or lower).
   179. The derivative of any of embodiments 156-178, wherein the food intake for hours 24-48 is reduced by at least 0.40 kg (ΔFI₀₋₂₄ₕ is −0.40 kg or lower).
   180. The derivative of any of embodiments 156-179, wherein the food intake for hours 24-48 is reduced by at least 0.50 kg (ΔFI₀₋₂₄ₕ is −0.50 kg or lower).
   181. The derivative of any of embodiments 156-180, wherein the food intake for hours 24-48 is reduced by at least 0.60 kg (ΔFI₀₋₂₄ₕ is −0.60 kg or lower).
   182. The derivative of any of embodiments 1-181, which is stable in a pH 7.4 phosphate buffer at 5° C. for at least 6 weeks.
   183. The derivative of any of embodiments 1-182, which is stable in a pH 7.4 phosphate buffer at 25° C. for at least 6 weeks.
   184. The derivative of any of embodiments 1-183, which is stable in a pH 7.4 phosphate buffer at 37° C. for at least 6 weeks.
   185. The derivative of any of embodiments 182-184, wherein the phosphate buffer is 50 mM sodium phosphate containing 70 mM NaCl and 0.05 v/v % polysorbate 80.
   186. The derivative of any of embodiments 182-185, wherein the concentration of the derivative is 1 mg/ml.
   186a. The derivative of any of embodiments 182-186, wherein the concentration of the derivative is approximately 0.5 mM).
   187. The derivative of any of embodiments 182-186a which is stable in a pH 7.4 phosphate buffer (50 mM sodium phosphate containing 70 mM NaCl and 0.05 v/v % polysorbate 80) at 25° C. for at least three days in a concentration of (i) 25 μM, (ii) 100 μM, and/or (iii) 500 μM.
   188. The derivative of any of embodiments 182-187, wherein the term stable refers to a relative purity loss of no more than 2.0%.
   189. The derivative of any of embodiments 182-187, wherein the term stable refers to a relative purity loss of no more than 1.8%.
   190. The derivative of any of embodiments 182-187, wherein the term stable refers to a relative purity loss of no more than 1.6%.
   191. The derivative of any of embodiments 182-187, wherein the term stable refers to a relative purity loss of no more than 1.4%.
   192. The derivative of any of embodiments 182-187, wherein the term stable refers to a relative purity loss of no more than 1.2%.
   193. The derivative of any of embodiments 182-187, wherein the term stable refers to a relative purity loss of no more than 1.0%.
   194. The derivative of any of embodiments 182-187, wherein the term stable refers to a relative purity loss of no more than 0.80%.
   195. The derivative of any of embodiments 182-187, wherein the term stable refers to a relative purity loss of no more than 0.60%.
   196. The derivative of any of embodiments 182-187, wherein the term stable refers to a relative purity loss of no more than 0.40%.
   197. The derivative of any of embodiments 182-187, wherein the term stable refers to a relative purity loss of no more than 0.25%.
   198. The derivative of any of embodiments 182-197, wherein the relative purity loss (in %) is the ratio of absolute purity loss to the purity determined in the start sample, where absolute purity loss (in percentage points, pp) is determined as the difference in purity between the start sample and the sample incubated for the indicated time (such as 6 weeks) at the indicated temperature.
   199. The derivative of any of embodiments 182-198, wherein the purity of the start sample and the purity of the sample incubated for the indicated time at the indicated temperature is determined using RP-UPLC analysis, and wherein purity is defined as the area percentage of the compound peak in relation to the total area of all integrated peaks in the chromatogram.
   200. The derivative of any of embodiments 182-199, wherein the purity loss is determined essentially as described in Example 95.
   201. The derivative of any of embodiments 182-200, wherein the stability and/or purity is determined essentially as described in Example 95.
   202. The derivative of any of embodiments 1-201, which is stable in a pH 7.4 phosphate buffer at 5° C. for at least 2 weeks.
   203. The derivative of any of embodiments 1-202, which is stable in a pH 7.4 phosphate buffer at 25° C. for at least 2 weeks.
   204. The derivative of any of embodiments 1-203, which is stable in a pH 7.4 phosphate buffer at 37° C. for at least 2 weeks.
   205. The derivative of any of embodiments 202-204, where stability is determined as indicated in any of embodiments 185-201.
   206. The derivative of any of embodiments 202-205, wherein the term stable refers to a relative purity loss of no more than 0.20%.
   207. The derivative of any of embodiments 202-205, wherein the term stable refers to a relative purity loss of no more than 0.16%.
   208. A peptide of the general formula I′:

X1-X2-X3-Gly-Trp-X6-DMeAsp-X8-NH₂ (formula I′),

wherein

X1 is absent, Asp, D-Asp, Glu, or D-Glu;

X2 is Phe(4-sulfomethyl) or sTyr;

X3 is Nle, Leu, Ala, Ile, or Val;

X6 is Nle;

X8 is Phe, MePhe, 1Nal, Me1Nal, 2Nal, Me2Nal, Trp, or MeTrp; and

the NH₂ group at the C-terminus of formula I′ means that X8 is C-terminally amidated;

or a pharmaceutically acceptable salt, amide, or ester thereof.

209. The peptide of embodiment 208 which is an intermediate to the derivative of any of embodiments 1-207.
210. The peptide of any of embodiments 208-209, wherein X1 is Asp.
211. The peptide of any of embodiments 208-210, wherein X2 is Phe(4-sulfomethyl).
212. The peptide of any of embodiments 208-211, wherein X2 is sTyr.
213. The peptide of any of embodiments 208-212, wherein X3 is Nle or Leu.
214. The peptide of any of embodiments 208-213, wherein X3 is Nle.
215. The peptide of any of embodiments 208-213, wherein X3 is Leu.
216. The peptide of any of embodiments 208-215, wherein X8 is Phe, MePhe, 1Nal, Me1Nal, or Trp.
217. The peptide of any of embodiments 208-216, wherein X8 is Phe.
218. The peptide of any of embodiments 208-216, wherein X8 is MePhe.
219. The peptide of any of embodiments 208-216, wherein X8 is 1Nal.
220. The peptide of any of embodiments 208-216, wherein X8 is Me1Nal.
221. The peptide of any of embodiments 208-216, wherein X8 is Trp.
222. The peptide of any of embodiments 208-221, which has a maximum of six amino acid changes as compared to CCK-8s (SEQ ID NO: 1).
223. The peptide of any of embodiments 208-222, which has a maximum of five amino acid changes as compared to CCK-8s (SEQ ID NO: 1).
224. The peptide of any of embodiments 208-223, which has a maximum of four amino acid changes as compared to CCK-8s (SEQ ID NO: 1).
225. The peptide of any of embodiments 208-224, which has a maximum of three amino acid changes as compared to CCK-8s (SEQ ID NO: 1).
226. The peptide of any of embodiments 208-225, which has a minimum of three amino acid changes as compared to CCK-8s (SEQ ID NO: 1).
227. The peptide of any of embodiments 208-226, which has a minimum of four amino acid changes as compared to CCK-8s (SEQ ID NO: 1).
228. The peptide of any of embodiments 208-227, which has a minimum of five amino acid changes as compared to CCK-8s (SEQ ID NO: 1).
229. The peptide of any of embodiments 208-228, which has a minimum of six amino acid changes as compared to CCK-8s (SEQ ID NO: 1).
230. The peptide of any of embodiments 208-229, which has five amino acid changes as compared to CCK-8s (SEQ ID NO: 1).
231. The peptide of any of embodiments 208-230, which has four amino acid changes as compared to CCK-8s (SEQ ID NO: 1).
232. The peptide of any of embodiments 208-231, which has three amino acid changes as compared to CCK-8s (SEQ ID NO: 1).
233. A peptide selected from the following:

(Seq 21)
Asp-sTyr-Nle-Gly-Trp-Nle-DMeAsp-Trp-amide,
(Seq 22)
Asp-sTyr-Nle-Gly-Trp-Nle-DMeAsp-1Nal-amide,
(Seq 23)
Asp-Phe(4-sulfomethyl)-Nle-Gly-Trp-Nle-DMeAsp-
MePhe-amide,
(Seq 24)
Asp-sTyr-Leu-Gly-Trp-Nle-DMeAsp-Me1Nal-amide,
(Seq 25)
Asp-Phe(4-sulfomethyl)-Leu-Gly-Trp-Nle-DMeAsp-
MePhe-amide,
(Seq 27)
Asp-sTyr-Leu-Gly-Trp-Nle-DMeAsp-Phe-amide,
(Seq 28)
Asp-sTyr-Nle-Gly-Trp-Nle-DMeAsp-MePhe-amide,
(Seq 29)
Asp-sTyr-Leu-Gly-Trp-Nle-DMeAsp-MePhe-amide,

or a pharmaceutically acceptable salt, amide, or ester thereof.
234. The peptide of embodiment 233, which is a peptide of any of embodiments 208-232.
235. The peptide of any of embodiments 208-234 which is a CCK peptide.
236. The peptide of any of embodiments 208-235 which is an analogue of CCK-8s.
237. The peptide of any of embodiments 208-236, which consists of 7 or 8 amino acid residues.
238. The peptide of any of embodiments 208-237, which consists of 8 amino acid residues.
239. A method of preparing the peptide of any of embodiments 208-238, which is an SPPS method.
240. The method of embodiment 239, wherein Fmoc based chemistry is used.
241. The method of any of embodiments 239-240, which is performed essentially as described in the experimental section herein, in particular in the section headed “Methods for Synthesis, Purification and Analysis of Example Compounds”.
242. A method of preparing the derivative of any of embodiments 1-207, wherein the peptide backbone is prepared as described in any of embodiments 239-241, and the P-L part of formula I is added by SPPS.
243. The method of embodiment 242, wherein Fmoc based chemistry is used.
244. The method of any of embodiments 242-243, which is performed essentially as described in the experimental section herein, in particular in the section headed “Methods for Synthesis, Purification and Analysis of Example Compounds”, more in particular in the subsection headed “Attachment of linker and fatty acid derivative”.
245. The method of any of embodiments 242-244, wherein the derivative is cleaved from the resin and the side chains deprotected.
246. The method of embodiment 245, which is performed essentially as described in the experimental section herein, in particular in the section headed “Methods for Synthesis, Purification and Analysis of Example Compounds”, more in particular in the subsection headed “Cleavage from the resin and side-chain deprotection”.
247. The method of any of embodiments 242-246, which comprises a step of removal of a neopentyl group from Tyr(SO₃-neopentyl).
248. The method of embodiment 247, wherein the neopentyl group is removed from Tyr(SO₃-neopentyl) essentially as described in the section headed “Methods for Synthesis, Purification and Analysis of Example Compounds”, more in particular in the subsection headed “Removal of the neopentyl group from Tyr(SO₃-neopentyl)”.
249. The method of any of embodiments 242-246, which comprises a step of removal of a trichloroethyl (TCE) group from Phe(4-sulfomethyl-TCE).
250. The method of embodiment 249, wherein the trichloroethyl (TCE) group is removed from Phe(4-sulfomethyl-TCE) essentially as described in the section headed “Methods for Synthesis, Purification and Analysis of Example Compounds”, more in particular in the subsection headed “Removal of a trichloroethyl (TCE) group from Phe(4-sulfomethyl-TCE)”.
251. The method of any of embodiments 239-250, wherein the peptide or derivative is dissolved in neutral aqueous ammonium acetate and acetonitrile and purified by reversed-phase preparative HPLC.
252. The method of embodiment 251, wherein the purification is performed essentially as described in the section headed “Methods for Synthesis, Purification and Analysis of Example Compounds”, more in particular in the subsection headed “Purification and Quantification”.
253. A method of preparing the derivative of any of embodiments 1-207, which method comprises the following steps:

(i) preparation of the intermediate peptide of any of embodiments 208-234 using Solid Phase Peptide Synthesis (SPPS);

(ii) preparation of the P-L side chain of any of embodiments 1-20 in the form of its C-terminal carboxylic acid, and activation thereof with a coupling agent resulting in the formation of an acylating agent; and

(iii) attachment of the acylating agent from step (ii) to the N-terminus of the intermediate peptide from step (i).

254. The method of embodiment 253, wherein Fmoc based chemistry is used.
255. The method of any of embodiments 253-254, wherein the intermediate peptide is cleaved from the resin and optionally purified.
256. The method of any of embodiments 253-255, wherein the derivative is cleaved from the resin and the side chain deprotected.
257. The method of any of embodiments 253-256, which comprises a step of removal of a neopentyl group from Tyr(SO₃-neopentyl).
258. The method of embodiment 257, wherein the neopentyl group is removed from Tyr(SO₃-neopentyl) essentially as described in the section headed “Methods for Synthesis, Purification and Analysis of Example Compounds”, more in particular in the subsection headed “Removal of the neopentyl group from Tyr(SO₃-neopentyl)”.
259. The method of any of embodiments 253-258, which comprises a step of removal of a trichloroethyl (TCE) group from Phe(4-sulfomethyl-TCE).
260. The method of embodiment 259, wherein the trichloroethyl (TCE) group is removed from Phe(4-sulfomethyl-TCE) essentially as described in the section headed “Methods for Synthesis, Purification and Analysis of Example Compounds”, more in particular in the subsection headed “Removal of a trichloroethyl (TCE) group from Phe(4-sulfomethyl-TCE)”.
261. The method of any of embodiments 239-250, wherein the peptide or derivative is dissolved in neutral aqueous ammonium acetate and acetonitrile and purified by reversed-phase preparative HPLC.
262. The method of embodiment 261, wherein the purification is performed essentially as described in the section headed “Methods for Synthesis, Purification and Analysis of Example Compounds”, more in particular in the subsection headed “Purification and Quantification”.
263. The method of any of embodiments 253-262, wherein the attachment of step (iii) is effected by formation of an amide bond.
264. The method of any of embodiments 253-264, wherein the coupling agent is an N-hydroxysuccinimide ester.
265. The method of any of embodiments 253-264, wherein the preparation of the side chain is performed as described in WO 2009/083549 A1.
266. The method of any of embodiments 253-265, wherein the attachment of the side chain to the intermediate peptide is performed as described in WO 2009/083549 A1.
267. A pharmaceutical composition which comprises a derivative of any of embodiments 1-207 or a peptide of any of embodiments 208-238, together with a pharmaceutically acceptable excipient.
268. The pharmaceutical composition of embodiment 267, which is a solution.
269. The pharmaceutical composition of any of embodiments 267-268, which is an aqueous solution for subcutaneous injection.
270. The pharmaceutical composition of any of embodiments 267-269, which is a phosphate buffered solution of pH 7.4.
271. The pharmaceutical composition of any of embodiments 267-270 which further comprises a preservative.
272. The pharmaceutical composition of any of embodiments 267-271 wherein the preservative is selected from m-cresol and phenol.
273. The pharmaceutical composition of any of embodiments 267-272 wherein the concentration of the derivative is in the range of 0.10 mM to 10.0 mM.
274. An injection device containing the pharmaceutical composition of any of embodiments 253-259.
275. A derivative of any of embodiments 1-207 or a peptide of any of embodiments 208-238 for use as a medicament.
276. A derivative of any of embodiments 1-207 or a peptide of any of embodiments 208-238 for the treatment or prevention of overweight.
277. The derivative of embodiment 262 for reduction of appetite and/or reduction of food intake.
278. The derivative of any of embodiments 1-207 for
(i) Chronic weight management, preferably in addition to diet and exercise;
(ii) Treatment of obesity, preferably in adults with a body mass index (BMI) of 30 or greater;
(iii) Treatment of overweight, preferably in adults with a BMI of 27 or greater who, more preferably, have at least one weight-related co-morbidity or condition such as high cholesterol, obstructive sleep apnoea, hypertension, type 2 diabetes, or dyslipidaemia.
279. The derivative of any of embodiments 1-207 for use in combination with other weight loss agents.
280. A method for the treatment or prevention of overweight comprising the administration of a derivative of any of embodiments 1-207 or a peptide of any of embodiments 208-238.
281. A method for reduction of appetite comprising the administration of a derivative of any of embodiments 1-207 or a peptide of any of embodiments 208-238.
282. A method for reduction of food intake comprising administration of a derivative of any of embodiments 1-207 or a peptide of any of embodiments 208-238.
283. A method for
(i) chronic weight management, preferably in addition to diet and exercise;
(ii) treatment of obesity, preferably in adults with a body mass index (BMI) of 30 or greater;
(iii) treatment of overweight, preferably in adults with a BMI of 27 or greater who, more preferably, have at least one weight-related condition such as hypertension, type 2 diabetes, or dyslipidaemia;

the method comprising the administration of a derivative of any of embodiments 1-207 or a peptide of any of embodiments 208-238.

284. The method of any of embodiments 280-283, wherein the derivative is administered in combination with other weight loss agents.
oOo
i). A peptide derivative of the general formula I:

P-L-X1-X2-X3-Gly-Trp-X6-DMeAsp-X8-NH₂ (formula I),

wherein P is Chem. 1:

HOOC—(CH₂)ₓ—CO—*, Chem. 1:

wherein x is an integer in the range of 12-18; L is absent, or L comprises at least one linker element of formula Chem. 2, Chem. 3, Chem. 4, and/or Chem. 5:

*—NH—CH(COOH)—(CH₂)₂—CO—*, Chem. 2:

*—NH—(CH₂)₂—[O—(CH₂)₂]ₖ—O—[CH₂]ₙ—CO—*, Chem. 3:

wherein k is an integer in the range of 1-5, and n is an integer in the range of 1-5,

*—NH—CH(CH₂OH)—CO—*, Chem. 4:

and/or

*—NH—CH₂—CO—*; Chem. 5:

X1 is absent, Asp, D-Asp, Glu, or D-Glu;
X2 is Phe(4-sulfomethyl) or sTyr;

### X3 is Nle, Leu, Ala, Ile, or Val;

### X6 is Nle;

### X8 is Phe, MePhe, 1Nal, Me1Nal, 2Nal, Me2Nal, Trp, or MeTrp; and

the NH₂ group at the C-terminus of formula I means that X8 is C-terminally amidated; or a pharmaceutically acceptable salt, amide, or ester thereof.
ii). The derivative of embodiment i), wherein X1 is Asp; X2 is Phe(4-sulfomethyl) or sTyr;

### X3 is Nle or Leu; and X8 is Phe, MePhe, 1Nal, Me1Nal, or Trp.

iii). The derivative of any of embodiments i)-ii), wherein L consists of one Chem. 2a element and two Chem. 3a elements (Chem. 2a-2×Chem. 3a), interconnected via amide bonds and in the sequence indicated, L being connected at its *—NH end to the CO—* end of P, and at its CO—* end to the amino group of X1, or if X1 is absent to the amino group of X2, wherein Chem. 2a and Chem. 3a represent the following structures:

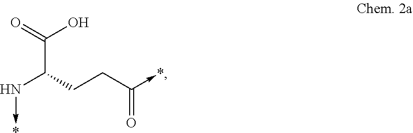

*—NH—(CH₂)₂—O—(CH₂)₂—O—CH—CO—* Chem. 3a:

iv). The derivative of any of embodiments i)-iii), wherein x is 14 or 16.
v). The derivative of any of embodiments i)-iv), which is selected from the following:

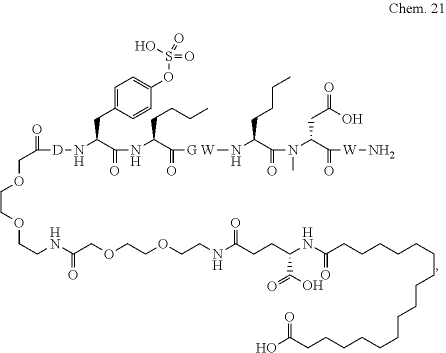

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Nle-Gly-Trp-Nle-DMeAsp-Trp-amide,

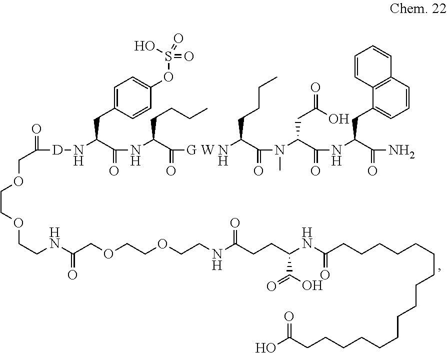

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Nle-Gly-Trp-Nle-DMeAsp-1Nal-amide,

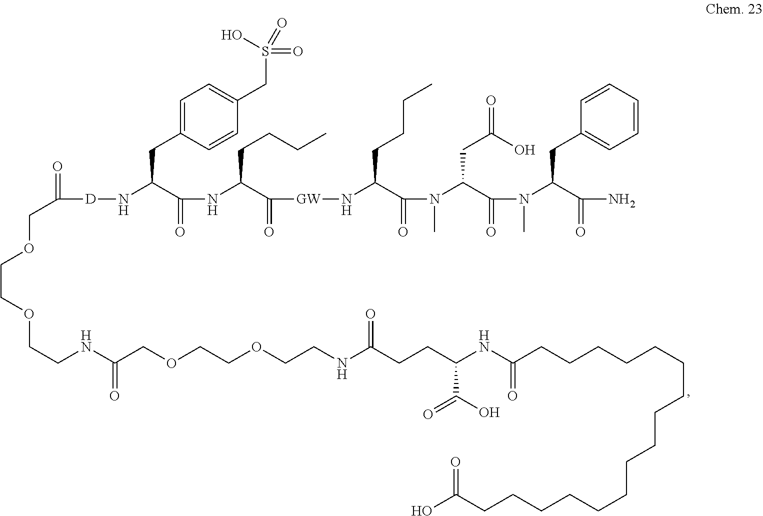

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-Phe(4-sulfomethyl)-Nle-Gly-Trp-Nle-DMeAsp-MePhe-amide,

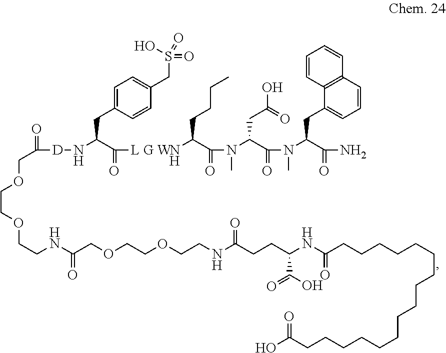

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Nle-DMeAsp-Me1Nal-amide,

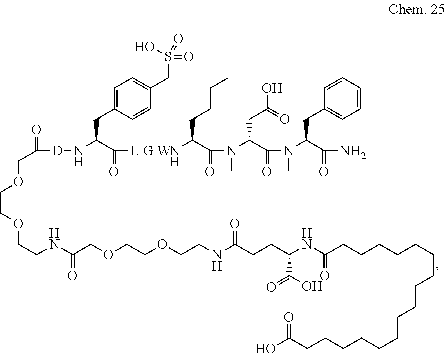

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-Phe(4-sulfomethyl)-Leu-Gly-Trp-Nle-DMeAsp-MePhe-amide,

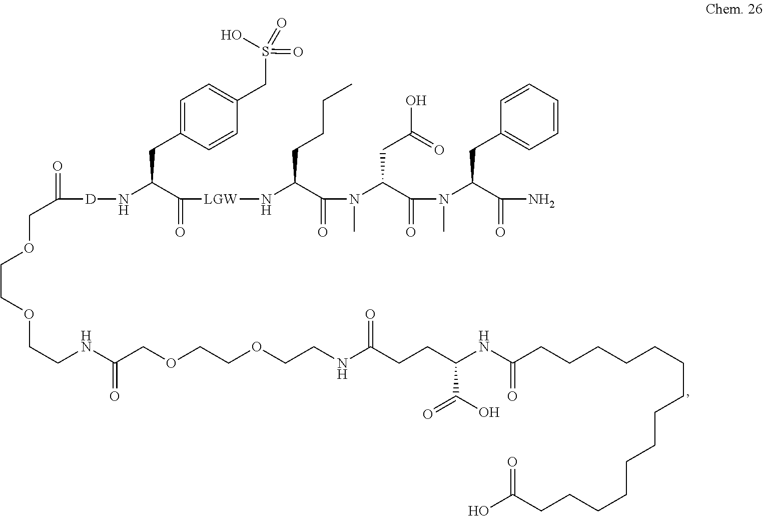

2-[2-[2-[[2-[2-[2-[[(4S)-4-Carboxy-4-(15-carboxypentadecanoylamino)butanoyl]amino]ethoxy]ethoxy]acetyl]amino]ethoxy]ethoxy]acetyl-Asp-Phe(4-sulfomethyl)-Leu-Gly-Trp-Nle-DMeAsp-MePhe-amide,

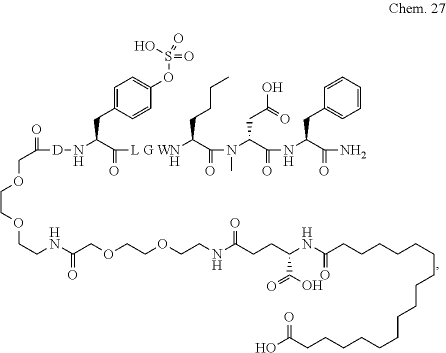

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Nle-DMeAsp-Phe-amide,

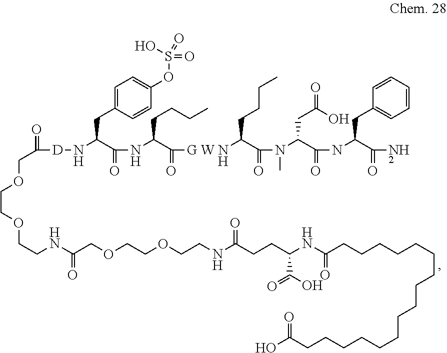

2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Nle-Gly-Trp-Nle-DMeAsp-MePhe-amide,

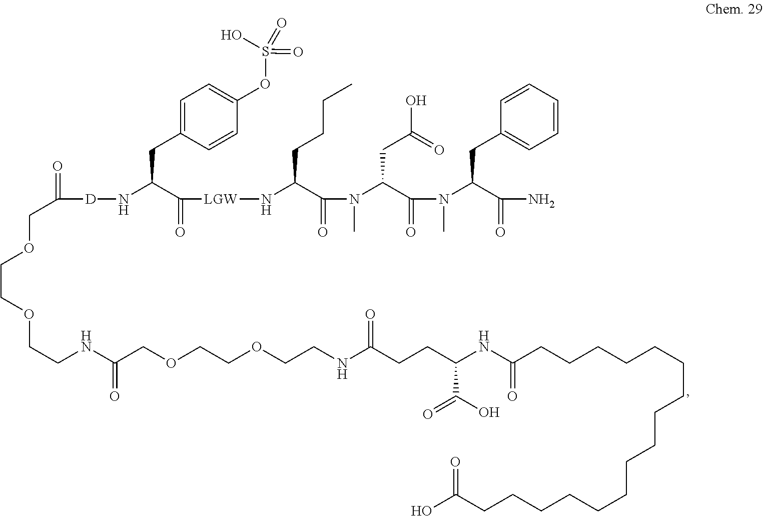

and
2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Nle-DMeAsp-MePhe-amide;
   or a pharmaceutically acceptable salt, amide, or ester thereof.
   vi). An intermediate product in the form of a peptide of the general formula I′:

X1-X2-X3-Gly-Trp-X6-DMeAsp-X8-NH₂ (formula I′),

wherein

X1 is absent, Asp, D-Asp, Glu, or D-Glu;

X2 is Phe(4-sulfomethyl) or sTyr;

X3 is NIe, Leu, Ala, Ile, or Val;

X6 is Nle;

X8 is Phe, MePhe, 1Nal, Me1Nal, 2Nal, Me2Nal, Trp, or MeTrp; and

the NH₂ group at the C-terminus of formula I′ means that X8 is C-terminally amidated;

or a pharmaceutically acceptable salt, amide, or ester thereof.

vii). The peptide of embodiment vi), wherein X1 is Asp; X2 is Phe(4-sulfomethyl) or sTyr; X3 is Nle or Leu; and X8 is Phe, MePhe, 1Nal, Me1Nal, or Trp.
iix). The peptide of any of embodiments vi)-vii), which is selected from the following:

(Seq 21)
Asp-sTyr-Nle-Gly-Trp-Nle-DMeAsp-Trp-amide,
(Seq 22)
Asp-sTyr-Nle-Gly-Trp-Nle-DMeAsp-1Nal-amide,
(Seq 23)
Asp-Phe(4-sulfomethyl)-Nle-Gly-Trp-Nle-DMeAsp-
MePhe-amide,
(Seq 24)
Asp-sTyr-Leu-Gly-Trp-Nle-DMeAsp-Me1Nal-amide,
(Seq 25)
Asp-Phe(4-sulfomethyl)-Leu-Gly-Trp-Nle-DMeAsp-
MePhe-amide,
(Seq 27)
Asp-sTyr-Leu-Gly-Trp-Nle-DMeAsp-Phe-amide,
(Seq 28)
Asp-sTyr-Nle-Gly-Trp-Nle-DMeAsp-MePhe-amide,
(Seq 29)
Asp-sTyr-Leu-Gly-Trp-Nle-DMeAsp-MePhe-amide;

or a pharmaceutically acceptable salt, amide, or ester thereof.
ix). A method of preparing the peptide of any of embodiments vi)-iix) or the derivative of any of embodiments i)-v), which is an SPPS method.
x). A method of preparing the derivative of any of embodiments i)-v), which comprises the following steps:

(i) the intermediate peptide of any of embodiments 6-8 is prepared using SPPS,

(ii) the P-L side chain of any of embodiments 1-4 in the form of its C-terminal carboxylic acid is prepared and activated with a coupling agent under formation of an acylating agent; and

(iii) the acylating agent is coupled to the N-terminus of the intermediate peptide via an amide bond.

xi). A pharmaceutical composition which comprises a derivative of any of embodiments i)-v) or a peptide of any of embodiments vi)-iix), together with a pharmaceutically acceptable excipient.
xii). The pharmaceutical composition of embodiment xi), which is a solution.
xiii). An injection device containing the pharmaceutical composition of any of embodiments xi)-xii).
xiv). A derivative of any of embodiments i)-v) or a peptide of any of embodiments vi)-iix) for use as a medicament.
xv). A derivative of any of embodiments i)-v) or a peptide of any of embodiments vi)-iix) for the treatment or prevention of overweight, and/or for reduction of appetite, and/or for reduction of food intake.

### EXAMPLES

### List of Abbreviations

Ado: 8-amino-3,6-dioxaoctanoic acid

### API: Active Pharmaceutical Ingredient

### AUC: Area Under the Curve

### BG: Blood Glucose

### BHK Baby Hamster Kidney

### BW: Body Weight

Boc: t-butyloxycarbonyl
BSA: Bovine serum albumin
Bzl: benzyl

### CAS: Chemical Abstracts Service

collidine: 2,4,6-trimethylpyridine
DCM: dichloromethane
DIC: diisopropylcarbodiimide
DIPEA: diisopropylethylamine (N-ethyl-N-isopropyl-propan-2-amine)
DMAP: 4-dimethylaminopyridine

### DMEM: Dulbecco's Modified Eagle's Medium

DMF: dimethylformamide
EDTA: ethylenediaminetetraacetic acid
EGTA: ethylene glycol tetraacetic acid

### FCS: Foetal Calf Serum

Fmoc: 9-fluorenylmethyloxycarbonyl
HEPES: 4-(2-hydroxyethyl)-1-piperazineethanesulfonic acid

### HPLC: High Performance Liquid Chromatography

### HSA: Human Serum Albumin

IBMX: 3-isobutyl-1-methylxanthine
i.v. intravenously

### LCMS: Liquid Chromatography Mass Spectrometry

### LLOQ: Lower Limit Of Quantification

### LYD: Landrace Yorkshire Duroc

MeOH: methanol

### NA: Not Applicable

NMP: N-methyl pyrrolidin-2-one
OtBu: tert-butyloxy
Oxyma Pure®: Cyano-hydroxyimino-acetic acid ethyl ester
Pbf: 2,2,4,6,7-pentamethyldihydrobenzofuran-5-sulfonyl

### PBS: Phosphate Buffered Saline

### PD: Pharmacodynamic

### Pen/Strep: Pencillin/Streptomycin

### PK: Pharmacokinetic(s)

### RP: Reversed Phase

### RP-HPLC: Reversed Phase High Performance Liquid Chromatography

### RT: Room Temperature

Rt: Retention time

### s.c.: Subcutaneously

### SD: Standard Deviation

### SEM: Standard Error of Mean

### SPA: Scintillation Proximity Assay

### SPPS: Solid Phase Peptide Synthesis

tBu: tert-butyl
TCE: 2,2,2-trichloroethyl
TFA: trifluoroacetic acid
THF: tetrahydrofuran
TIPS: triisopropylsilane

### TLC: Thin Layer Chromatography

Tris: tris(hydroxymethyl)aminomethane or 2-amino-2-hydroxymethyl-propane-1,3-diol
Trt: triphenylmethyl (trityl)

### UPLC: Ultra Performance Liquid Chromatography

For amino acid abbreviations, see the section headed “Amino acids, Peptides, and Peptide Derivatives” including Table 1.

### Methods for Synthesis, Purification and Analysis of Example Compounds

This section relates to methods for synthesising resin bound peptide (SPPS methods, including methods for de-protection of amino acids, methods for cleaving the peptide from the resin, and for its purification), as well as methods for detecting and characterising the resulting peptide (LCMS and UPLC methods).

For common procedures in solid-phase peptide synthesis using Fmoc protecting groups (Fmoc-SPPS), see also: “Fmoc Solid Phase Synthesis—A Practical Approach”, Edited by W. C. Chan and P. D. White, Oxford University Press, 2000.

### Solid-Phase Peptide Synthesis (SPPS)

SPPS was performed using Fmoc based chemistry, either on a Prelude Peptide Synthesiser from Protein Technologies (Tucson, Ariz. 85714, U.S.A.), or manually. A suitable resin for the preparation of C-terminal peptide amides is H-Rink Amide-ChemMatrix resin (loading e.g. 0.52 mmol/g) or Rink Amide AM polystyrene resin (Novabiochem: loading eg 0.62 mmol/g) or the like.

Fmoc-deprotection was achieved with 20% piperidine in NMP, for example for 2×5 min. Peptide couplings were performed by using DIC/Oxyma/collidine without pre-activation. Amino acid/Oxyma solutions (0.3 M/0.3 M in NMP or DMF at a molar excess of 3-10 fold compared to the resin) were added to the resin followed by the same molar excess of DIC (3 M in NMP or DMF) followed by the same molar excess of collidine (3 M in NMP or DMF). Typical coupling times applied were 1 h or 2 h. For difficult couplings, prolonged coupling times can be used.

If not stated otherwise, the Fmoc-protected amino acid derivatives used were the following: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH and Fmoc-Lys(Mtt)-OH supplied from e.g. Anaspec, Bachem, Iris Biotech or Novabiochem.

For incorporation of norleucine (Nle), 1-naphthylalanine (1Nal), 2-naphthylalanine (2Nal), and N-methylphenylalanine (MePhe), the corresponding Fmoc-amino acids were used. For incorporation of sulfated tyrosine (sTyr), Fmoc-Tyr(SO₃-neopentyl)-OH (CAS-no. 878408-63-0; from Novabiochem) was used. For incorporation of 4-sulfomethyl-phenylalanine (Phe(4-sulfomethyl)), Fmoc-Phe(4-sulfomethyl-TCE)-OH (CAS-no. 1146758-11-3; from Bachem) was used. For incorporation of D-N-methyl-Asp (DMeAsp), Fmoc-DMeAsp(OtBu)-OH, (CAS-1799443-40-5; from ChemPep, Wellington, Fla., USA) was used in most cases. Alternatively, incorporation of DMeAsp into the peptide backbone was done by N-methylation of a resin-bound DAsp residue according to the procedure described below. In the same manner, other N-methylated amino acid residues, such as N-methyl-1-naphthylalanine (Me1Nal) and N-methyl-2-naphthylalanine (Me2Nal), can be incorporated by N-methylation of the corresponding non-methylated resin-bound amino acid residue.

### N-Methylation of Resin-Bound Amino Acid DAsp

Methylation of resin-bound amino acids via an intermediate 2-nitrobenzenesulfonamide was previously reported: S. C. Miller, T. S. Scanlan, J. Am. Chem. Soc. 119 (9), 2301-2302 (1997). Alternatively, methylation of the intermediate 2-nitrobenzenesulfonamide can be done by a Mitsunobu reaction, as reported by J. Chatterjee et al., Acc. Chem. Res., 41(10), 1331-1342 (2008). The following procedure is a modified version of the procedure reported by Miller and Scanlan.

In a 5 ml plastic reactor (5 ml plastic syringe with internal frit, outlet stoppered during reactions), the peptide resin containing N-terminal Fmoc-DAsp(OtBu) (0.1 mmol, obtained by coupling of Fmoc-DAsp(OtBu)-OH to the peptide resin), was shaken with 20% piperidine in NMP for 20 min, then drained and washed repeatedly with NMP and DCM. A solution of 2,4,6-collidine (0.171 ml, 1.28 mmol) in DCM (1 ml) was added to the resin, followed by a solution of 2-nitrobenzenesulfonyl chloride (0.171 g, 0.77 mmol) in DCM (1.5 ml). The reactor was capped and shaken for 1 h. The resin was washed with NMP/DCM 1:1 (5×4 ml). A solution of methyl 4-nitrobenzensulfonate (261 mg, 1.2 mmol) in NMP (2 ml) was added to the resin, followed by addition of DBU (0.119 ml, 0.8 mmol). The reactor was capped and shaken for 1 h. The resin was drained and washed with NMP (3×2 ml), THF (3×2 ml) and NMP (2×2 ml). NMP (2 ml), 2-mercaptoethanol (0.070 ml, 1.0 mmol) and DBU (0.112 ml, 0.75 mmol) were added to the resin. The reactor was capped and shaken for 1 h. The resin was drained (yellow filtrate) and washed with THF (5×2 ml) to give a resin with N-terminal D-N-methyl-Asp(OtBu) ready for further peptide elongation.

### Attachment of Linker and Fatty Acid Derivative

The side chain attached to the N-terminus of the peptide backbone (i.e. the P-L part of formula I), such as 17-carboxyheptadecanoyl-gGlu-Ado-Ado (i.e. [Chem. 1 where x is 16)]-[Chem. 2 in the L-form]-[Chem. 3a]-[Chem. 3a]), and similar substituents, can be introduced by SPPS as described above, by using suitably protected building blocks, such as but not limited to Fmoc-8-amino-3,6-dioxaoctanoic acid (Fmoc-Ado-OH), Fmoc-Glu-OtBu, octadecanedioic acid mono-tert-butyl ester and hexadecanedioic acid mono-tert-butyl ester. Alkanedioic acid mono-tert-butyl esters can be prepared by reported literature methods, e.g. by refluxing the corresponding alkanedioic acid with N,N-dimethylformamide di-tert-butylacetal in toluene followed by chromatographic purification, or by refluxing the alkanedioic acid with Boc-anhydride, DMAP and tert-butanol in toluene followed by work-up, washing, silica filtration and crystallisation.

Alternatively, the side chain can be prepared separately as its C-terminal carboxylic acid and then activated with a coupling agent to give an acylating agent, e.g. an N-hydroxysuccinimide ester (NHS ester). This acylating agent can be used to attach the side chain to the intermediate CCK peptide, e.g. to the N-terminus of the CCK peptide, by formation of an amide bond. The attachment of the side chain to the intermediate peptide, as well as preparation of the side chain itself can be achieved using methods described in WO 2009/083549 A1.

Cleavage from the Resin and Side-Chain Deprotection

After completed solid-phase peptide synthesis, the resin was extensively washed with DCM. The resin was shaken for 1-2.5 h with a cleavage cocktail consisting of TFA/TIPS/water 95:2.5:2.5. The mixture was filtered and the filtrate was collected. The peptide was precipitated with excess diethylether, collected by centrifugation or filtration, washed with diethylether and collected by centrifugation.

Removal of Neopentyl Group from Tyr(SO₃-Neopentyl)

The crude peptide containing a neopentyl-protected sTyr residue (obtained from 0.1 mmol of resin after SPPS, TFA-cleavage and ether precipitation) was treated with a mixture of acetonitrile (7.5 ml) and 2 M aqueous ammonium acetate solution (7.5 ml), resulting in two liquid phases. The mixture was stirred overnight at 55° C. The deprotected peptide was then isolated by HPLC, as described below.

Removal of Trichloroethyl (TCE) Group from Phe(4-Sulfomethyl-TCE)

The crude peptide containing a TCE-protected Phe(4-sulfomethyl) residue (obtained from 0.1 mmol of resin after SPPS, TFA-cleavage and ether precipitation) was dissolved in acetic acid (3 ml). In a separate test tube with magnetic stirrer, zinc dust (80.5 mg, 1.23 mmol) was treated with 1 M aqueous HCl (2 ml). The stirring zinc suspension was warmed for a moment, resulting in moderate gas evolution. Stirring was stopped and the liquid phase was removed by means of a syringe. To the resulting freshly activated zinc dust, the peptide acetic acid solution was added, resulting in weak gas evolution. The mixture was stirred overnight. Remaining solid was removed by filtration or decantation. The resulting solution was carefully dropped into a solution of Na₂CO₃/NaHCO₃ (1:1) in water (10 ml) and acetonitrile (7.5 ml). The pH of the resulting solution was adjusted to 7.4-7.8 by careful addition of 26% aqueous NH₃ solution. The deprotected peptide was then isolated by HPLC, as described below.

For some compounds of the present invention, a modified procedure was used as follows. The crude peptide containing a TCE-protected Phe(4-sulfomethyl) residue (obtained from 0.1 mmol of resin after SPPS, TFA-cleavage and ether precipitation) was dissolved in acetic acid (2.25 ml). In a separate test tube with magnetic stirrer, zinc dust (81 mg, 1.24 mmol) was stirred with 2 ml of 1M aq. HCl for 5 minutes, resulting in moderate gas evolution. The liquid phase was removed by means of a syringe. To the resulting freshly activated zinc dust, the peptide solution in acetic acid was added. A solution of ammonium acetate (15 mg, 0.2 mmol) in water (0.75 ml) was added. The resulting mixture was stirred for 1 h. LCMS-analysis of the mixture after 1 h indicated completed removal of TCE. The reaction mixture was treated with water (20 ml) and acetonitrile (7 ml). pH was adjusted to 7.6 with 26% aqueous NH3 (approx. 5 ml). The resulting solution was diluted with water to give a total volume of 100 ml, and then purified by preparative HPLC, as described below.

### Purification and Quantification

The crude deprotected peptide dissolved in neutral aqueous ammonium acetate and acetonitrile is purified by reversed-phase preparative HPLC (Waters Deltaprep 4000) on a column containing C18-silica gel. Elution is performed with an increasing gradient of acetonitrile in water containing 0.01 M ammonium acetate. Relevant fractions are checked by analytical UPLC or LCMS. Fractions containing the pure target peptide are mixed and diluted with water. The resulting solution is analysed (HPLC, LCMS) and the product is quantified using a chemiluminescent nitrogen specific HPLC detector (Antek 8060 HPLC-CLND). The product is dispensed into glass vials. The vials are capped with Millipore glassfibre prefilters. Freeze-drying provides the peptide ammonium salt as a white solid.

Transformation of a Peptide Ammonium Salt into a Sodium Salt

If desired, peptide sodium salts can be obtained from their corresponding ammonium salts, e.g. by the following procedure. A Waters “Sep-Pak C18 35 cc—10 g” reversed phase cartridge is washed first with isopropanol/water 9:1 (90 ml) and then with 0.2M NaCl in water/acetonitrile 9:1 (90 ml). The peptide ammonium salt (500 mg) is dissolved in 0.2M NaCl solution in water/acetonitrile 9:1 (90 ml), and pH is adjusted to 7.5 by adding small aliquots of 1M aqueous NaOH. The resulting peptide-NaCl solution is slowly loaded onto the cartridge and the resulting eluate is collected in fractions of 20 ml. The cartridge is washed with 0.2M NaCl solution in water/acetonitrile 9:1 (60 ml; pH adjusted to 7.5 with aqueous NaOH prior to use) and then with water (60 ml) and the resulting eluate is collected in 20 ml fractions. The peptide is eluted with acetonitrile/water 7:3 (60 ml) and the eluate is collected in 20 ml fractions. Relevant fractions are analysed by UPLC and LCMS. Fractions containing the pure peptide are freeze-dried to give the peptide sodium salt.

### Methods for Detection and Characterisation

LCMS method “LCMS31”:

| | |
|---|---|
| System | System: Waters Acquity UPLC SQD 2000 |
| | Column: Acquity UPLC BEH 1.7μ C18 100 Å |
| | 2.1 × 50 mm |
| | Detector: UV: PDA, SQD 2000 |
| Detector | Ionisation method: electrospray positive |
| setup | Scanning range: 100-1500 |
| | Cone Voltage: 25 V |
| | Scantime 0.5 |
| Conditions | Linear gradient: 10% to 90% B |
| | Gradient run-time: 9 min |
| | Total run-time: 10 min |
| | Flow rate: 0.3 ml/min |
| | Column temperature: 40° C. |
| | PDA: 210-400 nm |
| Eluents | Solvent A: 99.9% water, 0.1% TFA |
| | Solvent B: 99.9% acetonitrile, 0.1% TFA |

LCMS method “LCMS34”:

| | |
|---|---|
| System | LC-system: Waters Acquity UPLC H Class |
| | Column:: Waters Acquity BEH, C-18, 1.7 μm, 2.1 |
| | mm × 50 mm |
| | Detector:: Waters Xevo G2-XS QTof |
| Detector | Ionisation method: electrospray, positive mode |
| setup | Scanning range: 50-4000 amu |
| | Operating mode: MS resolution mode |
| | Voltage: Capillary 3.00 kV |
| | Sample cone 80 V |
| | Source 60 V |
| | Temperature: Source 150° C. |
| | Desolvation 500° C. |
| | Scantime 0.500 s |
| | Interscandelay: 0.014 s |
| Conditions | Linear gradient: 5% to 95% solvent B, 90% to 0% solvent A, |
| | 5% solvent C |
| | Gradient run-time: 4.0 minutes |
| | Total run-time: 7.0 minutes |
| | Flow rate: 0.4 ml/min |
| | Column temperature: 40° C. |
| Eluents | Solvent A: 99.90% water, 0.1% formic acid |
| | Solvent B: 99.90% acetonitrile, 0.1% formic acid |
| | Solvent C: 99.99% water, 0.01% TFA |

LCMS method “LCMS01-neg”:

| | |
|---|---|
| System | LC-system: Waters Acquity UPLC |
| | Column: Waters Acquity UPLC BEH, C-18, 1.7 μm, 2.1 |
| | mm × 50 mm |
| | Detector: Waters (Micromass) LCT Premier XE |
| Detector | Ionisation method: electrospray, negative mode |
| setup | Scanning range: 500-2000 amu |
| | Cone Voltage: 50 V |
| | Scantime 1 |
| | Interscandelay: 0.0 |
| Conditions | Linear gradient: 5% to 95% B |
| | Gradient run-time: 4.0 minutes |
| | Total run-time: 7.0 minutes |
| | Flow rate: 0.4 ml/min |
| | Column temperature: 40° C. |
| Eluents | Solvent A: 99.9% water, 0.1% formic acid |
| | Solvent B: 99.9% acetonitrile, 0.1% formic acid |

UPLC method “UPLC17”:

| | |
|---|---|
| System | System: Waters Acquity UPLC system |
| | Column: ACQUITY UPLC BEH Shield RP18, 1.7 um, 2.1 |
| | mm × 150 mm column |
| | Detector: Waters Acquity TUV Detector |
| Detector | 214 nm and 254 nm |
| setup | |
| Conditions | Step gradient: 10-20% B over 3 minutes, then 20-80% B |
| | over 17 minutes, then 80-90% B over 1 minute |
| | Step gradient run-time: 21 minutes |
| | Total run-time: 26 minutes |
| | Flow rate: 0.40 ml/min fixed |
| | Column temperature: 60° C. |
| Eluents | Solvent A: 0.2M sodium sulfate, 0.02M disodium |
| | hydrogenphosphate, 0.02M sodium dihydrogenphosphate, |
| | 90% water and 10% acetonitrile, pH 7.2 |
| | Solvent B: 70% acetonitrile, 30% water |

UPLC method “UPLC60”:

| | |
|---|---|
| System | System: Waters Acquity UPLC system (different instruments |
| | of the same type were used) |
| | Column: ACQUITY UPLC BEH Shield RP18, 1.7 um, 2.1 |
| | mm × 150 mm column |
| | Detectors: Waters Acquity TUV Detector |
| Detector | 214 nm |
| setup | |
| Conditions | Step gradient: 10-20% B over 3 minutes, then 20-50% B |
| | over 17 minutes, then 50-80% B over 1 minute |
| | Step gradient run-time: 21 minutes |
| | Total run-time: 26 minutes |
| | Flow rate: 0.40 ml/min fixed |
| | Column temperature: 60° C. |
| Eluents | Solvent A: 20 mM sodium sulfate, 2 mM disodium |
| | hydrogenphosphate, 2 mM sodium dihydrogenphosphate, 90% |
| | water and 10% acetonitrile, pH 7.2 |
| | Solvent B: 70% acetonitrile, 30% water |

UPLC method “UPLC61”:

| | |
|---|---|
| System | System: Waters Acquity UPLC system (different instruments |
| | of the same type were used) |
| | Column: ACQUITY UPLC BEH Shield RP18, 1.7 um, 2.1 |
| | mm × 150 mm column |
| | Detectors: Waters Acquity TUV Detector |
| Detector | 214 nm and 254 nm |
| setup | |
| Conditions | Step gradient: 10-20% B over 3 minutes, then 20-80% B |
| | over 17 minutes, then 80-90% B over 1 minute |
| | Step gradient run-time: 21 minutes |
| | Total run-time: 26 minutes |
| | Flow rate: 0.40 ml/min fixed |
| | Column temperature: 60° C. |
| Eluents | Solvent A: 20 mM sodium sulfate, 2 mM disodium |
| | hydrogenphosphate, 2 mM sodium dihydrogenphosphate, 90% |
| | water and 10% acetonitrile, pH 7.2 |
| | Solvent B: 70% acetonitrile, 30% water |

### Example COMPOUNDS OF THE INVENTION

The following example compounds were synthesised and purified using state of the art methodology. They were analysed and can be synthesised and purified as described in the above section headed “Methods for Synthesis, Purification and Analysis of Example Compounds”.

### Example 1

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Nle-Gly-Trp-Nle-DMeAsp-Trp-amide

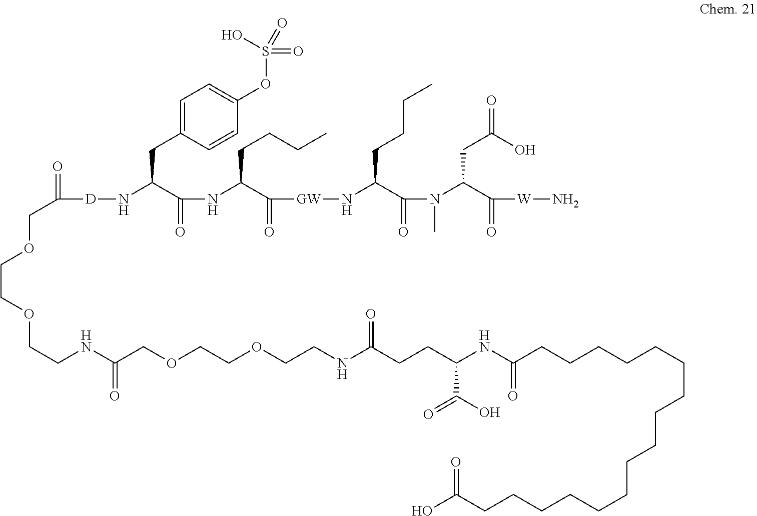

LCMS31: Rt=5.6 min; m/z=939 (calculated: (m+2)/2)=939.1)

UPLC17: Rt=8.5 min

### Example 2

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Nle-Gly-Trp-Nle-DMeAsp-1Nal-amide

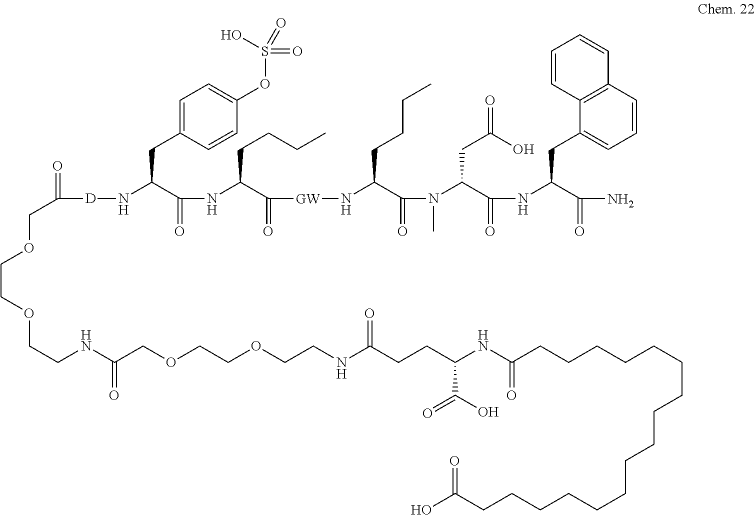

LCMS31: Rt=5.9 min; m/z=944 (calculated: (m+2)/2)=944.6)

UPLC17: Rt=9.1 min

### Example 3

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-Phe(4-sulfomethyl)-Nle-Gly-Trp-Nle-DMeAsp-MePhe-amide

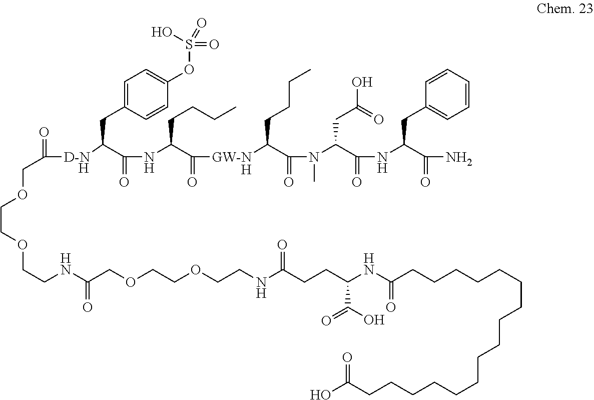

LCMS31: Rt=5.8 min; m/z=926 (calculated: (m+2)/2)=925.6)

UPLC61: Rt=7.0 min

### Example 4

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Nle-DMeAsp-Me1Nal-amide

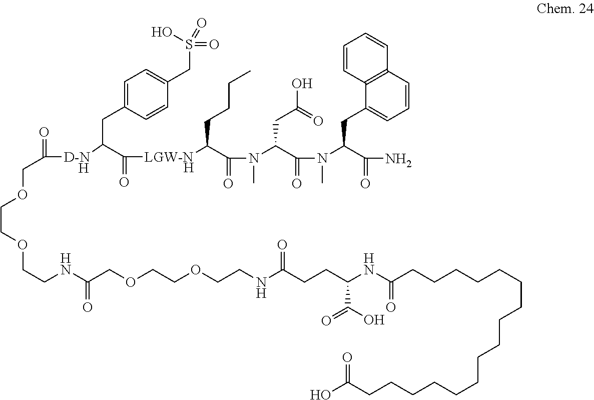

LCMS31: Rt=5.9 min; m/z=952 (calculated: (m+2)/2)=951.6)

UPLC61: Rt=7.2 min

### Example 5

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-Phe(4-sulfomethyl)-Leu-Gly-Trp-Nle-DMeAsp-MePhe-amide

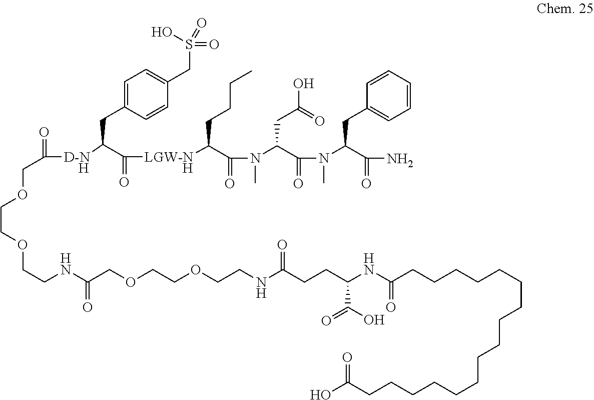

LCMS31: Rt=5.7 min; m/z=926 (calculated: (m+2)/2)=925.6)

UPLC61: Rt=6.3 min

### Example 6

### 2-[2-[2-[[2-[2-[2-[[(4S)-4-Carboxy-4-(15-carboxypentadecanoylamino)butanoyl]amino]ethoxy]ethoxy]acetyl]amino]ethoxy]ethoxy]acetyl-Asp-Phe(4-sulfomethyl)-Leu-Gly-Trp-Nle-DMeAsp-MePhe-amide

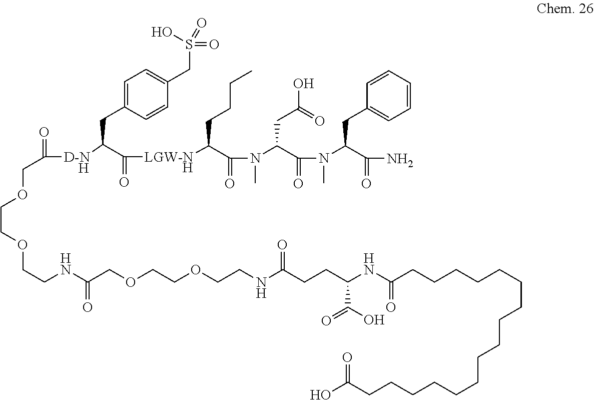

LCMS31: Rt=5.2 min; m/z=911 (calculated: (m+2)/2)=911.5)

UPLC61: Rt=5.1 min

### Example 7

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Nle-DMeAsp-Phe-amide

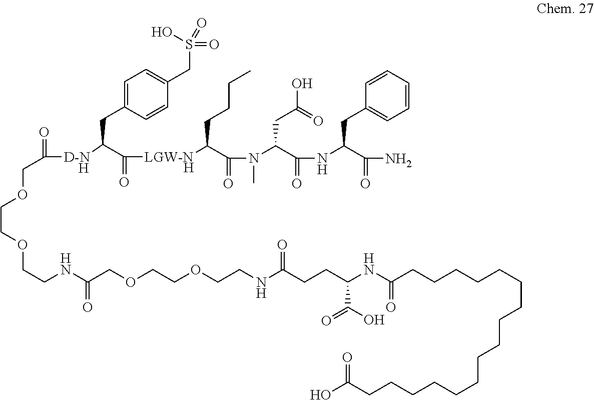

LCMS31: Rt=5.7 min; m/z=919 (calculated: (m+2)/2)=919.5)

UPLC61: Rt=6.7 min

### Example 8

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Nle-Gly-Trp-Nle-DMeAsp-MePhe-amide

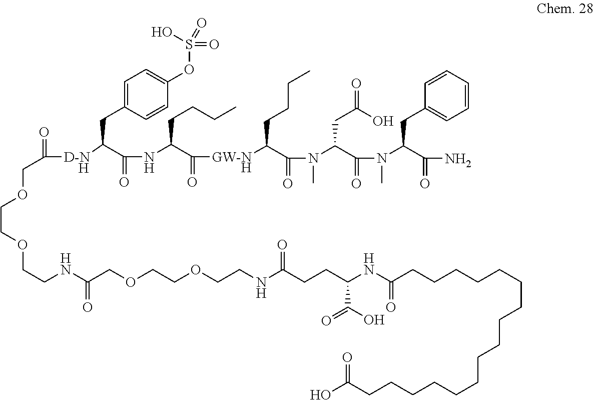

LCMS31: Rt=5.9 min; m/z=927 (calculated: (m+2)/2)=926.6)

UPLC61: Rt=7.2 min

### Example 9

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Nle-DMeAsp-MePhe-amide

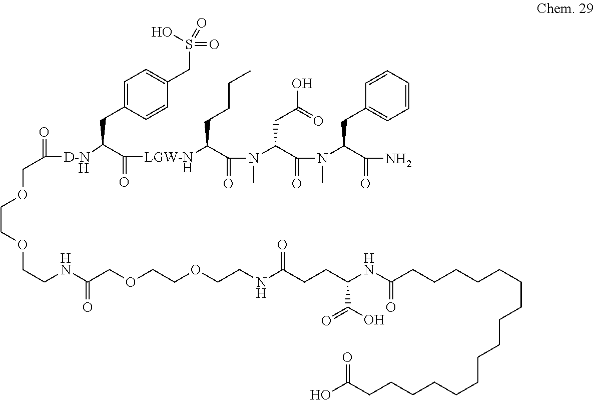

LCMS31: Rt=5.9 min; m/z=926 (calculated: (m+2)/2)=926.6)

UPLC61: Rt=7.1 min

### Example 10

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Ile-DMeAsp-MePhe-amide

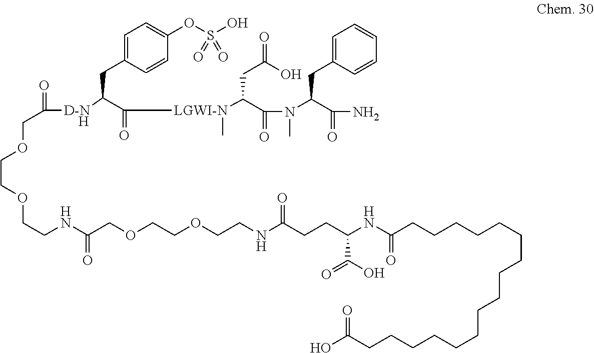

LCMS31: Rt=5.6 min; m/z=926 (calculated: (m+2)/2)=926.6)

UPLC61: Rt=6.2 min

### Example 11

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Gln-DMeAsp-MePhe-amide

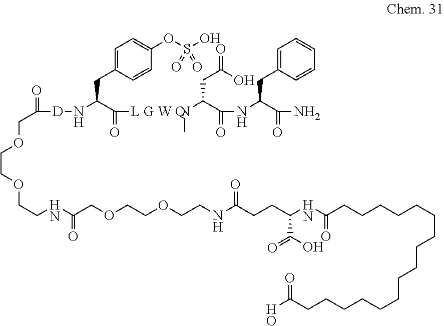

LCMS31: Rt=5.2 min; m/z=934 (calculated: (m+2)/2)=934.0)

UPLC61: Rt=5.4 min

### Example 12

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Met(O2)-DMeAsp-MePhe-amide

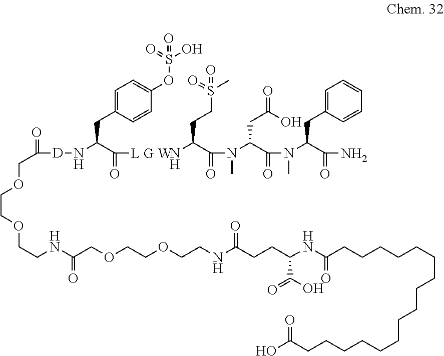

LCMS31: Rt=5.4 min; m/z=952 (calculated: (m+2)/2)=951.6)

UPLC61: Rt=5.6 min

### Example 13

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Leu-DMeAsp-MePhe-amide

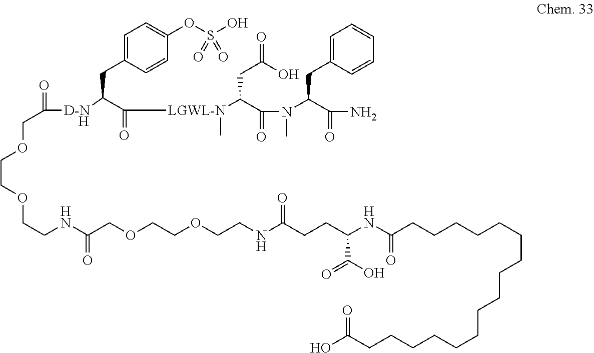

LCMS31: Rt=5.6 min; m/z=927 (calculated: (m+2)/2)=926.6)

UPLC61: Rt=6.5 min

### Example 14

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Val-DMeAsp-MePhe-amide

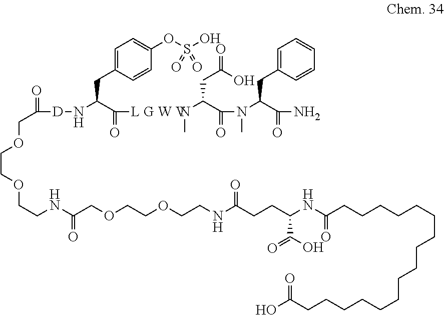

LCMS31: Rt=5.5 min; m/z=919 (calculated: (m+2)/2)=919.5)

UPLC61: Rt=7.5 min

### Example 15

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Pro-DMeAsp-MePhe-amide

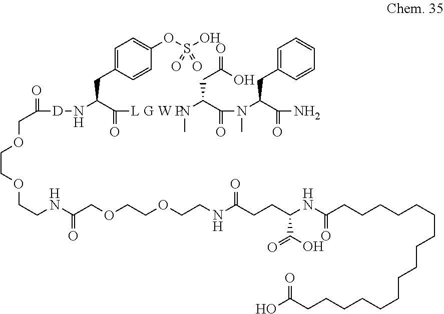

LCMS31: Rt=5.4 min; m/z=919 (calculated: (m+2)/2)=918.5)

UPLC61: Rt=7.1 min

### Example 16

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Lys-Gly-Trp-Nle-DMeAsp-MePhe-amide

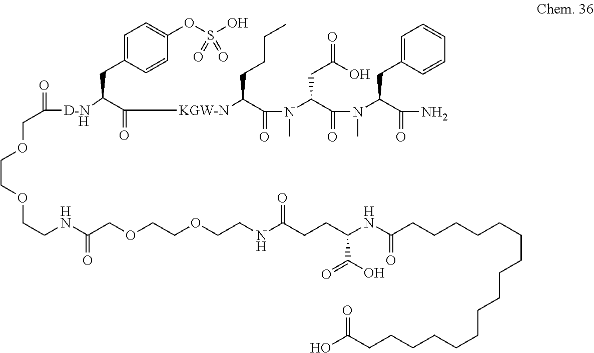

LCMS31: Rt=5.3 min; m/z=934 (calculated: (m+2)/2)=934.1)

UPLC61: Rt=7.7 min

### Example 17

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Arg-Gly-Trp-Nle-DMeAsp-MePhe-amide

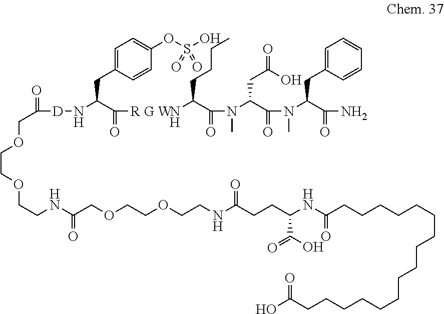

LCMS31: Rt=5.3 min; m/z=948 (calculated: (m+2)/2)=948.1)

UPLC61: Rt=7.7 min

### Example 18

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Pro-Gly-Trp-Nle-DMeAsp-MePhe-amide

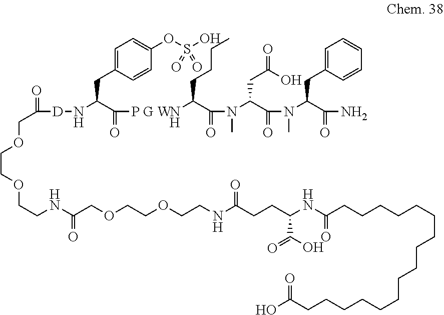

LCMS31: Rt=5.4 min; m/z=919 (calculated: (m+2)/2)=918.5)

UPLC61: Rt=7.3 min

### Example 19

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Lys-2-[2-(2-aminoethoxy)ethoxy]acetyl-Asp-Phe(4-sulfomethyl)-Nle-Gly-Trp-Nle-DMeAsp-MePhe-amide

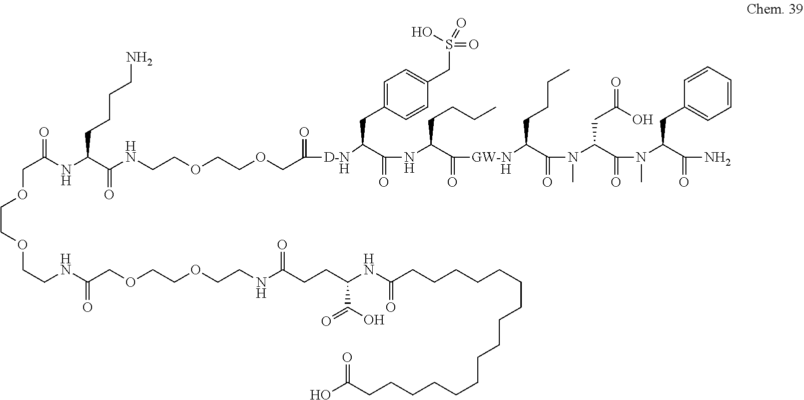

LCMS31: Rt=5.4 min; m/z=1062 (calculated: (m+2)/2)=1062.2)

UPLC61: Rt=8.9 min

### Example 20

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Met-Gly-Trp-Nle-DMeAsp-MePhe-amide

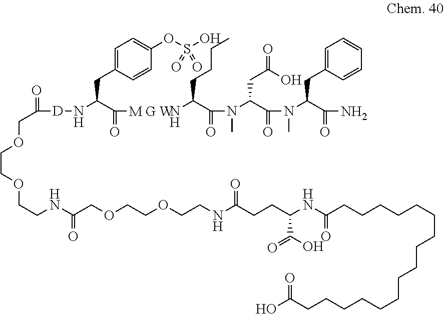

LCMS01-neg: Rt=4.1 min; m/z=1868 (calculated: (m−1)/1)=1868.2)

UPLC61: Rt=5.6 min

### Example 21

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Nle-Gly-Trp-Met-DMeAsp-MePhe-amide

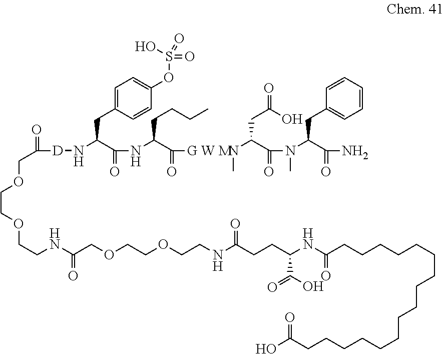

LCMS01-neg: Rt=3.8 min; m/z=1868 (calculated: (m−1)/1)=1868.2)

UPLC61: Rt=5.7 min

### Example 22

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-sTyr-Nle-Gly-Trp-Nle-DMeAsp-MePhe-amide

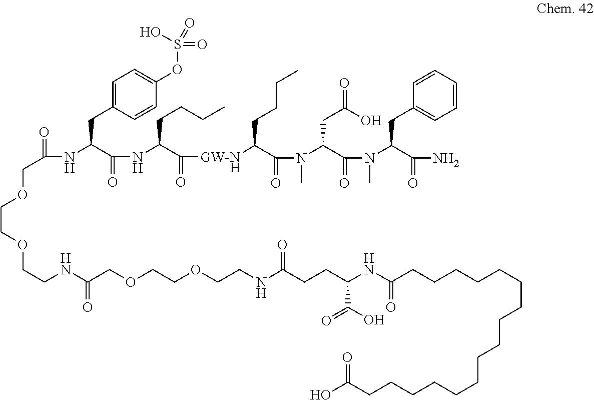

LCMS01-neg: Rt=4.2 min; m/z=1735 (calculated: (m−1)/1)=1735.0)

UPLC61: Rt=7.5 min

### Example 23

### 3-[2-[2-[2-[2-[2-[2-[2-[2-[2-[2-[3-[2-[2-[2-[2-[2-[2-[2-[2- [2-[2-[[(4S)-4-Carboxy-4-(17-carboxyheptadecanoylamino)butanoyl]amino]ethoxy]ethoxy]ethoxy]ethoxy]ethoxy]ethoxy]ethoxy]ethoxy]ethoxy]ethoxy]propanoylamino]ethoxy]ethoxy]ethoxy]ethoxy]ethoxy]ethoxy]ethoxy]ethoxy]ethoxy]ethoxy]propanoyl-Asp-sTyr-Nle-Gly-Trp-Nle-DMeAsp-MePhe-amide

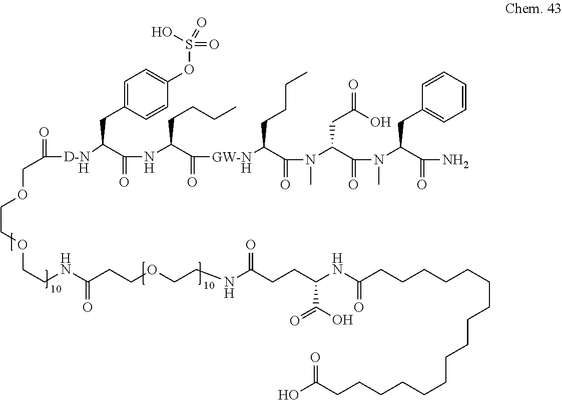

LCMS01-neg: Rt=3.6 min; m/z=1291 (calculated: (m−2)/2)=1291.0)

UPLC61: Rt=7.6 min

### Example 24

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Met-Gly-Trp-Met-DMeAsp-MePhe-amide

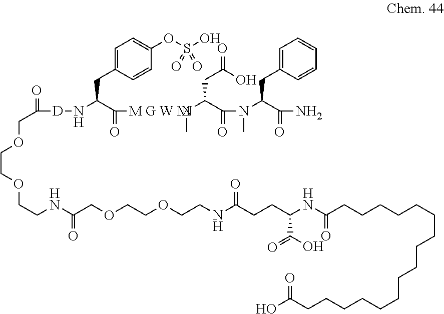

LCMS01-neg: Rt=3.8 min; m/z=1886 (calculated: (m−1)/1)=1886.2)

UPLC61: Rt=4.8 min

### Example 25

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-DAsp-Phe(4-sulfomethyl)-Nle-Gly-Trp-Nle-DMeAsp-MePhe-amide

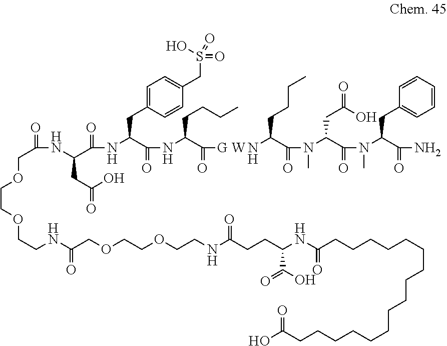

LCMS31: Rt=5.5 min; m/z=925 (calculated: (m+2)/2)=925.6)

UPLC61: Rt=7.7 min

### Example 26

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-bAsp-Phe(4-sulfomethyl)-Nle-Gly-Trp-Nle-DMeAsp-MePhe-amide

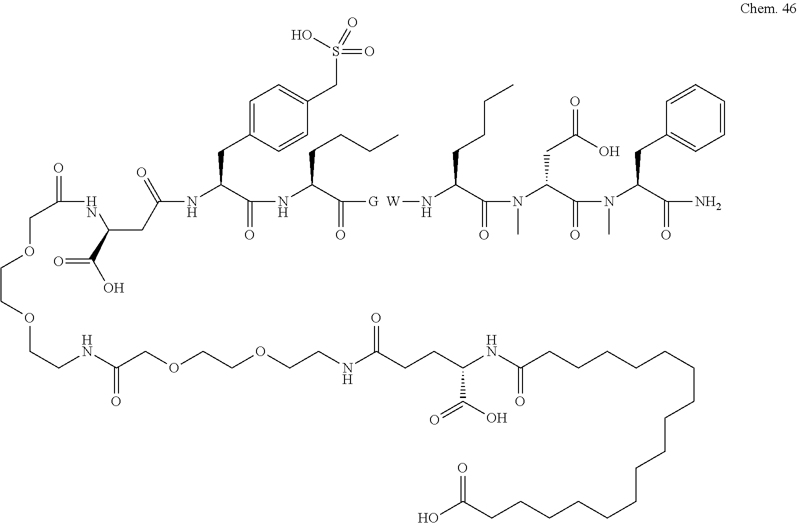

LCMS31: Rt=5.5 min; m/z=925 (calculated: (m+2)/2)=925.6)

UPLC61: Rt=7.6 min

### Example 27

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-DbAsp-Phe(4-sulfomethyl)-Nle-Gly-Trp-Nle-DMeAsp-MePhe-amide

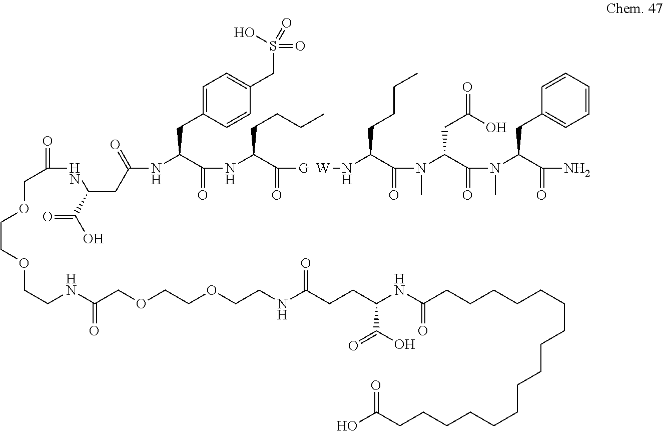

LCMS31: Rt=5.5 min; m/z=925 (calculated: (m+2)/2)=925.6)

UPLC61: Rt=7.7 min

### Example 28

### 2-[2-[2-[[2-[2-[2-[[(4S)-4-Carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl-Asp-sTyr-Leu-Gly-Trp-Nle-DMeAsp-MePhe-amide

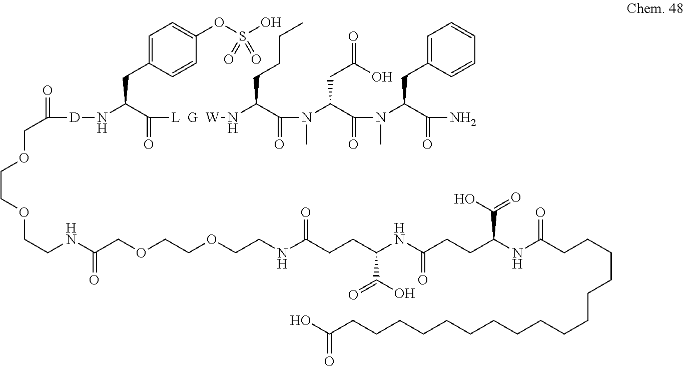

LCMS31: Rt=5.4 min; m/z=991 (calculated: (m+2)/2)=991.1)

UPLC61: Rt=7.0 min

### Example 29

### 17-Carboxyheptadecanoyl-Gly-Ser-Ser-Gly-Ser-Ser-Asp-sTyr-Leu-Gly-Trp-Nle-DMeAsp-MePhe-amide

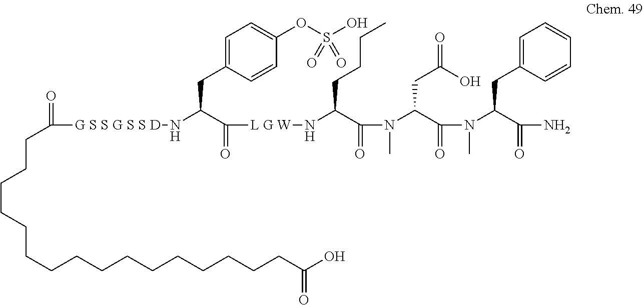

LCMS31: Rt=5.4 min; m/z=948 (calculated: (m+2)/2)=948.1)

UPLC61: Rt=9.2 min

### Example 30

### 2-[2-[2-[[2-[2-[2-(17-Carboxyheptadecanoylamino)ethoxy]ethoxy]acetyl]amino]ethoxy]ethoxy]-Asp-sTyr-Leu-Gly-Trp-Nle-DMeAsp-MePhe-amide

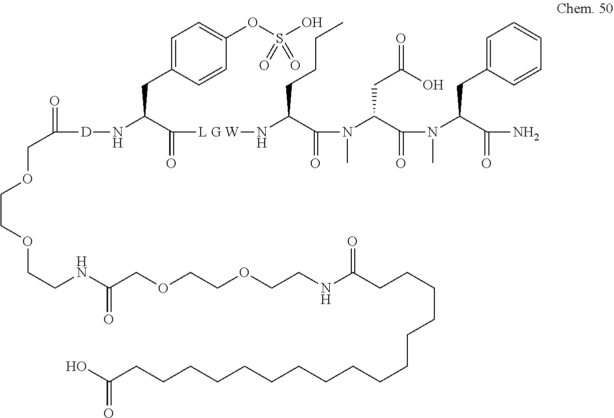

LCMS31: Rt=5.9 min; m/z=862 (calculated: (m+2)/2)=862.0)

UPLC61: Rt=9.8 min

### Example 31

### 2-[2-[2-[[2-[2-[2-[[2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl]amino]ethoxy]ethoxy]acetyl]amino]ethoxy]ethoxy]acetyl-Asp-sTyr-Leu-Gly-Trp-Nle-DMeAsp-MePhe-amide

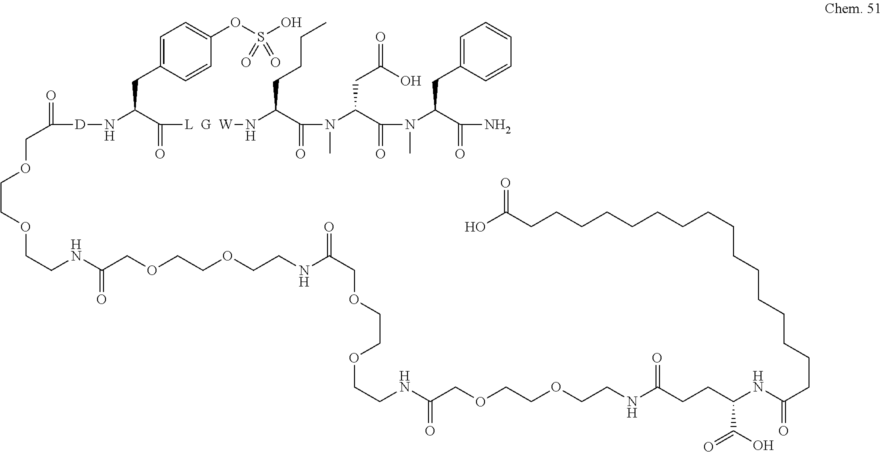

LCMS31: Rt=5.4 min; m/z=1071 (calculated: (m+2)/2)=1071.7)

UPLC61: Rt=7.9 min

### Example 32

### (4S)-4-Carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl-Asp-sTyr-Leu-Gly-Trp-Nle-DMeAsp-MePhe-amide

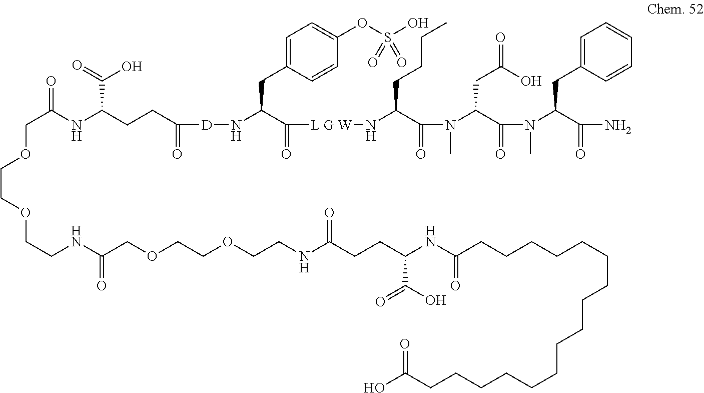

LCMS31: Rt=5.5 min; m/z=991 (calculated: (m+2)/2)=991.1)

UPLC61: Rt=6.3 min

### Example 33

### 17-Carboxyheptadecanoyl-gGlu-gGlu-gGlu-Asp-sTyr-Leu-Gly-Trp-Nle-DMeAsp-MePhe-amide

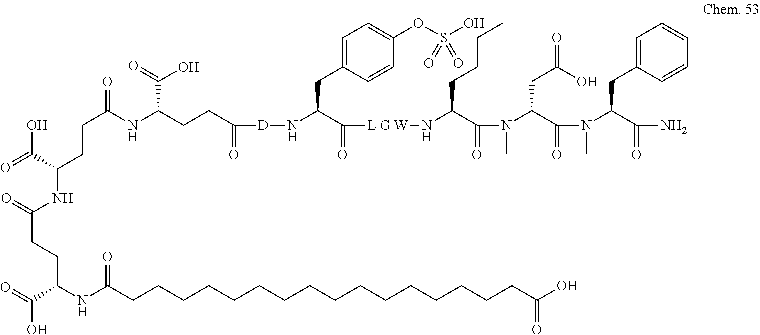

LCMS31: Rt=5.5 min; m/z=910 (calculated: (m+2)/2)=910.5)

UPLC61: Rt=6.3 min

### Example 34

### 2-[2-[2-[[2-[2-[2-[[(4S)-4-Carboxy-4-(13-carboxytridecanoylamino)butanoyl]amino]ethoxy]ethoxy]acetyl]amino]ethoxy]ethoxy]acetyl-Asp-Phe(4-sulfomethyl)-Leu-Gly-Trp-Nle-DMeAsp-MePhe-amide

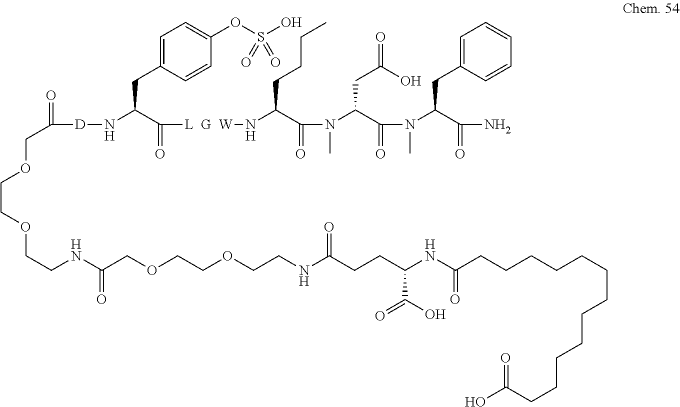

LCMS31: Rt=4.6 min; m/z=897 (calculated: (m+2)/2)=897.5)

UPLC61: Rt=5.4 min

### Example 35

### 2-[2-[2-[[2-[2-[2-[[(4S)-4-Carboxy-4-(19-carboxynonadecanoylamino)butanoyl]amino]ethoxy]ethoxy]acetyl]amino]ethoxy]ethoxy]acetyl-Asp-Phe(4-sulfomethyl)-Leu-Gly-Trp-Nle-DMeAsp-MePhe-amide

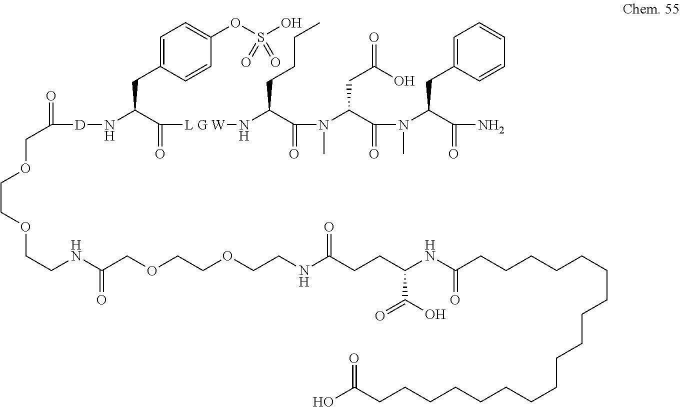

LCMS31: Rt=6.1 min; m/z=939 (calculated: (m+2)/2)=939.6)

UPLC61: Rt=8.9 min

### Example 36

### 2-[2-[2-[[2-[2-[2-[[(4S)-4-Carboxy-4-(13-carboxytridecanoylamino)butanoyl]amino]ethoxy]ethoxy]acetyl]amino]ethoxy]ethoxy]acetyl-Glu-sTyr-Leu-Gly-Trp-Nle-DMeAsp-MePhe-amide

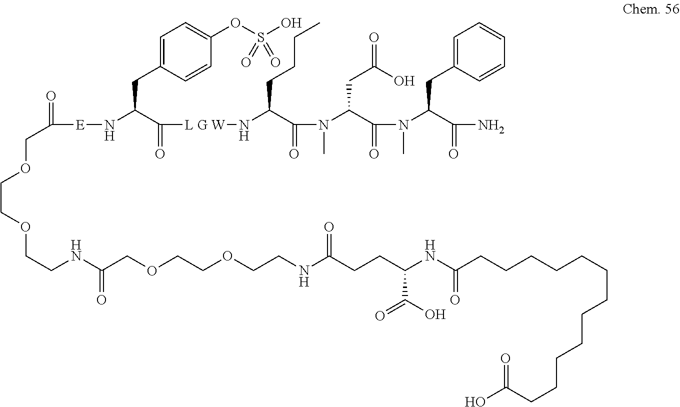

LCMS31: Rt=4.7 min; m/z=905 (calculated: (m+2)/2)=905.5)

UPLC61: Rt=4.8 min

### Example 37

### 2-[2-[2-[[2-[2-[2-[[(4S)-4-Carboxy-4-(19-carboxynonadecanoylamino)butanoyl]amino]ethoxy]ethoxy]acetyl]amino]ethoxy]ethoxy]acetyl-Glu-sTyr-Leu-Gly-Trp-Nle-DMeAsp-MePhe-amide

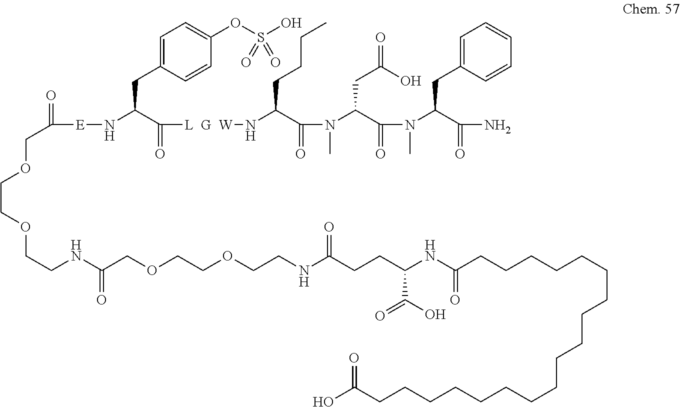

LCMS31: Rt=6.1 min; m/z=947 (calculated: (m+2)/2)=947.6)

UPLC61: Rt=8.4 min

### Example 38

### 17-Carboxyheptadecanoyl-Asp-sTyr-Leu-Gly-Trp-Nle-DMeAsp-MePhe-amide

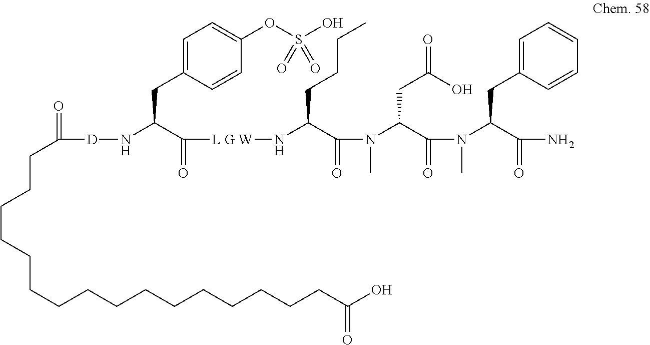

LCMS31: Rt=6.2 min; m/z=1432 (calculated: (m+1)/1)=1432.7)

UPLC61: Rt=9.4 min

### Example 39

### 2-[2-[2-[[2-[2-[2-[[(4S)-4-Carboxy-4-(15-carboxypentadecanoylamino)butanoyl]amino]ethoxy]ethoxy]acetyl]amino]ethoxy]ethoxy]acetyl-Glu-sTyr-Leu-Gly-Trp-Nle-DMeAsp-MePhe-amide

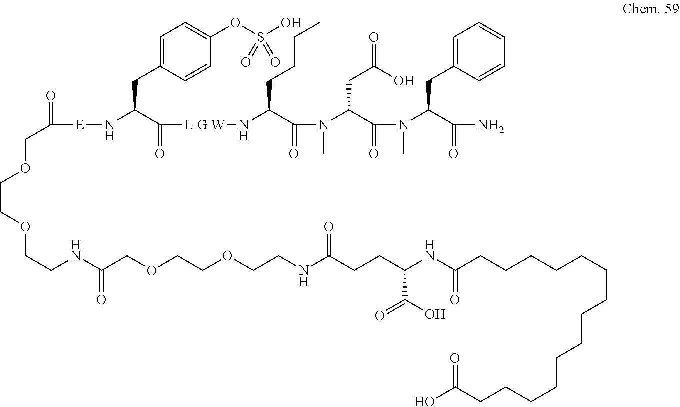

LCMS31: Rt=5.1 min; m/z=919 (calculated: (m+2)/2)=919.5)

UPLC61: Rt=5.9 min

### Example 40

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Ala-DMeAsp-MePhe-amide

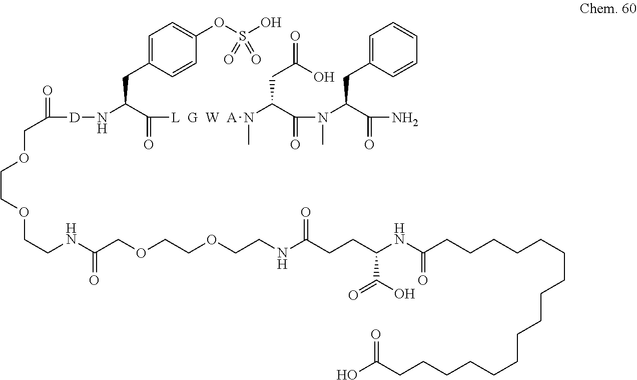

LCMS31: Rt=5.3 min; m/z=906 (calculated: (m+2)/2)=905.5)

UPLC61: Rt=6.5 min

### Example 41

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Glu-sTyr-Leu-Gly-Trp-Gln-DMeAsp-Phe-amide

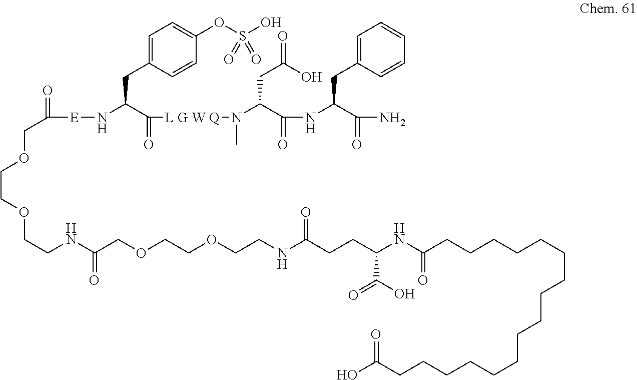

LCMS31: Rt=5.2 min; m/z=934 (calculated: (m+2)/2)=934.0)

UPLC61: Rt=6.2 min

### Example 42

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Glu-sTyr-Leu-Gly-Trp-Gln-DMeAsp-MePhe-amide

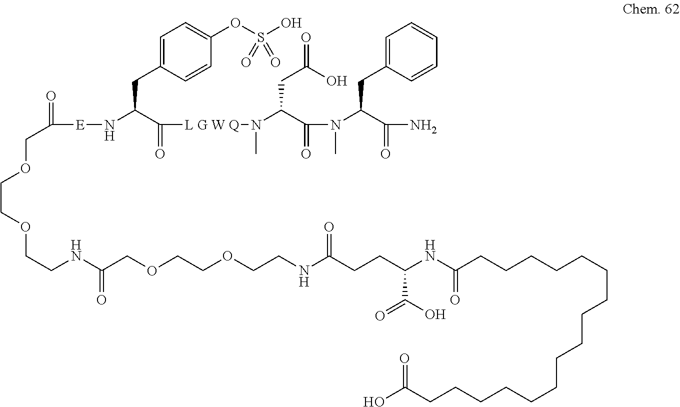

LCMS31: Rt=5.2 min; m/z=941 (calculated: (m+2)/2)=941.1)

UPLC61: Rt=6.2 min

### Example 43

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Glu-sTyr-Leu-Gly-Trp-Nle-DMeAsp-MePhe-amide

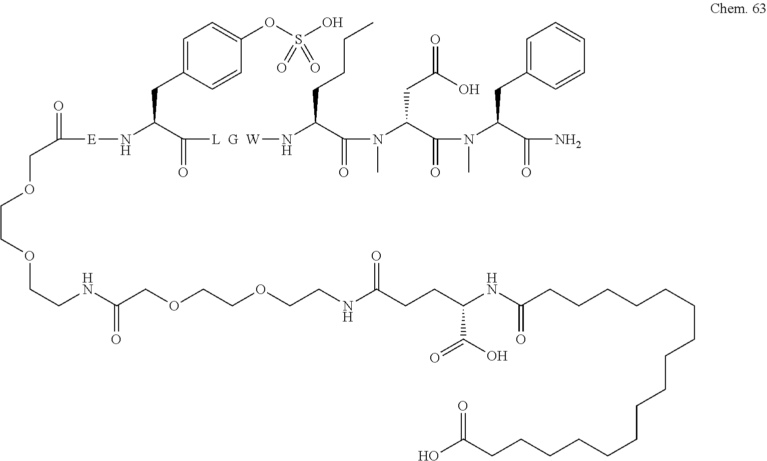

LCMS31: Rt=5.6 min; m/z=933 (calculated: (m+2)/2)=933.6)

UPLC61: Rt=7.7 min

### Example 44

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Phe-Gly-Trp-Nle-DMeAsp-MePhe-amide

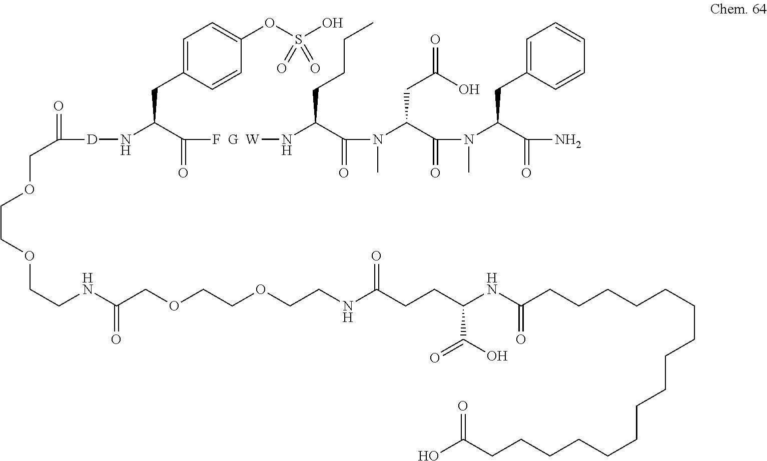

LCMS31: Rt=5.7 min; m/z=943 (calculated: (m+2)/2)=943.6)

UPLC61: Rt=8.0 min

### Example 45

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Ser-Gly-Trp-Nle-DMeAsp-MePhe-amide

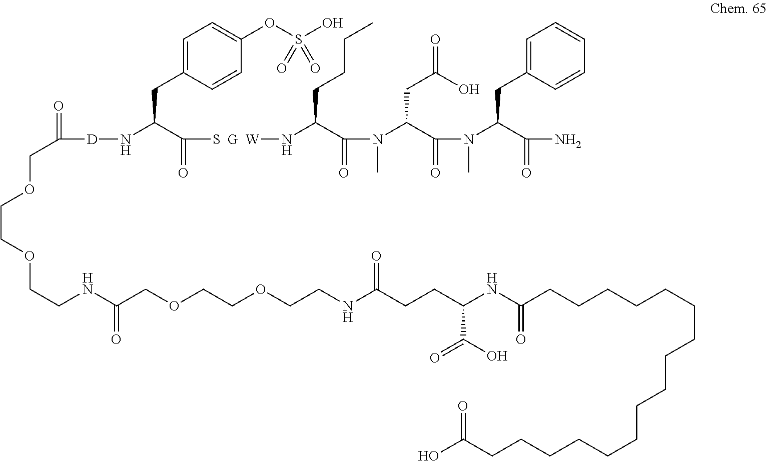

LCMS31: Rt=5.3 min; m/z=913 (calculated: (m+2)/2)=913.5)

UPLC61: Rt=7.2 min

### Example 46

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Ala-Gly-Trp-Nle-DMeAsp-MePhe-amide

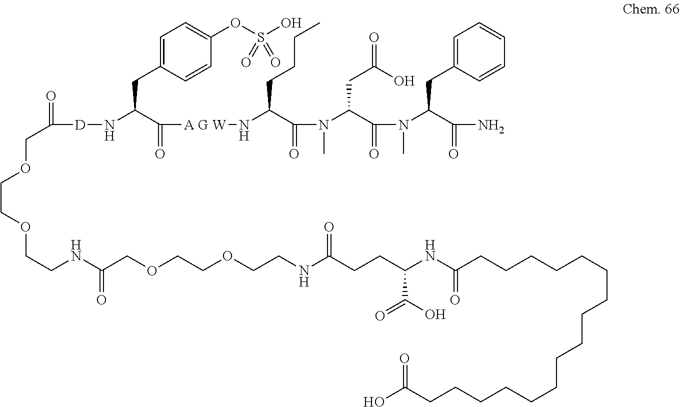

LCMS31: Rt=5.4 min; m/z=905 (calculated: (m+2)/2)=905.5)

UPLC61: Rt=5.6 min

### Example 47

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Ile-Gly-Trp-Nle-DMeAsp-MePhe-amide

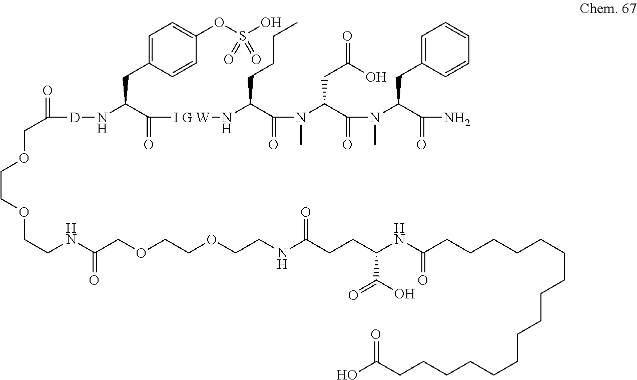

LCMS31: Rt=5.6 min; m/z=926 (calculated: (m+2)/2)=926.6)

UPLC61: Rt=6.0 min

### Example 48

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-His-Gly-Trp-Nle-DMeAsp-MePhe-amide

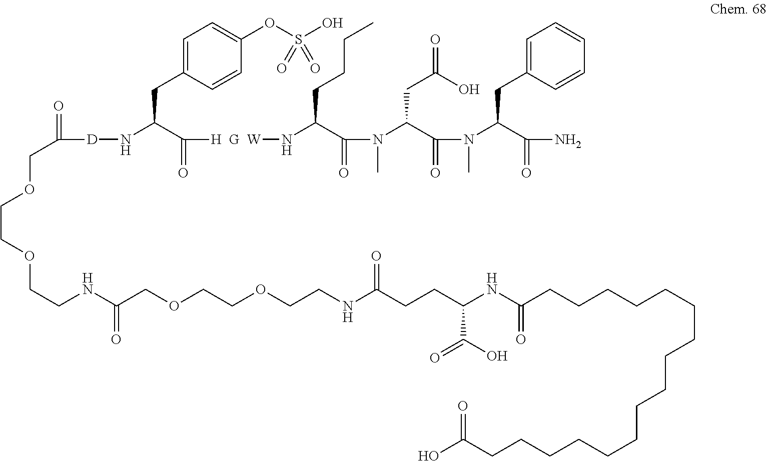

LCMS31: Rt=5.3 min; m/z=938 (calculated: (m+2)/2)=938.5)

UPLC61: Rt=5.6 min

### Example 49

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-DGlu-sTyr-Leu-Gly-Trp-Nle-DMeAsp-MePhe-amide

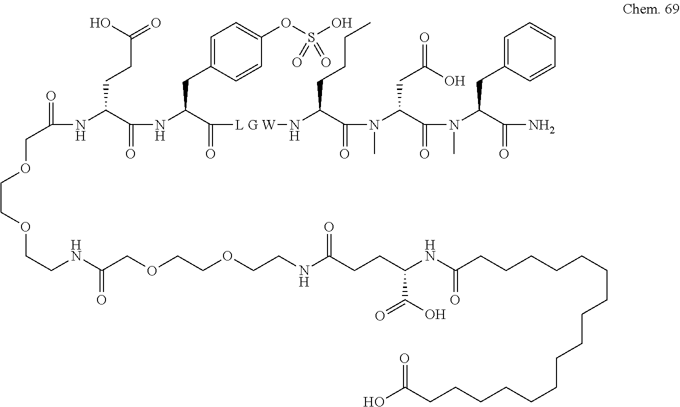

LCMS31: Rt=5.6 min; m/z=933 (calculated: (m+2)/2)=933.6)

UPLC61: Rt=7.4 min

### Example 50

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Leu-Gly-Trp-Nle-DMeAsp-2Nal-amide

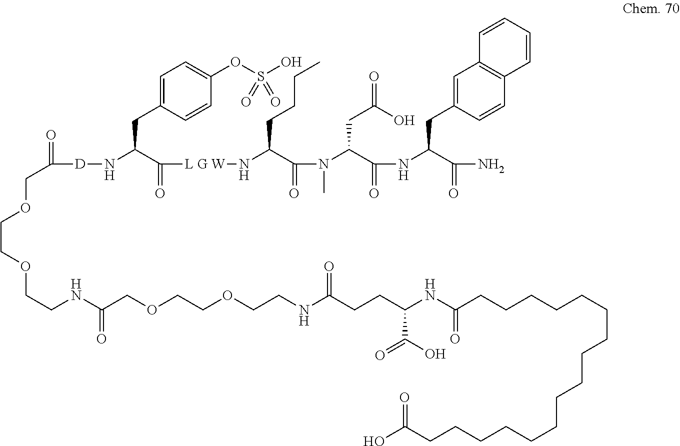

LCMS31: Rt=5.8 min; m/z=945 (calculated: (m+2)/2)=944.6)

UPLC61: Rt=8.0 min

### Example 51

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Asp-sTyr-Met-Gly-Trp-Met-DMeAsp-Phe-amide

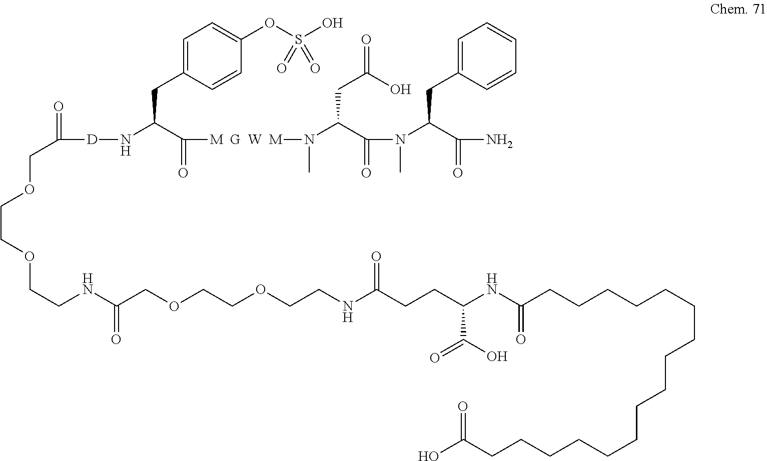

LCMS31: Rt=5.4 min; m/z=937 (calculated: (m+2)/2)=937.6)

UPLC61: Rt=6.5 min

### Example 52

### 2-[2-[2-[[2-[2-[2-[[(4S)-4-Carboxy-4-(17-carboxyheptadecanoylamino)butanoyl]amino]ethoxy]ethoxy]acetyl]amino]ethoxy]ethoxy]acetyl-Glu-sTyr-Leu-Gly-Trp-Nle-DMeAsp-Phe-amide

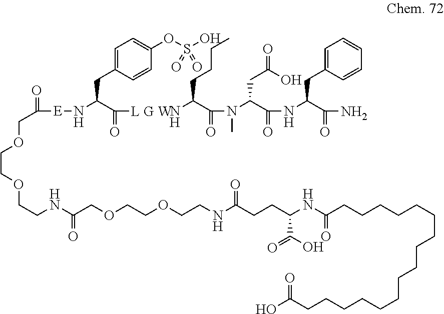

LCMS31: Rt=5.5 min; m/z=926 (calculated: (m+2)/2)=926.6)

UPLC61: Rt=7.1 min

### Example 53

### Asp-sTyr-Nle-Gly-Trp-Nle-DMeAsp-Trp-amide

### Seq 21:

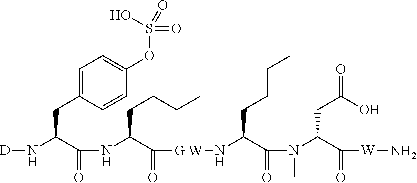

### Example 54

### Asp-sTyr-Nle-Gly-Trp-Nle-DMeAsp-1Nal-amide

### Seq 22:

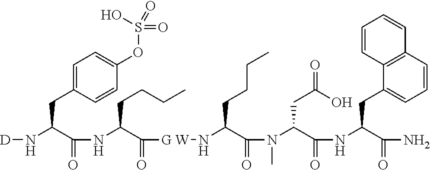

UPLC60: Rt=6.8 min

### Example 55

### Asp-Phe(4-sulfomethyl)-Nle-Gly-Trp-Nle-DMeAsp-MePhe-amide

### Seq 23:

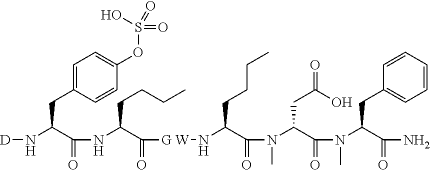

LCMS31: Rt=3.7 min; m/z=1133 (calculated: (m+1)/1)=1134.3)

UPLC61: Rt=4.9 min

### Example 56

### Asp-sTyr-Leu-Gly-Trp-Nle-DMeAsp-Me1Nal-amide

### Seq 24:

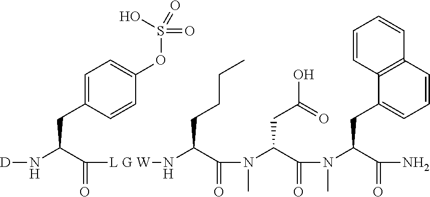

### Example 57

### Asp-Phe(4-sulfomethyl)-Leu-Gly-Trp-Nle-DMeAsp-MePhe-amide

### Seq 25:

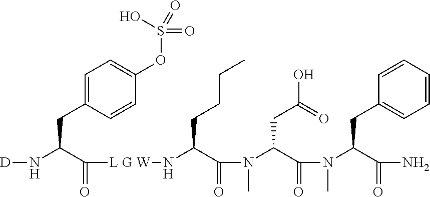

LCMS31: Rt=3.7 min; m/z=1133 (calculated: (m+1)/1)=1134.3)

UPLC61: Rt=4.7 min

### Example 58

### Asp-sTyr-Leu-Gly-Trp-Nle-DMeAsp-Phe-amide

### Seq 27:

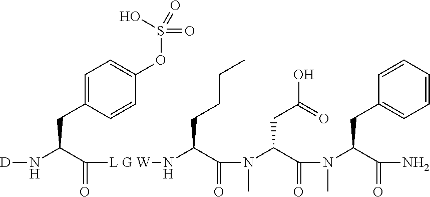

LCMS31: Rt=3.6 min; m/z=1121 (calculated: (m+1)/1)=1122.2)

UPLC61: Rt=4.4 min

### Example 59

### Asp-sTyr-Nle-Gly-Trp-Nle-DMeAsp-MePhe-amide

### Seq 28:

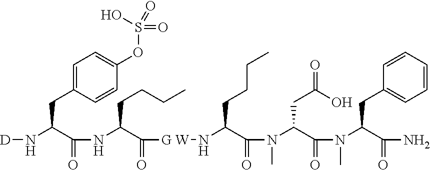

LCMS31: Rt=3.8 min; m/z=1136 (calculated: (m+1)/1)=1136.2)

UPLC61: Rt=5.0 min

### Example 60

### Asp-sTyr-Leu-Gly-Trp-Nle-DMeAsp-MePhe-amide

### Seq 29:

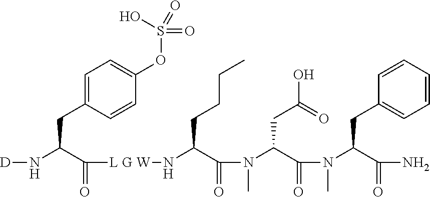

LCMS31: Rt=3.7 min; m/z=1135 (calculated: (m+1)/1)=1136.2)

UPLC61: Rt=5.2 min

### Example 61

### Asp-sTyr-Leu-Gly-Trp-Ile-DMeAsp-MePhe-amide

### Seq 26:

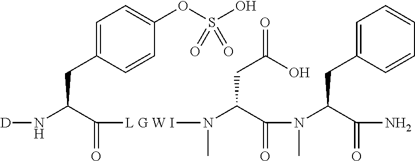

LCMS31: Rt=3.6 min; m/z=1136 (calculated: (m+2)/2)=1136.2)

UPLC61: Rt=4.8 min

### Example 62

### Asp-sTyr-Leu-Gly-Trp-Gln-DMeAsp-MePhe-amide

### Seq 30:

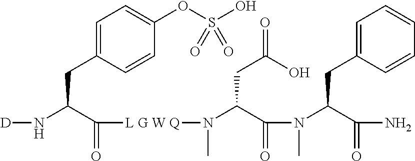

UPLC60: Rt=2.9 min

### Example 63

### Asp-sTyr-Leu-Gly-Trp-Met(O2)-DMeAsp-MePhe-amide

### Seq 31:

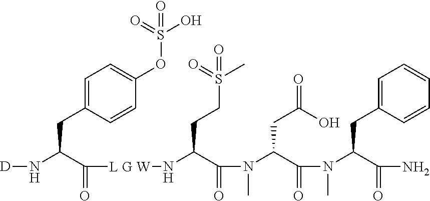

UPLC60: Rt=3.2 min

### Example 64

### Asp-sTyr-Leu-Gly-Trp-Leu-DMeAsp-MePhe-amide

### Seq 32:

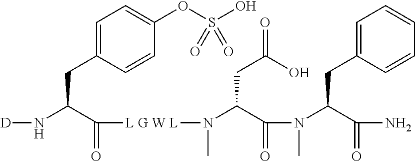

LCMS31: Rt=3.8 min; m/z=1136 (calculated: (m+2)/2)=1136.2)

UPLC60: Rt=4.8 min

### Example 65

### Asp-sTyr-Leu-Gly-Trp-Val-DMeAsp-MePhe-amide

### Seq 33:

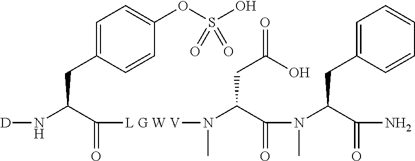

LCMS31: Rt=3.4 min; m/z=1121 (calculated: (m+1)/1)=1122.2)

UPLC61: Rt=4.0 min

### Example 66

### Asp-sTyr-Leu-Gly-Trp-Pro-DMeAsp-MePhe-amide

### Seq 34:

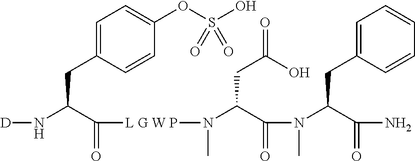

LCMS31: Rt=3.3 min; m/z=1120 (calculated: (m+1)/1)=1120.2)

UPLC60: Rt=3.0 min

### Example 67

### Asp-sTyr-Lys-Gly-Trp-Nle-DMeAsp-MePhe-amide

### Seq 35:

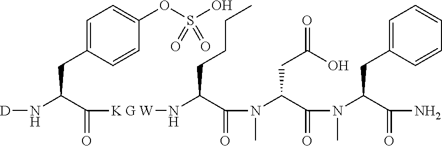

LCMS34: Rt=2.3 min; m/z=1150 (calculated: (m+1)/1)=1151.3)

UPLC60: Rt=3.9 min

### Example 68

### Asp-sTyr-Arg-Gly-Trp-Nle-DMeAsp-MePhe-amide

### Seq 36:

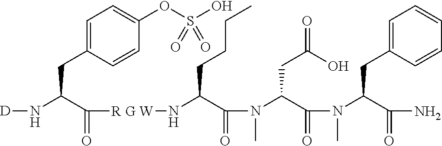

LCMS34: Rt=2.3 min; m/z=1178 (calculated: (m+1)/1)=1179.3)

UPLC60: Rt=4.0 min

### Example 69

### Asp-sTyr-Pro-Gly-Trp-Nle-DMeAsp-MePhe-amide

### Seq 37:

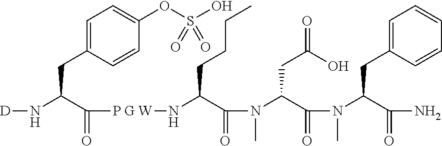

UPLC60: Rt=3.6 min

### Example 70

### Asp-sTyr-Met-Gly-Trp-Nle-DMeAsp-MePhe-amide

### Seq 38:

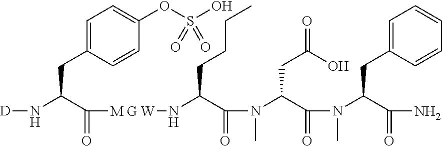

### Example 71

### Asp-sTyr-Nle-Gly-Trp-Met-DMeAsp-MePhe-amide

### Seq 39:

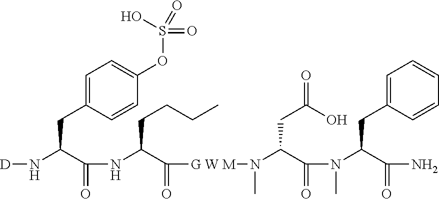

### Example 72

### sTyr-Nle-Gly-Trp-Nle-DMeAsp-MePhe-amide

### Seq 40:

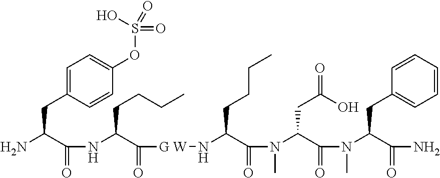

LCMS31: Rt=3.8 min; m/z=1021 (calculated: (m+1)/1)=1021.2)

UPLC61: Rt=6.9 min

### Example 73

### Asp-sTyr-Met-Gly-Trp-Met-DMeAsp-MePhe-amide

### Seq 41:

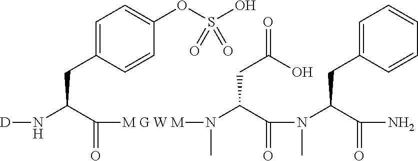

LCMS31: Rt=3.4 min; m/z=1172 (calculated: (m+1)/1)=1172.3)

UPLC60: Rt=3.6 min

### Example 74

### DAsp-Phe(4-sulfomethyl)-Nle-Gly-Trp-Nle-DMeAsp-MePhe-amide

### Seq 42:

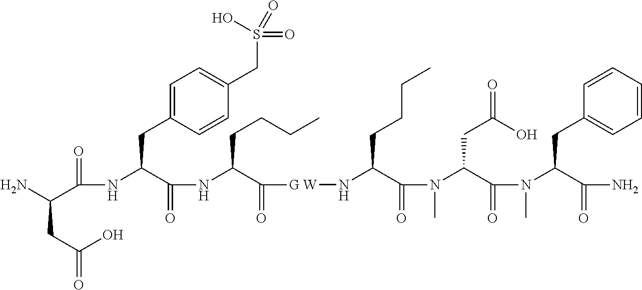

### Example 75

### bAsp-Phe(4-sulfomethyl)-Nle-Gly-Trp-Nle-DMeAsp-MePhe-amide

### Seq 43:

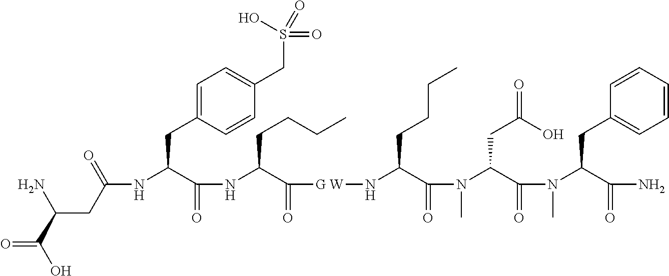

LCMS31: Rt=3.7 min; m/z=1134 (calculated: (m+1)/1)=1134.3)

UPLC60: Rt=5.5 min

### Example 76

### DbAsp-Phe(4-sulfomethyl)-Nle-Gly-Trp-Nle-DMeAsp-MePhe-amide

### Seq 44:

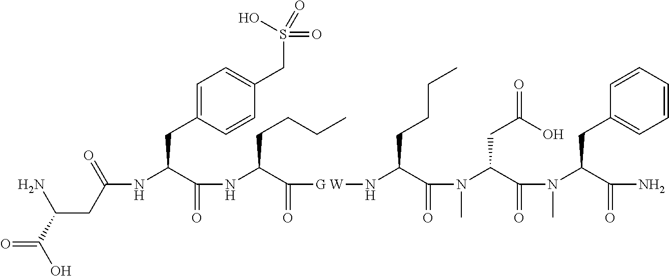

### Example 77

### Glu-sTyr-Leu-Gly-Trp-Nle-DMeAsp-MePhe-amide

### Seq 45:

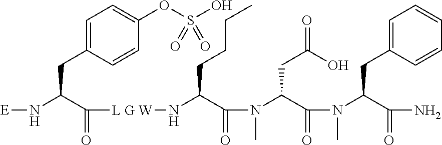

LCMS31: Rt=3.7 min; m/z=1150 (calculated: (m+1)/1)=1150.3)

UPLC61: Rt=4.9 min

### Example 78

### Asp-sTyr-Phe-Gly-Trp-Nle-DMeAsp-MePhe-amide

### Seq 46:

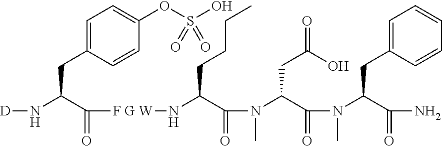

### Example 79

### Asp-sTyr-Ser-Gly-Trp-Nle-DMeAsp-MePhe-amide

### Seq 47:

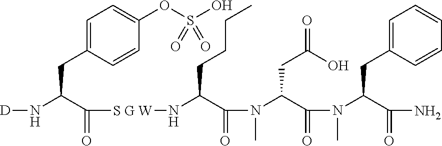

UPLC60: Rt=3.4 min

### Example 80

### Asp-sTyr-Ala-Gly-Trp-Nle-DMeAsp-MePhe-amide

### Seq 48:

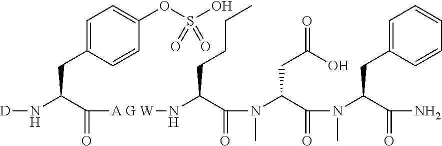

UPLC60: Rt=3.5 min

### Example 81

### Asp-sTyr-Ile-Gly-Trp-Nle-DMeAsp-MePhe-amide

### Seq 49:

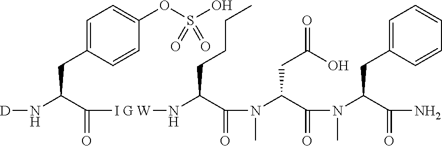

UPLC60: Rt=4.7 min

### Example 82

### Asp-sTyr-His-Gly-Trp-Nle-DMeAsp-MePhe-amide

### Seq 50:

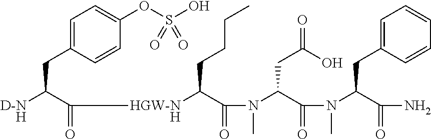

LCMS34: Rt=2.3 min; m/z=1159 (calculated: (m+1)/1)=1160.2)

UPLC60: Rt=3.5 min

### Example 83

### Gly-Ser-Ser-Gly-Ser-Ser-Asp-sTyr-Leu-Gly-Trp-Nle-DMeAsp-MePhe-amide

### Seq 51:

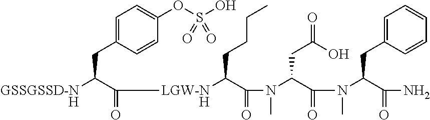

### Example 84

### Asp-sTyr-Leu-Gly-Trp-Ala-DMeAsp-MePhe-amide

### Seq 52:

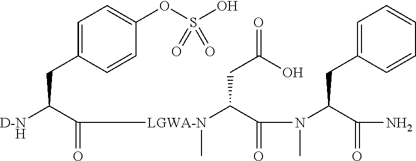

LCMS31: Rt=3.2 min; m/z=1094 (calculated: (m+1)/1)=1094.2)

UPLC60: Rt=3.2 min

### Example 85

### Glu-sTyr-Leu-Gly-Trp-Gln-DMeAsp-Phe-amide

### Seq 53:

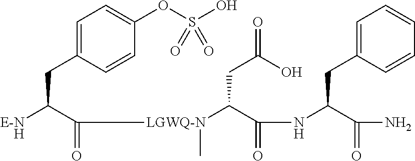

LCMS31: Rt=2.9 min; m/z=1151 (calculated: (m+1)/1)=1151.2)

UPLC60: Rt=2.7 min

### Example 86

### Glu-sTyr-Leu-Gly-Trp-Gln-DMeAsp-MePhe-amide

### Seq 54:

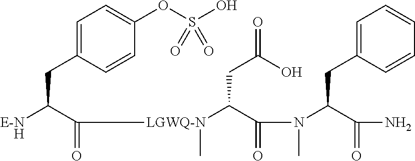

LCMS31: Rt=3.0 min; m/z=1165 (calculated: (m+1)/1)=1165.2)

UPLC61: Rt=3.0 min

### Example 87

### DGlu-sTyr-Leu-Gly-Trp-Nle-DMeAsp-MePhe-amide

### Seq 55:

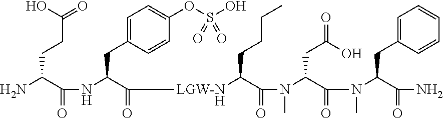

UPLC60: Rt=5.2 min

### Example 88

### Asp-sTyr-Leu-Gly-Trp-Nle-DMeAsp-2Nal-amide

### Seq 56:

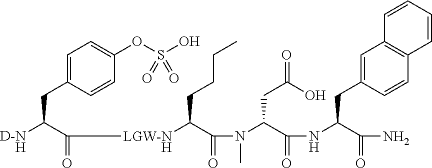

### Example 89

### Asp-sTyr-Met-Gly-Trp-Met-DMeAsp-Phe-amide

### Seq 57:

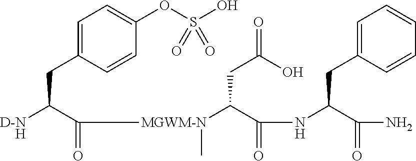

LCMS31: Rt=3.3 min; m/z=1158 (calculated: (m+1)/1)=1158.3)

UPLC60: Rt=3.0 min

### Example 90

### Glu-sTyr-Leu-Gly-Trp-Nle-DMeAsp-Phe-amide

### Seq 58:

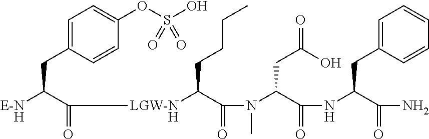

LCMS31: Rt=3.6 min; m/z=1136 (calculated: (m+1)/1)=1136.2)

UPLC61: Rt=4.3 min

### Example 91

### In Vitro Potency—Activity on the Cholecystokinin-1 Receptor and the Cholecystokinin-2 Receptor

This example reports the results of screening tests for in vitro activity, or potency, of peptides and peptide derivatives of the invention on the human cholecystokinin-1 receptor (CCK-1R) and on the human cholecystokinin-2 receptor (CCK-2R) in whole cell functional assays. The in vitro potency is a measure of the capability of the tested compounds to stimulate/activate these receptors.

The in vitro potency of the peptides and peptide derivatives listed in Table 2 below was determined as described in the following. Human native CCK-8s (Cholecystokinin Octapeptide (sulfated), ammonium salt, Bachem, H-2080, H-Asp-Tyr(SO₃H)-Met-Gly-Trp-Met-Asp-Phe-NH₂ (SEQ ID NO: 1)) and Gastrin-17 (Gastrin I (human), Bachem, H-3085, Pyr-Gly-Pro-Trp-Leu-Glu-Glu-Glu-Glu-Glu-Ala-Tyr-Gly-Trp-Met-Asp-Phe-NH₂ (SEQ ID NO: 2), non-sulfated) were included as reference compounds.

### Principle

Accumulation of inositol (1) phosphate (IP1) following stimulation of the receptors was measured using the IP-One HTRF® (HTRF=Homogeneous Time-Resolved Fluorescence) assay kit, which is commercially available from CisBio Bioassays, France (catalogue no. 62IPAPEB). For more details see www.htrf.com/GPCRs/IP-One.

CCK-1R and CCK-2R are both G protein-coupled receptors (GPCR), more in particular they are Gq-coupled 7TM receptors, where 7TM stands for “seven-transmembrane” as these receptors pass through the cell membrane seven times.

Activation of Gq protein coupled receptors results in a transient increase of intracellular Ca²⁺, triggered by inositol (1,4,5) tri-phosphate (IP3). After the GPCR Gq activation, the lifetime of IP3 is very short (less than 30 sec) before being transformed into inositol (1,4) di-phosphate (IP2) and then IP1. IP1 is accumulated in the cell when LiCl is added to the medium. After activation of the GPCR, IP1 can be precisely quantified using the assay referred to above. This assay is based on a monoclonal antibody specific for IP1, labelled with Lumi4TM-Tb cryptate. This antibody competes with native IP1 produced by cells and IP1 coupled to the dye d2. The specific signal is inversely proportional to the concentration of IP1 in the cell lysate. Data reduction using the fluorescence ratio (10⁴×665 nm/620 nm, i.e. 10⁴ times the fluorescence at 665 nm divided by the fluorescence at 620 nm) eliminates possible photophysical interference and means the assay is unaffected by the usual buffer conditions and coloured compounds.

### Cell Culture and Preparation

The cells used in these assays were 1321-N1 cells stably expressing the CCK-1R or the CCK-2R (Perkin Elmer product numbers ES-530-A and ES-531-A, respectively see www.perkinelmer.com/Catalog/Product/ID/ES-530-A or -ES-531-A, respectively).

The cells were cultured in an incubator (95% RH (relative humidity), 5% (of standard atmospheric pressure) CO₂, 37° C.) in cell culture medium (DMEM (Lonza catalogue no. BE-12-604F/U1), with 10% (v/v) Foetal Calf Serum (FCS, Gibco catalogue no. 16140-071), 1% (w/w) Sodium pyruvate (Gibco catalogue no. 11360-039), 500 μg/ml Geneticin® (G418) (Gibco catalogue no. 10131-027), 25 μg/ml Zeocin™ (Invitrogen catalogue no. R250-05), 1% (w/w) Pen/Strep (Gibco catalogue no. 15140-122)). The cells were aliquoted and stored in liquid nitrogen.

### Procedure

Before each assay a cell aliquot was thawed and washed twice in cell plating medium (DMEM (Lonza, see above), with 10% (v/v) FCS (Gibco, see above), 1% (w/w) Sodium pyruvate (Gibco, see above), 1% (w/w) Pen/Strep (Gibco, see above)) before being suspended in cell plating medium and seeded at 2×10⁴ cells/well (CCK-1R) or 1×10⁴ cells/well (CCK-2R) in a 384 well assay plate pre-coated with poly-D-lysin (Corning catalogue no. 356661). The assay plate was then incubated overnight in a CO₂ permeable plastic bag in an incubator (95% RH, 5% CO₂, 37° C.). On the day of the experiment, the assay plate with the cells was washed three times with Dulbecco's Phosphate Buffered Saline w/o Calcium or Magnesium (DPBS; BioWhittaker catalogue no. BE-17-512F). Thirty μl of reference or test compounds in serial dilutions from 10⁻⁶ M (one μM) to 10⁻¹² M (one pM) in assay buffer (IPOne stimulation buffer (part of IP-One HTRF® assay kit, see above; consisting of 10 mM Hepes, 1 mM CaCl₂, 0.5 MgCl₂, 4.2 mM KCl, 146 mM NaCl, 5.5 mM glucose, 50 mM LiCl, pH 7.4) with 0.005 v/v % Tween® 20 (Merck catalogue no. 8.22184.1) and 0.1 v/v % Pluronic F-68 (Gibco catalogue no. 24040-032)) was immediately added (using technical duplicates, i.e. two wells of each sample, one data point being the average of two wells), and the plate was incubated for 1 hour at 37° C. in an incubator. After lysing the cells and adding IP1-d2 conjugate and anti-IP1-cryptate conjugate (lysis buffer, IP1-d2 conjugate and anti-IP1 cryptate Tb conjugate were all included with the IP-One HTRF® Assay kit referred to above), the assay plate was incubated 1 hour at room temperature. Then, the plate was read on a fluorescence plate reader (Mithras LB 940 or similar) using filters for excitation at 320 nm and emissions at 665 nm and 620 nm.

### Calculations

As a measure of compound potency, calculations were performed in order to determine the half maximal effective concentration (the EC₅₀ value). EC₅₀ refers to the concentration of a compound which induces a response half way between the basal response and the maximal response. Thus, a lower EC₅₀ value corresponds to a higher potency of the compound.

The data from the fluorescence reader were given as the fluorescence ratio (10⁴×665 nm/620 nm). It is dimensionless. The fluorescence ratio was plotted as a function of the concentration of compound. Dose-response curves were obtained by a nonlinear three-parameter logistic equation (log(agonist) vs response) with hillslope=−1, and shared top and bottom using the software programme GraphPad Prism (Prism 6, GraphPad Software). The highest dose tested was 10⁻⁶ M (1×10⁶ pM).

The preference of a specific compound for the CCK-1 receptor, or the CCK-1R selectivity, may be defined as EC₅₀ (CCK-2R)/EC₅₀ (CCK-1R). If the CCK-1R selectivity is >1 the compound has preference for the CCK-1R, and if it is <1 the preference is for the CCK-2R.

### Results

A minimum of three biological replicates was measured for each test compound on separate assay plates. The reported EC₅₀ values (in pM) shown in Table 2 below are average results of screening studies. Where possible the CCK-1R selectivity (EC₅₀ (CCK-2R)/EC₅₀ (CCK-1R)) is also reported.

TABLE 2
In vitro potencies (EC₅₀ in pM)

| | | | CCK-1R selectivity |
|---|---|---|---|
| | EC₅₀ (CCK-1R) | EC₅₀ (CCK-2R) | (EC₅₀ (CCK-2R)/ |
| Compound | [pM] | [pM] | EC₅₀ (CCK-1R)) |
| Derivatives | | | |
| Example 1 | 240 | >1000000 | very selective |
| Example 2 | 370 | 180000 | 480 |
| Example 3 | 310 | >1000000 | very selective |
| Example 4 | 530 | 340000 | 630 |
| Example 5 | 430 | 81000 | 190 |
| Example 6 | 200 | >1000000 | very selective |
| Example 7 | 470 | 130000 | 290 |
| Example 8 | 290 | 170000 | 570 |
| Example 9 | 370 | 170000 | 450 |
| Example 10 | 180 | 260000 | 1400 |
| Example 11 | 170 | >1000000 | very selective |
| Example 12 | 140 | >1000000 | very selective |
| Example 13 | 270 | 79000 | 290 |
| Example 14 | 140 | 730000 | 5100 |
| Example 15 | 170 | 150000 | 910 |
| Example 16 | 360 | 310000 | 860 |
| Example 17 | 210 | 270000 | 1300 |
| Example 18 | 180 | 300000 | 1700 |
| Example 19 | 210 | >1000000 | very selective |
| Example 20 | 310 | 270000 | 860 |
| Example 21 | 310 | 190000 | 620 |
| Example 22 | 370 | 440000 | 1200 |
| Example 23 | 320 | >1000000 | very selective |
| Example 24 | 290 | 190000 | 650 |
| Example 25 | 370 | >1000000 | very selective |
| Example 26 | 360 | >1000000 | very selective |
| Example 27 | 430 | >1000000 | very selective |
| Example 28 | 280 | 96000 | 340 |
| Example 29 | 610 | 17000 | 28 |
| Example 30 | 540 | 35000 | 66 |
| Example 31 | 280 | 110000 | 380 |
| Example 32 | 290 | 46000 | 160 |
| Example 33 | 270 | 36000 | 130 |
| Example 34 | 210 | >1000000 | very selective |
| Example 35 | 380 | >1000000 | very selective |
| Example 36 | 260 | 150000 | 560 |
| Example 37 | 550 | 32000 | 58 |
| Example 38 | 720 | >1000000 | very selective |
| Example 39 | 270 | 110000 | 420 |
| Example 40 | 170 | 880000 | 5300 |
| Example 41 | 210 | >1000000 | very selective |
| Example 42 | 220 | >1000000 | very selective |
| Example 43 | 420 | 62000 | 150 |
| Example 44 | 410 | 390000 | 960 |
| Example 45 | 310 | 940000 | 3000 |
| Example 46 | 220 | 97000 | 440 |
| Example 47 | 360 | 210000 | 580 |
| Example 48 | 330 | >1000000 | very selective |
| Example 49 | 340 | 46000 | 130 |
| Example 50 | 300 | 260000 | 870 |
| Example 51 | 270 | 430000 | 1600 |
| Example 52 | 370 | 94000 | 250 |
| Peptides | | | |
| Example 54 | 140 | 290000 | 2100 |
| Example 55 | 250 | >1000000 | very selective |
| Example 57 | 240 | >1000000 | very selective |
| Example 58 | 210 | >1000000 | very selective |
| Example 59 | 210 | >1000000 | very selective |
| Example 60 | 180 | 380000 | 2100 |
| Example 61 | 240 | >1000000 | very selective |
| Example 62 | 150 | >1000000 | very selective |
| Example 63 | 370 | >1000000 | very selective |
| Example 64 | 250 | >1000000 | very selective |
| Example 65 | 340 | >1000000 | very selective |
| Example 66 | 340 | >1000000 | very selective |
| Example 67 | 260 | >1000000 | very selective |
| Example 68 | 210 | 910000 | 4300 |
| Example 69 | 190 | >1000000 | very selective |
| Example 72 | 330 | >1000000 | very selective |
| Example 73 | 270 | >1000000 | very selective |
| Example 75 | 190 | >1000000 | very selective |
| Example 77 | 210 | 920000 | 4500 |
| Example 78 | 220 | >1000000 | very selective |
| Example 79 | 190 | >1000000 | very selective |
| Example 80 | 160 | >1000000 | very selective |
| Example 81 | 130 | >1000000 | very selective |
| Example 82 | 430 | >1000000 | very selective |
| Example 84 | 220 | >1000000 | very selective |
| Example 85 | 180 | >1000000 | very selective |
| Example 86 | 320 | >1000000 | very selective |
| Example 87 | 150 | >1000000 | very selective |
| Example 89 | 170 | >1000000 | very selective |
| Example 90 | 150 | >1000000 | very selective |
| CCK-8s | 120 | 420 | 3.5 |
| Gastrin-17 | 530000 | 390 | 0.00075 |

The data in Table 2 show that the peptides and peptide derivatives of the invention potently activate the CCK-1R while they are considerably less potent on the CCK-2R. Thus, these compounds are highly CCK-1R selective compounds.

As regards the reference compounds, the data in Table 2 shows that (i) CCK-8s can activate both the CCK-1R and the CCK-2R but with a slightly higher preference (3.5 fold) for the CCK-1R; (ii) Gastrin-17 has a much higher preference for the CCK-2R compared to the CCK-1R; and (iii) Gastrin-17 and CCK-8s are almost equally potent on the CCK-2R.

### Example 92

### In Vitro Receptor Binding—Affinity for the Cholecystokinin-1 Receptor and the Cholecystokinin-2 Receptor

This example reports the results of screening tests for in vitro receptor binding, or affinity, of peptides and peptide derivatives of the invention to the human cholecystokinin-1 receptor (CCK-1R) and to the human cholecystokinin-2 receptor (CCK-2R) in plasma membrane based receptor binding assays. The in vitro binding affinity is a measure of the strength of attraction between a receptor and its ligand.

The in vitro affinity of the peptides and peptide derivatives shown in Table 3 below was determined as described in the following. Human native CCK-8s (Cholecystokinin Octapeptide (sulfated), ammonium salt, Bachem, H-2080, H-Asp-Tyr(SO₃H)-Met-Gly-Trp-Met-Asp-Phe-NH₂ (SEQ ID NO: 1) and Gastrin-17 (Gastrin I (human), Bachem, H-3085, Pyr-Gly-Pro-Trp-Leu-Glu-Glu-Glu-Glu-Glu-Ala-Tyr-Gly-Trp-Met-Asp-Phe-NH₂ (SEQ ID NO: 2), non-sulfated) were included as reference compounds.

### Principle

The binding affinity of the CCK derivatives to the human CCK-1R and CCK-2R was measured by way of their ability to displace [¹²⁵I]-CCK-8s from the receptors (stocks of frozen plasma membranes) in a SPA receptor binding assay. Displacement of radio ligand was measured as reduction in light emission from SPA beads.

### Cell Culture and Membrane Purification

The cells used in these assays were 1321-N1 cells stably expressing the CCK-1R or the CCK-2R (Perkin Elmer product numbers ES-530-A and ES-531-A, respectively see www.perkinelmer.com/Catalog/Product/ID/ES-530-A or -ES-531-A, respectively).

The cells were cultured in an incubator (95% RH (relative humidity), 5% (of standard atmospheric pressure) CO₂, 37° C.) in cell culture medium (DMEM (Lonza catalogue BE-12-604F/U1), with 10% (v/v) Foetal Calf Serum (FCS, Gibco catalogue 16140-071), 1% (w/w) Sodium pyruvate (Gibco catalogue 11360-039), 500 μg/ml Geneticin® (G418) (Gibco catalogue 10131-027), 25 μg/ml Zeocin™ (Invitrogen catalogue R250-05), 1% (w/w) Pen/Strep (Gibco catalogue 15140-122)).

Plasma membranes were prepared by Evotec AG Hamburg by use of the ParrBomb (#4635, Parr Instrument Company). Cells were lysed with a lysis buffer (50 mM Tris, 2.5 mM EDTA, pH7) with added protease inhibitors (Complete Proteaseinhibitors EDTA-free (#05056489001, Roche), 1 tablet per 50 mL lysis buffer). The membrane fraction was separated by centrifugation. First, the cell lysate was centrifuged at 1000×g for 10 minutes at 4° C. The pellet was discarded and the supernatant was then centrifuged at 100.000×g for 30 minutes at 4° C. The supernatant was discarded and the pellet was re-suspended in 1 ml/5×108 cells ice cold membrane buffer (50 mM Tris, 320 mM Sucrose and Complete Proteaseinhibitors EDTA-free (#05056489001, Roche); pH7.4) and homogenised with dounce homogeniser. The protein concentration was determined using the Pierce™ BCA Protein Assay Kit according to kit manual, and the membrane preparation was adjust to the desired protein concentration with membrane buffer and aliquoted into pre-cooled tubes, snap-frozen in liquid nitrogen, and stored at −80° C.

### Procedure

CCK receptor binding assays were performed in 96-well Optiplates (Cat #6005290, PerkinElmer, Waltham, Mass., USA) in a total volume of 200 μl per well. CCK derivatives in 80% DMSO stock solutions of 800 μM were serially diluted fivefold in assay buffer (50 mM Tris/HCl, 4.5 mM MgCl₂, 4.5 mM MgCl2, 0.02% Tween 20, 0.25% ovalbumin, 0.1% pluronic F-68, pH 7.4) to final assay concentrations ranging from 10⁻⁵ M (ten μM) to 10⁻¹² M (one pM). Wheatgerm Agglutinin coated PVT SPA beads (Cat # RPNQ 0001, PerkinElmer) were reconstituted in assay buffer and mixed with the radio ligand, human [¹²⁵I]-CCK8s-peptide (Cat# NEX203, PerKinElmer), and membrane preparation to give a final assay concentration of 0.5 mg/well SPA beads, approximately 50 pM. of the radio ligand (corresponding to approximately 50.000 dpm/well), and 1 μg total membrane protein per well.

Fifty μl of reference or test compounds were added to the Optiplates (using technical duplicates, i.e. two wells of each sample, one data point being the average of two wells), followed by addition of 150 μl of the mixture of SPA bead, radio ligand, and plasma membrane. The plates were sealed and incubated in the dark at +22° C. for 2 hours on a IKA MTS plate shaker (IKA-Werke D-79219 Staufen) set at 300 rpm and thereafter centrifuged at 1500 rpm for 10 minutes in a Heraeus Multifuge 3s centrifuge. SPA bead light emission was measured in a TopCount NXT (PerkinElmer) for 2 min/well after a delay of 60 min.

### Calculations

As a measure of compound affinity, calculations were performed in order to determine the half maximal inhibitory concentration (the IC₅₀ value). IC₅₀ refers to the concentration of a compound which is required for 50% inhibition of maximal radio ligand binding to the receptor. Thus, a lower IC₅₀ value corresponds to a higher affinity of the compound.

The data from the TopCounter were given as counts per minute (CPM). The CPM was plotted as a function of the concentration of compound. Dose-response curves were obtained, and IC₅₀ values were calculated, by a nonlinear three-parameter logistic equation (log(agonist) vs response) with hillslope=−1, and shared top and bottom using the software programme GraphPad Prism v 6.0 (Graph Pad software, La Jolla, Calif., USA). The highest dose tested was 10⁻⁵ M (1×10⁷ pM).

The preference of a specific compound for the CCK-1 receptor, or the CCK-1R selectivity, may be defined as IC₅₀ (CCK-2R)/IC₅₀ (CCK-1R). If the CCK-1R selectivity is >1 the compound has preference for the CCK-1R, and if it is <1 the preference is for the CCK-2R.

### Results

A minimum of three biological replicates was measured for each test compound on separate assay plates. The reported IC₅₀ values (in pM) shown in Table 3 below are average results of screening studies. Where possible the CCK-1R selectivity based on binding (binding selectivity) (IC₅₀ (CCK-2R)/IC₅₀ (CCK-1R)) is also reported.

TABLE 3
In vitro affinities (IC₅₀ in pM)

| | | | CCK-1R binding |
|---|---|---|---|
| | | | selectivity |
| | IC₅₀ (CCK-1R) | IC₅₀ (CCK-2R) | (IC₅₀ (CCK-2R)/ |
| Compound | [pM] | [pM] | IC₅₀ (CCK-1R)) |
| Derivatives | | | |
| Example 1 | 910 | 3000000 | 3300 |
| Example 2 | 130 | 1000000 | 8000 |
| Example 3 | 260 | >10000000 | very selective |
| Example 4 | 43 | 2100000 | 48000 |
| Example 5 | 120 | 380000 | 3200 |
| Example 6 | 200 | >10000000 | very selective |
| Example 7 | 99 | 5700000 | 58000 |
| Example 8 | 59 | 1800000 | 31000 |
| Example 9 | 81 | 4600000 | 57000 |
| Example 10 | 100 | 2300000 | 23000 |
| Example 11 | 84 | >10000000 | very selective |
| Example 12 | 640 | >10000000 | very selective |
| Example 13 | 89 | 420000 | 4700 |
| Example 14 | 310 | 6400000 | 21000 |
| Example 15 | 190 | 1000000 | 5500 |
| Example 16 | 99 | >10000000 | very selective |
| Example 17 | 55 | 880000 | 16000 |
| Example 18 | 78 | 5200000 | 67000 |
| Example 19 | 81 | 7400000 | 92000 |
| Example 20 | 66 | 320000 | 4900 |
| Example 21 | 67 | 1400000 | 20000 |
| Example 22 | 39 | 6500000 | 170000 |
| Example 23 | 130 | >10000000 | very selective |
| Example 24 | 56 | 6800000 | 120000 |
| Example 25 | 72 | >10000000 | very selective |
| Example 26 | 63 | >10000000 | very selective |
| Example 27 | 130 | >10000000 | very selective |
| Example 28 | 100 | 720000 | 7100 |
| Example 29 | 72 | 370000 | 5100 |
| Example 30 | 43 | 770000 | 18000 |
| Example 31 | 70 | 480000 | 6800 |
| Example 32 | 87 | 380000 | 4300 |
| Example 33 | 92 | 220000 | 2300 |
| Example 34 | 160 | >10000000 | very selective |
| Example 35 | 42 | >10000000 | very selective |
| Example 36 | 190 | 1200000 | 6600 |
| Example 37 | 46 | 430000 | 9300 |
| Example 38 | 110 | 7700000 | 71000 |
| Example 39 | 99 | 590000 | 5900 |
| Example 40 | 650 | >10000000 | very selective |
| Example 41 | 180 | >10000000 | very selective |
| Example 42 | 97 | >10000000 | very selective |
| Example 43 | 63 | 2400000 | 39000 |
| Example 44 | 65 | 5600000 | 87000 |
| Example 45 | 58 | 4700000 | 82000 |
| Example 46 | 68 | 280000 | 4100 |
| Example 47 | 56 | 340000 | 6000 |
| Example 48 | 52 | 9000000 | 170000 |
| Example 49 | 64 | 700000 | 11000 |
| Example 50 | 140 | 1900000 | 14000 |
| Example 51 | 52 | 10000000 | 190000 |
| Example 52 | 49 | 480000 | 9800 |
| Peptides | | | |
| Example 54 | 27 | 2300000 | 84000 |
| Example 55 | 46 | >10000000 | very selective |
| Example 57 | 45 | >10000000 | very selective |
| Example 58 | 35 | >10000000 | very selective |
| Example 59 | 47 | >10000000 | very selective |
| Example 60 | 41 | 950000 | 23000 |
| Example 61 | 78 | 6100000 | 78000 |
| Example 62 | 75 | >10000000 | very selective |
| Example 63 | 540 | >10000000 | very selective |
| Example 64 | 42 | >10000000 | very selective |
| Example 65 | 620 | >10000000 | very selective |
| Example 66 | 110 | >10000000 | very selective |
| Example 67 | 30 | >10000000 | very selective |
| Example 68 | 34 | 1500000 | 45000 |
| Example 69 | 43 | >10000000 | very selective |
| Example 72 | 60 | >10000000 | very selective |
| Example 73 | 34 | >10000000 | very selective |
| Example 75 | 49 | 2900000 | 59000 |
| Example 77 | 41 | >10000000 | very selective |
| Example 78 | 29 | 920000 | 32000 |
| Example 79 | 35 | 3600000 | 100000 |
| Example 80 | 55 | >10000000 | very selective |
| Example 81 | 38 | >10000000 | very selective |
| Example 82 | 45 | >10000000 | very selective |
| Example 84 | 530 | >10000000 | very selective |
| Example 85 | 240 | >10000000 | very selective |
| Example 86 | 85 | >10000000 | very selective |
| Example 87 | 32 | >10000000 | very selective |
| Example 89 | 43 | >10000000 | very selective |
| Example 90 | 29 | >10000000 | very selective |
| CCK-8s | 180 | 470 | 2.6 |
| Gastrin-17 | 340000 | 6400 | 0.019 |

The data in Table 3 show that the peptides and peptide derivatives of the invention have high affinity for the CCK-1R while they have considerably less affinity for the CCK-2R. Thus, these compounds are highly CCK-1R selective compounds.

As regards the reference compounds, the data in Table 3 shows that (i) CCK-8s has high affinity for both the CCK-1R and the CCK-2R but with a slightly higher preference (2.6 fold) for the CCK-1R; (ii) Gastrin-17 has much higher preference for the CCK-2R compared to the CCK-1R; and (iii) CCK-8s has a 10 times higher affinity for the CCK-2R compared to Gastrin-17.

### Example 93

### Pharmacokinetic (PK) Studies in Mini-Pig

The purpose of this study is to determine the protraction in vivo of a number of CCK derivatives of the invention after intravenous (i.v.) administration to mini-pigs, i.e. the prolongation of their time in the body and thereby their time of action. This was done in pharmacokinetic (PK) studies, where the terminal half-life of the derivative in question was determined. By terminal half-life is generally meant the period of time it takes to halve a certain plasma concentration, measured after the initial distribution phase.

The derivatives of the compounds listed in Table 4 were subjected to PK studies as described in the following.

Female Göttingen mini-pigs obtained from Ellegaard Göttingen Minipigs (Dalmose, Denmark) at approximately 6 months of age and weighing approximately 17 kg were used in the studies. The mini-pigs were dosed (i.v. via a central catheter in vena cava caudalis. The catheter was flushed with 10 ml saline post administration. Blood was sampled from the central catheter. Test substances were dissolved in a vehicle consisting of 50 mM sodium phosphate, 70 mM sodium chloride, 0.05% v/v % polysorbate 80 (Sigma LOT#SLBF8247V), pH adjusted to 7.4. The pigs were dosed with 5 nmol/kg body weight of the CCK derivative. Blood samples were taken at the following time points: pre-dose, 5 minutes, 15 minutes, 30 minutes, 45 minutes, 1, 1.5, 2, 3, 4, 6, 8, 10, 24, 48, 72, 96, 120, 168, 192, 216, 240, 264, and 288 hours post dosing. Additional blood samples were collected for the derivatives of Examples 2, 4, 7, 8, and 9 at the following time points: 5, 12, 22, 28 and 144 hours. The blood samples were collected into test tubes containing EDTA buffer for stabilisation and kept on ice for max. 20 minutes before centrifugation. The centrifugation procedure to separate plasma was: 4° C., approx. 2500 g for 10 minutes. Plasma was collected and immediately transferred to Micronic tubes stored at −20° C. until assayed.

### Plasma Analysis

Plasma was pipetted into Micronic tubes on dry ice, and kept at −20° C. until analysed for plasma concentration of the respective CCK derivative using LC-MS. The plasma samples (including standard curve samples used for quantitation of unknowns and prepared from blank plasma spiked with CCK compound at a concentration range of 0.5-2000 nM for the compounds of Examples 3, 5, 6 or 0.5-1000 nM for the remaining Example compounds) were protein precipitated using three volumes of methanol and centrifuged (16000×g, 4° C., 20 min). The supernatants were injected into the chromatographic system (TurboFlow Transcend 1250 & 10 valve VIM, Thermo Fisher Scientific) which consisted of an initial Turboflow Cyclone purification column 0.5×50 mm (Thermo Fischer Scientific) and an eluting Aeris peptide 3.6 μm XB—C18 column 2.1×50 mm (Phenomenex) kept at 60° C.

The CCK derivatives of Examples 2-9 were eluted using LC-MS method I: A chromatographic gradient system with mobile phases consisting of water with 0.1 v/v % formic acid (mobile phase A) and water/acetonitrile 10/90 v/v with 0.1 v/v % formic acid (mobile phase B). The 2 minute gradient started at 60% mobile phase B and ended at 100% mobile phase B with a flow of 0.4 mL/min. The CCK derivatives were detected and quantified after on-line infusion of the LC flow to the LTQ OrbiTrap Discovery mass spectrometer (Thermo Fischer Scientific) equipped with an electrospray interface operated in negative mode, ESI− (Sheath gas flow rate at 40, Aux gas flow rate at 30, Sweep gas flow rate at 2, Spray voltage at 2.8 kV, Capillary temp. at 275° C., S-lens RF level at −125 and Heater temp. at 325° C.).

The remaining Example compounds were eluted using LC-MS method II: A chromatographic gradient system with mobile phases consisting of water with 1 v/v % formic acid (mobile phase A) and water/acetonitrile 10/90 v/v with 1 v/v % formic acid (mobile phase B). The initial 0.25 minute gradient started at 0% mobile phase B and ended at 70% mobile phase B followed by the second 2.2 minute gradient started at 70% mobile phase B and ended at 75% mobile phase B with a flow of 0.4 mL/min. These CCK derivatives were detected and quantified as described above except that the Spray voltage was 4.0 kV.

Individual plasma concentration-time profiles were analysed by a non-compartmental model in Phoenix v. 6.3 (Pharsight Inc., Mountain View, Calif., USA).

### Results

The following results were obtained:

TABLE 4
Plasma terminal half-life (T½) after
i.v. administration of CCK derivatives to mini-pigs

| Derivative of Example no. | T½ (h)* |
|---|---|
| 3 | 113 |
| 5 | 106 |
| 6 | 37 |
| 2 | 140 |
| 4 | 147 |
| 7 | 115 |
| 8 | 103 |
| 9 | 112 |
| 19 | 36 |
| 23 | 161 |
| 29 | 119 |
| 34 | 2 |
| 35 | 153 |
| 38 | 3 |

*Harmonic mean, n = 3 for the compounds of Examples 3, 5, and 6 and n = 2 for the remaining Example compounds

The results show a half-life of from 2 to 161 hours, which is a substantial improvement as compared to the half-life of native CCK peptides which is reported as less than 5 minutes (Gastroenterology, 1993, Vol. 105(6), pp. 1732-1736).

### Example 94

### Pharmacodynamic (PD) Studies in LYD Pig, Food Intake

The purpose of this experiment is to investigate the effect of a CCK derivative of the invention on food intake in pigs. This was done in a pharmacodynamic (PD) study as described below, in which food intake was measured from 0-48 hours after administration of a single subcutaneous dose of the CCK derivative of Example 3, as compared to baseline measurements. Additional PD studies are reported in Example 97.

Female Landrace Yorkshire Duroc (LYD) pigs or Large White hybrid, approximately 3 months of age, weighing approximately 30-35 kg were used (n=4 per group). The animals were housed in a group for approximately 1 week during acclimatisation to the animal facilities. During the experimental period the animals were placed in individual pens approximately 1 week before dosing and during the entire experiment for measurement of individual food intake.

The animals were fed ad libitum with pig fodder (Svinefoder Danish Top; supplier Danish Agro) at all times both during the acclimatisation and the experimental period. Food intake was monitored on line by logging the weight of fodder every 15 minutes using the Mpigwin system (Ellegaard Systems, Faaborg, Denmark). Any uneaten fodder was removed and weighed (manually) on the following morning, and replaced with fresh fodder. Baseline food intake was measured for each pig on three successive days before dosing. Mean baseline value for each pig was used in calculation of food intake relative to baseline.

The CCK derivative was dissolved in a phosphate buffer (50 mM sodium phosphate, 70 mM sodium chloride, 0.05 v/v % polysorbate 80 (Sigma, LOT#SLBF8247V), pH 7.4) at concentrations of approximately 5300 nmol/ml corresponding to doses of 100 nmol/kg. The phosphate buffer served as vehicle.

Animals were dosed with a single subcutaneous dose of the CCK derivative or vehicle (dose volume 0.02 ml/kg) on the morning of day 1, and food intake was measured for 48 h. On the last day of the study a blood sample for measurement of plasma exposure of the CCK derivative was taken from the heart in anaesthetised animals. Plasma content of the CCK derivative was measured using LC-MS method I, as described in Example 93.

Food intake (FI) was calculated in 24 h intervals (0-24 hours and 24-48 hours). In Table 5 below the resulting mean difference in food intake (ΔFI) is given for each time interval relative to mean baseline for each pig, determined as described above. The mean difference in food intake is calculated as follows:

ΔFI₀₋₂₄ₕ=FI₀₋₂₄ₕ−FI_{baseline},

and vice-versa for the 24-48 hours' time interval. A negative number indicates a reduction in FI.

TABLE 5
Effect on food intake in pig

Food intake

| | ΔFI_{0-24 hours} | ΔFI_{24-48 hours} |
|---|---|---|
| Compound | (kg) | (kg) |
| Example 3 | −0.98 | −0.63 |
| Vehicle | 0.14 | 0.28 |

The results in Table 5 show that a single s.c. injection of the CCK derivative of Example 3 in pigs reduces food intake for at least 48 h after dosing. The reductions were significant compared to vehicle.

### Example 95

### Chemical Stability

The purpose of this assay is to assess the chemical stability of the derivatives of the invention in aqueous solutions. The chemical stability was investigated by RP-UPLC separation and UV detection.

### Assay Description

Lyophilised samples of the derivative of Example 3 were dissolved in a 50 mM sodium phosphate buffer pH 7.4 containing 70 mM NaCl and 0.05 v/v % polysorbate 80, at a concentration of 1 mg/ml of the derivative (for the Example 3 derivative 1 mg/ml equates 0.54 mM). If necessary, pH was adjusted to 7.4. Samples were incubated at 5° C., 25° C. and 37° C. for 2 weeks and 6 weeks, followed by RP-UPLC analysis.

RP-UPLC analysis was performed using a Waters Acquity system with the following parameters:

| | |
|---|---|
| Instrument | Waters Acquity UPLC |
| Column | Acquity BEH C18 column, |
| | (2.1 × 150 mm, 1.7 μm) |
| Column temperature | 35° C. |
| Flow (ml/min) | 0.3 ml/min |
| UV wavelength (nm) | 276 nm |
| Eluent A | 0.2M Na₂SO₄, 0.02M Na₂HPO₄, 0.02M |
| | NaH₂PO₄, pH 7.2, 10% CH₃CN |
| Eluent B | 70% (v/v) CH₃CN |

The following UPLC gradient was used:

| Time (min) | Flow (ml/min) | % A | % B |
|---|---|---|---|
| 0 | 0.3 | 70 | 30 |
| 2 | 0.3 | 70 | 30 |
| 2.5 | 0.3 | 60 | 40 |
| 4 | 0.3 | 55 | 45 |
| 6 | 0.3 | 50 | 50 |
| 7.5 | 0.3 | 30 | 70 |
| 8 | 0.3 | 30 | 70 |

### Calculations

Purity of a compound is defined as the area percentage of the compound peak in relation to the total area of all integrated peaks in the chromatogram and is calculated by the software of the apparatus. Absolute purity loss (in percentage points, pp) is determined as the difference in purity between the start sample and the sample incubated for a certain time at a certain temperature. Relative purity loss (in %) is the ratio of absolute purity loss to the purity determined in the start sample.

### Results

The results are summarised in Table 6.

TABLE 6
Chemical stability, Example 3 compound

| | | | Absolute | Relative purity |
|---|---|---|---|---|
| Incubation | Incubation | | purity loss | loss |
| temperature | time (weeks) | Purity (%) | (pp) | (%) |
| 5° C. | 0 | 82.71 | — | — |
| 5° C. | 2 | 82.43 | 0.28 | 0.34 |
| 5° C. | 6 | 82.50 | 0.21 | 0.25 |
| 25° C. | 0 | 82.71 | — | — |
| 25° C. | 2 | 82.58 | 0.13 | 0.16 |
| 25° C. | 6 | 82.41 | 0.30 | 0.36 |
| 37° C. | 0 | 82.71 | — | — |
| 37° C. | 2 | 82.54 | 0.17 | 0.21 |
| 37° C. | 6 | 82.07 | 0.64 | 0.77 |

The results in Table 6 show that the compound of Example 3 is very stable at all three temperatures tested and for up to at least 6 weeks.

In addition, short term chemical stability was also demonstrated for the Example 3 compound incubated in the same buffer at 25° C. for three days at 25 μM, 100 μM and 500 μM concentrations. The compound showed virtually no degradation under the conditions tested.

The chemical stability of the Example 3 compound was also compared to the chemical stability of CCK-8s as described in the following.

### Method Description

Lyophilised samples of the derivative or peptide were dissolved in 8 mM sodium phosphate buffer pH 7.4 at a concentration of 1 mM of the derivative or peptide. The resulting samples were stored in HPLC vials at 5° C. and 37° C. for 2 weeks. The samples were analysed in the beginning of the experiment (t=0), after one week and after two weeks by RP-UHPLC with a mobile phase containing phosphate and with a gradient of acetonitrile. UV detection at 215 nm was used. Samples after one week and two weeks were analysed twice.

### Calculations and Results

The calculations were done as described above, and the results are summarised in Table 7 (average of two determinations). The purities of the two compounds at the start of the experiment (incubation time 0) was for CCK-8s 97.94% and for the compound of Example 3 95.90%.

TABLE 7
Chemical stability of CCK-8s and the Example 3 compound

| | | | Absolute | Relative |
|---|---|---|---|---|
| | Incubation | Incubation | purity | purity |
| Compound | temperature | time (weeks) | loss (pp) | loss (%) |
| CCK-8s | 5° C. | 1 | −0.04 | 0.0 |
| CCK-8s | 5° C. | 2 | 0.09 | 0.1 |
| CCK-8s | 37° C. | 1 | 1.42 | 1.4 |
| CCK-8s | 37° C. | 2 | 3.33 | 3.4 |
| Example 3 | 5° C. | 1 | 0.43 | 0.4 |
| Example 3 | 5° C. | 2 | 0.52 | 0.5 |
| Example 3 | 37° C. | 1 | 1.32 | 1.4 |
| Example 3 | 37° C. | 2 | 2.18 | 2.2 |

The results in Table 7 indicate that the compound of Example 3 is significantly more stable than CCK-8s after two weeks at 37° C., and that the stability of the Example 3 compound under these conditions is about 50% higher than the stability of CCK-8s.

### Example 96

### Effect of CCK Derivative on Food Intake and Body Weight in Diet-Induced Obese Mini-Pigs—Mono Therapy and in Combination with GLP-1

The purpose of this experiment is to investigate the effect of a CCK derivative of the invention on food intake and body weight (BW) in diet-induced obese (DIO) mini-pigs. This was done in a sub-chronic pharmacodynamic study as described below, in which the CCK derivative of Example 3 was dosed once daily for 13 weeks in monotherapy (two different dose levels) and in combination with liraglutide. Liraglutide is a mono-acylated GLP-1 derivative for once daily administration which is marketed as of 2009 by Novo Nordisk A/S (see e.g. WO 98/08871 A1, Example 37).

Female, ovariectomised Göttingen mini-pigs, approximately 18 months of age, weighing on average approximately 90 kg were used (n=7-8 per group). The animals were fed ad libitum with mini-pig chow (SDS, Special Diets Services, Scanbur, Denmark) during the 8 months fattening period and ad libitum with Altromin 9023 (Brogaarden, Denmark) for approximately 3 months up to and during the experimental period. The animals were single-housed at all times for measurement of individual food intake. Food intake was measured on line by logging the weight of fodder every 15 minutes using the MP-1 system (Embrose, Faaborg, Denmark). Any spillage of food was manually collected, weighed and subtracted from the food intake measured by the MP-1 system, giving the corrected 24 h food intake. Food intake was measured daily during the entire experiment and BW was measured twice weekly on a large animal scale.

Baseline food intake and BW were measured for 4 weeks, where after the pigs were randomised into 4 treatment groups that were treated for 13 weeks in total: (i) a vehicle group (n=8), (ii) a CCK derivative low dose group (doses gradually adjusted from to 2 nmol/kg during the study, n=7), (iii) a CCK derivative high dose group (10 nmol/kg, n=8), and (iv) a combination group where the CCK derivative (1.5 nmol/kg) was added on top of liraglutide (1.6 nmol/kg) after a run-in period of 8.5 weeks on liraglutide monotherapy (titrated from 0.53 nmol/kg to the full dose of 1.6 nmol/kg over the first 16 days). After the treatment period food intake and BW were followed during a 3 week wash out period. First day of dosing was designated Day 1. On Days 50-51 the pigs were anaesthetised and subsequently allowed to recover until Day 59 where the CCK derivative was added to the liraglutide treatment in group (iv). On Days 78-88 the pigs went through a period with anaesthesia and fasting before metabolic tests, and on Day 91 the last dose was given. The periods with fasting and anaesthesia have been omitted from the calculations and the statistical analysis.

The CCK derivative was dissolved in the following buffer: 8 mM phosphate, 184 mM propylene glycol, 58 mM phenol, pH 7.4 concentrations ranging from 2000-10000 nmol/mL corresponding to dose volumes ranging from 0.00075-0.001 mL/kg. Liraglutide was dissolved in 8 mM phosphate, 184 mM propylenglycol, 58 mM phenol, pH 8.15 (1600 nmol/mL), dose volume was 0.001 mL/kg and this formulation also served as a vehicle for all 3 treatment groups. Animals were dosed subcutaneously once daily with vehicle, CCK derivative, liraglutide, or liraglutide in combination with the CCK derivative (given as two separate injections in each side of the neck). After the last dose blood samples were obtained in EDTA coated tubes at selected time points for up to 480 hours after dosing. Blood was sampled through central-venous catheters implanted under general anaesthesia after 11 weeks of treatment.

The resulting plasma was pipetted into Micronic tubes on dry ice, and kept at −20° C. until analysed for plasma exposure of liraglutide and the CCK derivative. Plasma content of the CCK derivative was measured using LC-MS method II, as described in Example 93. Plasma content of liraglutide was measured using LOCI (Luminescence Oxygen Channeling Immunoasssay). LOCI is generally described for the determination of insulin by Poulsen and Jensen in Journal of Biomolecular Screening 2007, vol. 12, p. 240-247. The donor beads were coated with streptavidin, while acceptor beads were conjugated with a monoclonal antibody recognising a C-terminal epitope of the peptide. Another monoclonal antibody, specific for the N-terminus, was biotinylated. The three reactants were combined with the analyte and formed a two-sited immuno-complex. Illumination of the complex released singlet oxygen atoms from the donor beads, which were channelled into the acceptor beads and triggered chemiluminescence which was measured in an Envision plate reader. The amount of light was proportional to the concentration of the compound.

Accumulated 24 h corrected food intake and delta BW (in %) were calculated during both the monotherapy treatment period from Day 1-49 (Table 8) and during the combination period from Day 59-77 (Table 9) and compared between the groups separately at each time point using one-way ANOVA followed by Sidak's multiple comparison test (GraphPad Prism v. 6.07 for Windows, GraphPad Software, La Jolla Calif. USA).

In addition, in group (iv) the effect of add-on of the CCK derivative to liraglutide treatment was evaluated by comparing BW on Day 59 (last BW measured before add on) with BW on Day 77 (BW after 19 days of combination treatment) and by comparing mean daily food intake during the last 19 days of the liraglutide monotherapy period (Day 31-49, the period just before anaesthesia on Days 50-51) with mean daily food intake during the first 19 days of the combination period (Day 59-77, the period just before anaesthesia on Days 78-80) using paired t-test (GraphPad Prism v. 6.07 for Windows, GraphPad Software, La Jolla Calif. USA). In both periods, the food intake was considered stable, and the BW had stabilised on the liraglutide treatment. The results are shown in Table 10.

Plasma exposure was evaluated as AUC(0-24 h) after the last dose corresponding approximately to steady state levels in all groups (see Table 9). Only animals with functional catheters and an appropriately covered 24 h profile were included in these data (n=5-6). In Table 8 below the accumulated 24 h corrected food intake and delta BW are given for the monotherapy period, and in table 9 the same parameters are given for the combination period in addition to AUC(0-24 h) after the last dose.

TABLE 8
Accumulated food intake and delta BW in DIO
mini-pigs (monotherapy period from Day 1-49)

| | Parameter | |
|---|---|---|
| | Accumulated food | |
| group | intake (kg) | Delta BW (%) |
| Vehicle | 59.1 | 9.0 |
| CCK derivative low dose | 31.9*** | −3.7***^{,b} |
| CCK derivative high dose | 26.5*** | −6.8***^{, bbb} |
| Liraglutide | 30.4*** | 0.7*** |

***p < 0 .001 vs. vehicle,
^{b}p < 0.05 vs. liraglutide,
^{bbb} p < 0.001 vs. liraglutide

TABLE 9
Accumulated food intake, delta BW, and AUCₑₓₚₒₛᵤᵣₑ 0-24 h after
the last dose in DIO mini-pigs (combination period, Day 59-77)

| | Parameter | | |
|---|---|---|---|
| | | | AUCₑₓₚₒₛᵤᵣₑ (0-24 h) |
| | Accumulated food | Delta BW | after last dose |
| group | intake (kg) | (%) | (pM*h) |
| Vehicle | 19.3 | 3.6 | NA |
| CCK derivative | 14.8^{bbb} | 1.7^{bbb} | 4204000 |
| low dose | | | |
| CCK derivative | 11.5**^{,b} | 0.7**^{,bbb} | 27120000 |
| high dose | | | |
| Liraglutide and | 5.3*** | −5.2*** | CCK |
| CCK derivative | | | derivative: 3332000 |
| combination | | | Liraglutide: 399872 |

**p < 0.01 vs. vehicle,
***p < 0.001 vs. vehicle,
^{b}p < 0.05 vs. liraglutide + CCK derivative add on,
^{bbb}p < 0.001 vs. liraglutide + CCK derivative add on

TABLE 10
Average BW on Day 59 and Day 77 and mean daily food
intake from Day 31-49 and Day 59-77 in DIO minipigs
treated with liraglutide alone (Day 31-49) or in combination
with the CCK derivative (Day 59-77)

group

| | Liraglutide | Liraglutide + add |
|---|---|---|
| Parameter | monotherapy | on CCK derivative |
| BW | 90.2 | 85.5*** |
| Mean daily food intake (kg) | 0.66 | 0.28*** |

Paired t-test,
***p < 0.001 vs. liraglutide monotherapy.

The results in Tables 8 and 9 show that daily, sub-chronic dosing of the CCK derivative of Example 3 reduced food intake and body weight significantly compared to vehicle, and in a dose-dependent manner. The dose response effect was most pronounced with respect to BW reduction, although no significant differences were found in either food intake or delta BW between the two CCK derivative dose groups.

In the monotherapy period, food intake was similar in all three treatment groups whereas both CCK derivative groups had a significantly greater weight loss than the liraglutide group. This indicates that the CCK derivative has additional weight loss benefits, not explained solely by the effect on food intake.

In the combination period (Table 9), both food intake and delta BW were significantly lower in the combination group compared to vehicle and the two CCK derivative groups, and these data together with data in Table 10 indicate that CCK derivative add-on to liraglutide treatment leads to further reductions in food intake and BW.

Plasma exposure of the Example 3 compound and liraglutide was confirmed in the end of the study.

### Example 97

### Additional Pharmacodynamic (PD) Studies in LYD Pig, Food Intake

Additional pharmacodynamic (PD) studies were conducted in which food intake was measured from 0-96 hours after administration of single subcutaneous doses of the CCK derivatives of Examples 11 and 19, as compared to baseline measurements.

The study conditions were as described in Example 94 except for the longer duration and except that (i) the CCK derivatives were dissolved in the phosphate buffer at concentrations of approximately 1200 nmol/ml corresponding to doses of 30 nmol/kg, (ii) the blood sample for measurement of plasma exposure of the CCK derivatives was taken from the v. Saphena in anaesthetised animals, and (iii) LC-MS method II of Example 93 was used for the plasma analysis.

Food intake (FI) was calculated in 24 h intervals. In Table 11 below the resulting mean difference in food intake (ΔFI) is given for each time interval relative to mean baseline for each pig, determined as described above. A negative number indicates a reduction in FI. The mean difference in food intake is calculated as follows:

ΔFI₀₋₂₄ₕ=FI₀₋₂₄ₕ−FI_{baseline},

ΔFI₂₄₋₄₈ₕ=FI₂₄₋₄₈ₕ−FI_{baseline},

ΔFI₄₈₋₇₂ₕ=FI₄₈₋₇₂ₕ−FI_{baseline},

ΔFI₇₂₋₉₆ₕ=FI₇₂₋₉₆ₕ−FI_{baseline}

TABLE 11
Effect on food intake in pig

| | Food intake | | | |
|---|---|---|---|---|
| | ΔFI_{0-24 hours} | ΔFI_{24-48 hours} | ΔFI_{48-72 hours} | ΔFI_{72-96 hours} |
| Compound | (kg) | (kg) | (kg) | (kg) |
| Example 11 | −0.62 | −0.72 | −0.27 | −0.19 |
| Example 19 | −0.99 | −0.68 | −0.26 | −0.08 |

The results in Table 11 show that a single s.c. injection of the CCK derivatives of Examples 11 and 19 in pigs reduces food intake for at least 96 h after dosing.

### Example 98

### Pharmaceutical Compositions

A solution suitable for s.c. injection of the compound of Example 3 with a strength of 10.0 mM (Formulation A) was prepared by performing the following steps:

1500 mL vehicle was prepared by mixing:

8.12 g phenol

21.0 g propylene glycol

345 mg sodium dihydrogen phosphate, H₂O

1691 g disodium hydrogen phosphate, 2H₂O

1425 g milli Q water

pH was adjusted to 7.4 using 0.2N HCl/NaOH, and water was added to a total mass of 1500 g.
100 mL vehicle was added to an amount of lyophilised powder of the compound of Example 3 as a sodium salt corresponding to 1.0 millimole and the mixture was stirred for at least 10 minutes until the powder was completely dissolved. pH was adjusted to 7.4+/−0.2 using 0.2N HCl/NaOH, and the solution was sterile filtered.

Formulations B and C were prepared in the same manner:

### Formulation B:

5.0 mM (9.25 mg/mL) of the compound of Example 3
pH 7.4
8.0 mM phosphate
14 mg/mL propylene glycol
58 mM phenol
Disodium phosphate was used for the phosphate buffer and pH was adjusted using dilute HCl/NaOH.

### Formulation C:

5.0 mM (9.25 mg/mL) of the compound of Example 3
pH 7.4

### 8.0 mM Tris(hydroxymethyl)aminomethane

14 mg/mL propylene glycol
58 mM phenol.
pH was adjusted using dilute HCl/NaOH.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A peptide derivative comprising the formula P-L-X1-X2-X3-Gly-Trp-X6-DMeAsp-X8-NH₂,
wherein P is HOOC—(CH₂)ₓ—CO—*, wherein x is an integer in the range of 12-18;
wherein L is absent or comprises at least one linker element selected from the group consisting of
*—NH—CH(COOH)—(CH₂)₂—CO—*,
*—NH—(CH₂)₂—[O—(CH₂)₂]ₖ—O—[CH₂]ₙ—CO—*,
*—NH—CH(CH₂OH)—CO—*,
*—NH—CH₂—CO—*,
and
*—NH—CH[(CH₂)₄—NH₂]—CO—*,
wherein k is an integer in the range of 1-11, and wherein n is an integer in the range of 1-5;
wherein X1 is absent or is selected group from the group consisting of Asp, DAsp, bAsp, DbAsp, Glu, and DGlu;
wherein X2 is selected from the group consisting of Phe(4-sulfomethyl) and sTyr;
wherein X3 is selected from the group consisting of Nle, Leu, Ala, Ile, Lys, Arg, Pro, Met, Phe, Ser, His, and Val;
wherein X6 is selected from the group consisting of Nle, Ile, Gln, Met, Met(O2), Leu, Val, Pro, Hpg, and Ala;
wherein X8 is selected from the group consisting of Phe, MePhe, 1Nal, Me1Nal, 2Nal, Me2Nal, Trp, and MeTrp; and
wherein the peptide derivative is C-terminally amidated by the NH₂ group;
or a pharmaceutically acceptable salt, amide, or ester thereof.

2. The peptide derivative of claim 1, wherein L comprises at least one linker element selected from the group consisting of *—NH—CH(COOH)—(CH₂)₂—CO—*, *—NH—(CH₂)₂—[O—(CH₂)₂]ₖ—O—[CH₂]ₙ—CO—*, *—NH—CH(CH₂OH)—CO—*, *—NH—CH₂—CO—*, and *—NH—CH[(CH₂)₄—NH₂]—CO—*, wherein k is an integer in the range of 1-11, and wherein n is an integer in the range of 1-5.

3. The peptide derivative of claim 1, wherein L comprises at least one linker element selected from the group consisting of *—NH—CH(COOH)—(CH₂)₂—CO—*, *—NH—(CH₂)₂—[O—(CH₂)₂]ₖ—O—[CH₂]ₙ—CO—*, and *—NH—CH[(CH₂)₄—NH₂]—CO—*, wherein k is an integer in the range of 1-11, and wherein n is an integer in the range of 1-5.

4. The peptide derivative of claim 1, wherein L comprises linker element *—NH—(CH₂)₂—[O—(CH₂)₂]ₖ—O—[CH₂]ₙ—CO—*, wherein k is an integer in the range of 1-11, and wherein n is an integer in the range of 1-5.

5. The peptide derivative of claim 1, wherein
X1 is absent or selected from the group consisting of Asp, DAsp, bAsp, DbAsp, Glu, and DGlu;
X2 is selected from the group consisting of Phe(4-sulfomethyl) and sTyr;
X3 is selected from the group consisting of Nle, Leu, Ala, Ile, Lys, Arg, Pro, Met, Phe, Ser, and His;
X6 is selected from the group consisting of Nle, Ile, Gln, Met, Met(O2), Leu, Val, Pro, and Ala; and
X8 is selected from the group consisting of Phe, MePhe, 1Nal, Me1Nal, 2Nal, and Trp.

6. The peptide derivative of claim 1, wherein x is an integer in the range of 14-18.

7. The peptide derivative of claim 6, wherein x is an integer in the range of 14-16.

8. The peptide derivative of claim 1, wherein the peptide derivative is selected from the group consisting of: 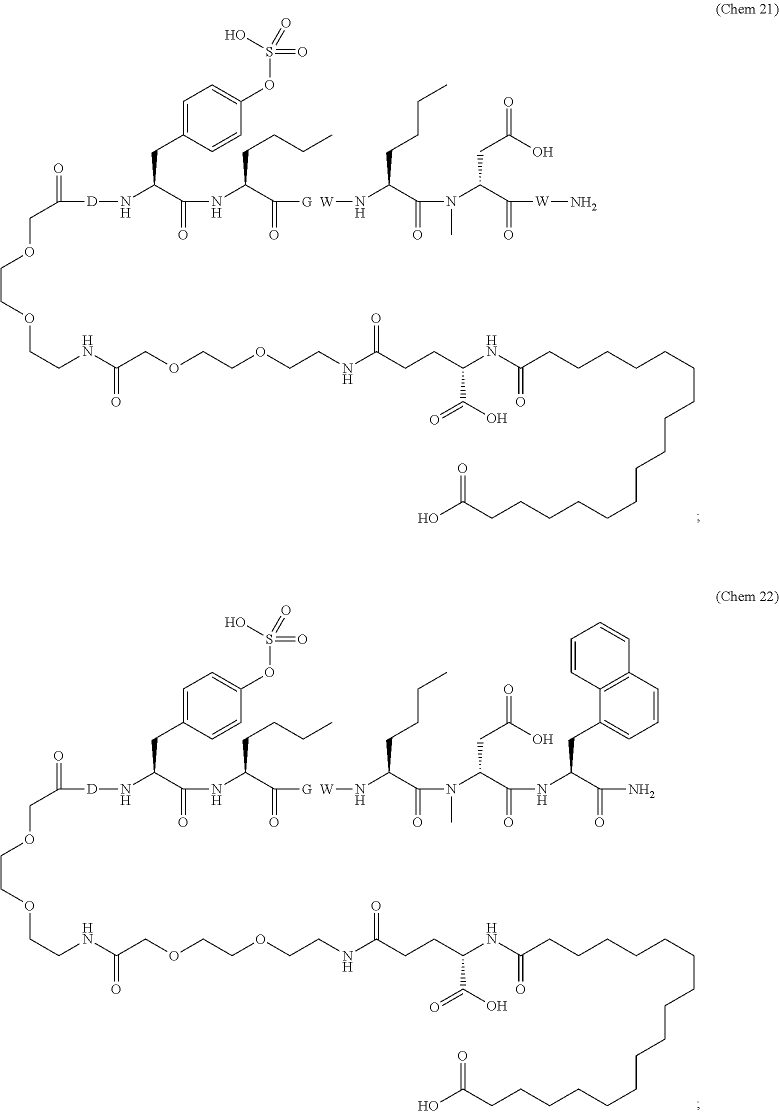 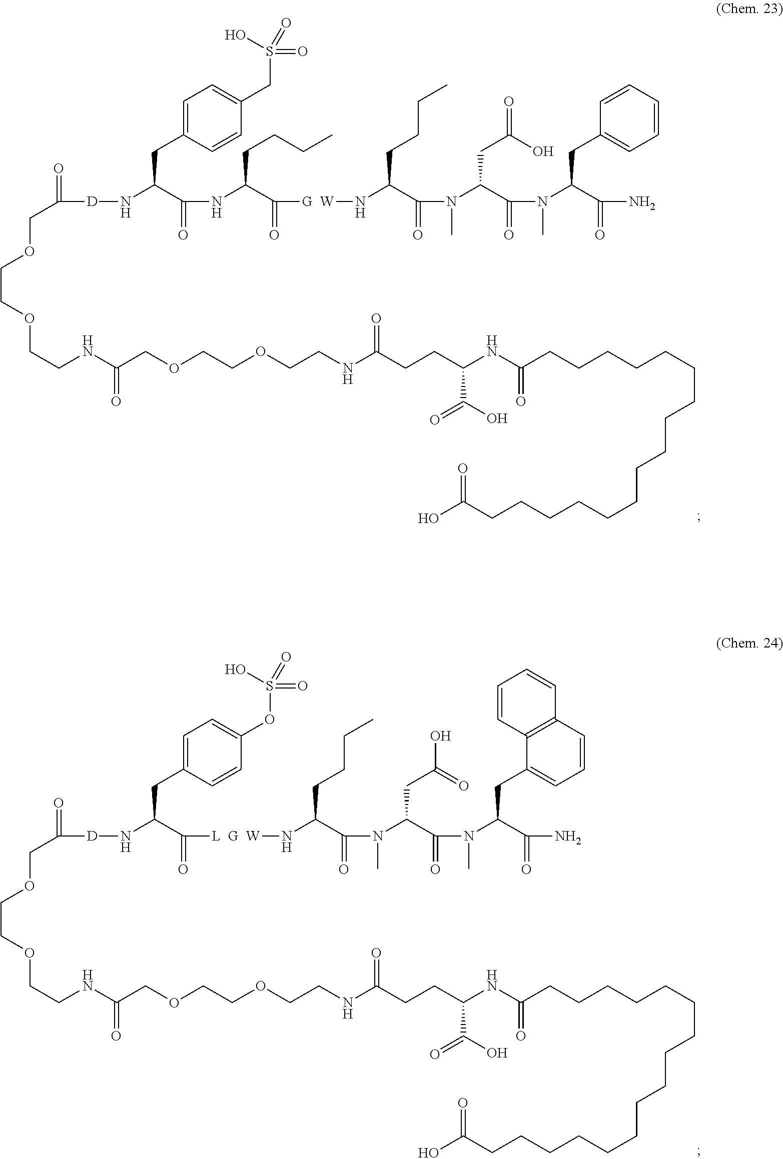 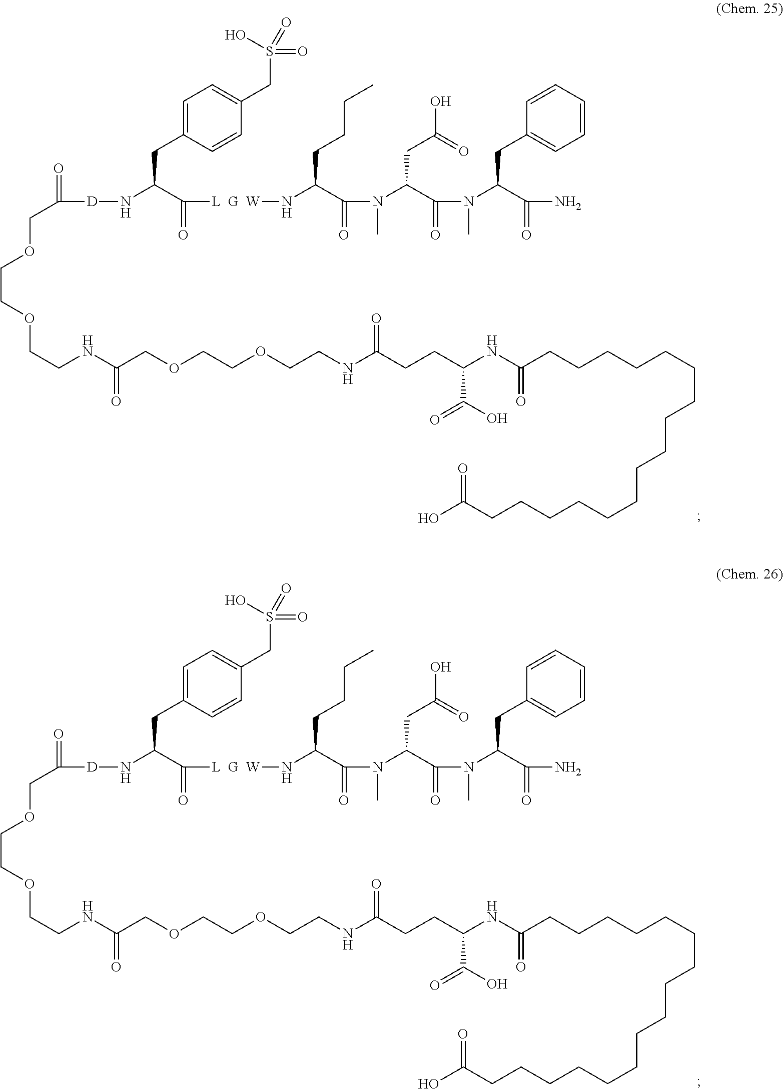 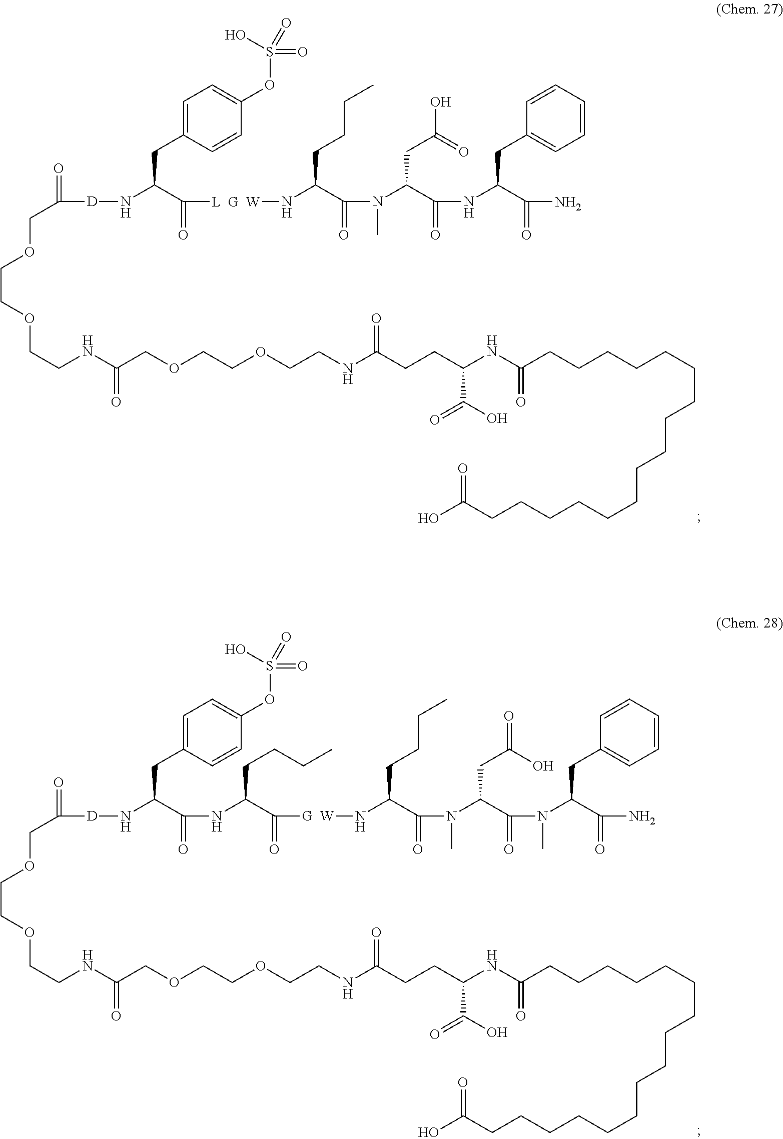 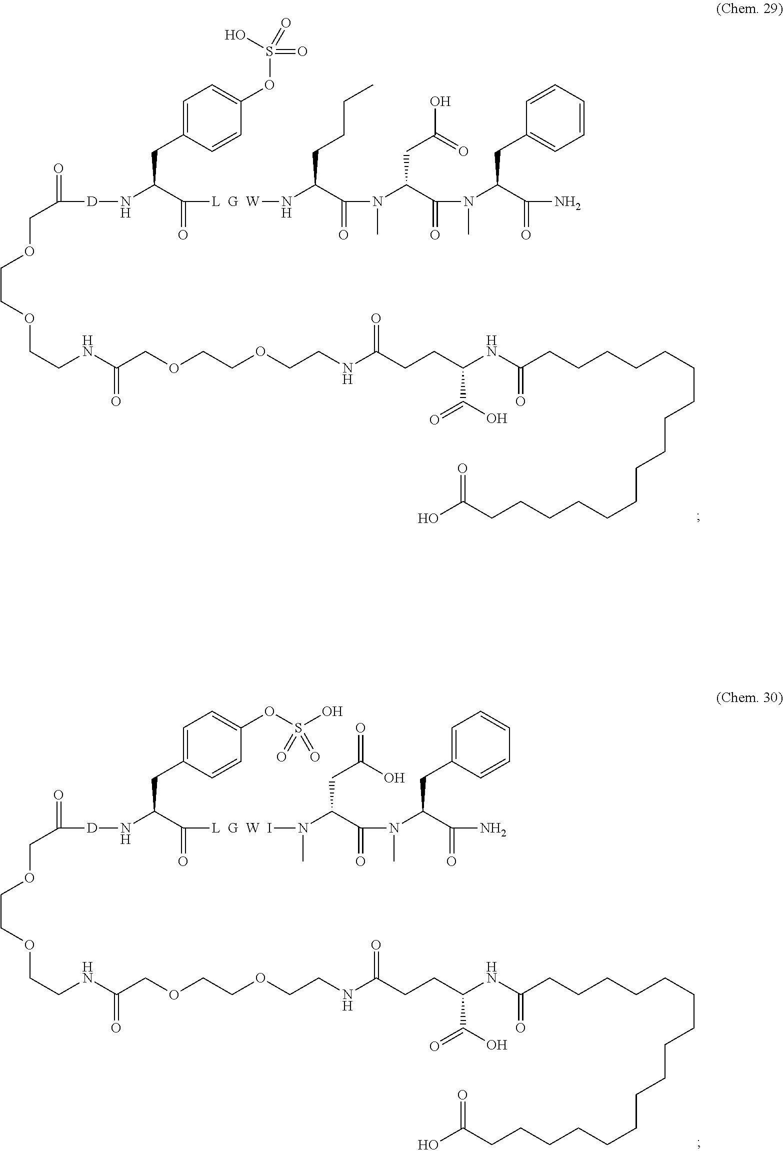 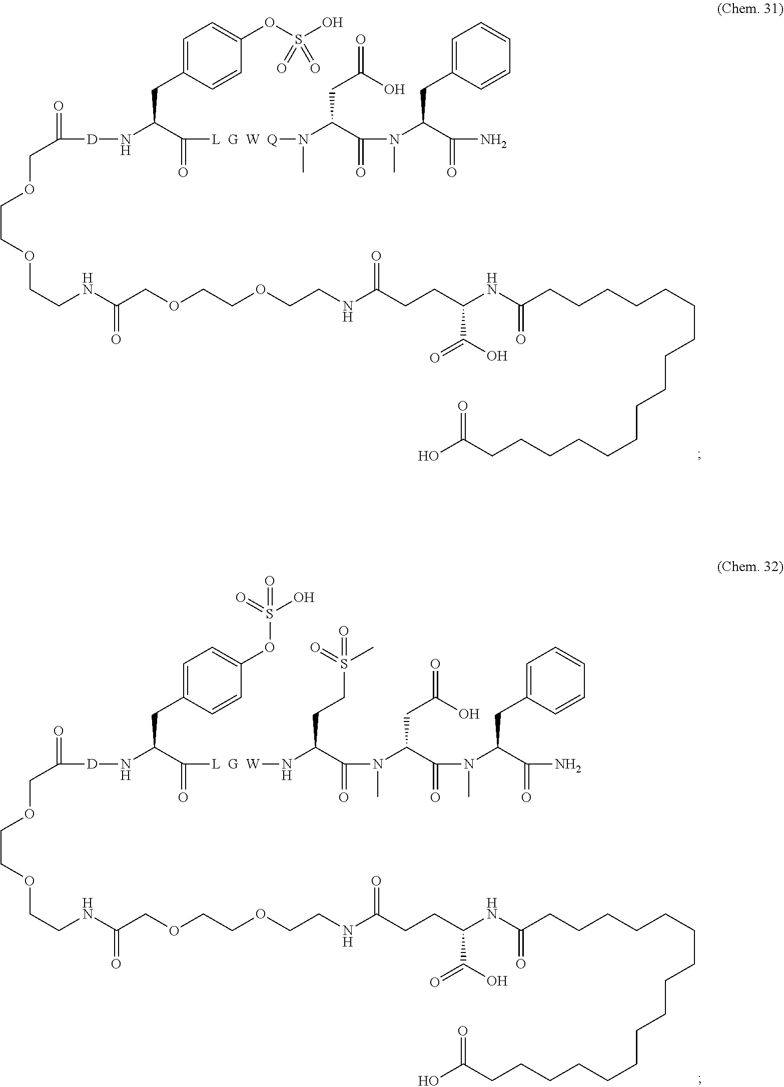 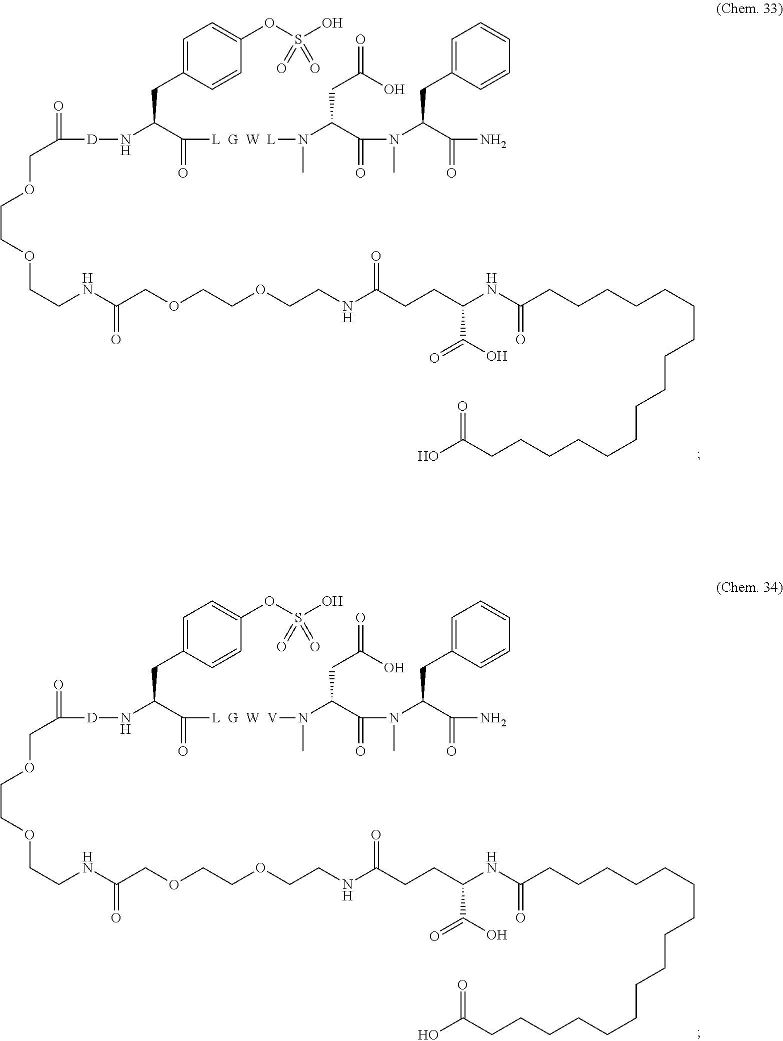 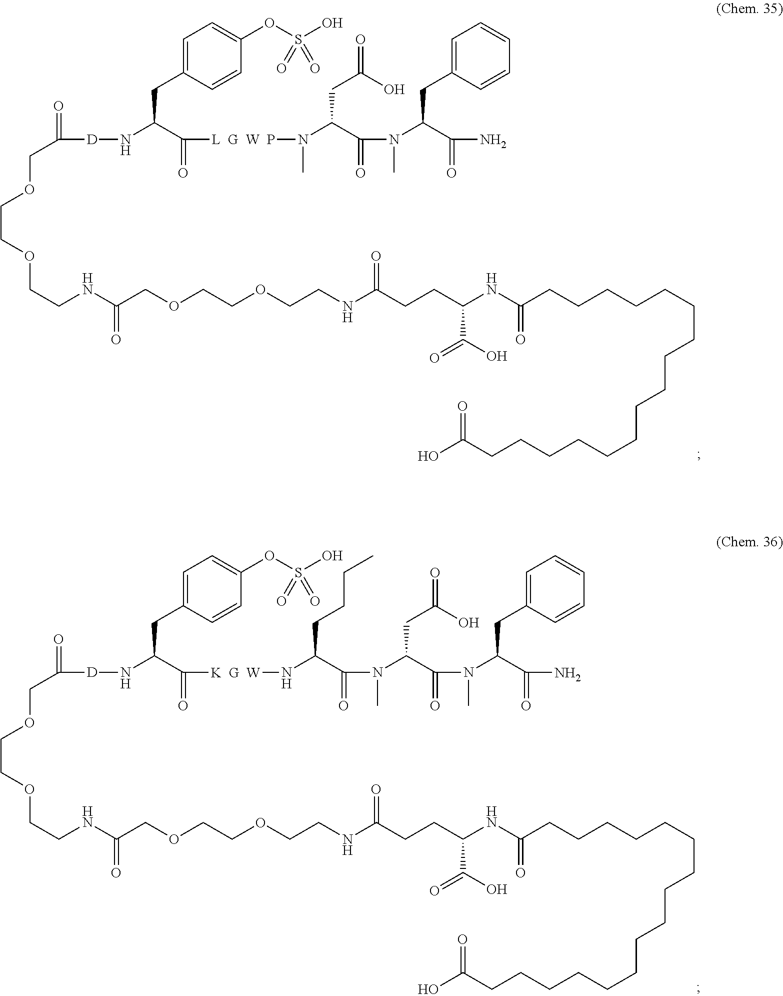 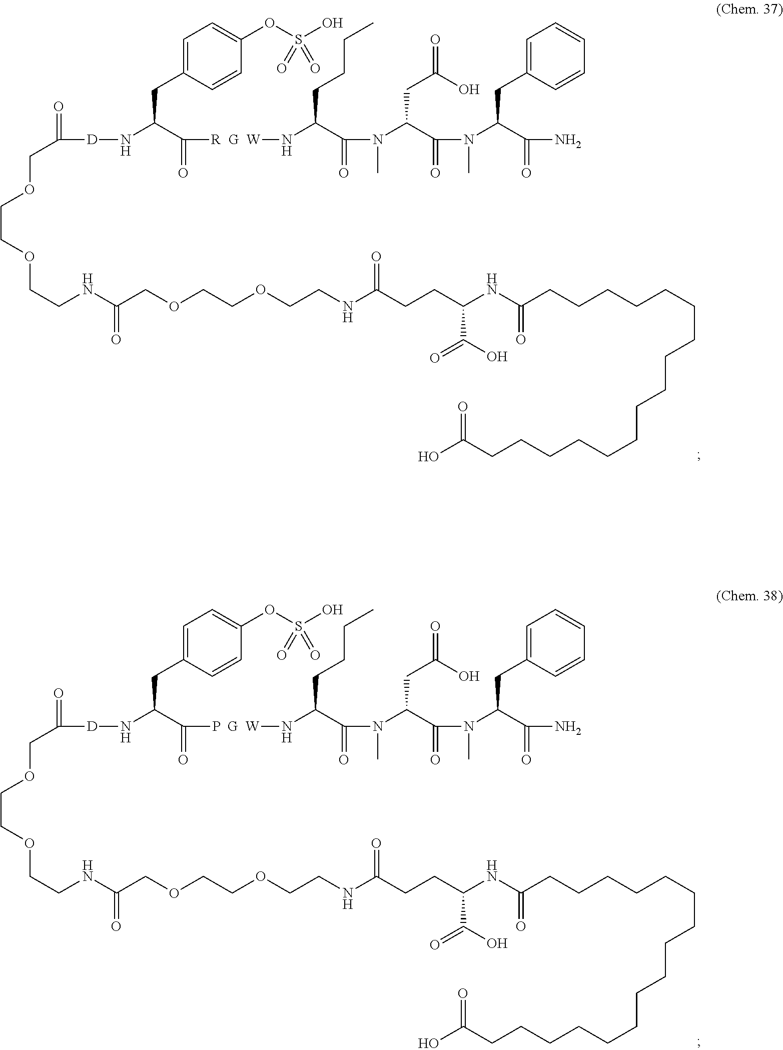 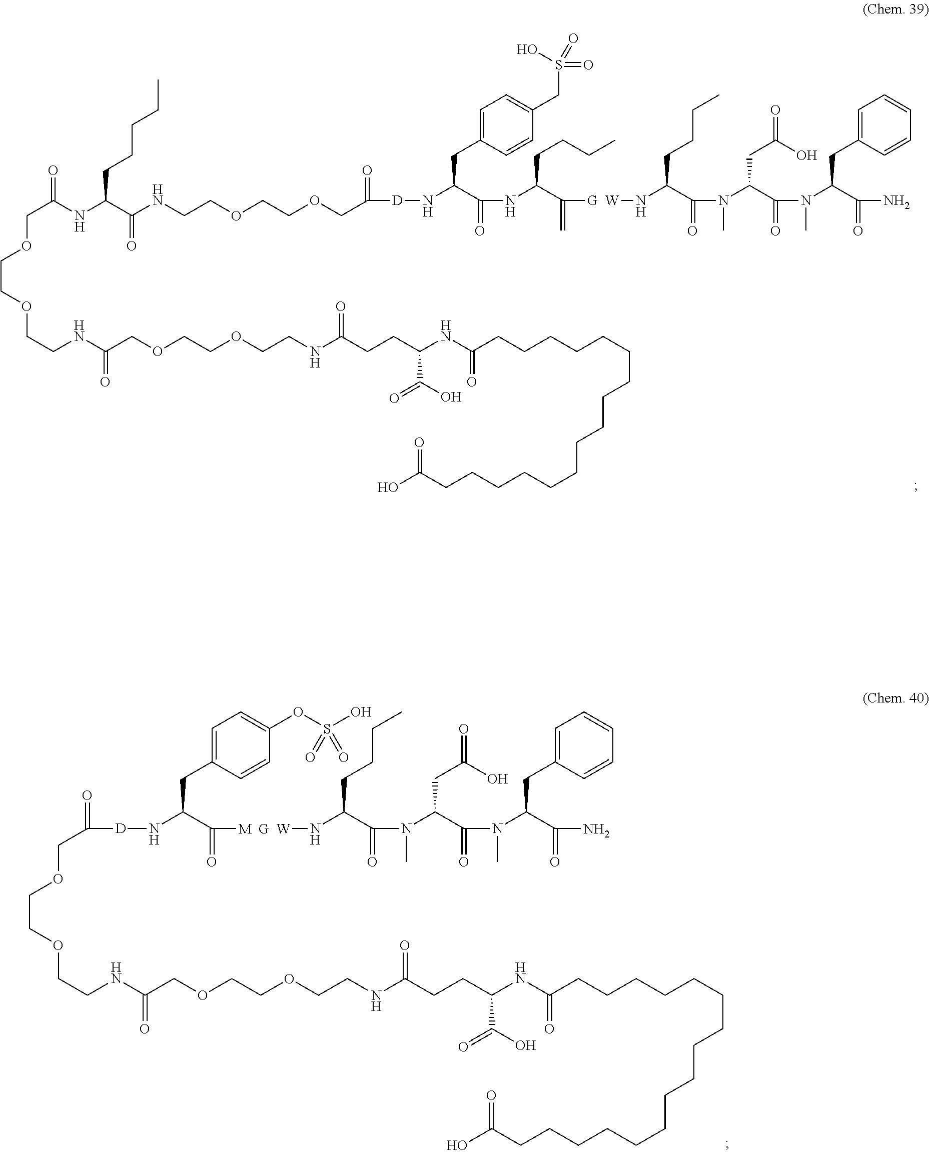 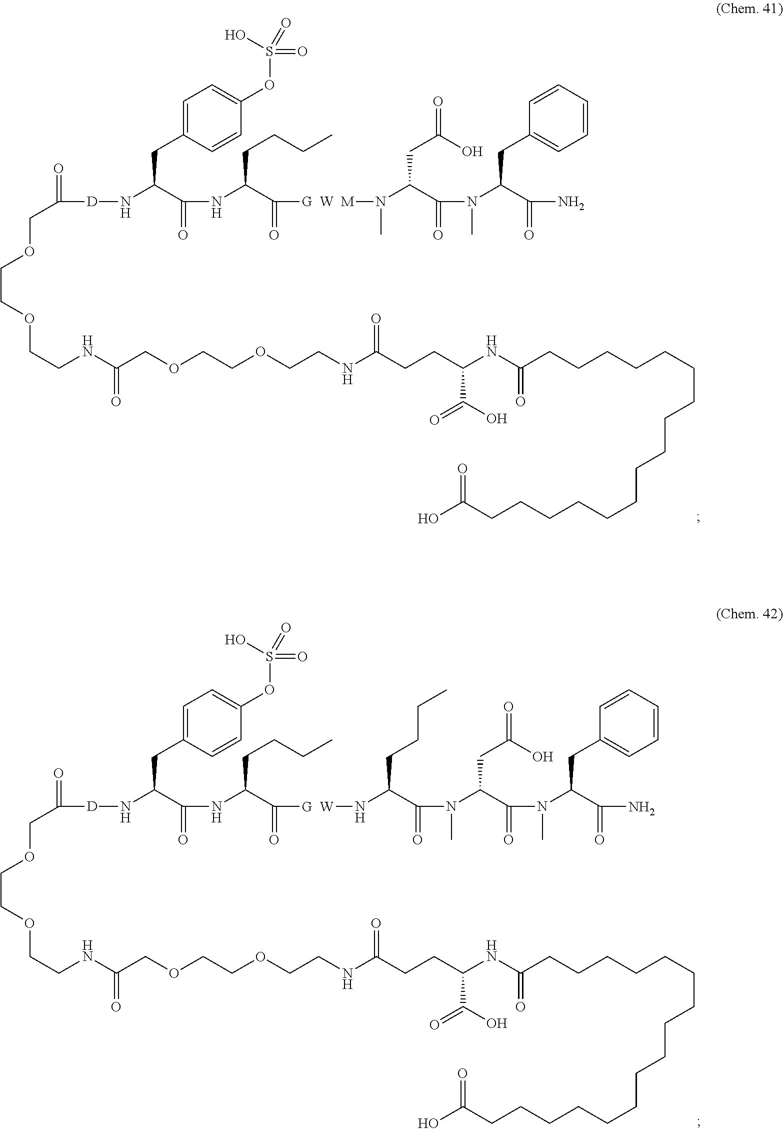 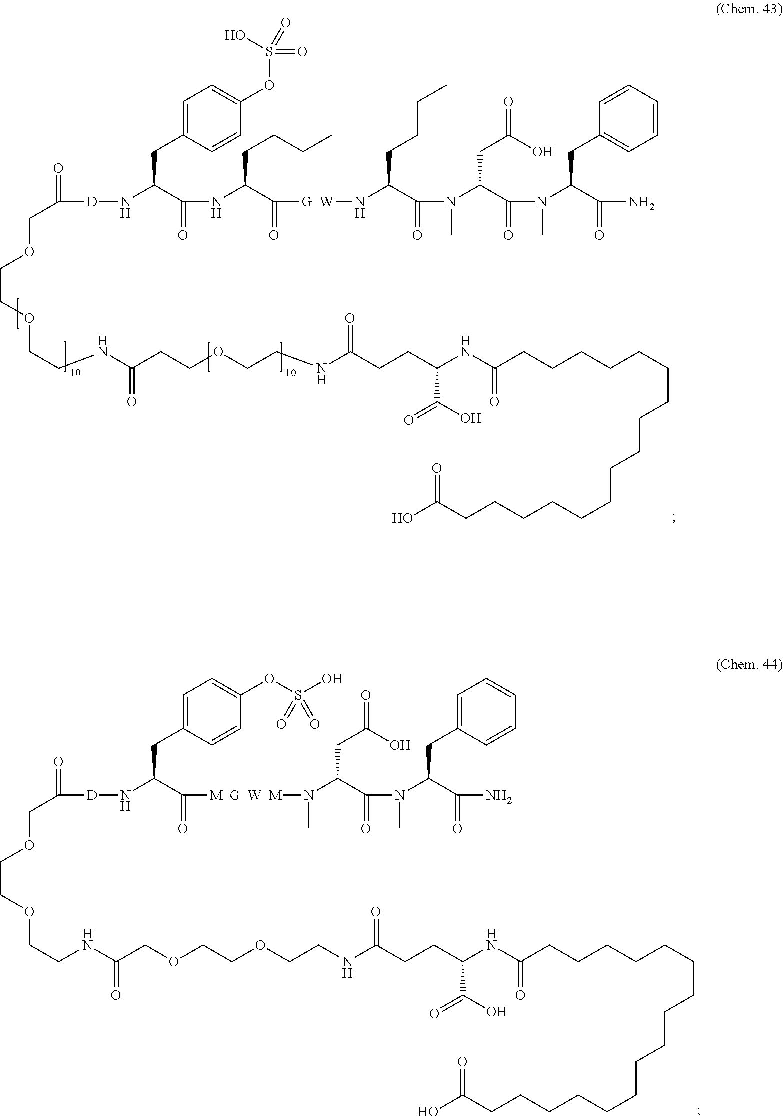 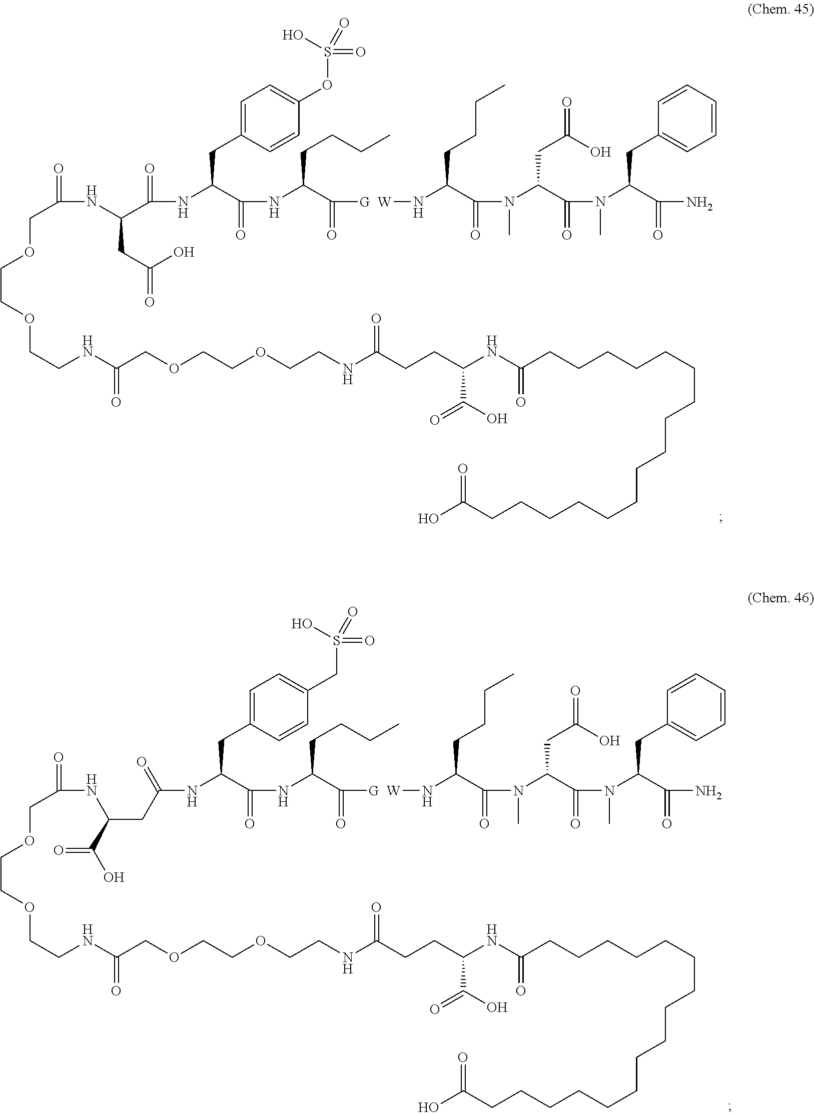 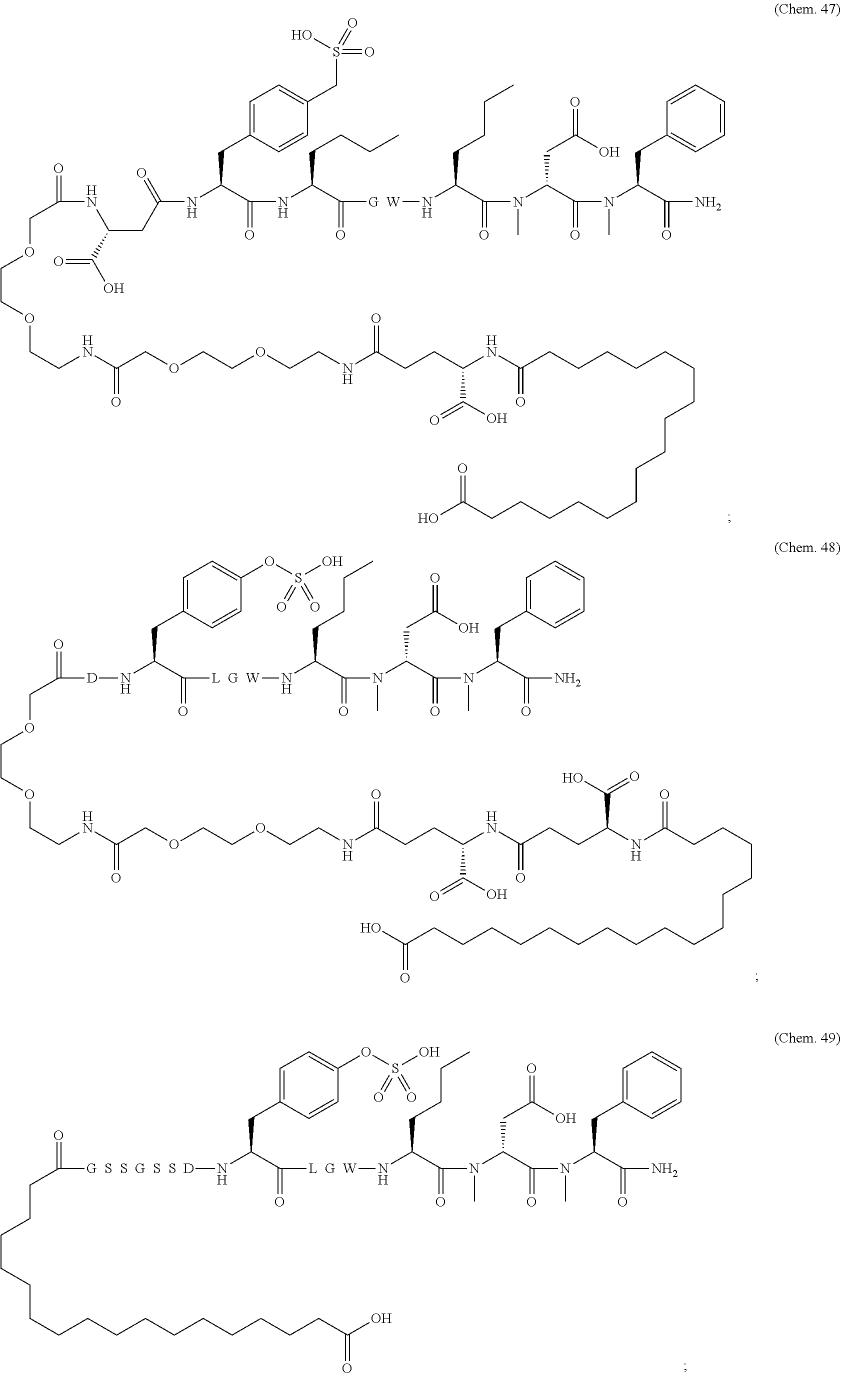 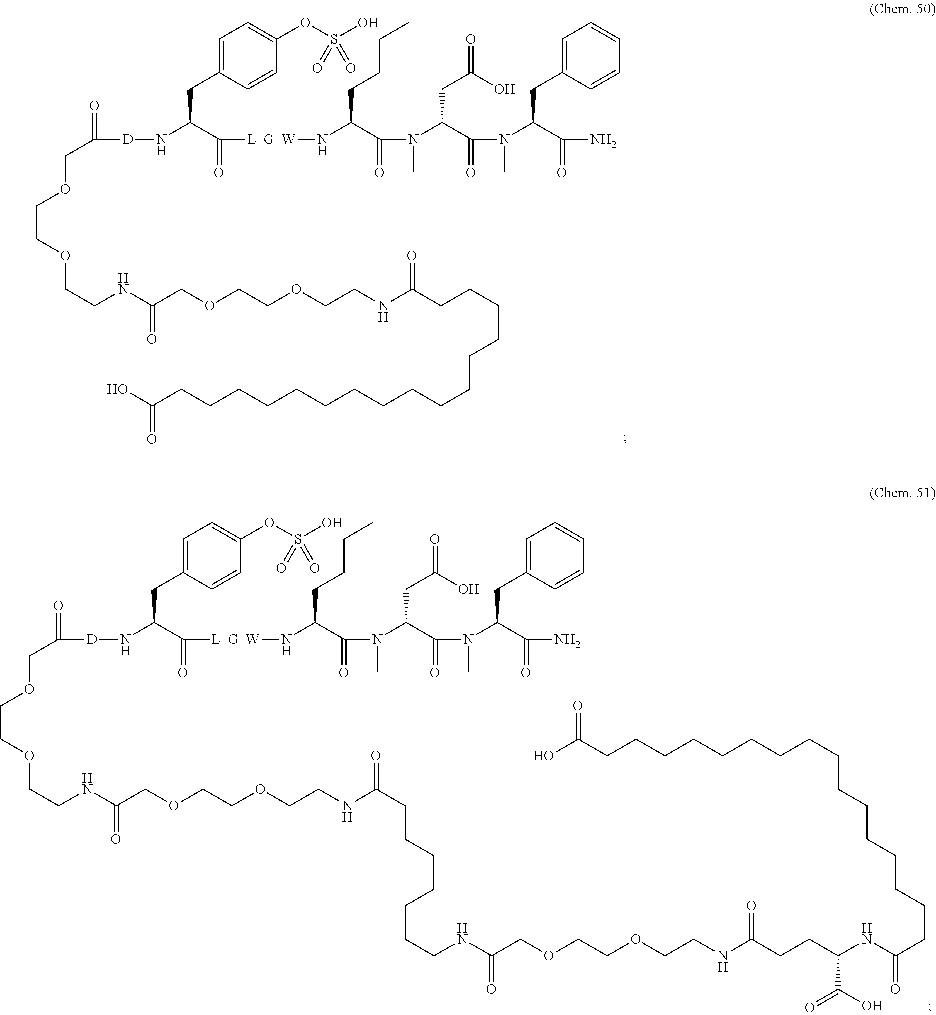 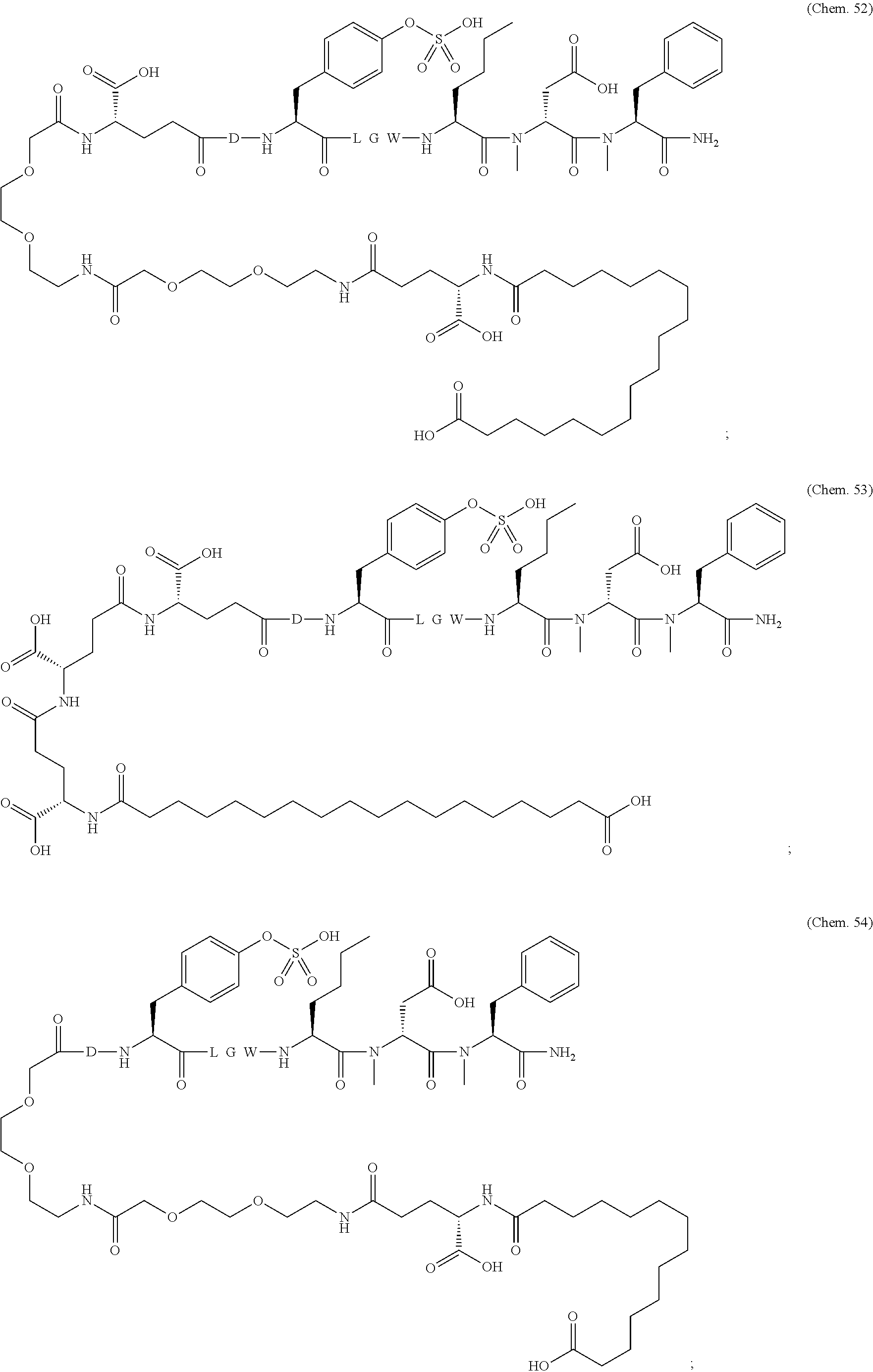 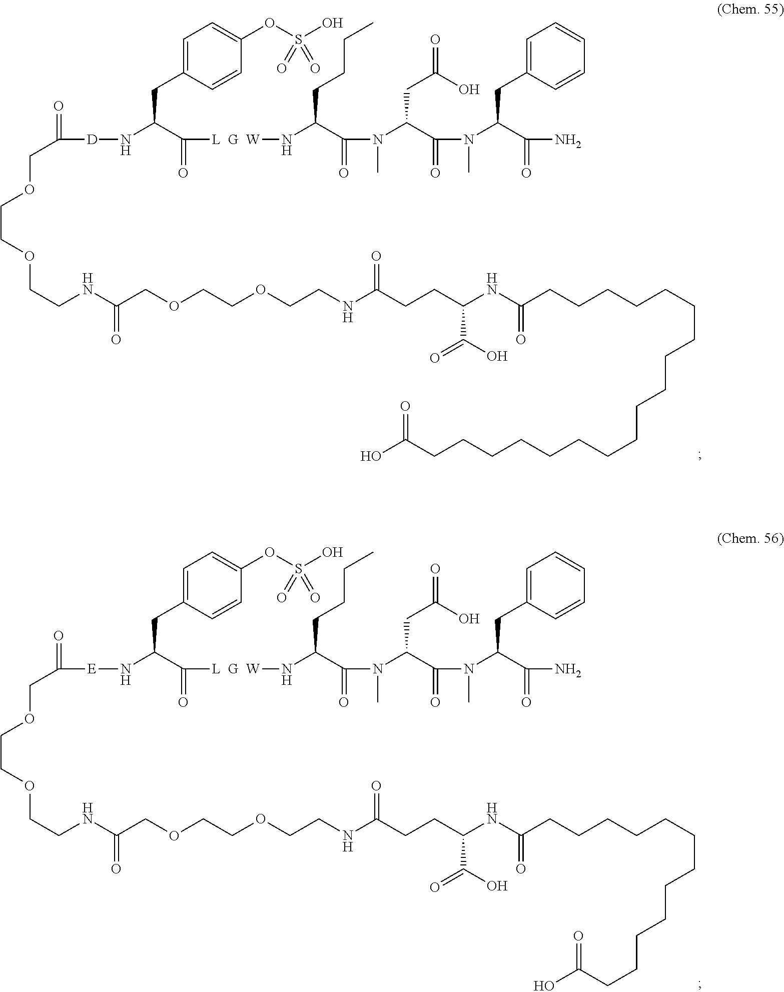 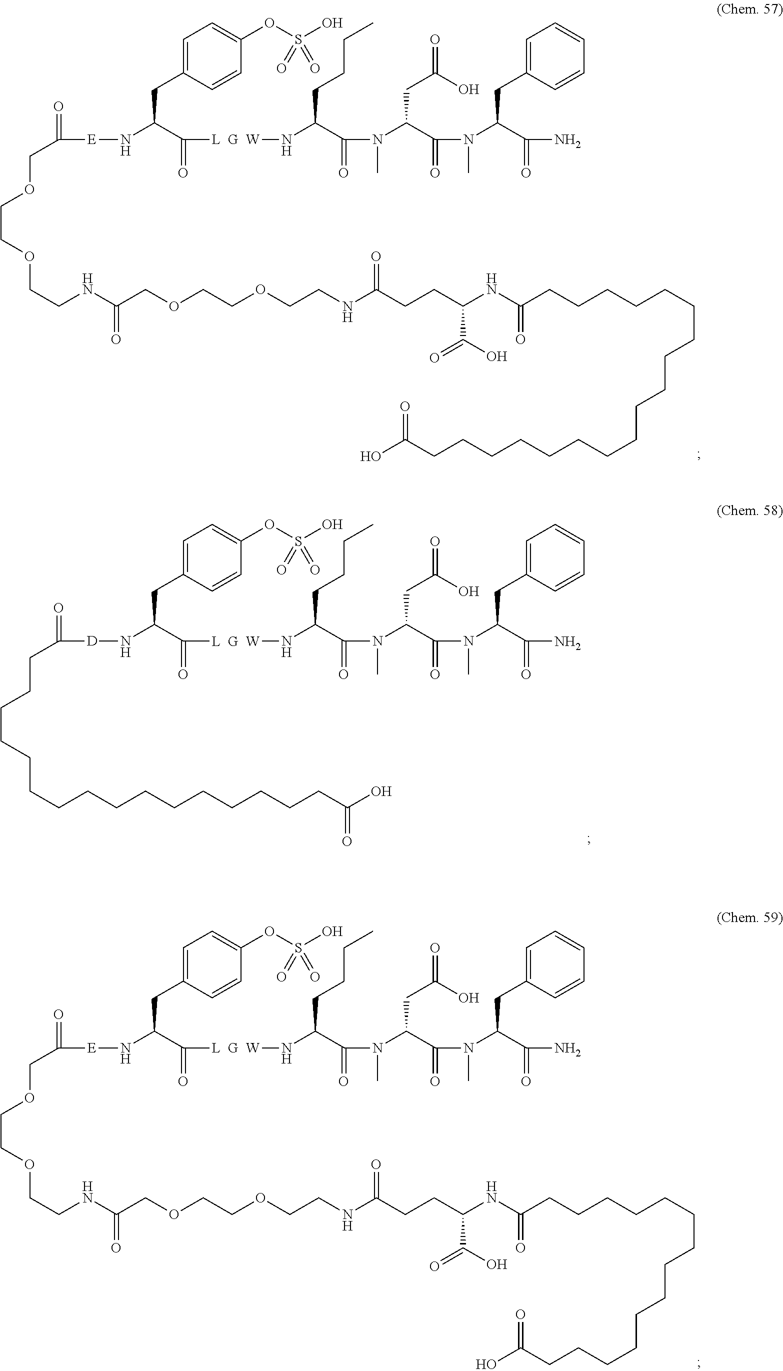 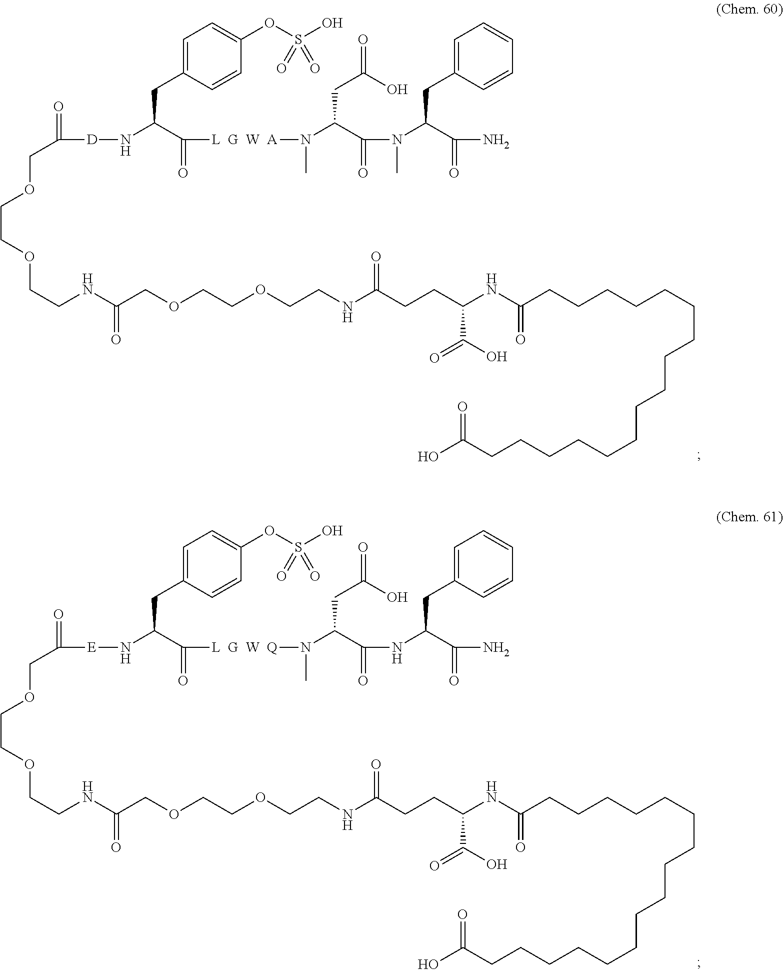 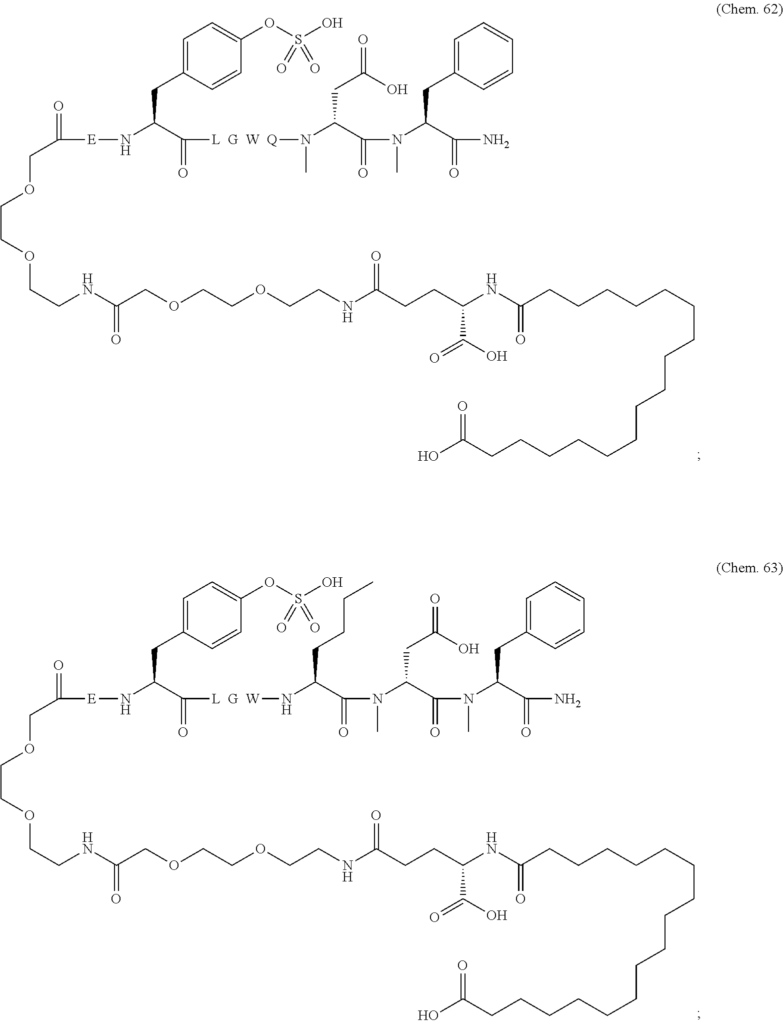 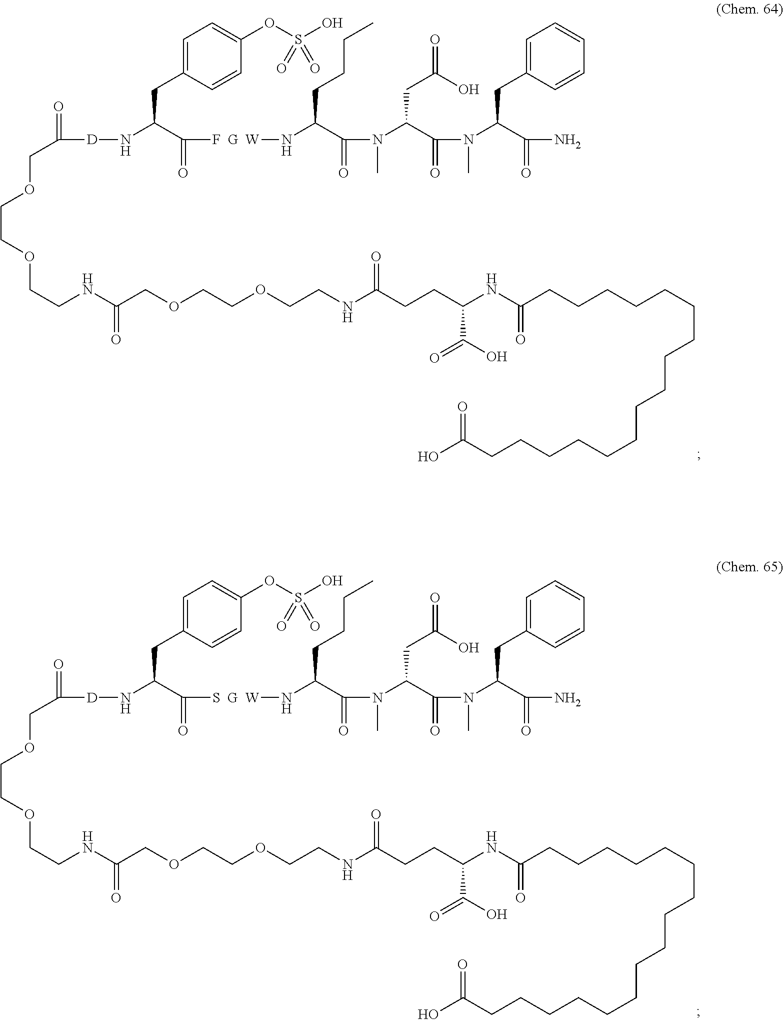 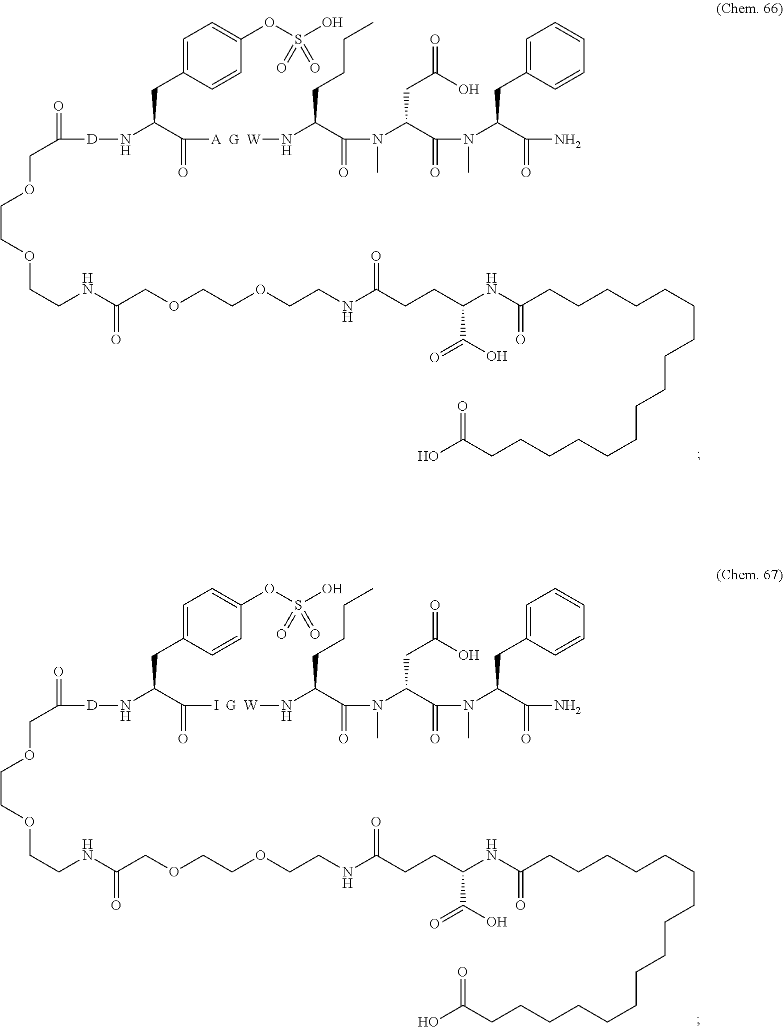 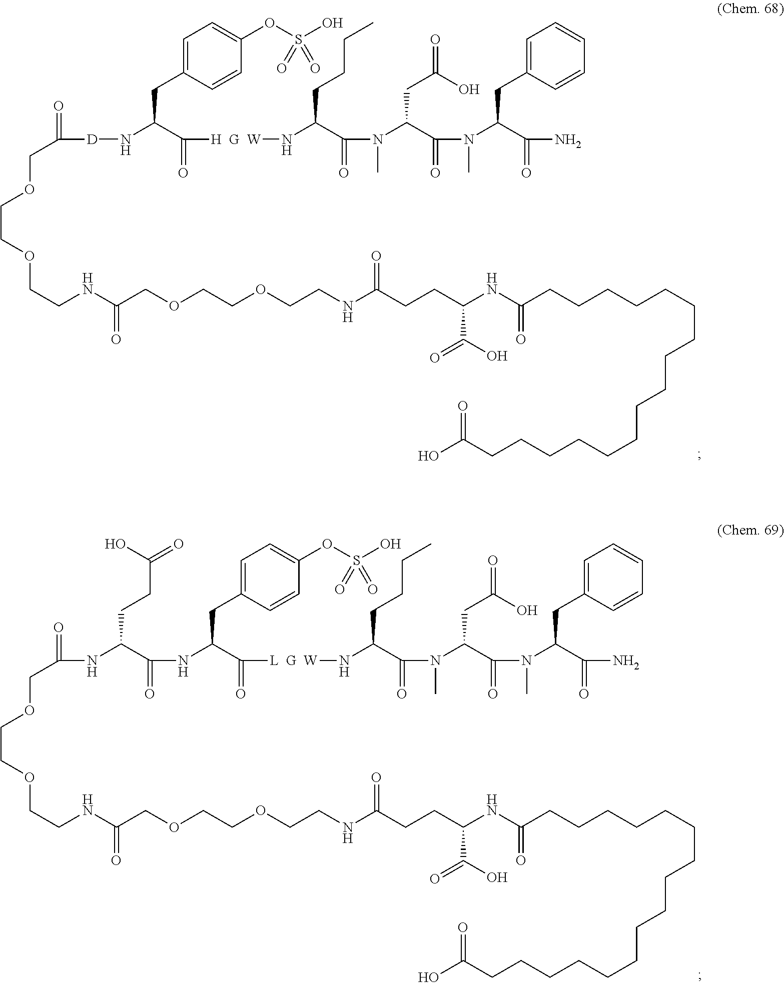 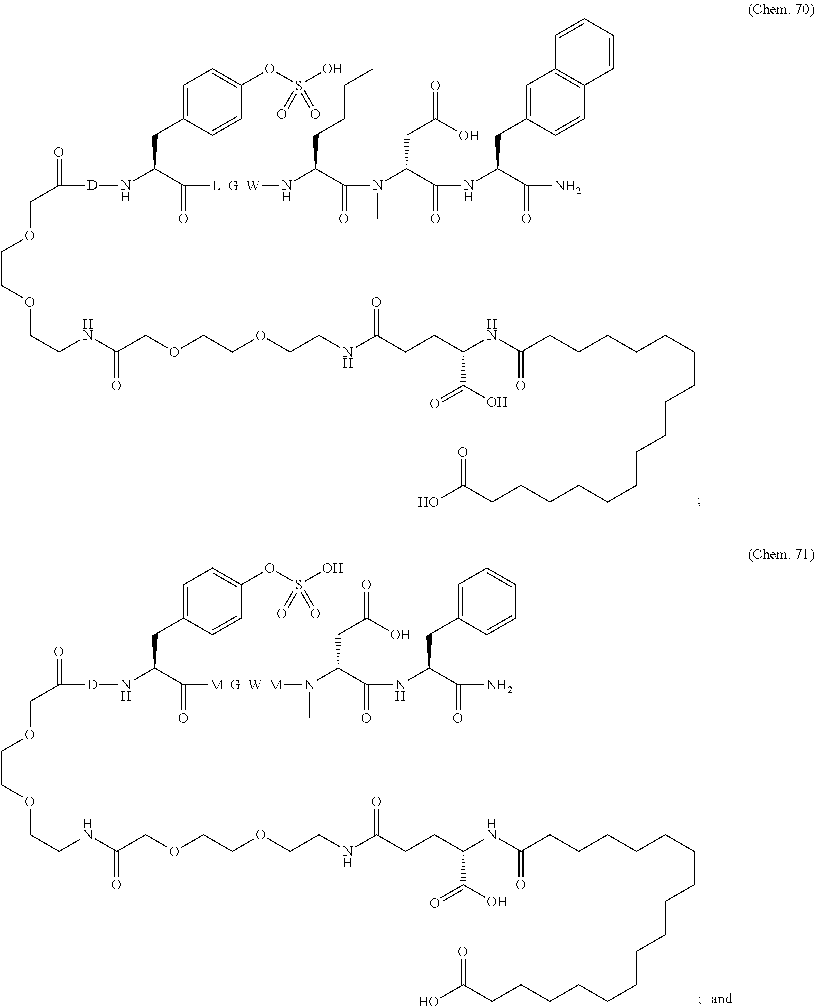 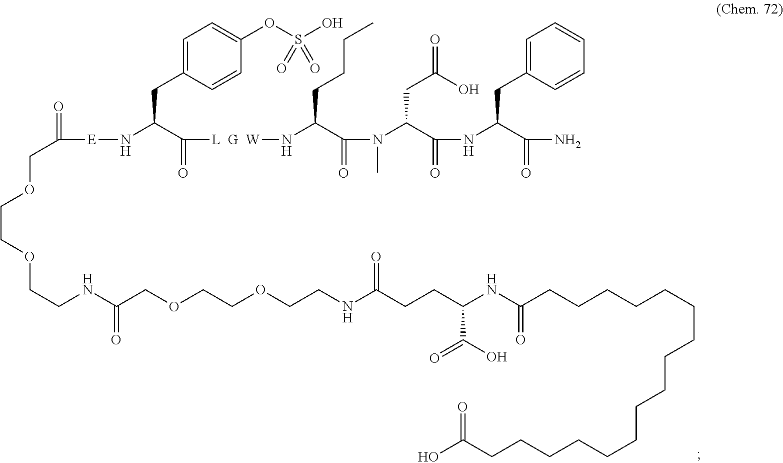
or a pharmaceutically acceptable salt, amide, or ester thereof.

9. The peptide derivative of claim 8, wherein the peptide derivative is

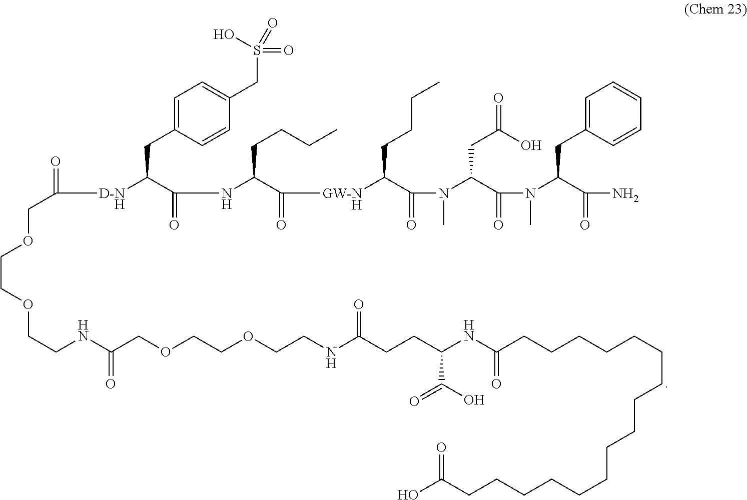

10. A peptide comprising the amino acid sequence X1-X2-X3-Gly-Trp-X6-DMeAsp-X8-NH₂,
wherein
X1 is absent or is selected from the group consisting of Asp, DAsp, bAsp, DbAsp, Glu, and DGlu;
X2 is selected from the group consisting of Phe(4-sulfomethyl) and sTyr;
X3 is selected from the group consisting of Nle, Leu, Ala, Ile, Lys, Arg, Pro, Met, Phe, Ser, His, and Val;
X6 is selected from the group consisting of Nle, Ile, Gln, Met, Met(O2), Leu, Val, Pro, Hpg, and Ala;
X8 is selected from the group consisting of Phe, MePhe, 1Nal, Me1Nal, 2Nal, Me2Nal, Trp, and MeTrp; and
wherein the peptide is C-terminally amidated by the NH₂ group;
or a pharmaceutically acceptable salt, amide, or ester thereof.

11. The peptide of claim 10, wherein the peptide is selected from the group consisting of
Asp-sTyr-Nle-Gly-Trp-Nle-DMeAsp-Trp-amide,
Asp-sTyr-Nle-Gly-Trp-Nle-DMeAsp-1Nal-amide,
Asp-Phe(4-sulfomethyl)-Nle-Gly-Trp-Nle-DMeAsp-
MePhe-amide,
Asp-sTyr-Leu-Gly-Trp-Nle-DMeAsp-Me1Nal-amide,
Asp-Phe(4-sulfomethyl)-Leu-Gly-Trp-Nle-DMeAsp-
MePhe-amide,
Asp-sTyr-Leu-Gly-Trp-Nle-DMeAsp-Phe-amide,
Asp-sTyr-Nle-Gly-Trp-Nle-DMeAsp-MePhe-amide,
Asp-sTyr-Leu-Gly-Trp-Nle-DMeAsp-MePhe-amide,
Asp-sTyr-Leu-Gly-Trp-Ile-DMeAsp-MePhe-amide,
Asp-sTyr-Leu-Gly-Trp-Gln-DMeAsp-MePhe-amide,
Asp-sTyr-Leu-Gly-Trp-Met(O2)-DMeAsp-MePhe-amide,
Asp-sTyr-Leu-Gly-Trp-Leu-DMeAsp-MePhe-amide,
Asp-sTyr-Leu-Gly-Trp-Val-DMeAsp-MePhe-amide,
Asp-sTyr-Leu-Gly-Trp-Pro-DMeAsp-MePhe-amide,
Asp-sTyr-Lys-Gly-Trp-Nle-DMeAsp-MePhe-amide,
Asp-sTyr-Arg-Gly-Trp-Nle-DMeAsp-MePhe-amide,
Asp-sTyr-Pro-Gly-Trp-Nle-DMeAsp-MePhe-amide,
Asp-sTyr-Met-Gly-Trp-Nle-DMeAsp-MePhe-amide,
Asp-sTyr-Nle-Gly-Trp-Met-DMeAsp-MePhe-amide,
sTyr-Nle-Gly-Trp-Nle-DMeAsp-MePhe-amide,
Asp-sTyr-Met-Gly-Trp-Met-DMeAsp-MePhe-amide,
DAsp-Phe(4-sulfomethyl)-Nle-Gly-Trp-Nle-DMeAsp-
MePhe-amide,
bAsp-Phe(4-sulfomethyl)-Nle-Gly-Trp-Nle-DMeAsp-
MePhe-amide,
DbAsp-Phe(4-sulfomethyl)-Nle-Gly-Trp-Nle-DMeAsp-
MePhe-amide,
Glu-sTyr-Leu-Gly-Trp-Nle-DMeAsp-MePhe-amide,
Asp-sTyr-Phe-Gly-Trp-Nle-DMeAsp-MePhe-amide,
Asp-sTyr-Ser-Gly-Trp-Nle-DMeAsp-MePhe-amide,
Asp-sTyr-Ala-Gly-Trp-Nle-DMeAsp-MePhe-amide,
Asp-sTyr-Ile-Gly-Trp-Nle-DMeAsp-MePhe-amide,
Asp-sTyr-His-Gly-Trp-Nle-DMeAsp-MePhe-amide,
Gly-Ser-Ser-Gly-Ser-Ser-Asp-sTyr-Leu-Gly-Trp-
Nle-DMeAsp-MePhe-amide,
Asp-sTyr-Leu-Gly-Trp-Ala-DMeAsp-MePhe-amide,
Glu-sTyr-Leu-Gly-Trp-Gln-DMeAsp-Phe-amide,
Glu-sTyr-Leu-Gly-Trp-Gln-DMeAsp-MePhe-amide,
DGlu-sTyr-Leu-Gly-Trp-Nle-DMeAsp-MePhe-amide,
Asp-sTyr-Leu-Gly-Trp-Nle-DMeAsp-2Nal-amide,
Asp-sTyr-Met-Gly-Trp-Met-DMeAsp-Phe-amide,
and
Glu-sTyr-Leu-Gly-Trp-Nle-DMeAsp-Phe-amide;
or a pharmaceutically acceptable salt, amide, or ester thereof.

12. The peptide of claim 11, wherein the peptide is Asp-Phe(4-sulfomethyl)-Nle-Gly-Trp-Nle-DMeAsp-MePhe-amide.

13. A pharmaceutical composition comprising the peptide derivative of claim 1 and a pharmaceutically acceptable excipient.

14. The pharmaceutical composition of claim 13, wherein the peptide derivative is

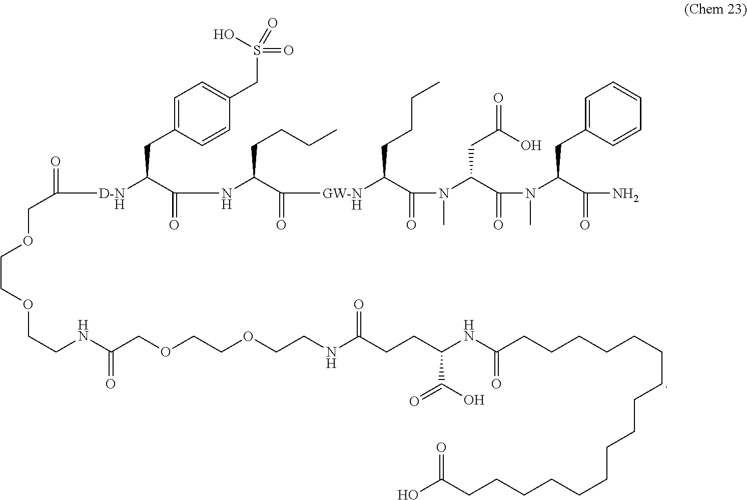

15. The pharmaceutical composition of claim 13, wherein the pharmaceutically acceptable excipient is selected from the group consisting of a buffer, an isotonic agent, and a preservative.

16. The pharmaceutical composition of claim 15, wherein the composition comprises the buffer, the isotonic agent, and the preservative;
wherein the buffer is selected from the group consisting of phosphate and Tris(hydroxymethyl)aminomethane;
wherein the isotonic agent is propylene glycol; and
wherein the preservative is selected from the group consisting of m-cresol and phenol.

17. The pharmaceutical composition of claim 16, wherein the pharmaceutical composition comprises about 5.0 mM peptide derivative, about 8.0 mM phosphate, about 14 mg/mL propylene glycol, about 58 mM phenol, and has about pH 7.4; and wherein the peptide derivative is

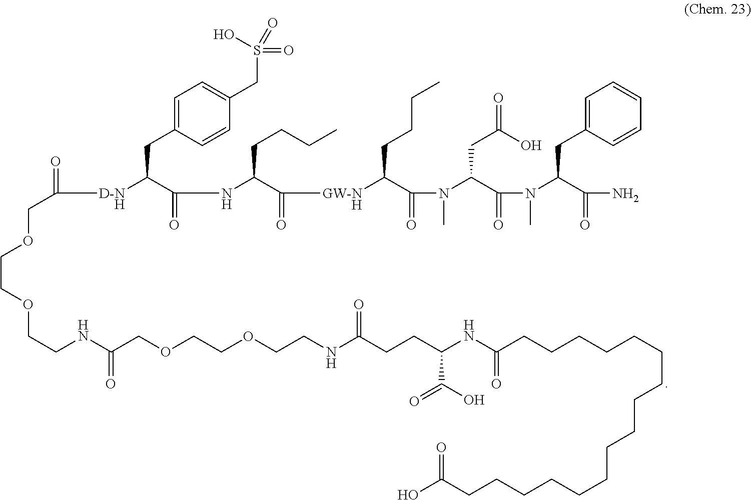

18. The pharmaceutical composition of claim 16, wherein the pharmaceutical composition comprises about 10.0 mM peptide derivative, about 8.0 mM phosphate, about 14 mg/mL propylene glycol, about 58 mM phenol, and has about pH 7.4; and wherein the peptide derivative is

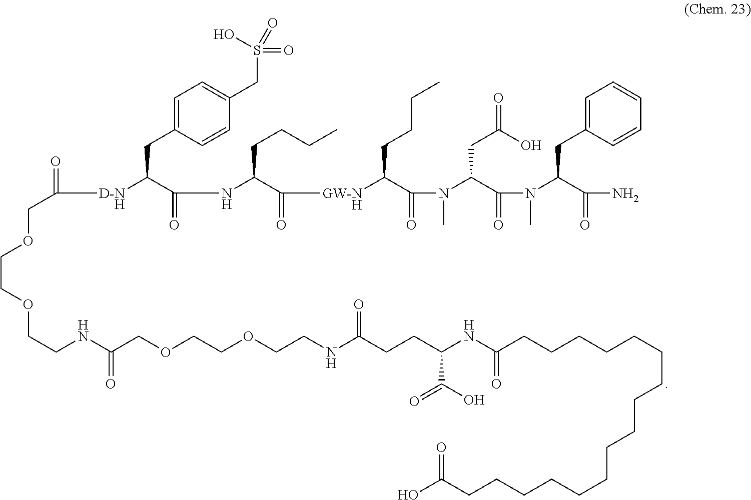

19. The pharmaceutical composition of claim 16, wherein the pharmaceutical composition comprises about 5.0 mM peptide derivative, about 8.0 mM Tris(hydroxymethyl)aminomethane, about 14 mg/mL propylene glycol, about 58 mM phenol, and has about pH 7.4; and wherein the peptide derivative is

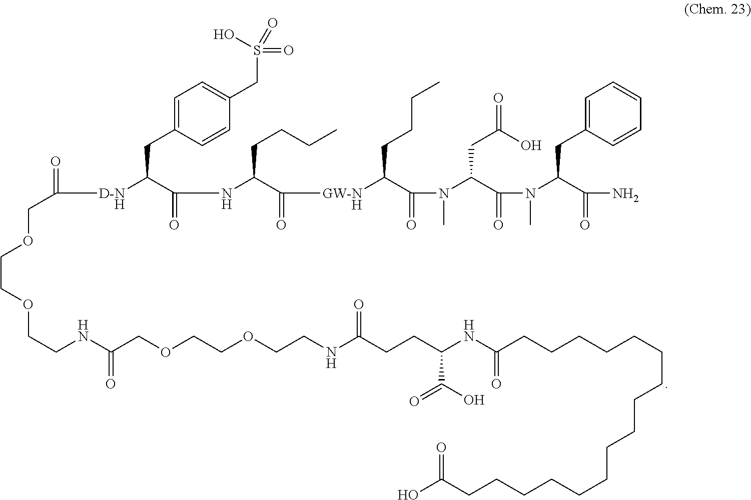

20. A method for treatment of overweight, comprising
administering a pharmaceutical composition comprising a peptide derivative and a pharmaceutically acceptable excipient to a subject in need thereof,
wherein the peptide derivative comprises the formula P-L-X1-X2-X3-Gly-Trp-X6-DMeAsp-X8-NH₂,
wherein P is HOOC—(CH₂)ₓ—CO—*, wherein x is an integer in the range of 12-18;
wherein L is absent or comprises at least one linker element selected from the group consisting of
*—NH—CH(COOH)—(CH₂)₂—CO—*,
*—NH—(CH₂)₂—[O—(CH₂)₂]ₖ—O—[CH₂]ₙ—CO—*,
*—NH—CH(CH₂OH)—CO—*,
*—NH—CH₂—CO—*,
and
*—NH—CH[(CH₂)₄—NH₂]—CO—*,
wherein k is an integer in the range of 1-11, and wherein n is an integer in the range of 1-5;
wherein X1 is absent or is selected group from the group consisting of Asp, DAsp, bAsp, DbAsp, Glu, and DGlu;
wherein X2 is selected from the group consisting of Phe(4-sulfomethyl) and sTyr;
wherein X3 is selected from the group consisting of Nle, Leu, Ala, Ile, Lys, Arg, Pro, Met, Phe, Ser, His, and Val;
wherein X6 is selected from the group consisting of Nle, Ile, Gln, Met, Met(O2), Leu, Val, Pro, Hpg, and Ala;
wherein X8 is selected from the group consisting of Phe, MePhe, 1Nal, Me1Nal, 2Nal, Me2Nal, Trp, and MeTrp; and
wherein the peptide derivative is C-terminally amidated by the NH₂ group;
or a pharmaceutically acceptable salt, amide, or ester thereof.

21. The method of claim 20, wherein the subject is suffering from obesity.

22. The method of claim 20, wherein L comprises at least one linker element selected from the group consisting of *—NH—CH(COOH)—(CH₂)₂—CO—*, *—NH—(CH₂)₂—[O—(CH₂)₂]ₖ—O—[CH₂]ₙ—CO—*, *—NH—CH(CH₂OH)—CO—*, *—NH—CH₂—CO—*, and *—NH—CH[(CH₂)₄—NH₂]—CO—*, wherein k is an integer in the range of 1-11, and wherein n is an integer in the range of 1-5.

23. The method of claim 20, wherein
X1 is absent or selected from the group consisting of Asp, DAsp, bAsp, DbAsp, Glu, and DGlu;
X2 is selected from the group consisting of Phe(4-sulfomethyl) and sTyr;
X3 is selected from the group consisting of Nle, Leu, Ala, Ile, Lys, Arg, Pro, Met, Phe, Ser, and His;
X6 is selected from the group consisting of Nle, Ile, Gln, Met, Met(O2), Leu, Val, Pro, and Ala; and
X8 is selected from the group consisting of Phe, MePhe, 1Nal, Me1Nal, 2Nal, and Trp.

24. The method of claim 20, wherein the peptide derivative is selected from the group consisting of: 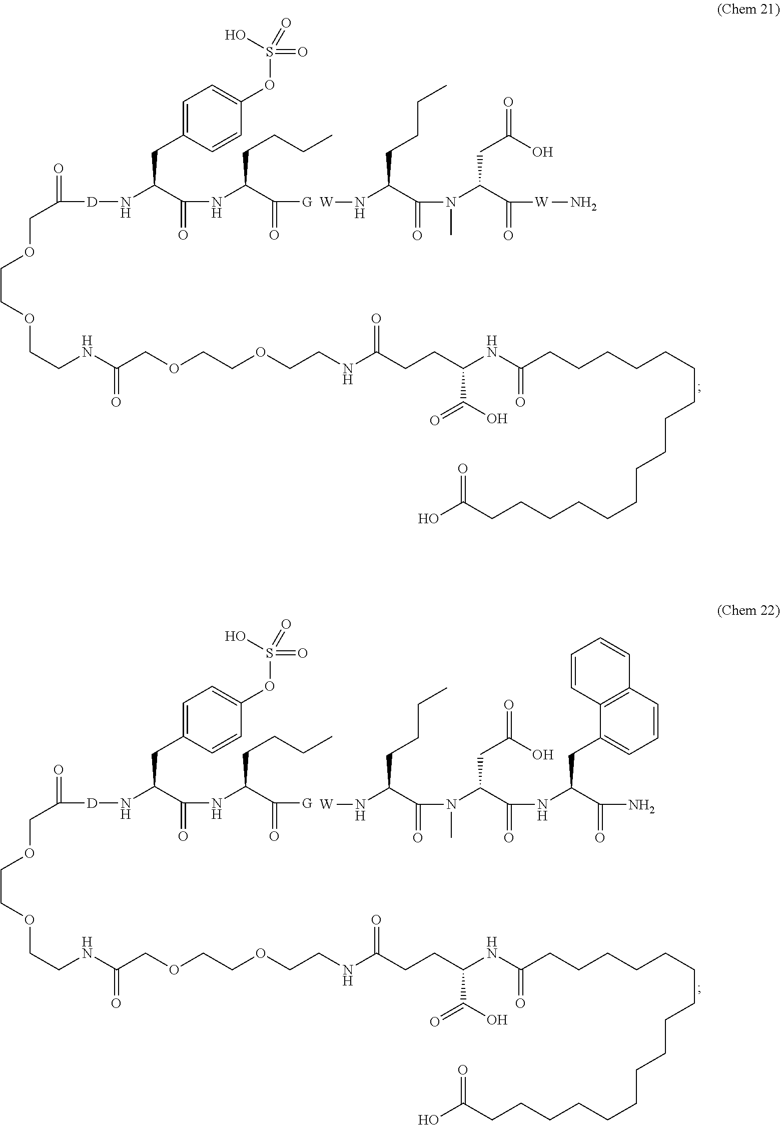 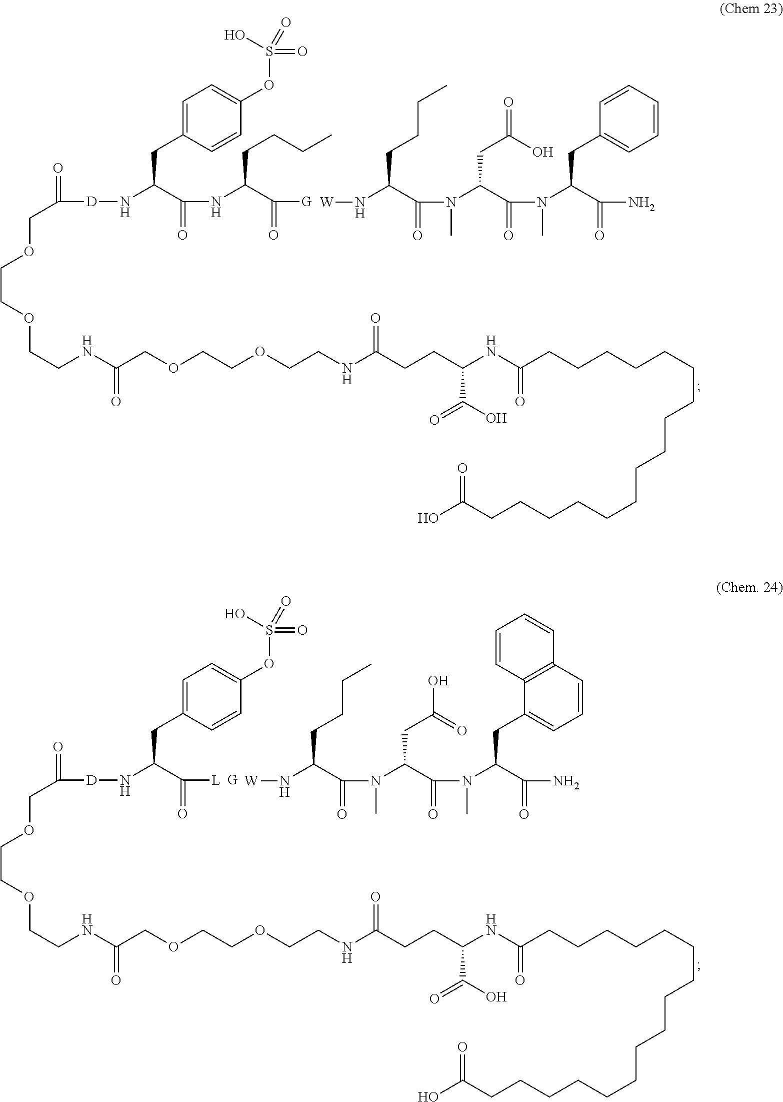 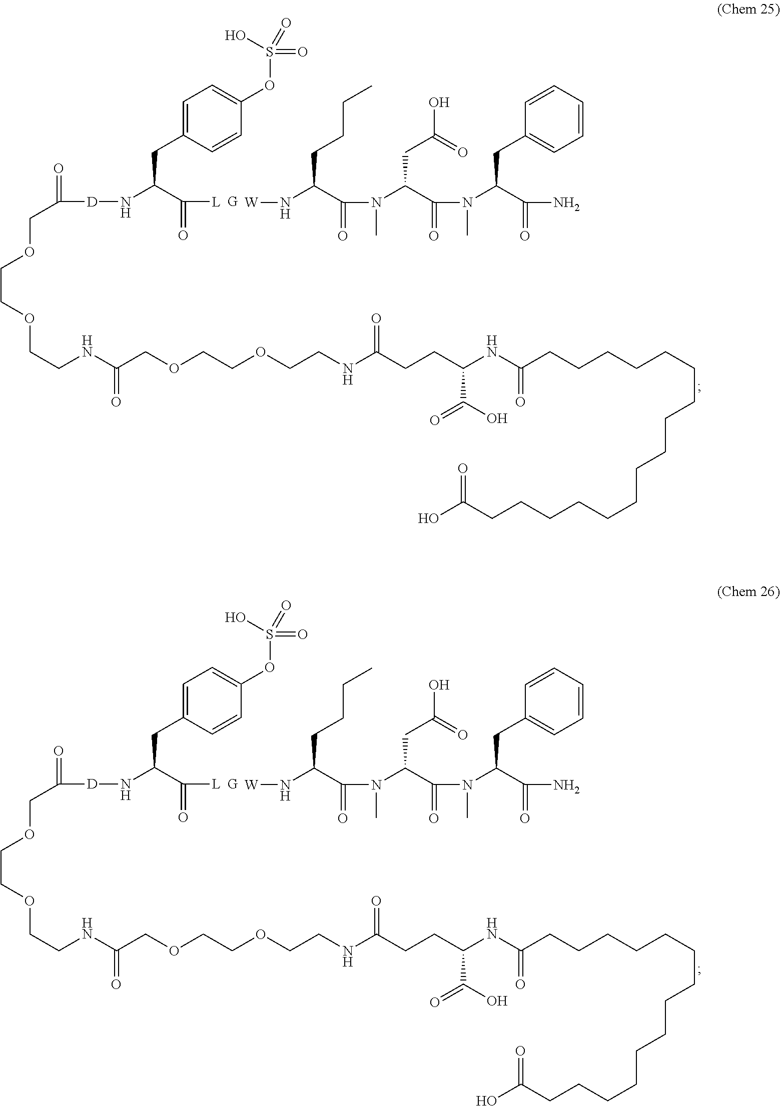 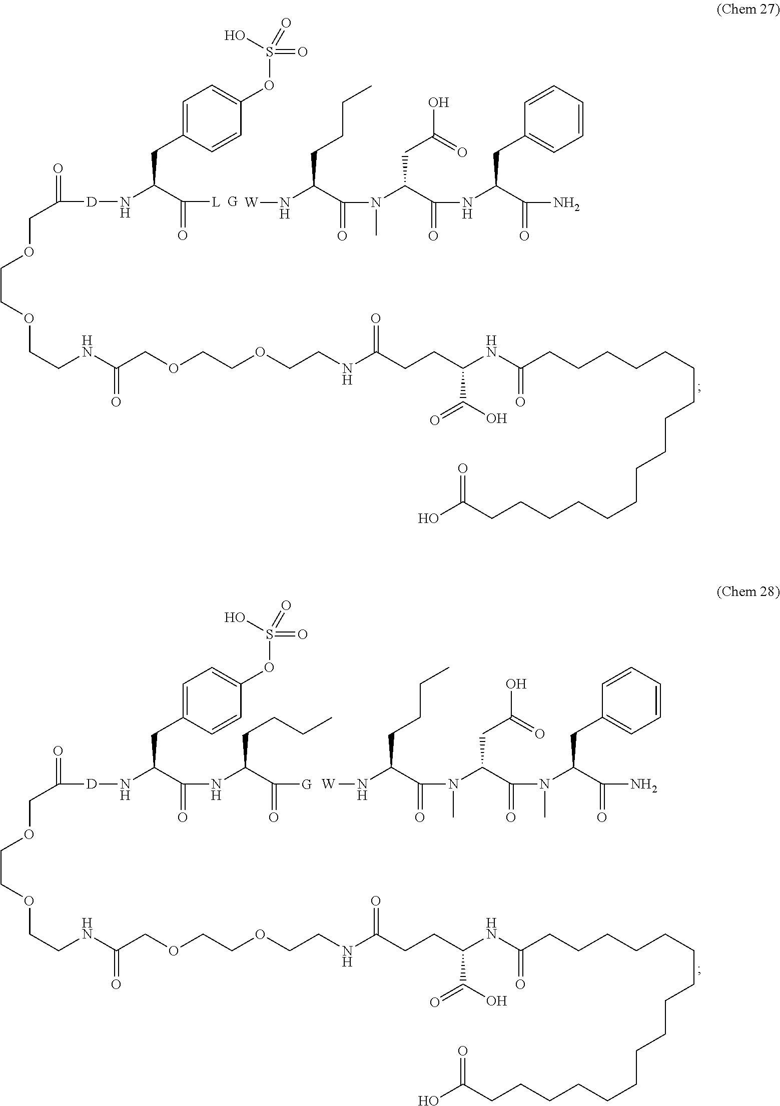 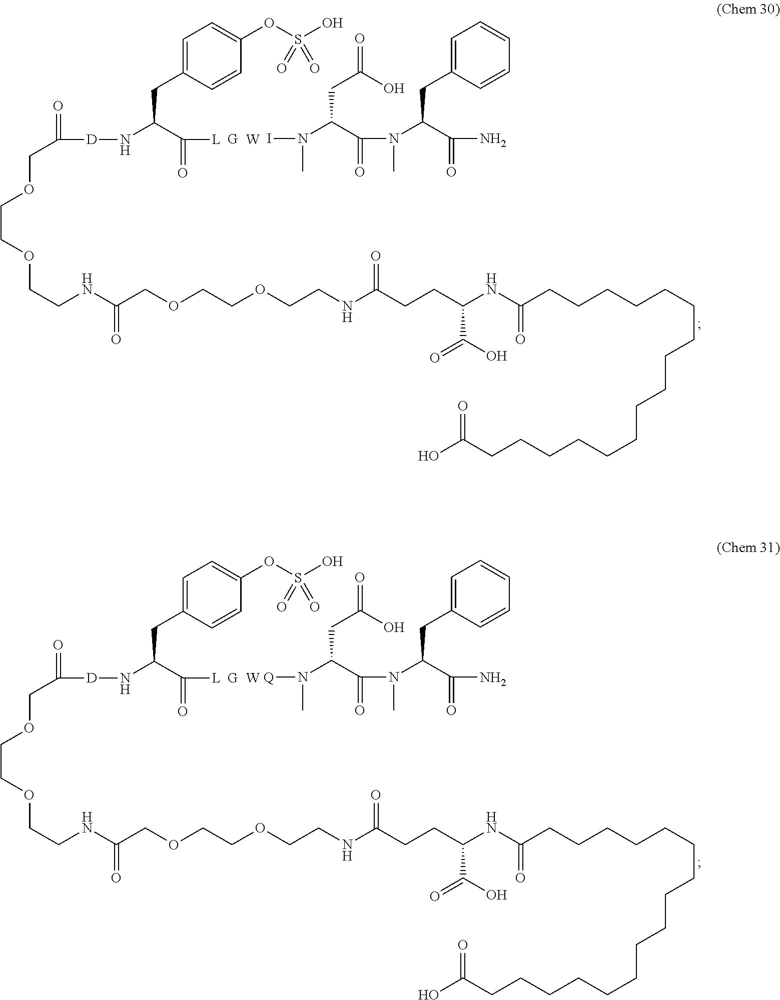 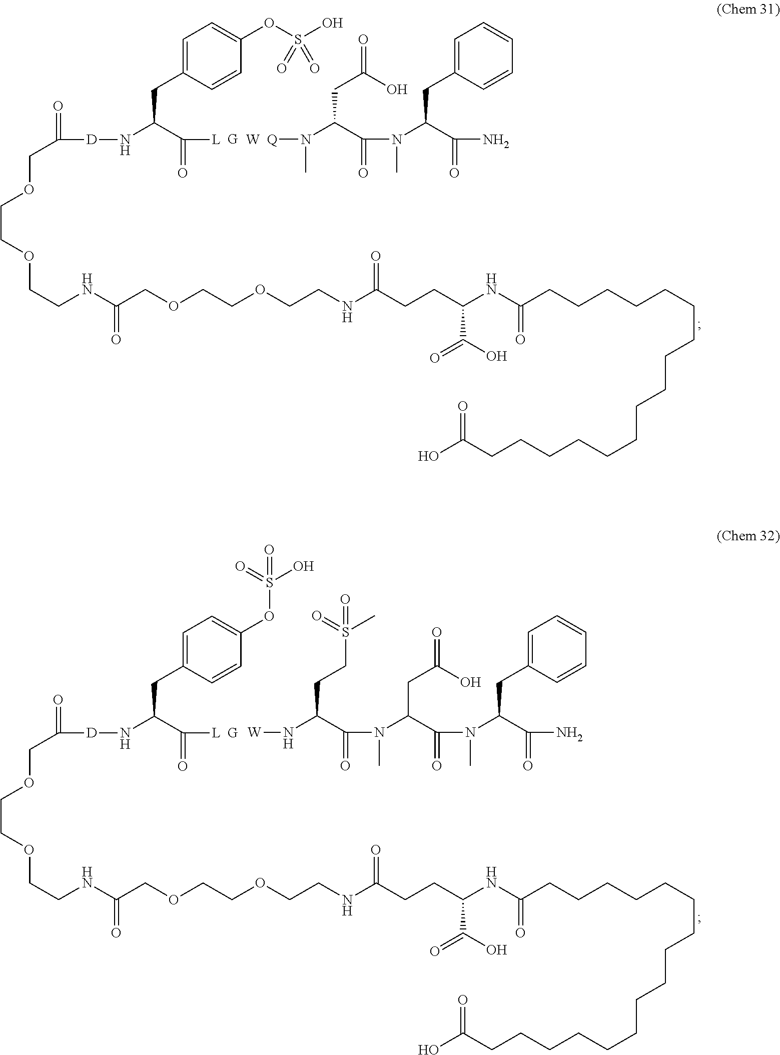 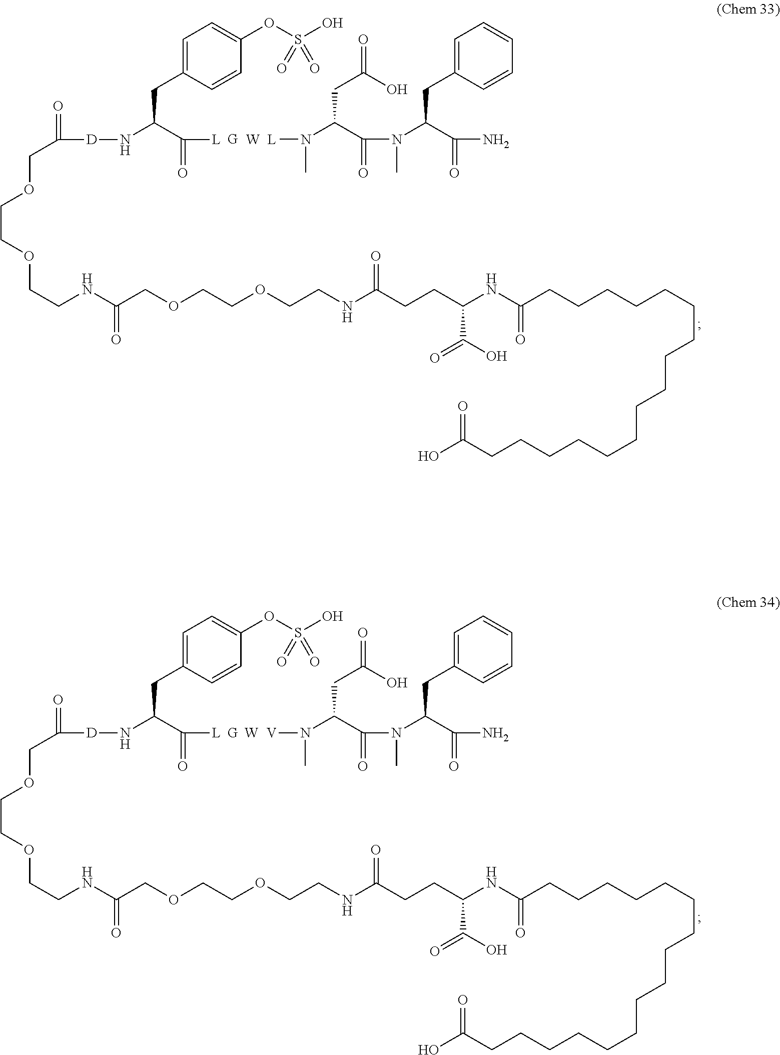 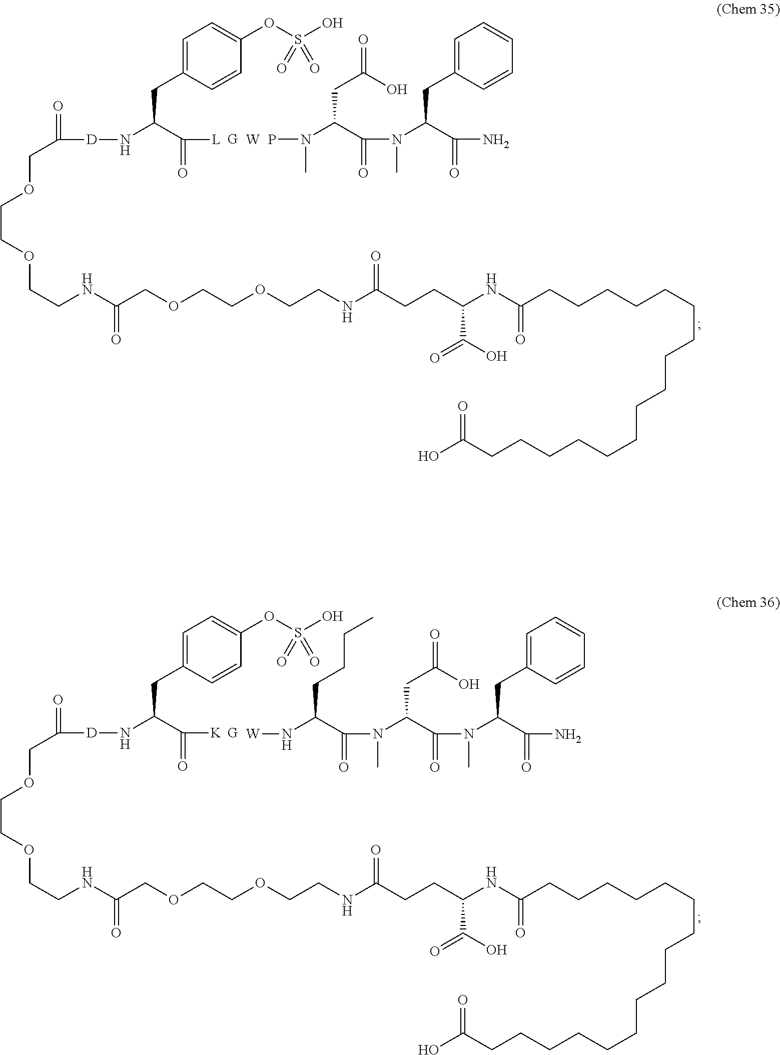 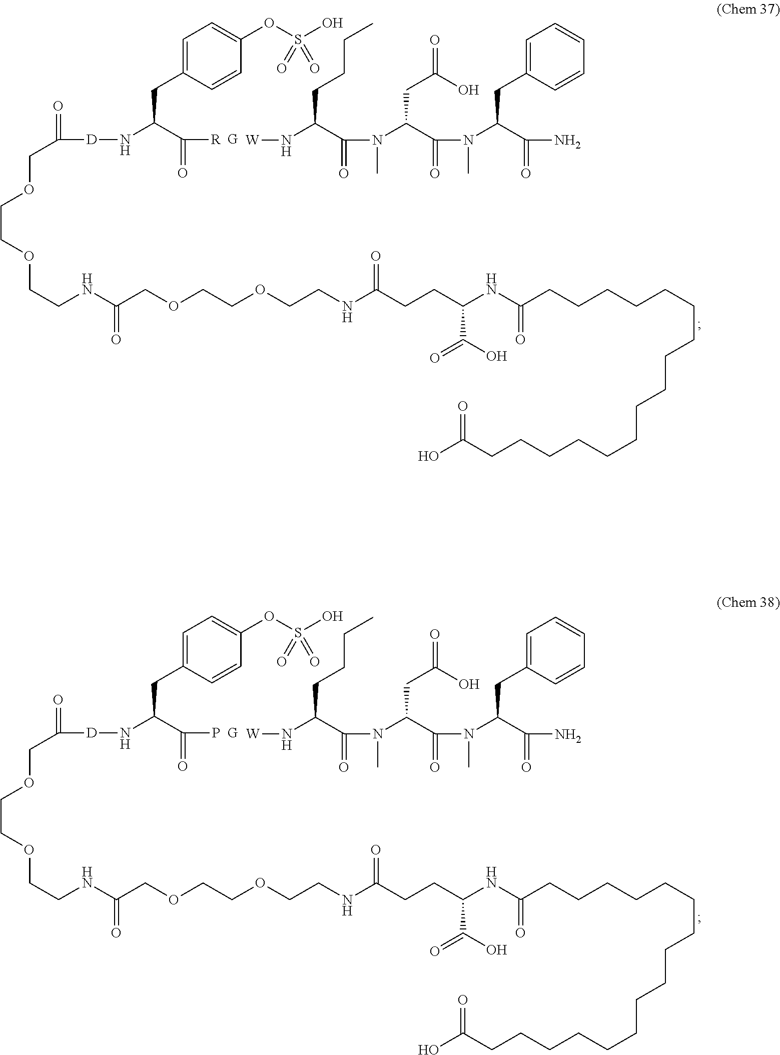 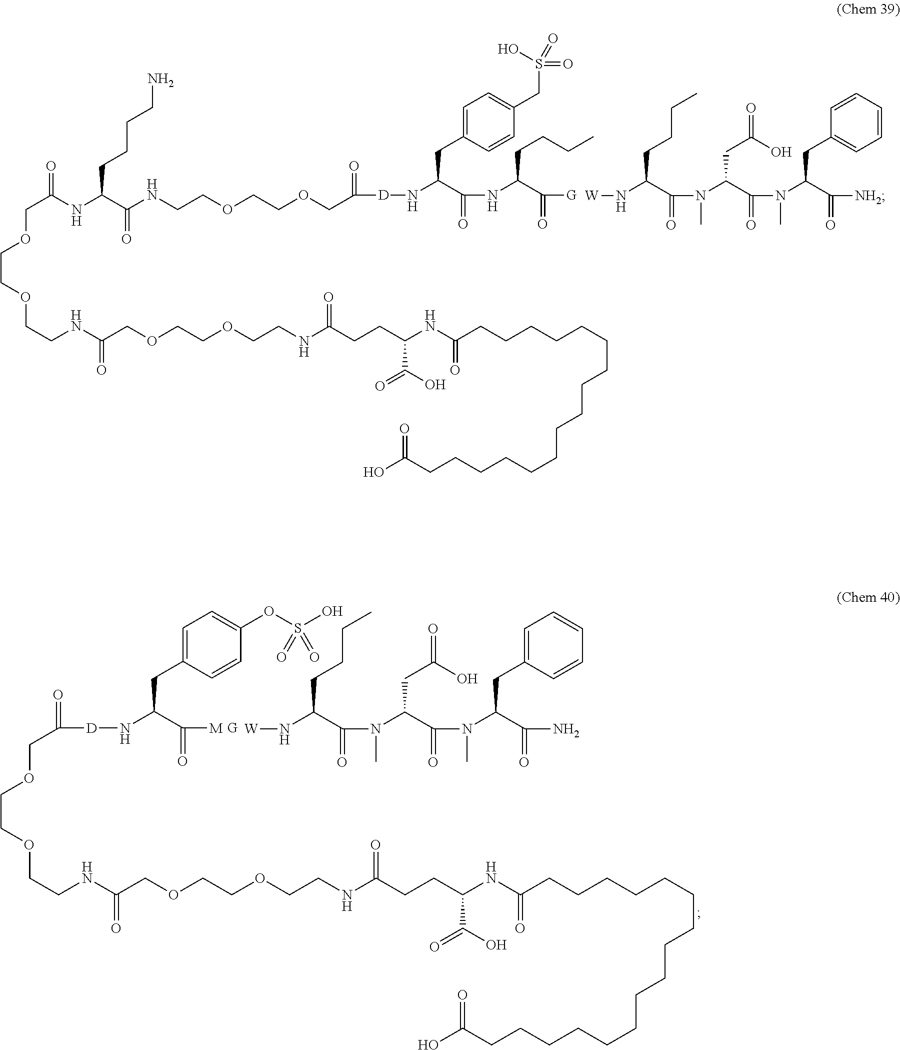 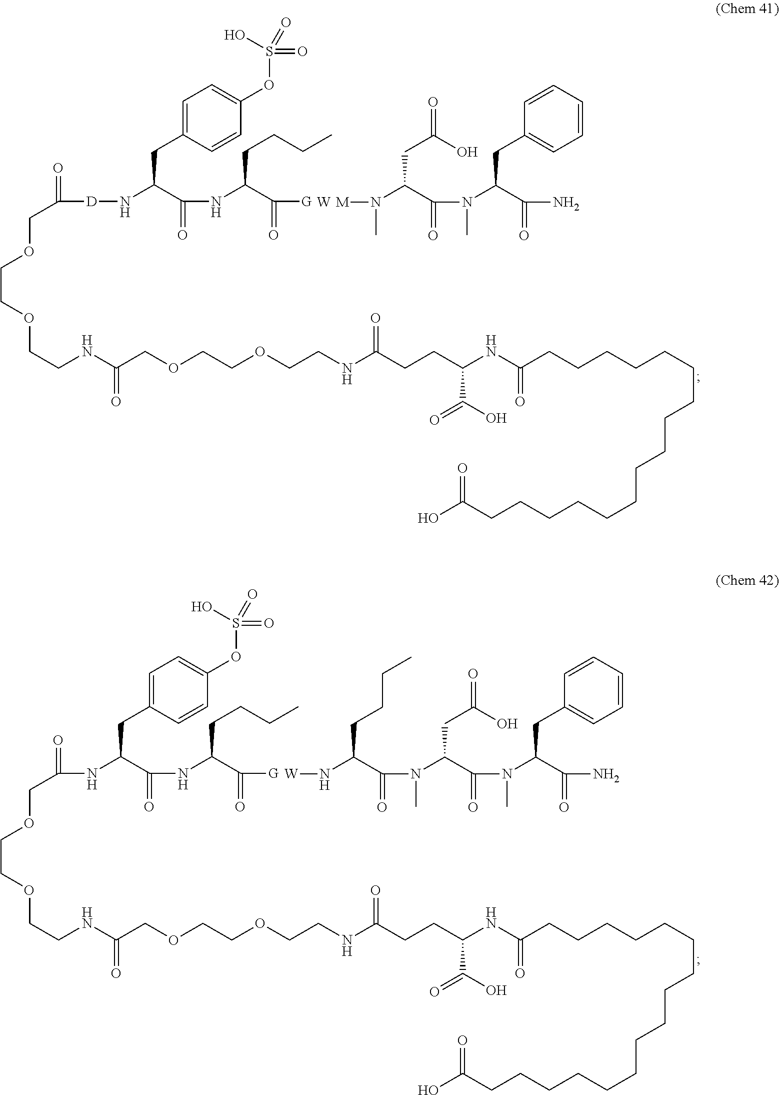 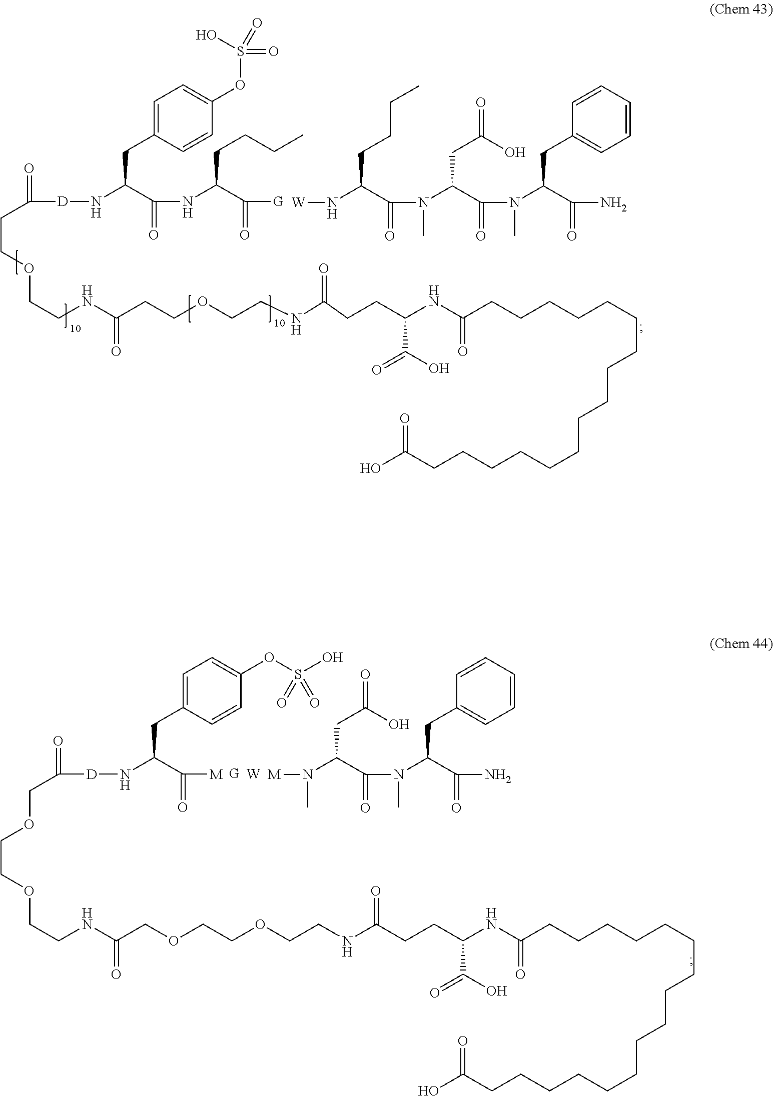 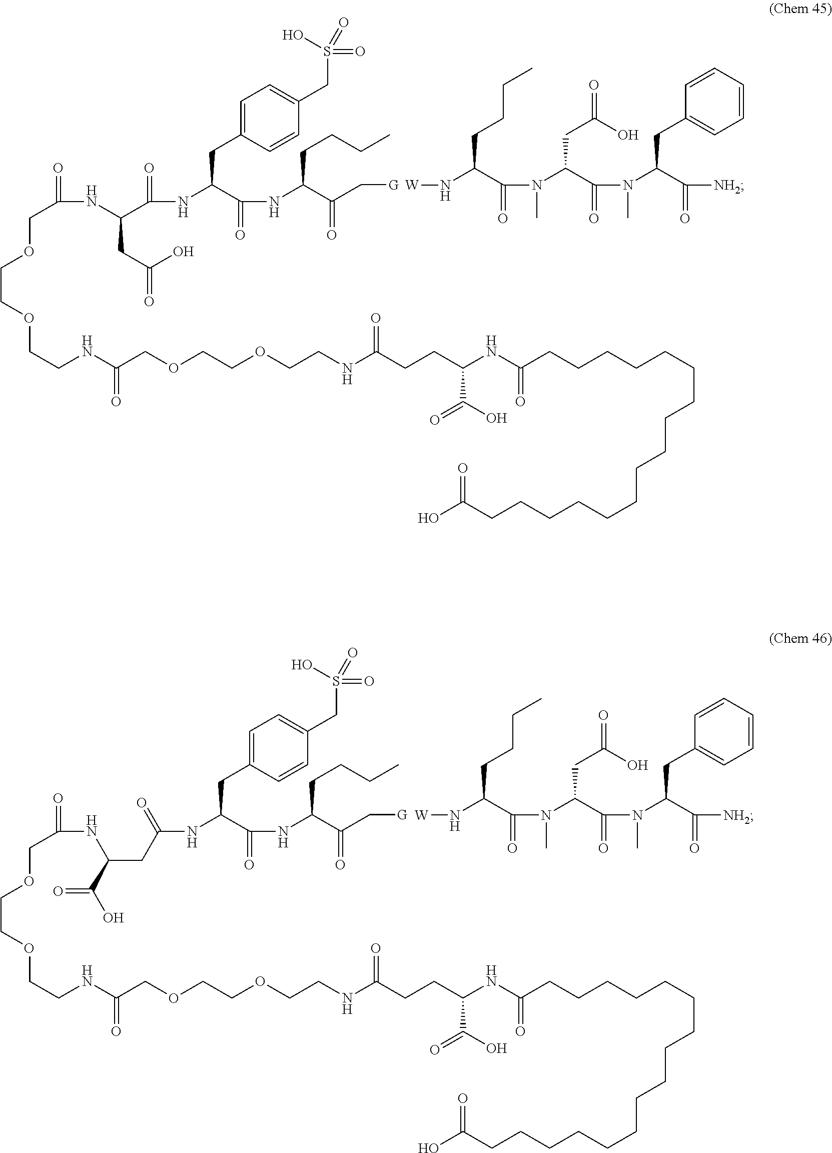 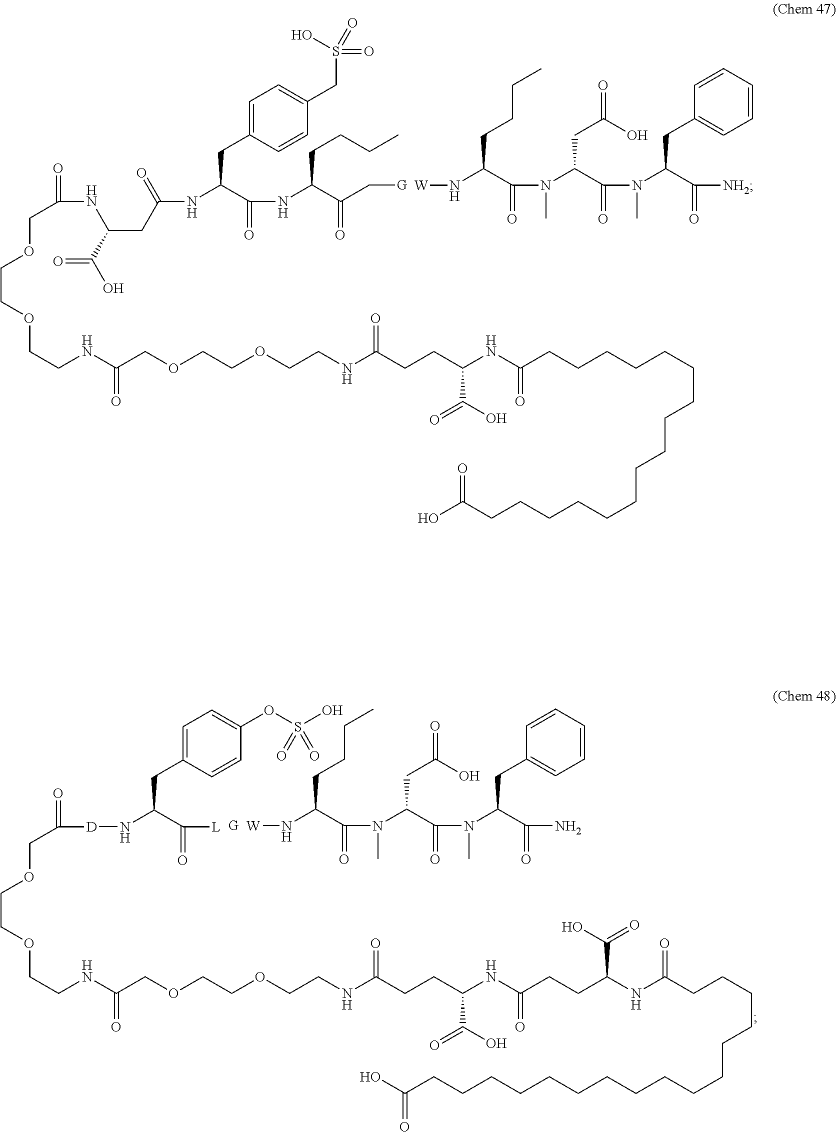 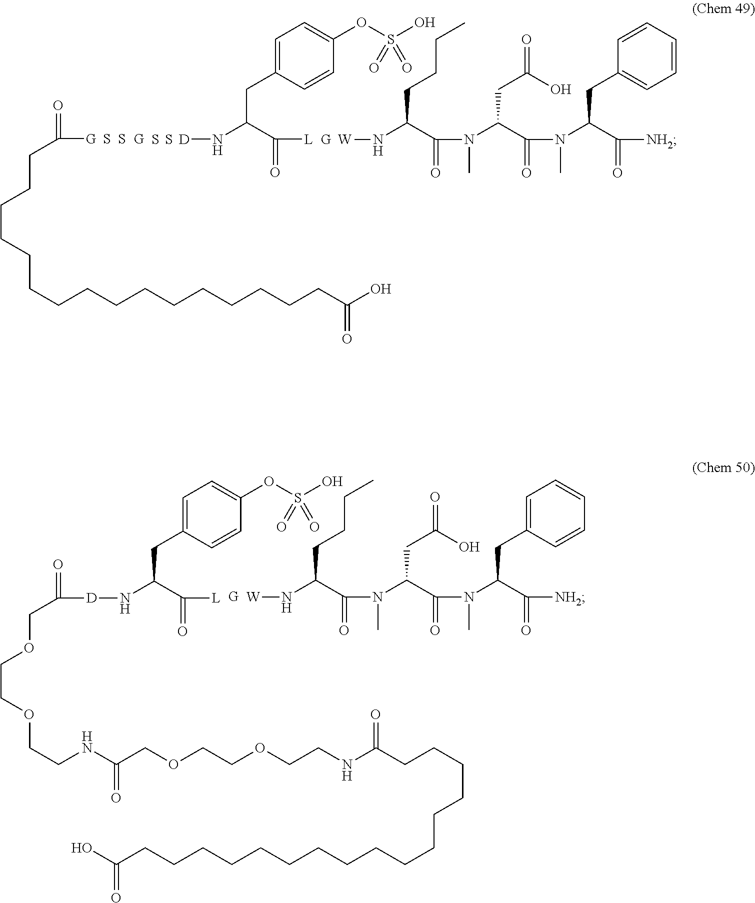 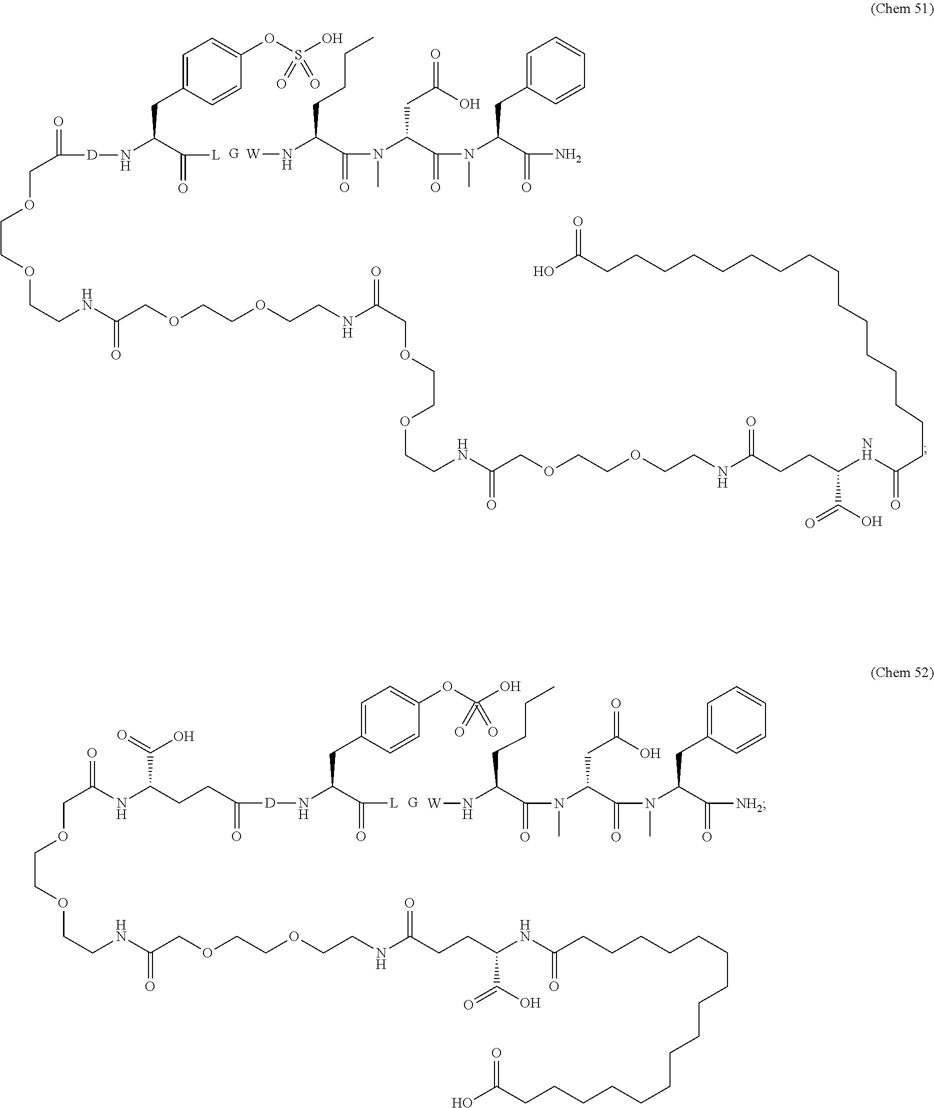 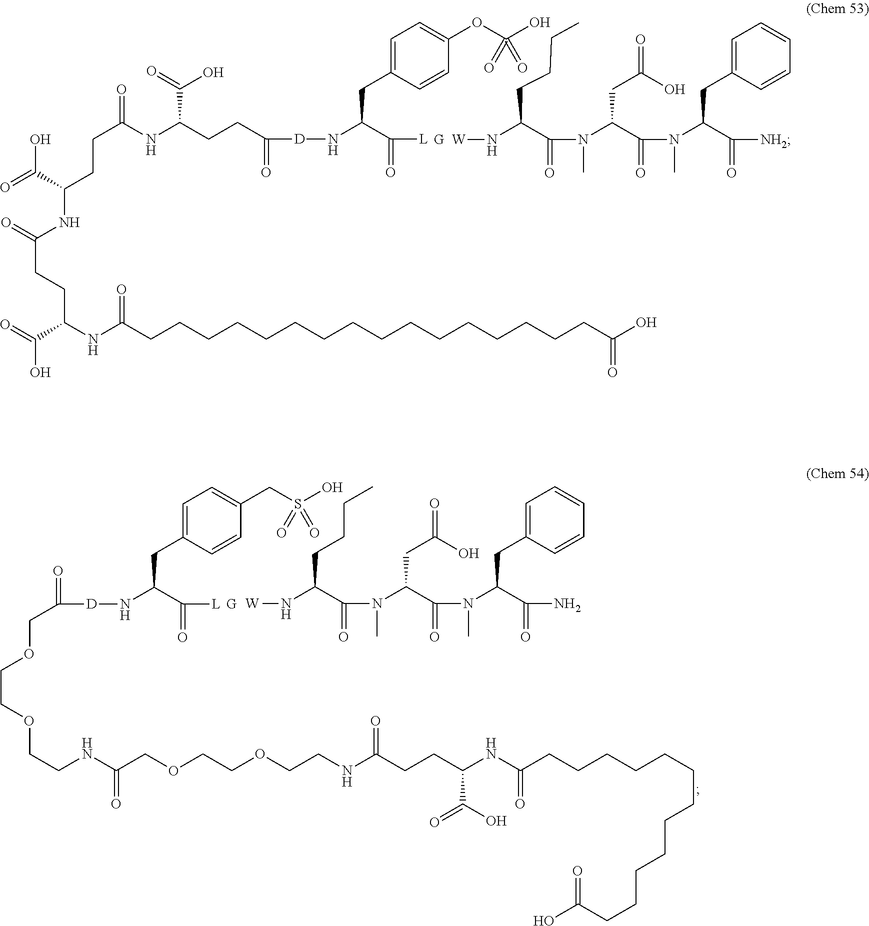 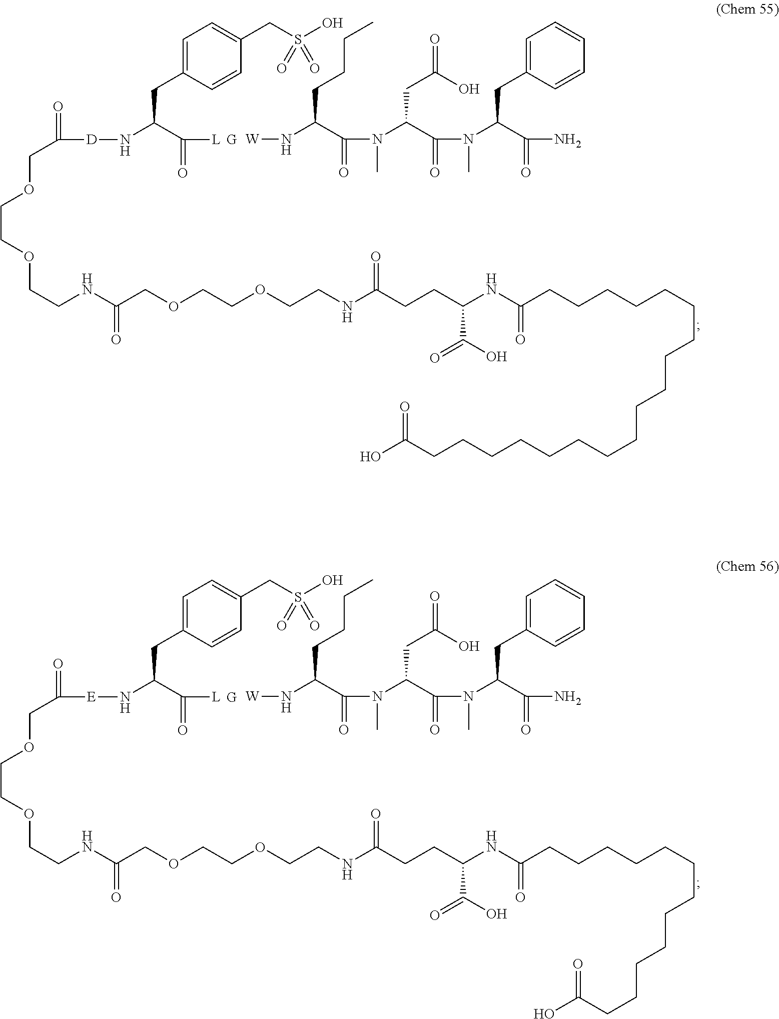 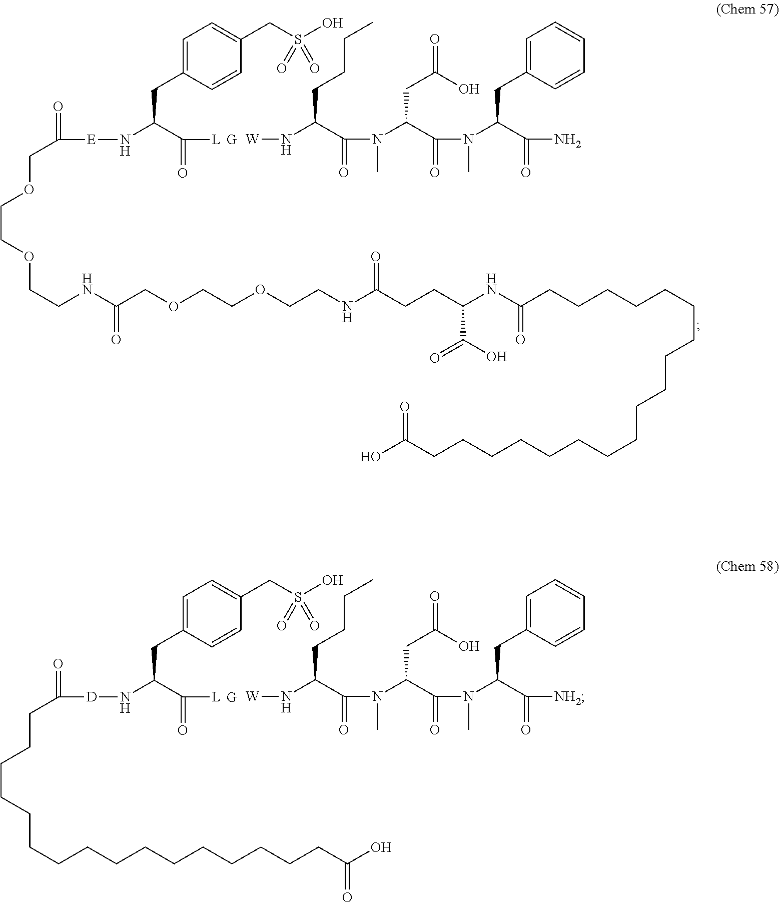 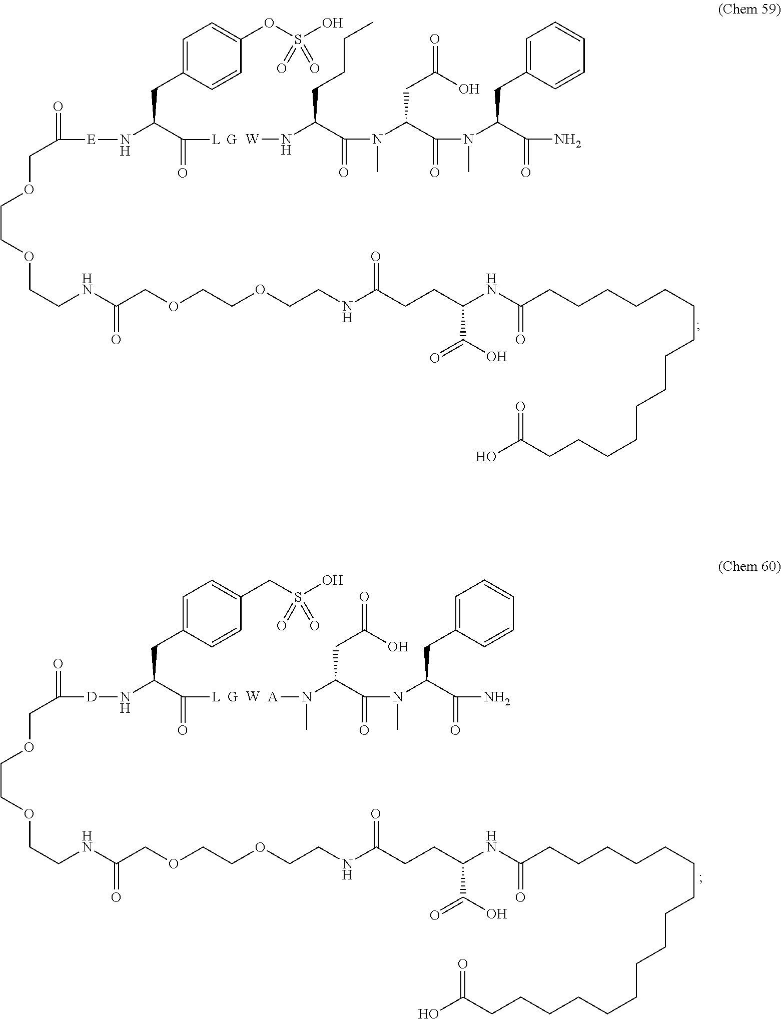 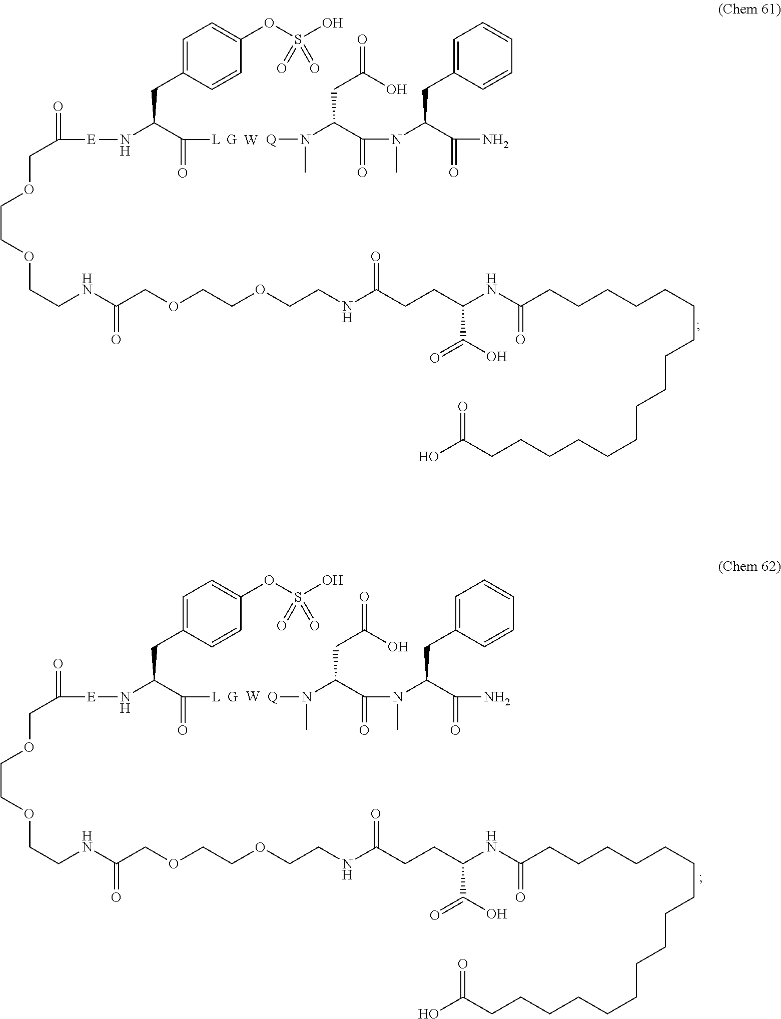 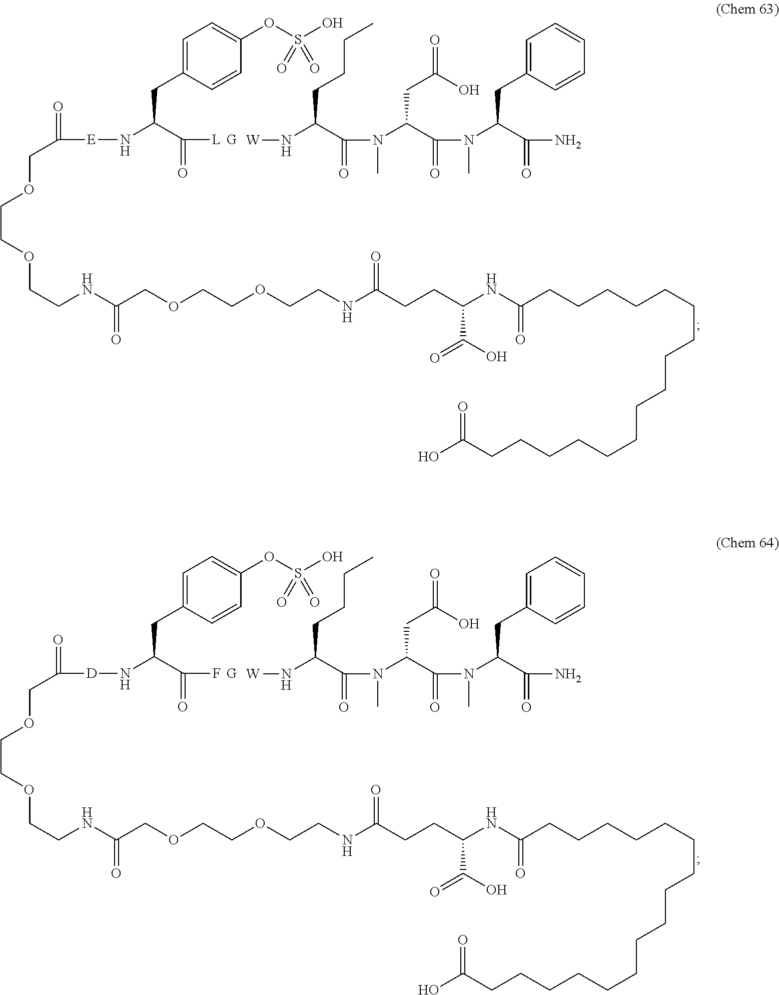 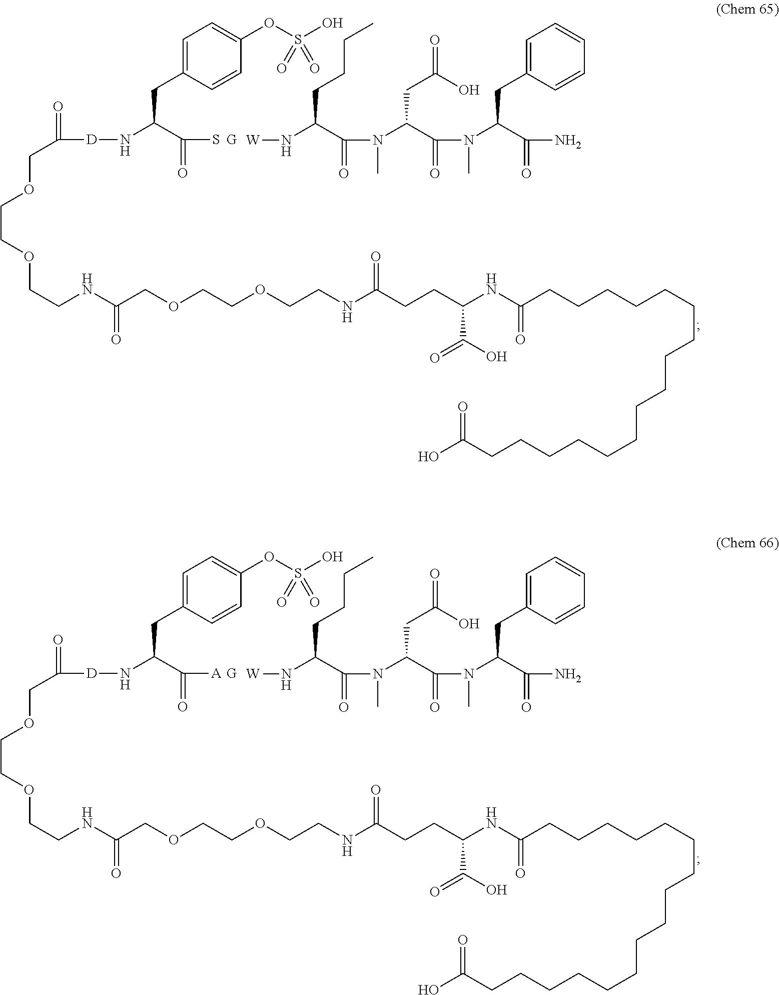 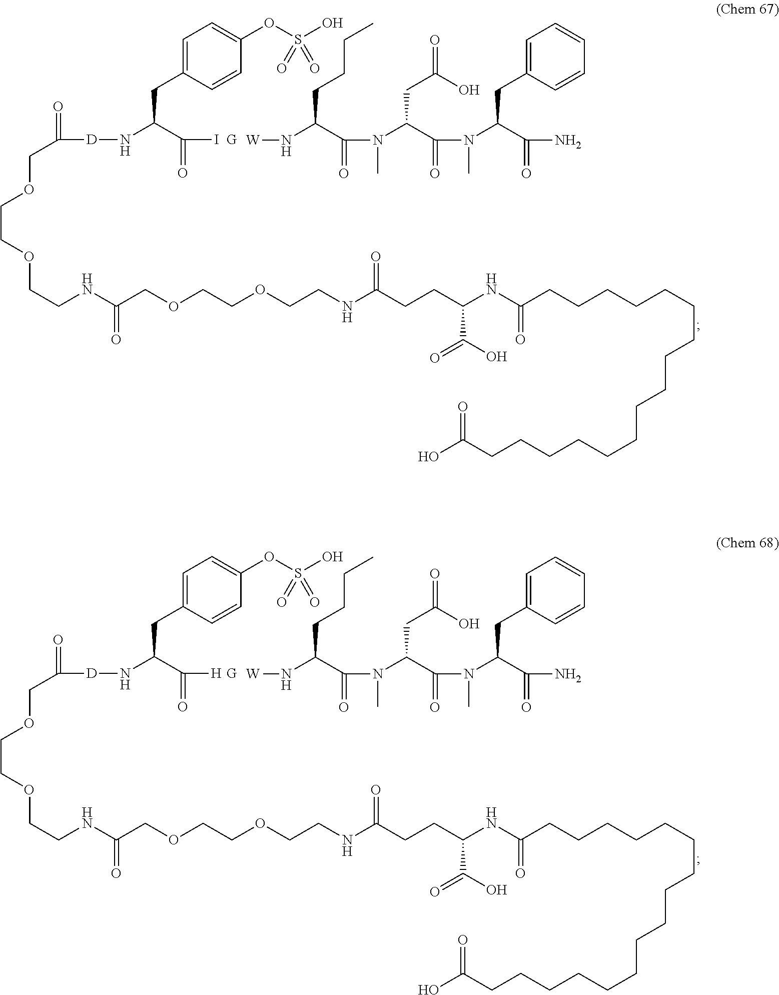 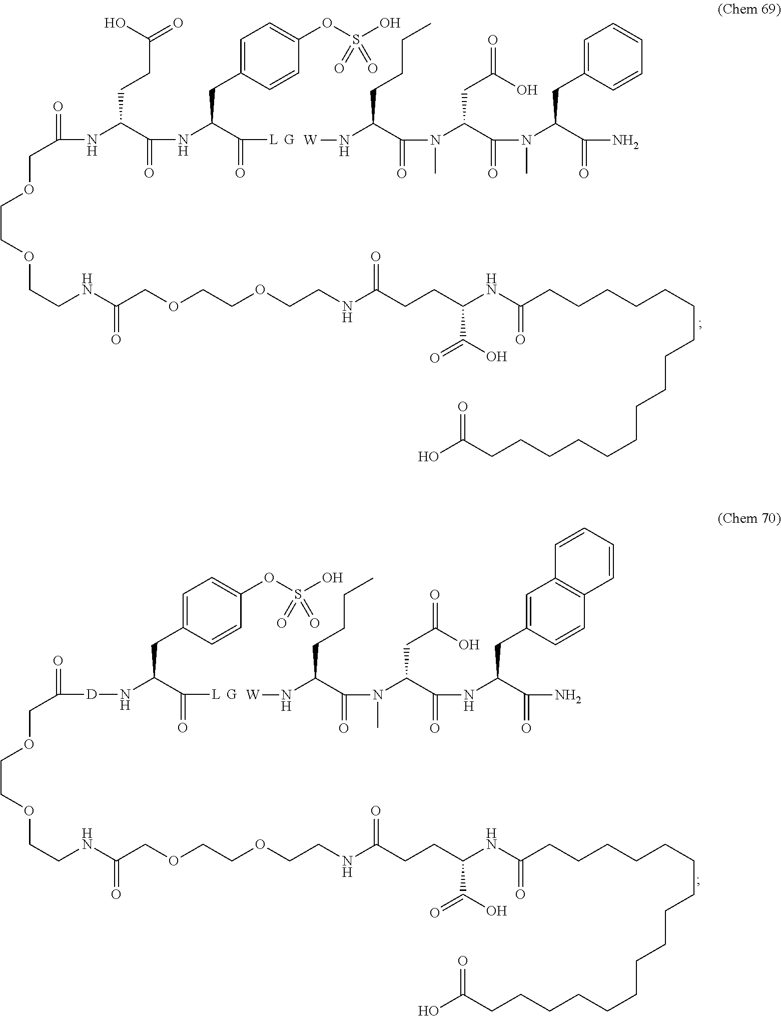 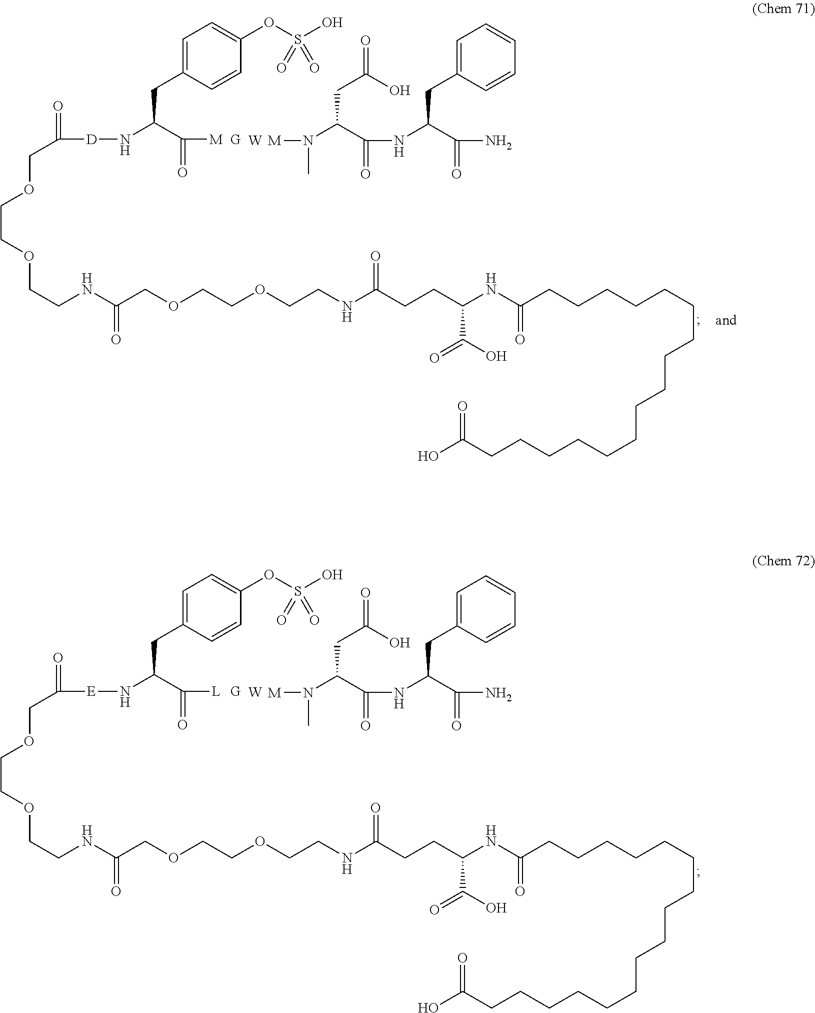
or a pharmaceutically acceptable salt, amide, or ester thereof.

25. The method of claim 24, wherein the peptide derivative is

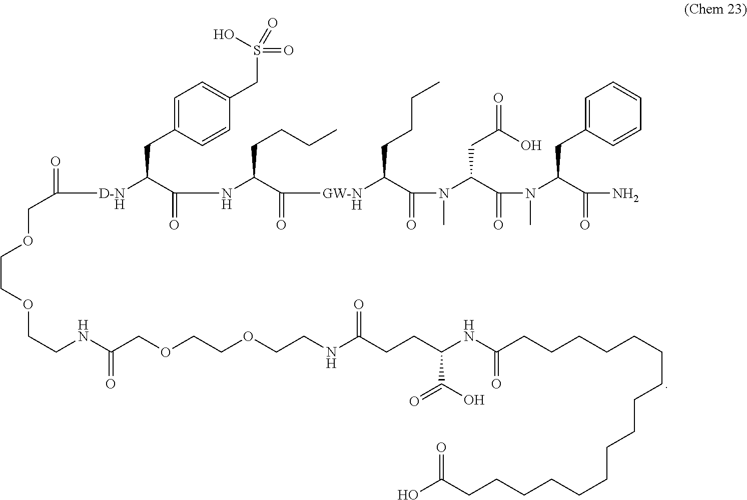

26. The method of claim 25, wherein the subject is suffering from obesity.
